(19) United States
(12) Patent Application Publication
(10) Pub. No.: US 2009/0124624 A1
(43) Pub. Date: May 14, 2009

(54) SUBSTITUTED 1-AMINOPHTHALAZINE DERIVATIVES, PREPARATION THEREOF AND THERAPEUTIC APPLICATION THEREOF

(75) Inventors: Jean Michel AUGEREAU, Toulouse (FR); Gilles COURTEMANCHE, Saubens (FR); Michel GESLIN, Villeneuve Tolosane (FR); Laurence SERVA, Frouzins (FR)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS, Paris (FR)

(21) Appl. No.: 12/098,635

(22) Filed: Apr. 7, 2008

Related U.S. Application Data
(63) Continuation of application No. PCT/FR2006/002271, filed on Oct. 10, 2006.

(30) Foreign Application Priority Data
Oct. 12, 2005 (FR) 0510408

(51) Int. Cl. A61K 31/502; C07D 237/30; A61P 3/04

(52) U.S. Cl. 514/248; 544/237

(57) ABSTRACT

The invention concerns 1-amino-phthalazine derivatives of general formula (I):

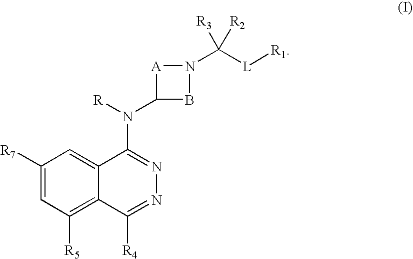

Wherein A, B, L, R, R₁, R₂, R₃, R₄, R₅ and R₇ are as defined herein. The invention also concerns the preparation of said compounds and their therapeutic use.

This application is a continuation of International application No. PCT/FR2006/002,271, filed Oct. 10, 2006, which is incorporated herein by reference in its entirety; which claims the benefit of priority of French Patent Application No. 05/10,408, filed Oct. 12, 2005.

The present invention relates to 1-aminophthalazine derivatives, to their preparation and to their therapeutic application.

The search for antagonists of the MCH (Melatonin-Concentrating Hormone) receptor 1, the MCH₁ receptor, has aroused the interest of many pharmaceutical companies. A certain number of patent applications have been filed, among which mention may be made of WO 01/21577 (Takeda), WO 02/06245 (Synaptic) and WO 03/106452 (Millennium). A certain number of publications have appeared, among which is Ma V. V. et al. (Amgen) 224th Nat. Meeting ACS Boston. Poster MEDI 343 (Aug. 21, 2002).

In the last ten years, it has been demonstrated that many neuropeptides are involved in the central regulations governing eating behavior and also the energy balance (Inui et al., TINS 1999; 22(2): 62-67). MCH is among these neuropeptides.

Two MCH receptors have recently been cloned: the MCH₁ receptor, previously known as the SLC-1 or GPR24 receptor (Chambers et al., Nature 1999; 400: 261-265), and the MCH₂ receptor, previously known as SLT (Mori et al., Biochem. Biophys. Res. Commun. 2001; 283: 1013-1018).

There is thus real interest in finding novel compounds for modulating the activity of the MCH₁ receptor.

WO 2005/103033 describes aminophthalazine derivatives capable of modulating the activity of the MCH₁ receptor.

It has now been found that 1-aminophthalazine-based compounds show high affinity and selectivity towards the MCH₁ receptor. These compounds have an in vitro and in vivo activity towards MCH₁ receptors.

One subject of the present invention is compounds corresponding to the formula general (I) below:

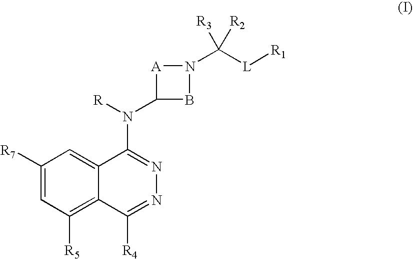

in which:
A represents a C₁₋₄-alkylene group optionally substituted with one or more groups R₉, which may be identical or different;
B represents a C₁₋₄-alkylene group optionally substituted with one or more groups R₁₀, which may be identical or different;
R₉ and R₁₀ each represent, independently of each other, a hydrogen atom or a C₁₋₅-alkyl group,
   or alternatively R₉ and R₁₀ together form a single bond or a C₁₋₄-alkylene group;
L represents a single bond or a C₁₋₂-alkylene, —CH═CH— or —C≡C— group; the C₁₋₂-alkylene and —CH═CH— groups being optionally substituted with one or more C₁₋₂-alkyl substituents;
   or alternatively L represents a cycloprop-1,2-diyl group;
L represents a hydrogen atom or a C₁₋₅-alkyl, C₁₋₃-fluoroalkyl, C₃₋₆-cycloalkyl, —C(O)C₁₋₃-alkyl, C₁₋₃-alkylene-C₃₋₆-cycloalkyl, —CH₂—C≡CH, C₂₋₃-alkylene-NRₐR_{b} or C₁₋₃-alkylene-X—C₁₋₃-alkyl group in which X represents O or SO₂;
R₁ represents an aryl or a heteroaryl; the aryl and heteroaryl groups being optionally substituted with one or more radicals Z, which may be identical or different;
R₂ and R₃ represent, independently of each other, a hydrogen atom or a C₁₋₃-alkyl or C₁₋₃-fluoroalkyl group,
   or alternatively R₂ and R₃ form, together with the carbon atom that bears them, a cycloprop-1,1-diyl group;
R₄ represents:
   a hydrogen atom or a C₁₋₅-alkyl, C₁₋₃-fluoroalkyl, C₃₋₆-cycloalkyl, C₁₋₃-alkylene-C₃₋₆-cycloalkyl, C₁₋₃-alkylene-O—C₁₋₃-alkyl or C₁₋₃-alkylene-O—C₄₋₆-cycloalkyl group,
   a C₁₋₃-alkylene-(OH) or C₁₋₃-alkylene-X—C₁₋₃-alkyl group in which X represents S, SO or SO₂,
   a heterocyclic group; the said heterocyclic group being optionally substituted with a C₁₋₃-alkyl, —C(O)C₁₋₃-alkyl, —C(O)C₁₋₃-fluoroalkyl, C₁₋₃-alkylene-C₃₋₄-cycloalkyl, C₁₋₃-alkylene-aryl or C₁₋₃-alkylene-heteroaryl group; the C₁₋₃-alkylene-aryl and C₁₋₃-alkylene-heteroaryl groups being optionally substituted with one or more radicals Z, which may be identical or different,
   or alternatively R₄ represents a C₁₋₃-alkylene-NRₐR_{b}, aryl, C₁₋₃-alkylene-aryl, —O-aryl, C₁₋₃-alkylene-O-aryl, C₁₋₃-alkylene-O—C₁₋₃-alkylene-aryl, heteroaryl or C₁₋₃-alkylene-heteroaryl group; the aryl, C₁₋₃-alkylene-aryl, —O-aryl, C₁₋₃-alkylene-O-aryl, C₁₋₃-alkylene-O—C₁₋₃-alkylene-aryl, heteroaryl and C₁₋₃-alkylene-heteroaryl groups being optionally substituted with one or more radicals Z, which may be identical or different;
R₅ represents a hydrogen or halogen atom or a C₁₋₅-alkyl, C₁₋₃-fluoroalkyl, C₁₋₅-alkoxy, C₁₋₃-fluoroalkoxy, C₁₋₃-alkylene-(OH), —CN or —X—C₁₋₃-alkyl group in which X represents S, SO or SO₂,
   or alternatively R₅ represents a group —NRₐR_{b}, C₁₋₃-alkylene-NRₐR_{b}, aryl, C₁₋₃-alkylene-aryl, —O-aryl or heteroaryl group; the aryl, C₁₋₃-alkylene-aryl, —O-aryl and heteroaryl groups being optionally substituted with one or more radicals Z, which may be identical or different;
R₇ represents a hydrogen or halogen atom or a C₁₋₅-alkyl, C₁₋₃-fluoroalkyl, C₁₋₅-alkoxy, —COOH, —C(O)OC₁₋₃-alkyl, C₁₋₃-fluoroalkoxy, C₁₋₃-alkylene-(OH), —CN or —X—C₁₋₃-alkyl group in which X represents S, SO or SO₂,
   or alternatively R₇ represents a group —NRₐR_{b}, C₁₋₃-alkylene-NRₐR_{b}, —C(O)—NRₐR_{b},
      —C(O)—C₁₋₃-alkyl, aryl, —O-aryl or heteroaryl group; the aryl, —O-aryl and heteroaryl groups being optionally substituted with one or more radicals Z, which may be identical or different;
Z represents a halogen atom or a C₁₋₅-alkyl, C₁₋₃-fluoroalkyl, C₃₋₆-cycloalkyl, C₁₋₃-alkylene-C₃₋₆-cycloalkyl, C₁₋₅-alkoxy, C₁₋₃-fluoroalkoxy, C₁₋₃-alkylene-O—C₁₋₃-alkyl, C₁₋₃-alkylene-(OH), NO₂, —CN, —SO₂NRaRb, —X—C₁₋₃-alkyl or C₁₋₃-alkylene-X—C₁₋₃-alkyl group in which X represents S, SO or SO₂,
   or alternatively Z represents a phenyl optionally substituted with a halogen atom or a C₁₋₅-alkyl, C₁₋₅-alkoxy or C₁₋₃-fluoroalkyl group,
   or alternatively Z represents an alkynyl radical of the type —C═C—R_{c}, in which R_{c} represents a hydrogen atom or a C₁₋₃-alkyl, C₁₋₆-alkylene-O—R_{d} or C₁₋₆-alkylene-NRₐR_{b} group,
   or alternatively Z represents a —C₄₋₆-alkylene-OR_{d} group,
   or alternatively Z represents a tetrazole group substituted with a C₁₋₃-alkyl group,
   or alternatively Z represents a group —NRₐR_{b}, C₁₋₃-alkylene-NRₐR_{b}, —C(O)—NRₐR_{b}, —C(O)—C₁₋₃-alkyl, —CO₂—C₁₋₄-alkyl or —C(O)—C₃₋₆-cycloalkyl group,
   or alternatively Z represents a —C₄₋₆-alkylene-NRₐR_{b} group,
   or alternatively Z represents an oxo radical,
   or alternatively Z represents an —O—C₁₋₅-alkylene-NRₐR_{b} group,
   or alternatively Z represents an —O—C₀₋₃-alkylene-heterocyclic group, optionally substituted with one or more C₁₋₃-alkyl, oxo or —C(O)—C₁₋₃-alkyl groups,
   or alternatively Z represents an —O—C₁₋₅-alkylene-O—R_{d} group,
   or alternatively Z represents an —O—C₀₋₃-alkylene-C₅₋₇-cycloalkyl group optionally substituted with an —O—R_{d},
   or alternatively Z represents a group —NRₑ—C₀₋₃-alkylene-heterocycle optionally substituted with one or more C₁₋₃-alkyl, oxo or —C(O)—C₁₋₃-alkyl groups,
   or alternatively Z represents a group —NRₑ—C₂₋₅-alkylene-O—R_{d},
   or alternatively Z represents a —O—C₁₋₃-alkylene-heteroaryl group optionally substituted with one or more C₁₋₃-alkyl groups,
   or alternatively Z represents a group —CONH—C₁₋₅-alkylene-NRₐR_{b},
   or alternatively Z represents a group —CONH—C₁₋₅-alkylene-O—R_{d},
   or alternatively Z represents an —O—C₁₋₃-alkylene-C(O)—NRₐR_{b},
   or alternatively Z represents a fused or spirane bicyclic diamino heterocycle,
   or alternatively two adjacent radicals Z together form a C₁₋₃-alkylenedioxy group;
Rₐ and R_{b} represent, independently of each other, a hydrogen atom or a C₁₋₃-alkyl or —C(O)—C₁₋₃-alkyl group,
   or alternatively Rₐ and R_{b} form, together with the nitrogen atom to which they are attached, a heterocycle optionally substituted with one or more C₁₋₃-alkyl, oxo, —NRₐR_{b}, hydroxyl, C₁₋₃-alkoxy, C₁₋₃-alkylene-(OH) or —C(O)—C₁₋₃-alkyl groups;
R_{d} represents a hydrogen atom or a C₁₋₃-alkyl group;
Rₑ represents a hydrogen atom or a C₁₋₃-alkyl group;
   it being understood that when R represents a hydrogen atom, then at least one of the conditions defined below is satisfied:
   R₄ represents a heterocyclic group, the said heterocycle being optionally substituted with a C₁₋₃-alkyl, —C(O)C₁₋₃-alkyl, —C(O)C₁₋₃-fluoroalkyl, C₁₋₃-alkylene-C₃₋₆-cycloalkyl, C₁₋₃-alkylene-aryl or C₁₋₃-alkylene-heteroaryl group; the C₁₋₃-alkylene-aryl and C₁₋₃-alkylene-heteroaryl groups being optionally substituted with one or more radicals Z, which may be identical or different, as defined above; or alternatively
   Z represents a phenyl substituted with a halogen atom or a C₁₋₅-alkyl, C₁₋₅-alkoxy or C₁₋₃-fluoroalkyl group, or alternatively
   Z represents an alkynyl radical of the type —C≡C—R_{c}, in which R_{c} represents a hydrogen atom or a C₁₋₃-alkyl, C₁₋₆-alkylene-O—R_{d} or C₁₋₆-alkylene-NRₐR_{b} group or alternatively
   Z represents a —C₄₋₆-alkylene-OR_{d} group, or alternatively
   Z represents a tetrazole group substituted with a C₁₋₃-alkyl group, or alternatively
   Z represents a —C₄₋₆-alkylene-NRₐR_{b} group, or alternatively
   Z represents a group —SO₂—NRₐR_{b}, or alternatively
   Z represents an —O—C₁₋₅-alkylene-NRₐR_{b} group, or alternatively
   Z represents an —O—C₀₋₃-alkylene-heterocyclic group optionally substituted with one or more C₁₋₃-alkyl, oxo or —C(O)—C₁₋₃-alkyl groups, or alternatively
   Z represents an —O—C₁₋₅-alkylene-O—R_{d} group, or alternatively
   Z represents an —O—C₀₋₃-alkylene-C₅₋₇-cycloalkyl group optionally substituted with an —O—R_{d} group, or alternatively
   Z represents a group —NRₑ—C₀₋₃-alkylene-heterocycle optionally substituted with one or more C₁₋₃-alkyl, oxo or —C(O)—C₁₋₃-alkyl groups, or alternatively
   Z represents a group —NRₑ—C₂₋₅-alkylene-O—R_{d}, or alternatively
   Z represents an —O—C₁₋₃-alkylene-heteroaryl group optionally substituted with one or more C₁₋₃-alkyl groups, or alternatively
   Z represents a group —CONH—C₁₋₅-alkylene-NRₐR_{b}, or alternatively
   Z represents a group —CONH—C₁₋₅-alkylene-O—R_{d}, or alternatively
   Z represents an —O—C₁₋₃-alkylene-C(O)—NRₐR_{b},
      in the groups Z mentioned above, the groups Rₐ, R_{b}, R_{d} and Rₑ are as defined previously, i.e.:
Rₐ and R_{b} represent, independently of each other, a hydrogen atom or a C₁₋₃-alkyl or —C(O)—C₁₋₃-alkyl group,
   or alternatively Rₐ and R_{b} form, together with the nitrogen atom to which they are attached, a heterocycle optionally substituted with one or more C₁₋₃-alkyl, oxo, —NRₐR_{b}, hydroxyl, C₁₋₃-alkoxy, C₁₋₃-alkylene-(OH) or —C(O)—C₁₋₃-alkyl groups;
R_{d} represents a hydrogen atom or a C₁₋₃-alkyl group;
Rₑ represents a hydrogen atom or a C₁₋₃-alkyl group;
   or alternatively Z represents a fused or spirane bicyclic diamino heterocycle,
   or alternatively Z represents a group —NRₐR_{b} in which Rₐ and R_{b} form, together with the nitrogen atom to which they are attached, a heterocycle substituted with one or more —NRₐR_{b}, hydroxyl, C₁₋₃-alkoxy, C₁₋₃-alkylene-(OH) or —C(O)—C₁₋₃-alkyl groups.

The compounds of formula (I) may comprise one or more asymmetric carbon atoms. They may thus exist in enantiomeric or diastereoisomeric form. These enantiomers and diastereoisomers, and also mixtures thereof, including racemic mixtures, form part of the invention.

The compounds of formula (I) may comprise one or more rings. They may thus exist in the form of axial/equatorial, endo/exo or cis/trans isomers. These isomers, and mixtures thereof, form part of the invention.

The compounds of formula (I) may comprise one or more olefinic functions. They may thus exist in the form of Z/E isomers. These isomers, and also mixtures thereof, form part of the invention.

The compounds of formula (I) may exist in the form of bases or of acid-addition salts. Such addition salts form part of the invention.

These salts are advantageously prepared with pharmaceutically acceptable acids, but the salts of other acids that are useful, for example, for purifying or isolating the compounds of formula (I) also form part of the invention.

The compounds of formula (I) may also exist in the form of hydrates and/or solvates, i.e. in the form of associations or combinations with water and/or with a solvent. Such hydrates and solvates also form part of the invention.

In the context of the present invention, the following definitions apply:
C_{t-z} in which t and z may take values from 0 to 6, a chain or a carbon-based ring possibly containing from t to z carbon atoms, for example C₀₋₃ can characterize a single bond or a carbon-based chain containing from 1 to 3 carbon atoms;
a halogen atom: a fluorine, a chlorine, a bromine or an iodine;
an alkyl group: a linear or branched, saturated monovalent aliphatic group. Examples that may be mentioned include methyl, ethyl, propyl, isopropyl, butyl, isobutyl, tert-butyl, pentyl, etc. groups;
an alkylene group: a linear or branched saturated divalent aliphatic group. By way of example, a C₁₋₃-alkylene group represents a linear or branched divalent carbon-based chain of 1 to 3 carbon atoms, such as a methylenyl (—CH₂—), an ethylenyl (—CH₂CH₂—), a 1-methylethylenyl (—CH(CH₃)CH₂—), a propylenyl (—CH₂CH₂CH₂—), etc.;
a cycloalkyl group: a saturated cyclic aliphatic group. Examples that may be mentioned include cyclopropyl, methylcyclopropyl, cyclobutyl, cyclopentyl, cyclohexyl, etc. groups;
an alkoxy group: an alkyl-O— radical in which the alkyl group is as defined above;
an alkylenedioxy group: an —O-alkylene-O— group, in which the alkylene group is as defined above. Examples that may be mentioned include methylenedioxy, ethylenedioxy and propylenedioxy groups;
a fluoroalkyl group: an alkyl group of which one or more hydrogen atoms have been replaced with a fluorine atom. Examples that may be mentioned include CF₃—, CF₃CH₂—, etc. groups;
a fluoroalkoxy group: an alkoxy group of which one or more hydrogen atoms have been replaced with a fluorine atom. Examples that may be mentioned include CF₃O—, CHF₂O—, etc. groups;
a heterocyclic group: a saturated 5- to 7-membered cyclic group comprising one or more heteroatoms such as nitrogen, oxygen or sulfur atoms. Examples that may be mentioned include pyrrolidinyl, piperidyl, tetrahydropyranyl, piperidonyl, morpholinyl, piperazinyl, N-methyl-piperazinyl, etc. groups;
   or alternatively a heterocyclic group may represent a 6- to 10-membered bridged heterocyclic group, in which at least two atoms of the bicyclic structure are linked via a single bond or chain that may be 1- to 4-membered.

Examples that may be mentioned include:

the 1-azabicyclo[2.2.2]octyl group:

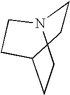

or the 8-azabicyclo[3.2.1]octyl group:

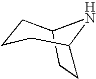

an aryl group: monocyclic or polycyclic aromatic system containing from 6 to 14 carbon atoms and preferably from 6 to 10 carbon atoms. When the system is polycyclic, at least one of the rings is aromatic. Examples that may be mentioned include phenyl, naphthyl, tetrahydronaphthyl, indanyl, etc. groups;
a heteroaryl group: 5- to 14-membered and preferably 5- to 10-membered monocyclic or polycyclic aromatic system comprising one or more heteroatoms such as nitrogen, oxygen or sulfur atoms. When the system is polycyclic, at least one of the rings is aromatic. The nitrogen atoms may be in the form of N-oxides. Examples of monocyclic heteroaryl groups that may be mentioned include thiazolyl, thiadiazolyl, thienyl, imidazolyl, triazolyl, tetrazolyl, pyridyl, furyl, oxazolyl, isoxazolyl, oxadiazolyl, pyrrolyl, pyrazolyl, pyrimidinyl and pyridazinyl groups. Examples of bicyclic heteroaryl groups that may be mentioned include indolyl, benzofuryl, chromen-2-on-yl, benzimidazolyl, benzothienyl, benzotriazolyl, benzothiazolyl, benzoxazolyl, quinolyl, isoquinolyl, indazolyl, indolizinyl, quinazolinyl, phthalazinyl, quinoxalinyl, naphthyridinyl, 2,3-dihydro-1H-indolyl, 2,3-dihydrobenzofuryl, tetrahydroquinolyl and tetrahydroisoquinolyl groups.

In the general formula (I), the ring:

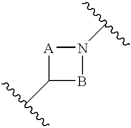

may represent, for example, an azetidinyl, pyrrolidinyl, piperidyl, azepanyl, 8-azabicyclo[3.2.1]octyl group, 8-azabicyclo[3.3.1]nonanyl, decahydroisoquinolyl or 3-azabicyclo[3.1.0]hexanyl group.

Among the compounds of formula (I) that are subjects of the invention, mention may be made of a first subgroup of compounds that are defined as follows:
A represents a C₁₋₄-alkylene group optionally substituted with one or more groups R₉, which may be identical or different;
B represents a C₁₋₄-alkylene group optionally substituted with one or more groups R₁₀, which may be identical or different;
R₉ and R₁₀ represent, independently of each other, a hydrogen atom;
L represents a single bond;
R represents a hydrogen atom or a C₁₋₅-alkyl group such as methyl or ethyl, or a —C(O)C₁₋₃-alkyl group such as —C(O)-methyl;
R₁ represents an aryl such as a phenyl or a naphthyl, or R₁ represents a heteroaryl group such as benzo-1,3-dioxolyl or pyrrolyl; the aryl and heteroaryl groups being optionally substituted with one or more radicals Z, which may be identical or different;
R₂ and R₃ represent, independently of each other, a hydrogen atom;
R₄ represents:
   a hydrogen atom or a C₁₋₅-alkyl group such as a methyl or an ethyl, C₁₋₃-fluoroalkyl group such as a trifluoromethyl, C₁₋₃-alkylene-O—C₁₋₃-alkyl such as a methylene-O-methyl group,
   a heterocyclic group such as a piperidyl or tetrahydropyranyl group; the said heterocyclic group being optionally substituted with a C₁₋₃-alkylene-aryl group such as -methylene-phenyl,
   or alternatively R₄ represents an aryl group such as a phenyl group; the said aryl group being optionally substituted with one or more radicals Z, which may be identical or different;
R₅ represents a hydrogen atom or a C₁₋₅-alkoxy group such as a methoxy group;
R₇ represents a halogen atom such as a chlorine or a C₁₋₅-alkoxy group such as a methoxy group;
Z represents a hydrogen atom, a halogen atom such as a fluorine, a C₁₋₅-alkoxy group such as a methoxy, or phenyl optionally substituted with a C₁₋₃-fluoroalkyl group such as a trifluoromethyl,
   or alternatively Z represents an alkynyl radical of the type —C≡C—R_{c}, in which R_{c} represents a hydrogen atom, C₁₋₆-alkylene-O—R_{d} or C₁₋₆-alkylene-NRₐR_{b} in which the C₁₋₆-alkylene groups are such as an isopropylene or a methylene,
   or alternatively Z represents a group —C₄₋₆-alkylene-OR_{d} in which the C₄₋₆-alkylene group is such as a butylene,
   or alternatively Z represents a tetrazole group substituted with a C₁₋₃-alkyl group such as a methyl group,
   or alternatively Z represents a group —NRₐR_{b},
   or alternatively Z represents a group —C₄₋₄-alkylene-NRₐR_{b} in which the C₄₋₆-alkylene group is such as a butylene,
   or alternatively Z represents a group —O—C₁₋₅-alkylene-NRₐR_{b} in which the C₁₋₅-alkylene group is such as an ethylene or a propylene,
   or alternatively Z represents an —O—C₀₋₃-alkylene-heterocycle group in which the alkylene group is absent or alternatively is such as a methylene, ethylene group, and the heterocycle is such as an azepanyl, pyrrolidinyl, piperidyl or tetrahydrofuryl group, the 1-azabicyclo[2.2.2]octyl group or the 8-azabicyclo[3.2.1]octyl group, the said —O—C₀₋₃-alkylene-heterocycle group being optionally substituted with one or more C₁₋₃-alkyl groups such as a methyl or an oxo group,
   or alternatively Z represents a group —O—C₁₋₅-alkylene-O—R_{d} in which the C₁₋₅-alkylene group is such as an ethylene or propylene group,
   or alternatively Z represents a group —NRₑ—C₂₋₅-alkylene-O—R_{d} in which the C₂₋₅-alkylene group is such as an ethylene group,
   or alternatively Z represents an —O—C₁₋₃-alkylene-heteroaryl group in which the C₁₋₃-alkylene group is such as a methylene or ethylene group and heteroaryl group is such as an imidazolyl or pyridyl group,
   or alternatively Z represents a group —CONH—C₁₋₅-alkylene-NRₐR_{b} in which the C₁₋₅-alkylene group is such as an ethylene group,
   or alternatively Z represents an —O—C₁₋₃-alkylene-C(O)—NRₐR_{b} in which C₁₋₃-alkylene group is such as a methylene,
   or alternatively Z represents a fused or spirane bicyclic diamino heterocycle chosen from:

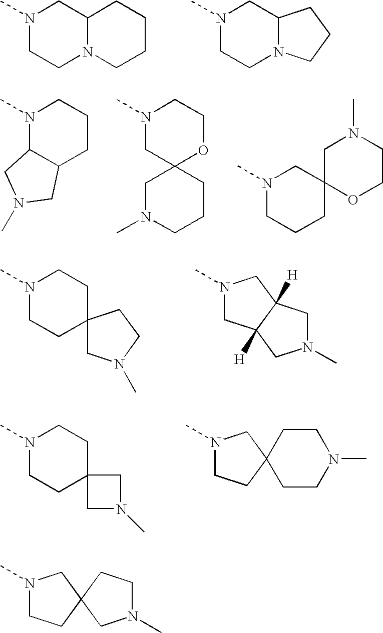

in which the dashed lines represent the point of attachment to the rest of the molecule of formula (I) and the solid lines represent a methyl substituent;
Rₐ and R_{b} represent, independently of each other a C₁₋₃-alkyl group such as a methyl,
   or alternatively Rₐ and R_{b} form, together with the nitrogen atom to which they are attached, a heterocycle such as a morpholinyl, piperidyl or pyrrolidinyl group, optionally substituted with one or more oxo, —NRₐR_{b} or hydroxyl groups;
R_{d} represents a hydrogen atom or a C₁₋₃-alkyl group such as a methyl;
Rₑ represents a C₁₋₃-alkyl group such as a methyl;
   it being understood that when R represents a hydrogen atom, then at least one of the conditions defined below is satisfied:
   R₄ represents a heterocyclic group such as a piperidyl or tetrahydropyranyl group, the said heterocycle being optionally substituted with a C₁₋₃-alkylene-aryl group such as methylene-phenyl, or alternatively
   Z represents a phenyl group substituted with a C₁₋₃-fluoroalkyl group such as a trifluoromethyl, or alternatively
   Z represents an alkynyl radical of the type —C≡C—R_{c}, in which R_{c} represents a hydrogen atom or a C₁₋₆-alkylene-O—R_{d} or C₁₋₆-alkylene-NRₐR_{b} group in which C₁₋₆-alkylene is such as an isopropyl or a methyl, or alternatively
   Z represents a —C₄₋₄-alkylene-OR_{d} group in which C₄₋₄-alkylene is such as a butylene or alternatively
   Z represents a tetrazole group substituted with a C₁₋₃-alkyl group such as a methyl, or alternatively
   Z represents a group —C₄₋₆-alkylene-NRₐR_{b} in which C₄₋₆-alkylene is such as a butylene, or alternatively
   Z represents a group —O—C₁₋₅-alkylene-NRₐR_{b}, in which C₁₋₅-alkylene is such as a propylene or ethylene, or alternatively
   Z represents an —O—C₀₋₃-alkylene-heterocycle group in which the alkylene group is absent or is such as a methylene, ethylene or propylene group and in which the heterocycle is such as an azepanyl, pyrrolidinyl, piperidyl or tetrahydrofuryl group, the 1-azabicyclo[2.2.2]octyl group or the 8-azabicyclo[3.2.1]octyl group, the said —O—C₀₋₃-alkylene-heterocycle being optionally substituted with one or more C₁₋₃-alkyl groups such as methyl or an oxo group, or alternatively
   Z represents a group —O—C₁₋₅-alkylene-O—R_{d} in which C₁₋₅-alkylene is such as an ethylene or propylene group, or alternatively
   Z represents a group —NRₑ—C₂₋₅-alkylene-O—R_{d} in which C₂₋₅-alkylene is such as an ethylene group, or alternatively
   Z represents an —O—C₁₋₃-alkylene-heteroaryl group in which C₁₋₃-alkylene is such as a methylene or ethylene and in which the heteroaryl group is such as an imidazolyl or pyridyl group, or alternatively
   Z represents a group —CONH—C₁₋₅-alkylene-NRₐR_{b} in which C₁₋₅-alkylene is such as an ethylene group, or alternatively
   Z represents an —O—C₁₋₃-alkylene-C(O)—NRₐR_{b} in which C₁₋₃-alkylene is such as a methylene,
      in the groups Z mentioned above, the groups Rₐ, R_{b}, R_{d} and Rₑ are as defined previously, i.e.:
Rₐ and R_{b} represent, independently of each other a C₁₋₃-alkyl group such as a methyl,
   or alternatively Rₐ and R_{b} form, together with the nitrogen atom to which they are attached, a heterocycle such as a piperidyl, morpholinyl or pyrrolidinyl, the said heterocyclic group being optionally substituted with one or more oxo, —NRₐR_{b} or hydroxyl groups;
R_{d} represents a hydrogen atom or a C₁₋₃-alkyl group such as a methyl;
Rₑ represents a C₁₋₃-alkyl group such as a methyl;
or alternatively Z represents a fused or spirane bicyclic diamino heterocycle chosen from:

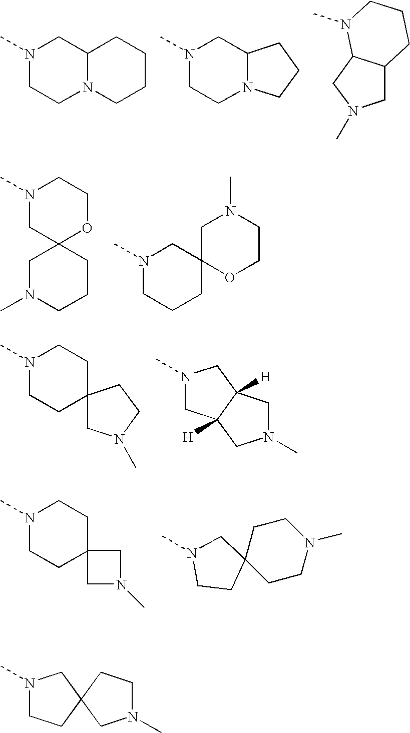

in which the dashed lines represent the point of attachment to the rest of the molecule of formula (I) and the solid lines represent a methyl substituent;
or alternatively Z represents a group —NRₐR_{b} in which Rₐ and R_{b} form, together with the nitrogen atom to which they are attached, a heterocycle such as a pyrrolidinyl
   or piperidinyl substituted with one or more —NRₐR_{b}, or hydroxyl groups.

Among the compounds of formula (I) that are subjects of the invention, mention may be made of a second subgroup of compounds that are defined as follows:
A represents an ethylene group;
B represents an ethylene group;
L represents a single bond;
R represents a hydrogen atom, a methyl or ethyl group or a —C(O)-methyl group;
R₁ represents a phenyl, naphthyl, benzo-1,3-dioxolyl or pyrrolyl group; the said phenyl, naphthyl, benzo-1,3-dioxolyl and pyrrolyl groups being optionally substituted with one or more radicals Z, which may be identical or different;
R₂ and R₃ represent, independently of each other, a hydrogen atom;
R₄ represents:
   a hydrogen atom or a methyl, ethyl, trifluoromethyl or methylene-O-methyl group,
   a piperidyl or tetrahydropyranyl group; the said piperidyl or tetrahydropyranyl groups being optionally substituted with a methylene-phenyl group,
   or alternatively R₄ represents a phenyl group; the said phenyl group being optionally substituted with one or more radicals Z, which may be identical or different;
R₅ represents a hydrogen atom or a methoxy group;
R₇ represents a chlorine atom or a methoxy group;
Z represents a hydrogen atom or a fluorine atom, a methoxy group or a phenyl group optionally substituted with a trifluoromethyl group,
   or alternatively Z represents an alkynyl radical of the type —C≡C—R_{c}, in which R₁ represents a hydrogen atom, C₁₋₆-alkylene-O—R_{d} or C₁₋₆-alkylene-NRₐR_{b} in which the C₁₋₆-alkylene groups are such as an isopropylene or a methylene,
   or alternatively Z represents a butylene-OR_{d} group,
   or alternatively Z represents a tetrazole group substituted with a methyl group
   or alternatively Z represents a group —NRₐR_{b},
   or alternatively Z represents a group butylene-NRₐR_{b},
   or alternatively Z represents an —O—C₁₋₅-propylene-NRₐR_{b} or —O—C₁₋₅-ethylene-NRₐR_{b} group,
   or alternatively Z represents an —O-heterocycle, —O-methylene-heterocycle, —O-ethylene-heterocycle group in which the heterocycle is such as an azepanyl, pyrrolidinyl, piperidyl or tetrahydrofuryl group, the 1-azabicyclo[2.2.2]octyl group or the 8-azabicyclo[3.2.1]octyl group, the said —O-heterocycle, —O-methylene-heterocycle and —O-ethylene-heterocycle being optionally substituted with one or more methyl or an oxo group,
   or alternatively Z represents a group —O-ethylene-OR_{d} or —O-propylene-OR_{d},
   or alternatively Z represents a group —NRₑ-ethylene-OR_{d},
   or alternatively Z represents an —O-methylene-heteroaryl or —O-ethylene-heteroaryl group in which the heteroaryl group is such as an imidazolyl or pyridyl group,
   or alternatively Z represents a group —CONH-ethylene-NRₐR_{b},
   or alternatively Z represents a group —O-methylene-C(O)—NRₐR_{b},
   or alternatively Z represents a fused or spirane bicyclic diamino heterocycle chosen from:

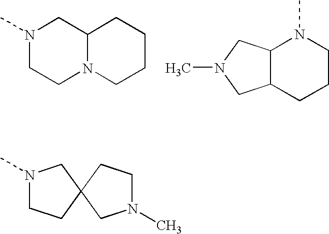

in which the dashed lines represent the point of attachment to the rest of the molecule of formula (I) and the solid lines represent a methyl substituent;
Rₐ and R_{b} represent, independently of each other, a methyl group,
   or alternatively Rₐ and R_{b} form, together with the nitrogen atom to which they are attached, a group morpholinyl, piperidyl or pyrrolidinyl group, optionally substituted with one or more oxo, —NRₐRₐ or hydroxyl groups;
R_{d} represents a hydrogen atom or a methyl group;
Rₑ represents a methyl group;
   it being understood that when R represents a hydrogen atom, then at least one of the conditions defined below is satisfied:
   R₄ represents a piperidyl or tetrahydropyranyl group, the said piperidyl or tetrahydropyranyl groups being optionally substituted with a methylene-phenyl group, or alternatively
   Z represents a phenyl group substituted with a trifluoromethyl group, or alternatively
   Z represents an alkynyl radical of the type —C≡C—R_{c} in which R_{c} represents a hydrogen atom, isopropylene-O—R_{d} or methylene-NRₐR_{b} group, or alternatively
   Z represents a butylene-OR_{d} group, or alternatively
   Z represents a tetrazole group substituted with a methyl group, or alternatively
   Z represents a butylene-NRₐR_{b}, or alternatively
   Z represents an —O-propylene-NRₐR_{b} group, or alternatively
   Z represents an —O-heterocycle, —O-methylene-heterocycle, —O-ethylene-heterocycle group in which the heterocycle is such as an azepanyl, pyrrolidinyl, piperidyl or tetrahydrofuryl group, the 1-azabicyclo[2.2.2]octyl group or the 8-azabicyclo[3.2.1]octyl group, the said —O—C₀₋₃-alkylene-heterocycle being optionally substituted with one or more methyl groups or an oxo group, or alternatively
   Z represents a group —O-ethylene-O—R_{d} or —O-propylene-O—R_{d}, or alternatively
   Z represents a group —NRₑ-ethylene-O—R_{d}, or alternatively
   Z represents an —O-methylene-heteroaryl or —O-ethylene-heteroaryl group in which the heteroaryl group is such as an imidazolyl or pyridyl group, or alternatively
   Z represents a —CONH-ethylene-NRₐR_{b} group, or alternatively
   Z represents an —O-methylene-C(O)—NRₐR_{b} group, in the groups Z mentioned above, the groups Rₐ, R_{b}, R_{d} and Rₑ are as defined previously, i.e.:
Rₐ and R_{b} represent, independently of each other, a methyl group,
   or alternatively Rₐ and R_{b} form, together with the nitrogen atom to which they are attached, a piperidyl, morpholinyl or pyrrolidinyl group, the said piperidyl, morpholinyl and pyrrolidinyl groups being optionally substituted with one or more oxo, —NRₐR_{b} or hydroxyl groups;
R_{d} represents a hydrogen atom or a methyl group;
Rₑ represents a methyl;
   or alternatively Z represents a fused or spirane bicyclic diamino heterocycle chosen from:

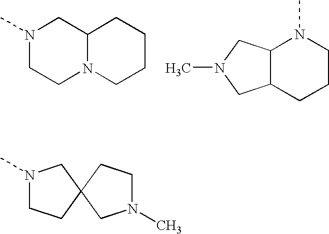

in which the dashed lines represent the point of attachment to the rest of the molecule of formula (I) and the solid lines represent a methyl substituent;
or alternatively Z represents a group —NRₐR_{b} in which Rₐ and R_{b} form, together with the nitrogen atom to which they are attached, a piperidyl or pyrrolidinyl group optionally substituted with one or more —NRₐR_{b} or hydroxyl groups

Among the compounds of formula (I) that are subjects of the invention, mention may be made of a third subgroup of compounds that is defined as follows:
A represents a C₁₋₄-alkylene group optionally substituted with one or more groups R₉, which may be identical or different;
B represents a C₁₋₄-alkylene optionally substituted with one or more groups R₁₀, which may be identical or different;
R₉ and R₁₀ represent, independently of each other, a hydrogen atom or a C₁₋₅-alkyl group,
   or alternatively R₉ and R₁₀ together form a single bond or a C₁₋₄-alkylene group;
L represents a single bond or a C₁₋₂-alkylene, —CH═CH— or —C≡C— group; the C₁₋₂-alkylene and —CH═CH— groups being optionally substituted with one or more C₁₋₂-alkyl substituents,
   or alternatively L represents a cycloprop-1,2-diyl group;
R represents a hydrogen atom or a C₁₋₅-alkyl, C₁₋₃-fluoroalkyl, C₃₋₆-cycloalkyl, —C(O)C₁₋₃-alkyl, C₁₋₃-alkylene-C₃₋₆-cycloalkyl, —CH₂—C≡CH, C₂₋₄-alkylene-NRₐR_{b} or C₁₋₃-alkylene-X—C₁₋₃-alkyl group in which X represents O or SO₂;
R₁ represents an aryl or a heteroaryl group; the aryl and heteroaryl groups being optionally substituted with one or more radicals Z, which may be identical or different;
R₂ and R₃ represent, independently of each other, a hydrogen atom or a C₁₋₃-alkyl or C₁₋₃-fluoroalkyl,
   or alternatively R₂ and R₃ form, together with the carbon atom that bears them, a cycloprop-1,1-diyl group;
R₄ represents:
   a hydrogen atom or a C₁₋₅-alkyl, C₁₋₃-fluoroalkyl, C₃₋₄-cycloalkyl, C₁₋₃-alkylene-C₃₋₆-cycloalkyl, C₁₋₃-alkylene-O—C₁₋₃-alkyl group,
   a C₁₋₃-alkylene-(OH) or C₁₋₃-alkylene-X—C₁₋₃-alkyl group in which X represents S, SO or SO₂,
   a heterocyclic group; the said heterocyclic group being optionally substituted with a C₁₋₃-alkyl, —C(O)C₁₋₃-alkyl, —C(O)C₁₋₃-fluoroalkyl, C₁₋₃-alkylene-C₃₋₆-cycloalkyl, C₁₋₃-alkylene-aryl or C₁₋₃-alkylene-heteroaryl group; the C₁₋₃-alkylene-aryl and C₁₋₃-alkylene-heteroaryl groups being optionally substituted with one or more radicals Z, which may be identical or different,
   or alternatively R₄ represents a C₁₋₃-alkylene-NRₐR_{b}, aryl, C₁₋₃-alkylene-aryl, —O-aryl, C₁₋₃-alkylene-O-aryl, C₁₋₃-alkylene-O—C₁₋₃-alkylene-aryl, heteroaryl or C₁₋₃-alkylene-heteroaryl group; the aryl, C₁₋₃-alkylene-aryl, —O-aryl, C₁₋₃-alkylene-O-aryl, C₁₋₃-alkylene-O—C₁₋₃-alkylene-aryl, heteroaryl and C₁₋₃-alkylene-heteroaryl groups being optionally substituted with one or more radicals Z, which may be identical or different;
R₅ represents a hydrogen or halogen atom or a C₁₋₅-alkyl, C₁₋₃-fluoroalkyl, C₁₋₅-alkoxy, C₁₋₃-fluoroalkoxy, C₁₋₃-alkylene-(OH), —CN or —X—C₁₋₃-alkyl group in which X represents S, SO or SO₂,
   or alternatively R₅ represents an —NRₐR_{b}, C₁₋₃-alkylene-NRₐR_{b}, aryl, C₁₋₃-alkylene-aryl, —O-aryl or heteroaryl group; the aryl, C₁₋₃-alkylene-aryl, —O-aryl and heteroaryl groups being optionally substituted with one or more radicals Z, which may be identical or different;
R₇ represents a hydrogen or halogen atom or a C₁₋₅-alkyl, C₁₋₃-fluoroalkyl, C₁₋₅-alkoxy, —COOH, —C(O)OC₁₋₃-alkyl, C₁₋₃-fluoroalkoxy, C₁₋₃-alkylene-(OH), —CN or —X—C₁₋₃-alkyl group in which X represents S, SO or SO₂,
   or alternatively R₇ represents a group —NRₐR_{b}, C₁₋₃-alkylene-NRₐR_{b} or —C(O)—NRₐR_{b},
      —C(O)—C₁₋₃-alkyl, aryl, —O-aryl or heteroaryl group; the aryl, —O-aryl and heteroaryl groups being optionally substituted with one or more radicals Z, which may be identical or different;
Z represents a halogen atom or a C₁₋₅-alkyl, C₁₋₃-fluoroalkyl, C₃₋₆-cycloalkyl, C₁₋₃-alkylene-C₃₋₆-cycloalkyl, phenyl, C₁₋₅-alkoxy, C₁₋₃-fluoroalkoxy, C₁₋₃-alkylene-O—C₁₋₃-alkyl, C₁₋₃-alkylene-(OH), NO₂, —CN, —SO₂NRₐR_{b}, —X—C₁₋₃-alkyl or C₁₋₃-alkylene-X—C₁₋₃-alkyl group in which X represents S, SO or SO₂,
   or alternatively Z represents a group —NRₐR_{b}, C₁₋₃-alkylene-NRₐR_{b}, —C(O)—NRₐR_{b}, —C(O)—C₁₋₃-alkyl, —C(O)O—C₁₋₄-alkyl or —C(O)—C₃₋₆-cycloalkyl,
   or alternatively Z represents an oxo radical,
   or alternatively Z represents an —O—C₁₋₅-alkylene-NRₐR_{b} group,
   or alternatively two adjacent radicals Z together form a C₁₋₃-alkylenedioxy group;
Rₐ and R_{b} represent, independently of each other, a hydrogen atom or a C₁₋₃-alkyl or —C(O)—C₁₋₃-alkyl group,
   or alternatively Rₐ and R_{b} form, together with the nitrogen atom that bears them, a heterocycle optionally substituted with one or more C₁₋₃-alkyl or oxo groups; it being understood that when R represents a hydrogen atom, at least one of the following two conditions is satisfied:
R₄ represents a heterocyclic group; the said heterocycle being optionally substituted,
Z represents an —SO₂—NRₐR_{b} group or an —O—C₁₋₅-alkylene-NRₐR_{b} group.

Among the compounds of formula (I) that are subjects of the invention, mention may be made of a fourth subgroup of compounds that are defined as follows:
A represents a C₁₋₄-alkylene group optionally substituted with one or more groups R₉, which may be identical or different;
B represents a C₁₋₄-alkylene group optionally substituted with one or more groups R₁₀, which may be identical or different;
R₉ and R₁₀ represent, independently of each other, a hydrogen atom or a C₁₋₅-alkyl group and in particular a methyl,
   or alternatively R₉ and R₁₀ together form a C₁₋₄-alkylene group;
L represents a single bond or —CH═CH—;
R represents a hydrogen atom or a C₁₋₅-alkyl, C₁₋₃-fluoroalkyl, C₃₋₆-cycloalkyl or C₁₋₃-alkylene-C₃₋₆-cycloalkyl group;
R₁ represents an aryl or a heteroaryl group; the aryl and heteroaryl groups being optionally substituted with one or more radicals Z, which may be identical or different;
R₂ and R₃ represent, independently of each other, a hydrogen atom or a C₁₋₃-alkyl group;
R₄ represents:
   a hydrogen atom or a C₁₋₅-alkyl, C₃₋₄-cycloalkyl, C₁₋₃-alkylene-C₃₋₆-cycloalkyl or C₁₋₃-alkylene-O—C₁₋₃-alkyl group,
   a C₁₋₃-alkylene-(OH) group,
   a heterocyclic group; the said heterocyclic group being optionally substituted with a C₁₋₃-alkyl, —C(O)C₁₋₃-alkyl, —C(O)C₁₋₃-fluoroalkyl, C₁₋₃-alkylene-C₃₋₆-cycloalkyl or C₁₋₃-alkylene-aryl group; the C₁₋₃-alkylene-aryl group being optionally substituted with one or more radicals Z, which may be identical or different,
   or alternatively R₄ represents a C₁₋₃-alkylene-NRₐR_{b}, aryl, C₁₋₃-alkylene-aryl, C₁₋₃-alkylene-O-aryl, C₁₋₃-alkylene-O—C₁₋₃-alkylene-aryl, heteroaryl or C₁₋₃-alkylene-heteroaryl group; the aryl, C₁₋₃-alkylene-aryl, C₁₋₃-alkylene-O-aryl, C₁₋₃-alkylene-O—C₁₋₃-alkylene-aryl, heteroaryl and C₁₋₃-alkylene-heteroaryl groups being optionally substituted with one or more radicals Z, which may be identical or different;
R₅ represents a hydrogen atom or a C₁₋₅-alkyl, C₁₋₅-alkoxy or aryl group; the aryl group being optionally substituted with one or more radicals Z, which may be identical or different;
R₇ represents a halogen atom or a C₁₋₅-alkyl group and in particular a methyl, C₁₋₅-alkoxy and in particular a methoxy, CN, COOH or —C(O)OC₁₋₃-alkyl group;
Z represents a halogen atom or a C₁₋₅-alkyl, C₁₋₃-fluoroalkyl, C₃₋₆-cycloalkyl, C₁₋₃-alkylene-C₃₋₆-cycloalkyl, phenyl, C₁₋₅-alkoxy, C₁₋₃-fluoroalkoxy, C₁₋₃-alkylene-O—C₁₋₃-alkyl, C₁₋₃-alkylene-(OH), NO₂, —CN, —SO₂NRₐR_{b}, —X—C₁₋₃-alkyl or C₁₋₃-alkylene-X—C₁₋₃-alkyl group in which X represents S, SO or SO₂,
   or alternatively Z represents a group —NRₐR_{b}, C₁₋₃-alkylene-NRₐR_{b}, —C(O)—NRₐR_{b}, —C(O)—C₁₋₃-alkyl, —C(O)O—C₁₋₄-alkyl or —C(O)—C₃₋₄-cycloalkyl group,
   or alternatively Z represents an oxo radical,
   or alternatively Z represents an —O—C₁₋₅-alkylene-NRₐR_{b} group,
   or alternatively two adjacent radicals Z together form a C₁₋₃-alkylenedioxy group;
Rₐ and R_{b} represent, independently of each other, a hydrogen atom or a C₁₋₃-alkyl or —C(O)—C₁₋₃-alkyl group,
   or alternatively Rₐ and R_{b} form, together with the nitrogen atom that bears them, a heterocycle optionally substituted with one or more C₁₋₃-alkyl or oxo groups; it being understood that at least one of the conditions stated previously is satisfied.

Among the compounds of formula (I) that are subjects of the invention, mention may be made of a fifth subgroup of compounds that are defined as follows:
A represents a C₁₋₄-alkylene and in particular an ethylene, optionally substituted with one or more groups R₉;
B represents a C₁₋₄-alkylene group and in particular an ethylene, optionally substituted with one or more groups R₁₀;
R₉ and R₁₀ represent, independently of each other, a hydrogen atom or a methyl,
   or alternatively R₉ and R₁₀ together form a C₁₋₄-alkylene group;
L represents a single bond or —CH═CH—;
R represents a hydrogen atom or a C₁₋₅-alkyl group and in particular an ethyl or C₁₋₃-alkylene-C₃₋₆-cycloalkyl group;
R₁ represents an aryl or a heteroaryl group; the aryl and heteroaryl groups being optionally substituted with one or more radicals Z, which may be identical or different;
R₂ and R₃ represent, independently of each other, a hydrogen atom or a C₁₋₃-alkyl group and in particular a methyl;
R₄ represents:
   a hydrogen atom or a C₁₋₅-alkyl, C₃₋₆-cycloalkyl, C₁₋₃-alkylene-C₃₋₆-cycloalkyl or C₁₋₃-alkylene-O—C₁₋₃-alkyl group and in particular a methylene-O-methyl group,
   a C₁₋₃-alkylene-(OH) group,
   a heterocyclic group optionally substituted with a C₁₋₃-alkyl, —C(O)C₁₋₃-alkyl, —C(O)C₁₋₃-fluoroalkyl, C₁₋₃-alkylene-C₃₋₆-cycloalkyl or C₁₋₃-alkylene-aryl group and in particular a methylene-phenyl; the C₁₋₃-alkylene-aryl group being optionally substituted with one or more radicals Z, which may be identical or different,
   or alternatively R₄ represents a group C₁₋₃-alkylene-NRₐR_{b}, aryl and in particular a phenyl, C₁₋₃-alkylene-aryl and in particular a C₁₋₃-alkylene-phenyl, C₁₋₃-alkylene-O-aryl and in particular a C₁₋₃-alkylene-O-phenyl, C₁₋₃-alkylene-O—C₁₋₃-alkylene-aryl and in particular a C₁₋₃-alkylene-O—C₁₋₃-alkylene-phenyl, heteroaryl and in particular a thienyl or a pyridyl; the aryl, C₁₋₃-alkylene-aryl, C₁₋₃-alkylene-O-aryl, C₁₋₃-alkylene-O—C₁₋₃-alkylene-aryl and heteroaryl groups being optionally substituted with one or more radicals Z, which may be identical or different;
R₅ represents a hydrogen atom;
R₇ represents a halogen atom, a methyl, a methoxy, CN, COOH or —C(O)O—C₁₋₃-alkyl;
Z represents a halogen atom or a C₁₋₅-alkyl, a phenyl, C₁₋₅-alkoxy and in particular methoxy, C₁₋₃-alkylene-O—C₁₋₃-alkyl, C₁₋₃-alkylene-(OH), —CN, —SO₂NRₐR_{b}, —X—C₁₋₃-alkyl or C₁₋₃-alkylene-X—C₁₋₃-alkyl group in which X represents S, SO or SO₂;
   or alternatively Z represents a group —NRₐR_{b}, C₁₋₃-alkylene-NRₐR_{b}, —C(O)—NRₐR_{b} or —C(O)—C₁₋₃-alkyl,
   or alternatively Z represents an oxo radical,
   or alternatively Z represents an —O—C₁₋₅-alkylene-NRₐR_{b} group,
   or alternatively two adjacent radicals Z together form a C₁₋₃-alkylenedioxy group, and in particular a methylenedioxy;
Rₐ and R_{b} represent, independently of each other, a hydrogen atom or a C₁₋₃-alkyl or —C(O)—C₁₋₃-alkyl group,
   or alternatively Rₐ and R_{b} form, together with the nitrogen atom that bears them, a heterocycle optionally substituted with one or more C₁₋₃-alkyl or oxo groups; it being understood that at least one of the conditions stated previously is satisfied.

Among the compounds of formula (I) that are subjects of the invention, mention may be made of a sixth subgroup of compounds that are defined as follows:
A represents an ethylene, optionally substituted with one or more groups R₉;
B represents an ethylene, optionally substituted with one or more groups R₁₀;
R₉ and R₁₀ represent, independently of each other, a hydrogen atom or a methyl,
L represents a single bond;
R represents a hydrogen atom, an ethyl or C₁₋₃-alkylene-C₃₋₄-cycloalkyl group;
R₁ represents an aryl or a heteroaryl group; the aryl and heteroaryl groups being optionally substituted with one or more radicals Z, which may be identical or different;
R₂ and R₃ represent, independently of each other, a hydrogen atom or a methyl group;
R₄ represents:
   a hydrogen atom or a C₁₋₅-alkyl, C₃₋₆-cycloalkyl, C₁₋₃-alkylene-C₃₋₆-cycloalkyl or methylene-O-methyl group,
   a C₁₋₃-alkylene-(OH) group,
   a heterocyclic group optionally substituted with a C₁₋₃-alkyl, —C(O)C₁₋₃-alkyl, —C(O)C₁₋₃-fluoroalkyl, C₁₋₃-alkylene-C₃₋₄-cycloalkyl or methylene-phenyl group; the methylene-phenyl group being optionally substituted with one or more radicals Z, which may be identical or different,
   or alternatively R₄ represents a C₁₋₃-alkylene-NRₐR_{b}, a phenyl, a C₁₋₃-alkylene-phenyl, a C₁₋₃-alkylene-O-phenyl, a C₁₋₃-alkylene-O—C₁₋₃-alkylene-phenyl, a thienyl or a pyridyl group; the phenyl, C₁₋₃-alkylene-phenyl, C₁₋₃-alkylene-O-phenyl, C₁₋₃-alkylene-O—C₁₋₃-alkylene-phenyl, thienyl and pyridyl groups being optionally substituted with one or more radicals Z, which may be identical or different;
R₅ represents a hydrogen atom;
R₇ represents a halogen atom, a methyl, a methoxy, CN;
Z represents a halogen atom or a C₁₋₅-alkyl, a phenyl, a methoxy, C₁₋₃-alkylene-O—C₁₋₃-alkyl, C₁₋₃-alkylene-(OH), —CN, —SO₂NRₐR_{b}, —X—C₁₋₃-alkyl or C₁₋₃-alkylene-X—C₁₋₃-alkyl group in which X represents S, SO or SO₂,
   or alternatively Z represents a group —NRₐR_{b}, C₁₋₃-alkylene-NRₐR_{b}, —C(O)—NRₐR_{b}
   or —C(O)—C₁₋₃-alkyl, or alternatively Z represents an oxo radical,
   or alternatively Z represents a group —O—C₁₋₅-alkylene-NRₐR_{b},
   or alternatively two adjacent radicals Z together form a methylenedioxy group;
Rₐ and R_{b} represent, independently of each other, a hydrogen atom or a C₁₋₃-alkyl or —C(O)—C₁₋₃-alkyl group,
   or alternatively Rₐ and R_{b} form, together with the nitrogen atom that bears them, a heterocycle optionally substituted with one or more C₁₋₃-alkyl or oxo groups; it being understood that at least one of the conditions stated previously is satisfied.

Among the compounds of formula (I) that are subjects of the invention, mention may be made especially of the compounds chosen from:
N-[1-(1,3-benzodioxol-5-ylmethyl)piperidin-4-yl]-4-(1-benzylpiperidin-4-yl)-7-methoxyphthalazin-1-amine trihydrochloride (compound 1)
N-(1-{4-[3-(dimethylamino)propoxy]benzyl}piperidin-4-yl)-7-methoxy-4-(4-methoxyphenyl)phthalazin-1-amine trihydrochloride (compound 3)
N-{1-[3-fluoro-4-(3-morpholin-4-ylpropoxy)benzyl]piperidin-4-yl}-7-methoxy-4-(4-methoxyphenyl)phthalazin-1-amine trihydrochloride (compound 4)
7-methoxy-4-(4-methoxyphenyl)-N-{1-[4-(2-piperidin-1-ylethoxy)benzyl]piperidin-4-yl}phthalazin-1-amine trihydrochloride (compound 5)
N-(1-{4-[3-(dimethylamino)propoxy]-3-fluorobenzyl piperidin-4-yl}-7-methoxy-4-(4-methoxyphenyl)phthalazin-1-amine trihydrochloride (compound 8)
7-methoxy-4-(4-methoxyphenyl)-N-{1-[4-(2-pyrrolidin-1-ylethoxy)benzyl]piperidin-4-yl}phthalazin-1-amine trihydrochloride (compound 9)
7-methoxy-4-(4-methoxyphenyl)-N-{1-[4-(2-morpholin-4-ylethoxy)benzyl]piperidin-4-yl}phthalazin-1-amine trihydrochloride (compound 10)
7-methoxy-4-(4-methoxyphenyl)-N-{1-[4-(3-piperidin-1-ylpropoxy)benzyl]piperidin-4-yl}phthalazin-1-amine trihydrochloride (compound 11)
N-[1-(1,3-benzodioxol-5-ylmethyl)piperidin-4-yl]-N-ethyl-7-methoxy-4-(4-methoxyphenyl)phthalazin-1-amine dihydrochloride (compound 12)
4-[(4-{[7-methoxy-4-(4-methoxyphenyl)phthalazin-1-yl]amino}piperidin-1-yl)-methyl]-N-(2-piperidin-1-ylethyl)benzamide trihydrochloride (compound 13)
N-[1-(1,3-benzodioxol-5-ylmethyl)piperidin-4-yl]-7-methoxy-4-(tetrahydro-2H-pyran-4-yl)phthalazin-1-amine dihydrochloride (compound 19)
7-methoxy-4-(4-methoxyphenyl)-N-{1-[4-(3-methoxypropoxy)benzyl]piperidin-4-yl}phthalazin-1-amine dihydrochloride (compound 20)
N-(1-{4-[3-(dimethylamino)propoxy]-3-fluorobenzyl}piperidin-4-yl)-4-ethyl-7-meth-oxyphthalazin-1-amine trihydrochloride (compound 21)
N-(1-{4-[3-(dimethylamino)propoxy]-3-fluorobenzyl-piperidin-4-yl}-7-methoxy-4-(methoxymethyl)phthalazin-1-amine trihydrochloride (compound 22)
N-{1-[3-fluoro-4-(3-pyrrolidin-1-ylpropoxy)benzyl]piperidin-4-yl}-7-methoxy-4-(4-methoxyphenyl)phthalazin-1-amine trihydrochloride (compound 23)
1-(3-{4-[(4-{[7-methoxy-4-(4-methoxyphenyl)phthalazin-1-yl]amino}piperidin-1-yl)methyl]phenoxy}propyl)pyrrolidin-2-one trihydrochloride (compound 24)
N-(1-{4-[2-(dimethylamino)ethoxy]benzyl}piperidin-4-yl)-7-methoxy-4-(4-methoxyphenyl)phthalazin-1-amine trihydrochloride (compound 25)
7-methoxy-4-(4-methoxyphenyl)-N-(1-{4-[(1-methylazepan-4-yl)oxy]benzyl}-piperidin-4-yl)phthalazin-1-amine trihydrochloride (compound 26)
7-methoxy-4-(4-methoxyphenyl)-N-(1-{4-[2-(1-methylpyrrolidin-2-yl)ethoxy]-benzyl}piperidin-4-yl)phthalazin-1-amine trihydrochloride (compound 27)
4-ethyl-N-[1-(4-ethynylbenzyl)piperidin-4-yl]-7-methoxyphthalazin-1-amine dihydrochloride (compound 28)
N-(1-{4-[3-(dimethylamino)prop-1-yn-1-yl]benzyl}piperidin-4-yl)-4-ethyl-7-meth-oxyphthalazin-1-amine trihydrochloride (compound 29)
3-{4-[(4-{[7-methoxy-4-(4-methoxyphenyl)phthalazin-1-yl]amino}piperidin-1-yl)-methyl]phenoxy}propan-1-ol dihydrochloride (compound 30)
4-ethyl-7-methoxy-N-{1-[4-(4-pyrrolidin-1-ylpiperidin-1-yl)benzyl]piperidin-4-yl}-phthalazin-1-amine trihydrochloride (compound 31)
N-(1-{4-[3-(dimethylamino)propoxy]benzyl}piperidin-4-yl)-7-methoxy-4-(methoxy-methyl)phthalazin-1-amine trihydrochloride (compound 32)
N-(1-{4-[(3S)-3-(dimethylamino)pyrrolidin-1-yl]benzyl}piperidin-4-yl)-7-methoxy-4-(methoxymethyl)phthalazin-1-amine trihydrochloride (compound 33)
N-(1-{4-[(3R)-3-(dimethylamino)pyrrolidin-1-yl]benzyl}piperidin-4-yl)-7-methoxy-4-(methoxymethyl)phthalazin-1-amine trihydrochloride (compound 34)
N-[1-(1,3-benzodioxol-5-ylmethyl)piperidin-4-yl]-7-methoxy-4-[4-(2-methyl-2H-tetrazol-5-yl)phenyl]phthalazin-1-amine dihydrochloride (compound 35)
7-methoxy-4-(methoxymethyl)-N-(1-{4-[(1-methylpyrrolidin-3-yl)methoxy]benzyl}-piperidin-4-yl)phthalazin-1-amine trihydrochloride (compound 36)
7-methoxy-4-(methoxymethyl)-N-(1-{4-[(8-methyl-8-azabicyclo[3.2.1]oct-3-yl)-oxy]benzyl}piperidin-4-yl)phthalazin-1-amine trihydrochloride (compound 37)
2-[{4-[(4-{[7-methoxy-4-(methoxymethyl)phthalazin-1-yl]amino}piperidin-1-yl)-methyl]phenyl}(methyl)amino]ethanol trihydrochloride (compound 38)
7-methoxy-4-(methoxymethyl)-N-{1-[4-(2-oxo-2-pyrrolidin-1-ylethoxy)benzyl]-piperidin-4-yl}phthalazin-1-amine dihydrochloride (compound 39)
3-{4-[(4-{[7-methoxy-4-(methoxymethyl)phthalazin-1-yl]amino}piperidin-1-yl)-methyl]phenoxy}propan-1-ol dihydrochloride (compound 40)
7-methoxy-4-(methoxymethyl)-N-(1-{4-[(1-methylazepan-4-yl)oxy]benzyl}piperidin-4-yl)phthalazin-1-amine trihydrochloride (compound 41)
7-methoxy-4-(methoxymethyl)-N-(1-{4-[2-(1-methylpiperidin-2-yl)ethoxy]benzyl}-piperidin-4-yl)phthalazin-1-amine trihydrochloride (compound 42)
2-{4-[(4-{[7-methoxy-4-(methoxymethyl)phthalazin-1-yl]amino}piperidin-1-yl)-methyl]phenoxy}ethanol dihydrochloride (compound 43)
7-methoxy-4-methyl-N-(1-{4-[(1-methylazepan-4-yl)oxy]benzyl}piperidin-4-yl)-phthalazin-1-amine trihydrochloride (compound 44)
3-{3-[(4-{[7-methoxy-4-(methoxymethyl)phthalazin-1-yl]amino}piperidin-1-yl)-methyl]phenoxy}propan-1-ol dihydrochloride (compound 45)
3-[4-({4-[(7-methoxy-4-methylphthalazin-1-yl)amino]piperidin-1-yl}methyl)phenoxy]propan-1-ol dihydrochloride (compound 46)
7-chloro-4-methyl-N-(1-{4-[(1-methylazepan-4-yl)oxy]benzyl}piperidin-4-yl)-phthalazin-1-amine trihydrochloride (compound 47)
3-[4-({4-[(7-chloro-4-methylphthalazin-1-yl)amino]piperidin-1-yl}methyl)phenoxy]-propan-1-ol dihydrochloride (compound 48)
(3S)-1-{4-[(4-{[7-methoxy-4-(methoxymethyl)phthalazin-1-yl]amino}piperidin-1-yl)methyl]phenyl}pyrrolidin-3-ol trihydrochloride (compound 49)
(3R)-1-{4-[(4-{[7-methoxy-4-(methoxymethyl)phthalazin-1-yl]amino}piperidin-1-yl)methyl]phenyl}pyrrolidin-3-ol trihydrochloride (compound 50)
7-methoxy-4-(methoxymethyl)-N-{1-[4-(4-pyrrolidin-1-ylbutyl)benzyl]piperidin-4-yl}phthalazin-1-amine trioxalate (compound 53)
2-{3-[(4-{[7-methoxy-4-(4-methoxyphenyl)phthalazin-1-yl]amino}piperidin-1-yl)-methyl]phenoxy}ethanol dihydrochloride (compound 54)
2-{3-[(4-{[7-methoxy-4-(methoxymethyl)phthalazin-1-yl]amino}piperidin-1-yl)-methyl]phenoxy}ethanol dihydrochloride (compound 55)
3-{3-[(4-{[7-methoxy-4-(4-methoxyphenyl)phthalazin-1-yl]amino}piperidin-1-yl)-methyl]phenoxy}propan-1-ol dihydrochloride (compound 56)
3-{4-[(4-{[7-methoxy-4-(trifluoromethyl)phthalazin-1-yl]amino}piperidin-1-yl)-methyl]phenoxy}propan-1-ol dihydrochloride (compound 57)
7-methoxy-4-(methoxymethyl)-N-{1-[4-(octahydro-2H-pyrido[1,2-a]pyrazin-2-yl)-benzyl]piperidin-4-yl}phthalazin-1-amine trihydrochloride (compound 58)
3-[4-({4-[(7-methoxyphthalazin-1-yl)amino]piperidin-1-yl}methyl)phenoxy]propan-1-ol dihydrochloride (compound 63)
7-Methoxy-4-(methoxymethyl)-N-{1-[4-(6-methyloctahydro-1H-pyrrolo[3,4-b]pyridin-1-yl)benzyl]piperidin-4-yl}phthalazin-1-amine trihydrochloride (compound 64)
7-methoxy-4-(4-methoxyphenyl)-N-{1-[4-(7-methyl-2,7-diazaspiro[4.4]non-2-yl)-benzyl]piperidin-4-yl}phthalazin-1-amine trihydrochloride (compound 65)
4-{4-[(4-{[7-methoxy-4-(methoxymethyl)phthalazin-1-yl]amino}piperidin-1-yl)-methyl]phenyl}butan-1-ol dihydrochloride (compound 66)
7-methoxy-4-(methoxymethyl)-N-[1-({1-[4-(trifluoromethyl)phenyl]-1H-pyrrol-3-yl}methyl)piperidin-4-yl]phthalazin-1-amine dihydrochloride (compound 67)
N-(1-{4-[2-(1H-imidazol-1-yl)ethoxy]benzyl}piperidin-4-yl)-7-methoxy-4-(4-meth is oxyphenyl)phthalazin-1-amine trihydrochloride (compound 68)
7-methoxy-4-(4-methoxyphenyl)-N-{1-[4-(pyridin-4-ylmethoxy)benzyl]piperidin-4-yl}phthalazin-1-amine trihydrochloride (compound 69)
7-methoxy-4-(4-methoxyphenyl)-N-{1-[4-(tetrahydrofuran-2-ylmethoxy)benzyl]-piperidin-4-yl}phthalazin-1-amine dihydrochloride (compound 70)

In accordance with the invention, the compounds of general formula (I) may be prepared according to the process illustrated by Scheme 1 below.

In the schemes which follow, the starting compounds and the reagents, when their mode of preparation is not described, are commercially available or described in the literature, or alternatively may be prepared according to methods described therein or that are known to those skilled in the art.

The compound of general formula (I) may be obtained according to Scheme 1 below.

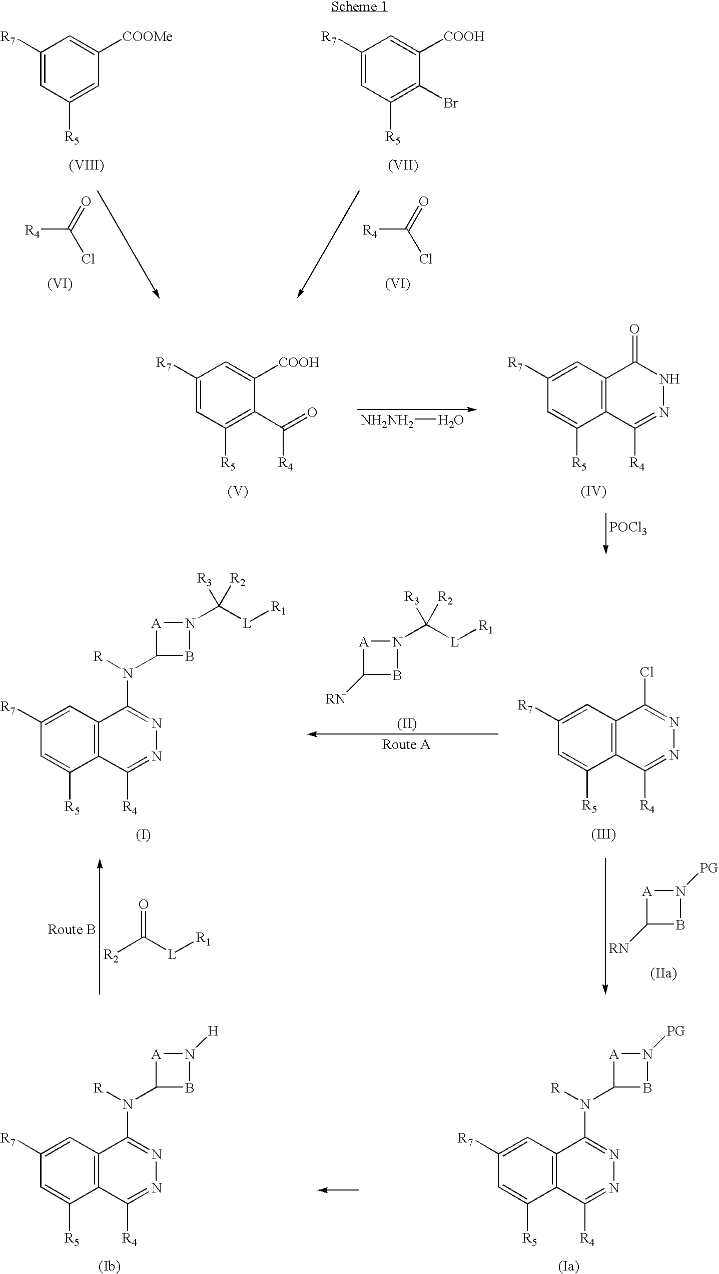

According to Scheme 1, the compound of general formula (I) is prepared from the compound of general formula (III), in which R₄, R₅ and R₇ are as defined in the general formula (I), according to route A, by nucleophilic substitution of the chlorine with the amine of general formula (II), in which R, R₁, R₂, R₃, L, A and B are as defined in the general formula (I). This reaction may be performed by heating the compounds of general formulae (II) and (III) in an alcohol such as n-butanol in the presence of ammonium chloride, according to the method described by Contreras et al. (J. Med. Chem. 2001, 44, 2707-2718), or alternatively may be catalyzed with a transition metal such as palladium, for example in the form of tris(dibenzylideneacetone)dipalladium in the presence of a ligand such as BINAP (2,2′-bis(diphenylphosphino)-1,1′-binaphthyl) and a base such as sodium or potassium tert-butoxide using toluene or dimethoxyethane as solvent, optionally in the presence of lithium bromide, according to the method described by Wolfe et al. (J.A.C.S., 1996, 118, 7215-7216), optionally modified (Tet. Lett. 1997, 38 (12), 2073-2074; Angew. Chem. Int. Ed. 2005, 44, 1371-1375.

When R₃ is a hydrogen atom, the compound of general formula (I) may also be prepared, according to route B, from the compound of general formula (Ib), in which A, B, R, R₄, R₅ and R₇ are as defined in the general formula (I) and in which the nitrogen of the nitrogenous ring is not substituted, via a reductive amination reaction with an aldehyde or a ketone of general formula R₁-L-C(O)R₂, in which L, R₁ and R₂ are as defined in the general formula (I). This reaction may be performed, for example, in the presence of sodium triacetoxyborohydride in a solvent, for instance dichloromethane or 1,2-dichloroethane according to one of the methods described in Abdel-Magid et al. (J. Org. Chem. 1996, 61, 3849-3862). The compound of general formula (Ib) is obtained by deprotection of the compound of general formula (Ia) that contains on the nitrogen of the nitrogenous ring a protecting group PG. This protecting group may be, for example, a benzyl, an ethoxycarbonyl or a tert-butoxycarbonyl and the deprotection may be performed according to the methods cited in Protective Groups in Organic Synthesis 3rd edition, John Wiley & Sons, New York 1999. The compound of general formula (Ia) is synthesized by reacting a compound of general formula (III) as defined above with a compound of general formula (IIa) according to the processes already described previously for the compound (I).

The amine of general formula (IIa) in which R represents a hydrogen atom, when it is not commercial, may be prepared by analogy with the methods described in the literature (Mach et al., J. Med. Chem. 1993, 36, 3707-3720, Dostert et al., Eur. J. Med. Chem. Ther. 1984, 19(2), 105-110). The amine of formula (II) in which R represents a hydrogen atom, when it is not commercial, may be prepared by analogy with the methods mentioned previously for (IIa) and also the methods described in the following publications: Moragues et al. (Farmaco. Ed. Sci. 1980, 35(11), 951-964) and Shum et al. (Nucleosides Nucleotides 2001, 20(4-7), 1067-1078).

The amines of formula (II) or (IIa) for which R represents a methyl may be prepared by reduction with lithium aluminum hydride (LiAlH₄) of the tert-butoxycarbonyl group introduced beforehand onto the primary amine according to the reduction method used by Gibson et al. (Tetrahedron Asymmetry 1995, 6, 1553-1556).

The amines of formula (II) or (IIa) for which R represents an alkyl other than methyl may be prepared by reduction of an amide (introduced beforehand onto the primary amine) with lithium aluminum hydride (LiAlH₄) or with the borane-tetrahydrofuran complex (BH₃-THF). For example, when R represents an ethyl group, the primary amine is acylated with acetyl chloride and the acetamide obtained is then reduced with lithium aluminum hydride.

The 1-chlorophthalazine of general formula (III) is obtained by heating the 2H-phthalazin-1-one of general formula (IV) in phosphoryl chloride, for example.

The 2H-phthalazin-1-one of general formula (IV) is prepared from the acid of 2-acylbenzoic type of general formula (V) by heating in an alcohol, for instance ethanol, in the presence of hydrazine hydrate, for example.

The acid of 2-acyl-benzoic type of general formula (V) may be synthesized from an acid of 2-bromobenzoic type of general formula (VII) via halogen-metal exchange followed by reaction with an acid chloride of general formula (VI) or with a Weinreb amide, of N-methoxy, N-methylamine type of formula (VI′):

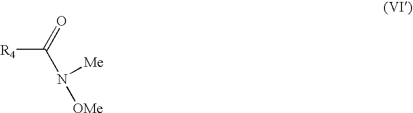

or an ester, such as for example ethyl trifluoroacetate. The halogen-metal exchange may be performed with n-butyllithium in tetrahydrofuran at −78° C. When R₄ represents a hydrogen atom, the acid chloride of general formula (VI) is replaced with dimethyl-formamide. The compounds of formula (VI′) may be obtained from the compound of formula (VI) and N-methoxy-N-methylamine, especially according to the method described by Weinreb (Tetrahedron Letters, (1981), 22(39): 3815-3818).

The acid of 2-acylbenzoic type of general formula (V) may also be obtained via other reactions, for instance the Friedel-Crafts reaction on a compound of general formula (VIII) in the presence of a compound of general formula (VI). This reaction is performed in the presence of aluminum chloride in a solvent such as dichloromethane.

The compound of general formula (I) may also be obtained according to scheme 2 below.

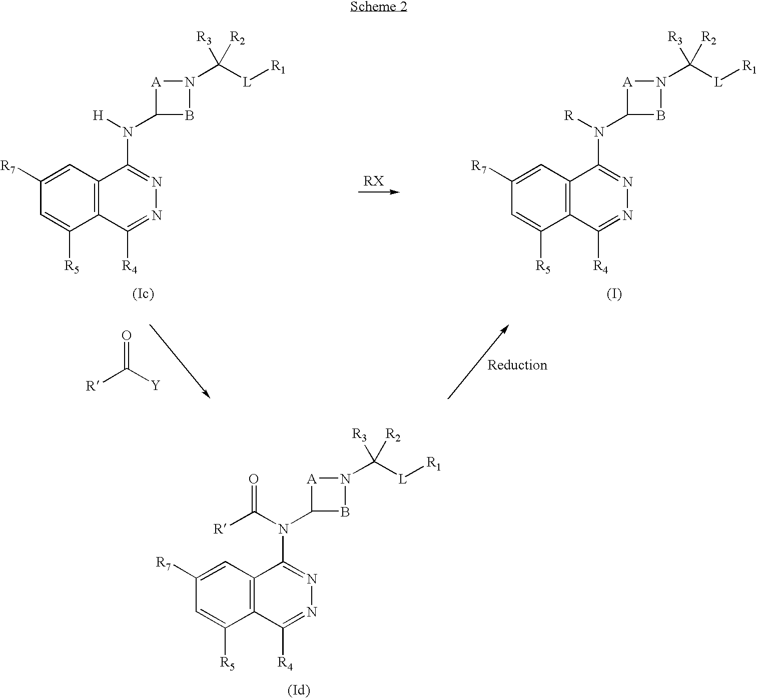

According to scheme 2, the compound of general formula (I) in which R is other than a hydrogen atom may also be prepared from the compound of general formula (Ic) in which R is a hydrogen atom, by alkylation with an alkyl halide (RX) in the presence of a base, for instance sodium hydride, in a solvent such as tetrahydrofuran. The compound of general formula (Ic) may also be acylated with an anhydride or an acid chloride of the type R′C(O)Y to form an amide of general formula (Id). This amide may be reduced with lithium aluminum hydride (LiAlH₄) or with the borane-tetrahydrofuran complex (BH₃-THF) to obtain a compound of general formula (I) in which R represents an alkyl group.

The compounds of formula (I) show high affinity and selectivity for the Melanin-Concentrating Hormone (MCH) receptor 1, MCH₁.

in vitro tests demonstrated the affinity of the compounds for the MCH receptors and in particular MCH₁.

Since MCH is an important regulator of food intake, small non-peptide molecules capable of antagonizing its stimulatory action on the MCH₁ receptor constitute a therapy of choice for treating metabolic problems caused by obesity, but also bulimia. Specifically, the use of an MCH₁ receptor antagonist such as SNAP-7941 (described by Synaptic Laboratories) confirms the important role of MCH in regulating the energy balance and the development of obesity (Katsuura et al., Curr. Med. Chem. 2003; 3: 217-227).

The compounds according to the invention thus represent a therapy of choice for the treatment of diseases presenting disorders of regulation of the energy balance and also for treating the development of obesity.

MCH is a functional antagonist of the melanocortin system, which counteracts the effects of the latter on food intake and on the hypothalamo-pituitary-adrenal axis (Ludwig et al., Am. J. Physiol. 1998; 274: E627-E633). It is also involved in regulating the hypothalamo-pituitary-adrenal axis and in the response to stress via the release of hypothalamic CRF (Kennedy et al., J. Neuroendocrinol. 2003; 15(3): 268-272). The use of an MCH₁ receptor antagonist has recently confirmed the anxiogenic effect of MCH. Specifically, SNAP-7941 has an anxiolytic and/or antidepressant profile in various animal models such as social conflict and forced swimming in rats and also maternal separation in guinea pigs (Katsuura et al., Curr. Med. Chem. 2003; 3: 217-227). MCH₁ receptor antagonist molecules are thus of therapeutic value in depression and/or anxiety.

MCH appears to be involved in other regulatory systems. By virtue of its localization in the testicles (Hervieu et al., Biology of Reproduction 1996; 5: 1161-1172) and in the hypothalamus (estrogen-dependent, Viale et al., Peptides 1999; 20: 553-559) and by virtue of its stimulatory effects on sexual activity in male rats (Gonzales et al., Peptides 1996; 17: 171-177) and its effects on the secretion of luteinizing hormone (Chiocchio et al., Biology of Reproduction 2001; 64: 1466-1472), MCH thus appears to play a role in reproductive functions.

It has also been observed that MCH is involved in behavior associated with cognitive functions by increasing the extinction of passive avoidance in rats, suggesting that an MCH₁ receptor antagonist might be useful in the case of memory disorders (MacBride et al., Peptides 1994; 15(4): 757-759). Thus, the compounds according to the invention may constitute a therapy of choice for treating memory disorders.

Finally, it has also been shown that MCH plays a role in urinary disorders and especially urinary incontinence (US2004/0038855A1).

Thus, the compounds of the invention find their use in therapy, especially in the treatment of obesity, cellulite, urinary incontinence, metabolic disorders and associated pathologies thereof such as diabetes, cardiovascular disorders, syndrome X, in the treatment of stress-related pathologies such as anxiety and depression, and also in the treatment of any other disease involving dysfunction related to the MCH₁ receptor, whether at the central and/or peripheral level.

Thus, according to another of its aspects, a subject of the invention is medicaments comprising a compound of formula (I) or an addition salt thereof with a pharmaceutically acceptable acid, or alternatively a hydrate or a solvate.

According to another of its aspects, the present invention relates to pharmaceutical compositions comprising, as active principle, a compound according to the invention. These pharmaceutical compositions comprise an effective dose of at least one compound according to the invention, or a pharmaceutically acceptable salt, a hydrate or solvate of the said compound, and also at least one pharmaceutically acceptable excipient. The said excipients are chosen according to the desired pharmaceutical form and mode of administration, from the usual excipients known to those skilled in the art.

In the pharmaceutical compositions of the present invention for oral, sublingual, subcutaneous, intramuscular, intravenous, topical, local, intratracheal, intranasal, transdermal or rectal administration, the active principle of formula (I) above, or the possible salt, solvate or hydrate thereof, may be administered in unit administration form, as a mixture with standard pharmaceutical excipients, to man and animals for the prophylaxis or treatment of the above disorders or diseases.

The appropriate unit administration forms comprise oral forms such as tablets, soft or hard gel capsules, powders, granules and oral solutions or suspensions, sublingual, buccal, intratracheal, intraocular and intranasal administration forms, forms for administration by inhalation, topical, transdermal, subcutaneous, intramuscular or intravenous administration forms, rectal administration forms and implants. For topical application, the compounds according to the invention may be used in creams, gels, ointments or lotions.

By way of example, a unit administration form of a compound according to the invention in tablet form may comprise the following components:

| | | |
|---|---|---|
| Compound according to the invention | 50.0 | mg |
| Mannitol | 223.75 | mg |
| Sodium croscarmellose | 6.0 | mg |
| Corn starch | 15.0 | mg |
| Hydroxypropylmethylcellulose | 2.25 | mg |
| Magnesium stearate | 3.0 | mg |

The said unit forms are dosed to allow a daily administration of from 0.5 mg to 800 mg and more particularly from 0.5 mg to 200 mg of active principle per individual, depending on the galenical form.

There may be cases where higher or lower doses are appropriate: such doses are not outside the context of the invention. According to the usual practice, the appropriate dose for each patient is determined by the doctor according to the mode of administration, the weight and the response of the said patient.

According to another of its aspects, the present invention also relates to a method for treating the pathologies indicated above, which comprises the administration, to the patient, of an effective dose of a compound according to the invention, or of a pharmaceutically acceptable salt, hydrate or solvate thereof.

The examples that follow describe the preparation of compounds in accordance with the invention. These examples are not limiting and serve merely to illustrate the present invention. The numbers of the illustrated compounds refer to those given in Table I.

The abbreviations and symbols used for the description of the synthetic procedures and for the description of the compounds are as follows:
DMF for dimethylformamide,
DMSO for dimethyl sulfoxide,
THF for tetrahydrofuran,
HCl for hydrochloric acid,
HBr for hydrobromic acid,
NaOH for sodium hydroxide,
Et for ethyl,
Me for methyl,
TFA for trifluoroacetic acid
NMP for N-methylpyrrolidinone

The melting points (m.p.) are expressed in degrees Celsius. They are measured either with a Köfler machine (indicated by (K) hereinbelow), or with a Mettler-Toledo FP62 machine (indicated by (M) hereinbelow), or with a Büchi B540 machine (indicated by (B) hereinbelow).

The analysis conditions by liquid chromatography coupled to mass spectrometry (LC/UV/MS) are as follows:
For the liquid chromatography part, 3 types of chromatographic system at different pH are used:

chromatographic systems at pH 3.1 (±0.1)
symmetry C18 (2.1×50 mm) 3.5 μm column, No. WAT200650
Eluent A=H₂O+0.005% TFA
Eluent B=CH₃CN+0.005% TFA.
gradient from 100% A to 90% B over 10 minutes, then elution with 90% B for 5 minutes.
flow rate 0.4 ml/min
injection of 2 μL of solution at a concentration of 0.5 mg/ml in methanol

chromatographic systems at pH 2.2 (±0.1)
Xterra MS C18 (2.1×50 mm) 3.5 μm column, No. 186000400
Eluent A=H₂O+0.1% HFBA (heptafluorobutyric acid)
Eluent B=90% CH₃CN+10% H₂O TFA+0.1% HFBA.
Gradient: 100% A for 2 minutes then from 100% A to 100% B over 10 minutes, then elution with 100% B for 5 minutes.
Flow rate 0.4 ml/minute
Injection of 2 μL of solution at a concentration of 0.5 mg/ml in methanol

chromatographic systems at pH 7 (±0.1)
Xterra MS C18 (2.1×50 mm) 3.5 μm column, No. 186000400
Eluent A=aqueous 0.01 M ammonium acetate solution
Eluent B=CH₃CN
Gradient from 100% A to 90% B over 10 minutes, then elution with 90% B for 5 minutes.
Flow rate 0.4 ml/minute; temperature 30° C.
Injection of 2 μL of solution at a concentration of 0.5 mg/ml in methanol

The products are detected by UV at 220 nm or at 210 nm.

For the mass spectrometry section:
ionization mode: positive electrospray (API-ES polarity+)
scanning from 120 to 1500 amu

The LC/MS analytical characteristics of the products are the m/z ratio of the ion MH⁺ or of the ion MNa⁺ and the retention time (Rt) of the corresponding peak, observed by UV and expressed in minutes. Certain polynitrogen products may be eluted on the HPLC column in two forms, one of which is in significantly major amount, depending on the degree of salification: in this case, both retention times are noted.

The proton nuclear magnetic resonance (¹H NMR) spectra were acquired at 250 MHz, 300 MHz, 400 MHz or 500 MHz on Brüker machines. The abbreviations used to characterize the signals are as follows: s=singlet, m=multiplet, d=doublet, t=triplet, q=quartet.

The quantification of the salts and solvates is determined by means of elemental analysis, assay of the water by the Karl-Fischer technique and integration of the characteristics signals of the solvents by ¹H NMR.

The compounds of the invention and their analytical characteristics (m.p., LC/MS, salts and solvates) are indicated in Table I.

### EXAMPLE 1

### N-[1-(1,3-Benzodioxol-5-ylmethyl)piperidin-4-yl]-4-(1-benzylpiperidin-4-yl)-7-methoxyphthalazin-1-amine Trihydrochloride (Compound 1)

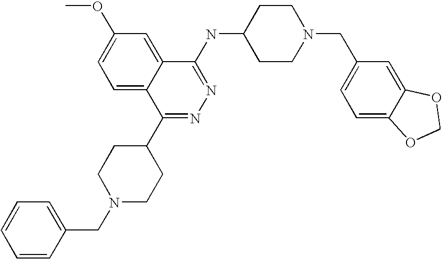

### 1.1 tert-Butyl[1-(1,3-benzodioxol-5-ylmethyl)piperidin-4-yl]carbamate

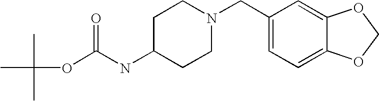

To a solution of 9.36 g (46.8 mmol) of tert-butyl (piperidin-4-yl)carbamate in 170 mL of 1,2-dichloroethane are added 7.02 g (46.8 mmol) of piperonal. The reaction mixture is placed under a nitrogen atmosphere and stirred for 30 minutes at room temperature. 13.9 g (63.5 mmol) of sodium triacetoxyborohydride are then added portionwise. After stirring at room temperature overnight, the reaction mixture is hydrolyzed with water and 2N sodium hydroxide solution and then extracted with dichloromethane. The combined organic phases are washed with water and with brine, and then dried over anhydrous sodium sulfate and evaporated under reduced pressure. 12.9 g of a white solid are obtained.

m.p.=96° C. (M)

MH⁺=335 (Rt=5.16 minutes pH 3.1)

¹H NMR (DMSO-d₆, 250 MHz) δ ppm: 1.35-1.45 (m, 2H); 1.38 (s, 9H); 1.65-1.75 (m, 2H); 1.85-2.0 (m, 2H); 2.65-2.8 (m, 2H); 3.1-3.3 (m, 1H); 3.33 (s, 2H); 5.99 (s, 2H); 6.7-6.8 (m, 1H); 6.8-6.9 (m, 2H).

### 1.2. 1-(1,3-Benzodioxol-5-ylmethyl)piperidin-4-ylamine

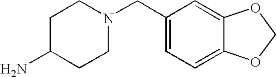

To a suspension of 12.9 g (38.8 mmol) of tert-butyl [1-(1,3-benzodioxol-5-ylmethyl)-piperidin-4-yl]carbamate in 130 mL of ethyl acetate are added 130 mL of a 2N solution of HCl in diethyl ether. The reaction mixture is stirred at room temperature for 12 hours. The precipitate formed is filtered off, rinsed with ethyl acetate and then dried in an oven (40° C.) under reduced pressure. 12.7 g of dihydrochloride are obtained in the form of a white solid.

A portion is converted into the corresponding base: 312 mg are stirred with 1.17 g (4 equivalents) of (polystyrylmethyl)trimethylammonium bicarbonate resin (Novabiochem) in 10 mL of dichloromethane for 5 hours. After filtering off the resin and rinsing with dichloromethane, the organic phase is evaporated. 174 mg of a white solid are obtained.

m.p.=69° C. (M)

¹H NMR (DMSO-d₆, 250 MHz) δ ppm: 1.10-1.30 (m, 2H); 1.60-1.75 (m, 2H); 1.85-2.0 (m, 2H); 2.45-2.55 (m, 1H); 2.65-2.75 (m, 2H); 2.7-3.4 (m, 2H, NH₂); 3.33 (s, 2H); 5.99 (s, 2H); 6.7-6.8 (m, 1H); 6.8-6.9 (m, 2H).

### 1.3. Methyl(piperidin-4-yl)carboxylate Hydrochloride

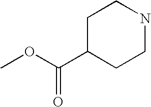

5.0 g (38.7 mmol) of isonipocotic acid are added slowly to a solution of 9.3 mL of thionyl chloride (129 mmol) in 50 mL of methanol, while keeping the temperature below −10° C. The mixture is stirred at room temperature for 22 hours. The reaction medium is evaporated under reduced pressure. 6.8 g of the expected derivative are obtained in the form of the hydrochloride.

¹H NMR (DMSO-d₆, 250 MHz) δ ppm: 1.7-1.85 (m, 2H); 1.9-2.05 (m, 2H); 2.6-2.75 (m, 1H); 2.8-3.0 (m, 2H); 3.15-3.3 (m, 2H); 3.64 (s, 3H); 8.9-9.3 (m, 2H, NH₂⁺).

### 1.4. Methyl (1-benzylpiperidin-4-yl)carboxylate

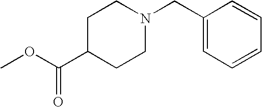

4.80 mL of benzyl bromide (37.9 mmol) are added slowly to a solution of 6.84 g (37.9 mmol) of methyl(piperidin-4-yl)carboxylate hydrochloride in 40 mL of dichloromethane. 10.7 mL (76.9 mmol) of triethylamine are then added slowly while maintaining the temperature at 10° C. After stirring overnight at room temperature, the reaction medium is hydrolyzed with water and saturated sodium hydrogen carbonate solution and is then extracted with dichloromethane. The combined organic phases are washed with water and with brine, and then dried over anhydrous sodium sulfate and evaporated under reduced pressure. The residue obtained is purified by chromatography on a column of silica gel (eluent: dichloromethane/methanol from 100/0 to 95/5 (v/v)). 7.03 g of product are obtained in the form of an orange oil.

MH⁺=234 (Rt=3.79 and 3.95 minutes, pH 3.1)

¹H NMR (DMSO-d₆, 250 MHz) δ ppm: 1.5-1.7 (m, 2H); 1.75-1.9 (m, 2H); 1.9-2.05 (m, 2H); 2.25-2.4 (m, 1H); 2.7-2.8 (m, 2H); 3.45 (s, 2H); 3.61 (s, 3H); 7.2-7.4 (m, 5H).

### 1.5 (1-Benzylpiperidin-4-yl)-N-methoxy-N-methylcarboxamide

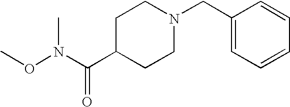

A solution of 4.17 g (42.8 mmol) of N,O-dimethylhydroxylamine hydrochloride in 195 mL of THF is cooled to −20° C., and 6.51 g (28 mmol) of methyl (1-benzylpiperidin-4-yl)carboxylate are added. Next, 85 mL of a 1M solution of isopropylmagnesium bromide in THF are added over 20 minutes while keeping the temperature in the region of 5° C. The mixture is stirred at −10° C. for 20 minutes. The reaction medium is hydrolyzed with 40 mL of 20% ammonium chloride solution and then extracted with ethyl acetate. The combined organic phases are washed with water and with brine, and then dried over anhydrous sodium sulfate and evaporated under reduced pressure. 6.9 g of an oily product are obtained.

¹H NMR (DMSO-d₆, 250 MHz) δ ppm: 1.5-1.75 (m, 4H); 1.9-2.05 (m, 2H); 2.55-2.7 (m, 1H); 2.8-2.9 (m, 2H); 3.09 (s, 3H); 3.46 (s, 2H); 3.67 (s, 3H); 7.2-7.4 (m, 5H).

### 1.6 2-[(1-Benzylpiperidin-4-yl)carbonyl]-5-ethoxybenzoic Acid

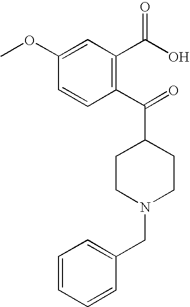

A solution of 2-bromo-5-methoxybenzoic acid 0.9 g (3.89 mmol) in 10 mL of THF is stirred under argon at −78° C., followed by dropwise addition of 7.4 mL (11.84 mmol) of a 1.6 M solution of n-butyllithium in hexane. The reaction mixture is stirred for one hour at −78° C., followed by dropwise addition of 0.97 g (3.7 mmol) of (1-benzylpiperidin-4-yl)-N-methoxy-N-methylcarboxamide in 5 mL of THF. The reaction mixture is stirred for one hour at −78° C. and then for 18 hours at room temperature. The mixture is hydrolyzed with 0.6 mL of water and then evaporated under reduced pressure. The residue obtained is purified by chromatography on a column of silica gel (solvent: dichloromethane/methanol from 100/0 to 85/15 (v/v)). 0.47 g of an oily product is obtained.

MH⁺=354 (Rt=5.08 minutes, pH 3.1)

¹H NMR (DMSO-d₆, 250 MHz) δ ppm: 1.3-1.5 (m, 2H); 1.6-1.75 (m, 2H); 1.85-2.0 (m, 2H); 2.3-2.5 (m, 1H); 2.75-2.9 (m, 2H); 3.45 (s, 2H); 3.85 (s, 3H); 7.15-7.45 (m, 8H).

### 1.7 4-(1-Benzylpiperidin-4-yl)-7-methoxy-2H-phthalazin-1-one

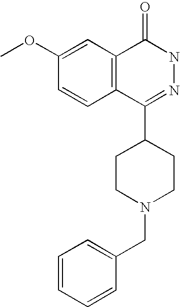

To a solution of 3.49 g (9.05 mmol) of 2-[(1-benzylpiperidin-4-yl)carbonyl]-5-methoxybenzoic acid in 45 mL of ethanol are added 4.9 mL (45 mmol) of hydrazine hydrate and the mixture is then stirred at reflux for 2 hours. The reaction medium is cooled and then filtered. The precipitate obtained is washed with methanol and then dried under reduced pressure at 40° C. 2 g of product are obtained in the form of a white solid.

m.p.=262° C. (M)

MH⁺=350 (Rt=5.06 minutes, pH 3.1)

¹H NMR (DMSO-d₆, 250 MHz) δ ppm: 1.7-1.9 (m, 4H); 2.1-2.25 (m, 2H); 2.9-3.0 (m, 2H); 3.1-3.25 (m, 1H); 3.53 (s, 2H); 3.94 (s, 3H); 7.2-7.4 (m, 5H); 7.5 (dd, J₁=7.5 Hz, J₂=2.5 Hz, 1H); 7.67 (d, J=2.5 Hz. 1H); 8.02 (d, J=7.5 Hz. 1H); 12.40 (s, 1H, NH).

### 1.8 4-(1-Benzylpiperidin-4-yl)-1-chloro-7-methoxyphthalazine

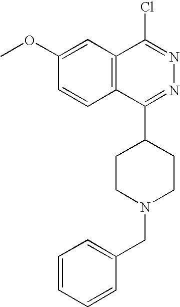

350 mg (1 mmol) of 4-(1-benzylpiperidin-4-yl)-7-methoxy-2H-phthalazin-1-one are dissolved in 5 mL of phosphoryl chloride. The solution is heated for 3 hours at 80° C. The reaction medium is cooled to room temperature and is then poured slowly into 200 mL of water with stirring. The mixture is then stirred at 5° C. and neutralized with 35% sodium hydroxide solution. The precipitate formed is filtered off, washed with water and dried in an oven. 370 mg of product are obtained in the form of a powder.

MH⁺=368 (Rt=5.57 minutes)

¹H NMR (DMSO-d₆, 250 MHz) δ ppm: 2.05-2.2 (m, 2H); 2.25-2.45 (m, 2H); 3.05-3.60 (m, 4H); 3.8-3.95 (m. 1H); 4.04 (s, 3H); 4.35 (s, 2H); 7.45-7.6 (m, 4H); 7.6-7.7 (m, 2H); 7.75 (dd, J₁=8.5 Hz, J₂=2.5 Hz, 1H); 8.45 (d, J=8.5 Hz, 1H).

### 1.9. N-[1-(1,3-Benzodioxol-5-ylmethyl)piperidin-4-yl]-4-(1-benzylpiperidin-4-yl)-7-methoxyphthalazin-1-amine Trihydrochloride (Compound 1)

To a suspension of 370 mg (1 mmol) of 4-(1-benzylpiperidin-4-yl)-1-chloro-7-methoxyphthalazine in 5 mL of n-butanol are added 234 mg (1 mmol) of 1-(1,3-benzodioxol-5-ylmethyl)piperidin-4-ylamine and 53 mg (1 mmol) of ammonium chloride. The mixture is heated at 140° C. for 18 hours. The reaction medium is cooled to room temperature, hydrolyzed with water and then basified to pH 10 with 1 N sodium hydroxide. The mixture is then extracted with ethyl acetate. The organic phase is washed with brine, dried over anhydrous sodium sulfate and then evaporated under reduced pressure. The residue obtained is purified by chromatography on a column of silica gel (solvent: dichloromethane/methanol from 100/0 to 85/15 (v/v)). 150 mg of an oily product are obtained.

MH⁺=566 (Rt=4.50 minutes, pH3.1)

¹H NMR (DMSO-d₆, 400 MHz) δ ppm: 1.6-1.7 (m, 2H); 1.75-1.85 (m, 2H); 1.85-2.15 (m, 6H); 2.15-2.3 (m, 2H); 2.85-3.0 (m, 2H); 3.2-3.45 (m, 3H); 3.45 (s, 2H); 3.55 (s, 2H); 3.94 (s, 3H); 4.1-4.2 (m, 1H); 6.00 (s, 2H); 6.75-6.9 (m, 4H); 7.2-7.3 (m, 1H); 7.3-7.4 (m, 4H); 7.45 (dd, J₁=8.5 Hz, J₂=2.5 Hz, 1H), 7.68 (d, J=2.5 Hz, 1H); 8.0 (d, J=8.5 Hz, 1H).

The trihydrochloride is obtained after treating the product dissolved in dichloromethane with a solution of hydrogen chloride in diethyl ether. After concentrating and adding diisopropyl ether, a suspension is obtained, which is filtered. The powder obtained is dried at 50° C. under reduced pressure.

### EXAMPLE 2

### 7-Methoxy-4-(4-methoxyphenyl)-N-methyl-N-[1-(2-naphthylmethyl)piperidin-4-yl]-phthalazin-1-amine dihydrochloride (Compound 2)

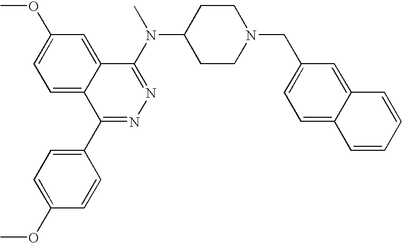

### 2.1. tert-Butyl[1-(2-naphthylmethyl)piperidin-4-yl]carbamate

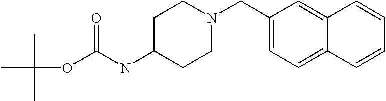

To a solution of 15 g (75 mmol) of tert-butyl(piperidin-4-yl)carbamate in 300 mL of 1,2-dichloroethane are added 11.7 g (75 mmol) of 2-naphtaldehyde. The reaction mixture is placed under a nitrogen atmosphere and stirred for 45 minutes at room temperature. Next, 20.6 g (97 mmol) of sodium triacetoxyborohydride are introduced portionwise. After stirring at room temperature overnight, the reaction mixture is hydrolyzed with water and basified with about 150 mL of aqueous 1 N sodium hydroxide solution. After extracting with dichloromethane, the combined organic phases are washed with water and with brine, dried over anhydrous magnesium sulfate and then under reduced pressure. The residue obtained is purified by chromatography on a column of silica gel (eluent: dichloromethane/methanol from 100/0 to 90/10 (v/v)). 22 g of a white solid are obtained.

LC/MS: MH⁺=341 (Rt=5.90 minutes, pH 3.1)

¹H NMR (DMSO-d₆, 300 MHz) δ ppm: 1.39 (s, 9H); 1.3-1.45 (m, 2H); 1.6-1.7 (m, 2H); 1.9-2.05 (m, 2H); 2.75-2.85 (m, 2H); 3.15-3.3 (m, 1H); 3.58 (s, 2H); 6.70-6.75 (m, 1H, NH); 7.45-7.55 (m, 3H); 7.75 (m, 1H); 7.85-7.95 (m, 3H).

### 2.2. N-Methyl-N-[1-(2-naphthylmethyl)piperidin-4-yl]amine

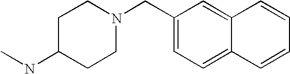

A solution of 1 g (2.94 mmol) of [1-(2-naphthylmethyl)piperidin-4-yl]carbamate tert-butyl in 5 mL of THF is added slowly to a suspension of 223 mg (5.9 mmol) of lithium aluminum hydride in 10 mL of THF at room temperature. The reaction medium is refluxed for 3 hours. It is then stirred at 0° C. and hydrolyzed with 0.2 mL of water, 0.2 mL of 1 N sodium hydroxide and then 0.6 mL of water. The gel obtained is filtered off and rinsed with dichloromethane. The filtrate is dried over anhydrous sodium sulfate and then evaporated under reduced pressure. 0.7 g of oil is obtained.

LC/MS: MH⁺=255 (Rt=2.84 minutes, pH 3.1)

¹H NMR (DMSO-d₆, 300 MHz) δ ppm: 1.15-1.3 (m, 2H); 1.7-1.8 (m, 2H); 1.9-2.05 (m, 2H); 2.15-2.3 (m, 1H); 2.24 (s, 3H); 2.7-2.8 (m, 2H); 3.3-3.4 (m, 1H); 3.58 (s, 2H); 7-4-7.55 (m, 3H); 7.75 (m, 1H); 7.85-7.95 (m, 3H).

### 2.3. N-4-Dimethoxy-N-methylbenzamide

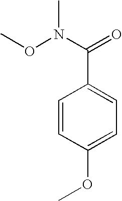

To a solution of 25 g (147 mmol) of 4-methoxybenzoyl chloride in 450 mL of dichloromethane, stirred at 0-5° C. under nitrogen, are added portionwise 14.7 g (151 mmol) of N₇O-dimethylhydroxylamine hydrochloride. 51 mL (370 mmol) of triethylamine are then added slowly to the mixture stirred at 0-5° C. The reaction medium is stirred at room temperature for 3 hours. The mixture is hydrolyzed with 250 mL of water and is then extracted with dichloromethane. The organic phase is washed with 100 mL of 1 N HCl, 150 mL of 1 N NaOH and then with water and with brine. It is dried over anhydrous sodium sulfate, filtered and evaporated to dryness. 26.9 g of an orange oil are obtained, which are purified on a column of silica (eluent: dichloromethane/meth-anol from 100/0 to 95/5 (v/v)). 25.4 g of a yellow oil are obtained.

LC/MS: MH⁺=196 (Rt=5.21 minutes, pH 3.1)

¹H NMR (DMSO-d₆, 250 MHz) δ ppm: 3.26 (s, 3H); 3.56 (s, 3H); 3.82 (s, 3H); 7.00 (d, J=8.5 Hz, 2H); 7.63 (d, J=8.5 Hz, 2H).

### 2.4. 5-Methoxy-2-(4-methoxybenzoyl)benzoic Acid

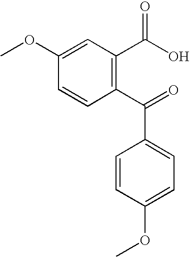

A solution of 18.5 g (80 mmol) of 2-bromo-5-methoxybenzoic acid in 150 mL of THF is stirred under nitrogen at −78° C. 100 mL (160 mmol) of a 1.6 M solution of n-butyllithium in hexane are added dropwise over about one hour while taking care to ensure that the temperature does not exceed −70° C. After the addition, the mixture is stirred at −78° C. for one hour, and a solution of 15.9 g (80 mmol) of N-4-dimethoxy-N-methylbenzamide in 20 mL of THF is then added dropwise. The reaction medium is stirred at −78° C. for one hour and then at room temperature for 18 hours. The mixture is hydrolyzed with 100 mL of water, basified to pH=12 with 2 N NaOH solution and extracted with tert-butyl methyl ether. The aqueous phase containing the carboxylate is acidified with 5 N HCl solution to pH=1 and extracted with dichloromethane. The dichloromethane phase is washed with brine, dried over anhydrous sodium sulfate, filtered and evaporated. The product is crystallized from isopropyl ether; after filtering off and drying, 14.6 g of white crystals are obtained.

m.p.=170° C. (M)

LC/MS: MH⁺=287 (Rt=7.07 minutes, pH 3.1)

¹H NMR (DMSO-d₆, 250 MHz) δ ppm: 3.85 (s, 3H); 3.90 (s, 3H); 7.03 (d, J=8.5 Hz, 2H); 7.24 (dd, J₁=8.5 Hz, J₂=2.5 Hz, 1H); 7.35 (d, J=8.5 Hz, 1H); 7.42 (d, J=2.5 Hz, 1H); 7.61 (d, J=8.5 Hz, 2H); 13.1 (s, 1H, COOH).

### 2.5. 7-Methoxy-4-(4-methoxyphenyl)-2H-phthalazin-1-one

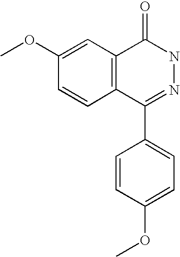

To a solution of 21.6 g (75 mmol) of 5-methoxy-2-(4-methoxybenzoyl)benzoic acid in 150 mL of ethanol are added 18.5 mL (380 mmol) of hydrazine hydrate and the mixture is refluxed for 2 hours. The reaction medium is cooled and then filtered. The precipitate obtained is washed with methanol and then dried under reduced pressure at 40° C. 19 g of a white solid are obtained.

m.p.=245° C. (M)

LC/MS: MH⁺=283 (Rt=6.98 minutes, pH 3.1)

¹H NMR (DMSO-d₆, 250 MHz) δ ppm: 3.86 (s, 3H); 3.98 (s, 3H); 7.12 (d, J=8.5 Hz, 2H); 7.48 (dd, J₁=9 Hz, J₂=2.7 Hz, 1H); 7.52 (d, J=8.5 Hz, 2H); 7.67 (d, J=9.0 Hz, 1H); 7.75 (d, J=2.7 Hz, 1H); 12.72 (s, 1H, NH).

### 2.6. 1-Chloro-7-methoxy-4-(4-methoxyphenyl)phthalazine

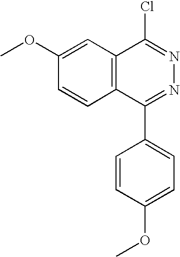

19 g (67 mmol) of 7-methoxy-4-(4-methoxyphenyl)-2H-phthalazin-1-one are dissolved in 100 mL of phosphoryl chloride. The solution is heated for 2 hours at 80° C. The reaction medium is cooled to room temperature and is then poured slowly into 500 mL of water at 40-50° C. with stirring. The mixture is then stirred at 5° C., neutralized by addition of 35% sodium hydroxide solution and then extracted with dichloromethane. The organic phase is washed with water and then with brine and dried over anhydrous sodium sulfate. After filtering and evaporating under reduced pressure, 19.3 g of a beige-colored powder are obtained.

m.p.=173° C. (M)

LC/MS: MH⁺=301 (Rt=8.52 minutes, pH 3.1)

¹H NMR (DMSO-d₆, 250 MHz) δ ppm: 3.90 (s, 3H); 4.07 (s, 3H); 7.19 (d, J=8.5 Hz, 2H); 7.60 (d, J=2.5 Hz, 1H); 7.68 (d, J=8.5 Hz, 2H); 7.72 (dd, J₁=9.2 Hz, J₂=2.5 Hz, 1H); 8.02 (d, J=9.2 Hz, 1H).

### 2.7. 7-Methoxy-4-(4-methoxyphenyl)-N-methyl-N-[1-(2-naphthylmethyl)piperidin-4-yl]phthalazin-1-amine dihydrochloride (Compound 2)

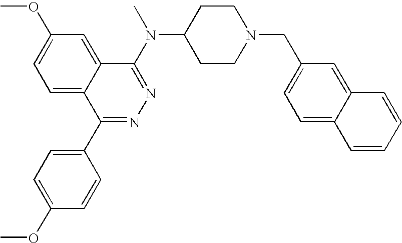

To a suspension of 830 mg (2.96 mmol) of 1-chloro-7-methoxy-4-(4-methoxyphenyl)phthalazine in 5 mL of 1-butanol are added 700 mg (2.75 mmol) of N-methyl-N-[1-(2-naphthylmethyl)piperidin-4-yl]amine and 147 mg (2.8 mmol) of ammonium chloride. The mixture is refluxed (water bath at 140° C.) for 27 hours. The reaction mixture is hydrolyzed and then basified with 1N sodium hydroxide solution, and then extracted with dichloromethane. The combined organic phases are washed with brine, dried over anhydrous sodium sulfate and then evaporated under reduced pressure. The residue obtained is purified by chromatography on a column of silica gel (eluent: dichloromethane/methanol from 100/0 to 90/10 (v/v)). 220 mg of an oily product are obtained.

The dihydrochloride is obtained after treating the product dissolved in ethanol with a solution of hydrogen chloride in diethyl ether. After concentrating and adding ethyl acetate, a suspension is obtained, which is filtered. The powder obtained is dried at 40° C. under reduced pressure in the presence of phosphorus pentoxide.

¹H NMR (DMSO-d₆, 250 MHz) δ ppm: 2.05-2.2 (m, 2H); 2.55-2.75 (m, 2H); 3.14 (s, 3H); 3.15-3.35 (m, 2H); 3-4-3.6 (m, 3H); 3.91 (s, 3H); 4.09 (s, 3H); 4.25-4.4 (m, 1H); 4-4-4.5 (m, 2H); 7.26 (d, J=8.7 Hz, 2H); 7.5-7.8 (m, 5H); 7.85 (d, J=7.5 Hz, 1H); 7.9-8.1 (m, 5H); 8.15 (m, 1H); 11.5 (m. 1H)

### EXAMPLE 3

### N-(1-[{4-[3-(Dimethylamino)propoxy]benzyl}piperidin-4-yl)-7-methoxy-4-(4-methoxy-phenyl)phthalazin-1-amine trihydrochloride (Compound 3)

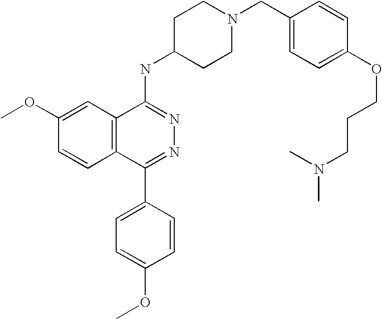

### 3.1. N-(1-Benzylpiperidin-4-yl)-7-methoxy-4-(4-methoxyphenyl)phthalazin-1-amine

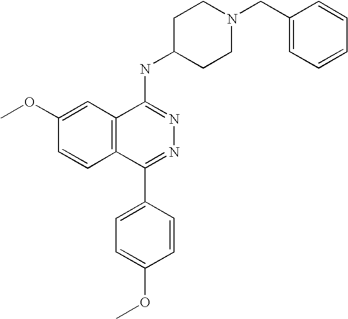

To a suspension of 8 g (26.6 mmol) of 1-chloro-7-methoxy-4-(4-methoxyphenyl)-phthalazine (preparation: see paragraph 2.6) in 50 mL of 1-butanol are added 10.1 g (53 mmol) of 4-amino-1-benzylpiperidine and 1.44 g (27 mmol) of ammonium chloride. The mixture is refluxed for 7 hours. The reaction medium is cooled to room temperature, hydrolyzed with 50 mL of saturated aqueous sodium hydrogen carbonate solution and then extracted with ethyl acetate. The organic phase is washed with brine, dried over anhydrous sodium sulfate and then evaporated under reduced pressure. The residue obtained is purified by chromatography on a column of silica gel (eluent: dichloromethane/methanol 95/5 (v/v)). The yellow oil thus obtained forms solid by trituration from isopropyl ether. After filtering off and drying, 10.5 g of a white powder are obtained.

m.p.=150° C. (M)

LC/MS: MH⁺=455 (Rt=4.71 minutes, pH 3.1)

¹H NMR (DMSO-d₆, 250 MHz) δ ppm: 1.7-1.8 (m, 2H); 2.0-2.2 (m, 4H); 2.90-3.0 (m, 2H); 3.54 (s, 2H); 3.86 (s, 3H); 4.0 (s, 3H); 4.2-4.3 (m, 1H); 7.0 (d, J=7.5 Hz. 1H, NH); 7.10 (d, J=8.5 Hz, 2H); 7.25-7.40 (m, 5H); 7.43 (dd; J₁=9.0 Hz; J₂=2.5 Hz, 1H); 7.53 (d, J=8.5 Hz, 2H); 7.75-7.77 (m, 2H).

### 3.2. 7-Methoxy-4-(4-methoxyphenyl)-N-(piperidin-4-yl)phthalazin-1-amine

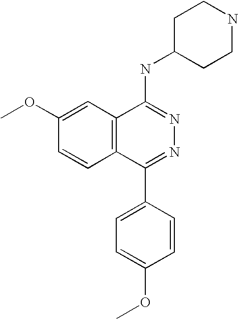

The manipulation is performed under an inert atmosphere (nitrogen). 1 g of 10% palladium-on-charcoal and 100 mL of water are placed in a 500 mL three-necked flask. To this mixture, stirred at 85° C., is added dropwise, over one hour, a solution prepared beforehand of 13.3 g of N-(1-benzylpiperidin-4-yl)-7-methoxy-4-(4-methoxyphenyl)-phthalazin-1-amine in 50 mL of ethanol and 4.4 mL of formic acid. The reaction mixture is stirred at reflux for 2 hours. After cooling to room temperature, the ethanol is evaporated off under reduced pressure. The mixture is then basified to pH 12 with 2 N sodium hydroxide and is then extracted with dichloromethane. The organic phase is washed with saturated aqueous sodium chloride, dried over anhydrous sodium sulfate and then evaporated under reduced pressure. The yellow oil obtained is triturated from diisopropyl ether to give a white solid. After filtering off and drying under vacuum in the presence of phosphorus pentoxide, 10.29 g of product are obtained.

m.p.=143° C. (M)

LC/MS: MH⁺=365 (Rt=4.88 minutes, pH 3.1)

¹H NMR (DMSO-d₆, 250 MHz) δ ppm: 1.5-1.65 (m, 2H); 2.0-2.1 (m, 2H); 2.6-2.7 (m, 2H); 3.0-3.1 (m, 2H); 3.3 (s, 1H, NH); 3.85 (s, 3H); 3.98 (s, 3H); 4.2-4.3 (m, 1H); 7.0 (d, J=7.5 Hz, 1H); 7.10 (d, J=8.5 Hz, 2H); 7.42 (dd, J₁=9.2 Hz, J₂=2.5 Hz, 1H); 7.52 (d, J=8.5 Hz, 2H); 7.75 (d, J=9.2 Hz, 1H); 7.78 (d, J=2.5 Hz, 1H).

### 3.3. N-(1-{4-[3-(Dimethylamino)propoxy]benzyl}piperidin-4-yl)-7-methoxy-4-(4-methoxyphenyl)phthalazin-1-amine trihydrochloride (compound 3)

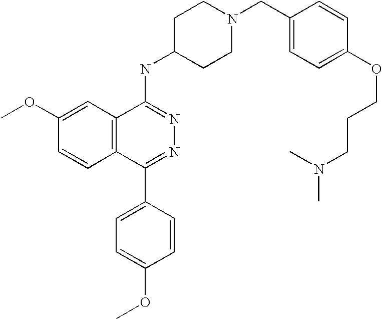

To a solution of 500 mg (1.4 mmol) of 7-methoxy-4-(4-methoxyphenyl)-N-(piperidin-4-yl)phthalazin-1-amine in 10 mL of 1,2-dichloroethane are added 284 mg (1.4 mmol) of 4-[3-(dimethylamino)propoxy]benzaldehyde. The mixture is stirred for 30 minutes under nitrogen and 380 mg (1.8 mmol) of sodium triacetoxyborohydride are then added. The reaction medium is stirred for 24 hours at room temperature, hydrolyzed with water and 1 N sodium hydroxide and then extracted with ethyl acetate. The organic extract is washed with saturated aqueous sodium chloride, dried over anhydrous sodium sulfate and then evaporated under reduced pressure. The residue is purified on a column of neutral alumina (eluent: dichloromethane/methanol from 100/0 to 98/2 (v/v)). 380 mg of brown product are obtained.

¹H NMR (DMSO-d₆, 250 MHz) δ ppm: 1.6-1.65 (m, 2H); 1.7-1.9 (m, 2H); 2.0-2.2 (m, 4H); 2.15 (s, 6H); 2.35 (t, J=5 Hz, 2H); 2.85-2.95 (m, 2H); 3.45 (s, 2H); 3.85 (s, 3H); 3.95-4.05 (m, 2H); 3.98 (s, 3H); 4.2-4.35 (m, 1H); 6.89 (d, J=8 Hz, 2H); 6.97 (d, J=7.5 Hz, 1H); 7.08 (d, J=8 Hz, 2H); 7.23 (d, J=8 Hz, 2H); 7.42 (dd, J₁=7.5 Hz, J₂=2.5 Hz, 1H); 7.52 (d, J=8 Hz, 2H); 7.7-7.75 (m, 2H);

The trihydrochloride is obtained after treating the product dissolved in dichloromethane with a solution of hydrogen chloride in diethyl ether. A suspension is obtained, which is filtered. The powder obtained is dried at 40° C. under reduced pressure in the presence of phosphorus pentoxide.

### EXAMPLE 4

### N-{1-[3-Fluoro-4-(3-morpholin-4-ylpropoxy)benzyl]piperidin-4-yl}-7-methoxy-4-(4-methoxyphenyl)phthalazin-1-amine trihydrochloride (compound 4)

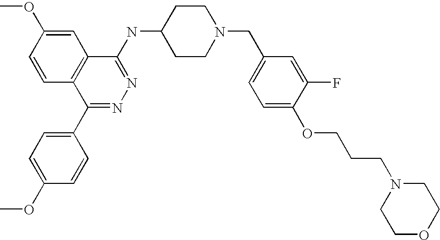

### 4.1. 4-(3-Chloropropyl)morpholine

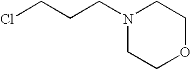

1 g (6.9 mmol) of 4-(3-hydroxypropyl)morpholine is stirred at 0° C. and 2.5 mL (34.4 mmol) of thionyl chloride are added. The mixture is stirred for 6 hours at room temperature, poured slowly onto an ice-cold solution of 1 N NaOH and then extracted with dichloromethane. The organic extract is dried over anhydrous sodium sulfate and then evaporated under reduced pressure. 1.1 g of an oily colorless product are obtained.

¹H NMR (DMSO-d₆, 250 MHz) δ ppm: 1.8-1.95 (m, 2H); 2.3-2.4 (m, 6H); 3.5-3.6 (m, 4H); 3.65-3.70 (m, 2H).

### 4.2. 3-Fluoro-4-(3-morpholin-4-ylpropoxy)benzaldehyde

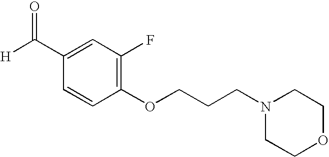

To a solution of 396 mg (2.4 mmol) of 4-(3-chloropropyl)morpholine and 339 mg (2.4 mmol) of 3-fluoro-4-hydroxybenzaldehyde in 10 mL of DMF are added 334 mg (2.4 mmol) of potassium carbonate. The mixture is stirred at 80° C. for 5 hours and, after cooling, water is added and the mixture is extracted with ethyl acetate. The organic extract is washed with water and then with saturated aqueous sodium chloride, dried over anhydrous sodium sulfate and then evaporated under reduced pressure. 400 mg of an oily product are obtained.

¹H NMR (DMSO-d₆, 250 MHz) δ ppm: 1.90-2.0 (m, 2H); 2.32-2.48 (m, 6H); 3.55-3.60 (m, 4H); 4.20-4.28 (m, 2H); 7.37-7.44 (m, 1H); 7.74 (d, J=11 Hz, 1H), 7.78 (d, J=7.5 Hz, 1H), 9.58 (s, 1H, CHO).

### 4.3. N-{1-[3-Fluoro-4-(3-morpholin-4-ylpropoxy)benzyl]piperidin-4-yl}-7-methoxy-4-(4-methoxyphenyl)phthalazin-1-amine trihydrochloride (compound 4)

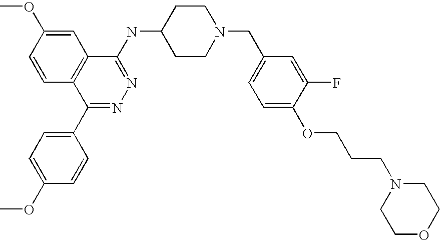

To a solution of 500 mg (1.37 mmol) of 7-methoxy-4-(4-methoxyphenyl)-N-(piperidin-4-yl)phthalazin-1-amine in 10 mL of 1,2-dichloroethane are added 367 mg (1.37 mmol) of 3-fluoro-4-(3-morpholin-4-ylpropoxy)benzaldehyde. The mixture is stirred for 30 minutes under nitrogen and 378 mg (1.78 mmol) of sodium triacetoxyborohydride are then added. The reaction medium is stirred overnight at room temperature and then hydrolyzed with water and 1 N sodium hydroxide and extracted with ethyl acetate. The organic extract is washed with saturated aqueous sodium chloride, dried over anhydrous sodium sulfate and then evaporated under reduced pressure. The residue is purified on a column of silica (eluent: dichloromethane/methanol from 100/0 to 85/15 (v/v)). 380 mg of a white solid are obtained.

¹H NMR (DMSO-d₆, 400 MHz) δ ppm: 1.62-1.75 (m, 2H); 1.87-1.94 (m, 2H); 2.05-2.16 (m, 4H); 2.32-2.45 (m, 6H); 2.87-2.93 (m, 2H); 3.46 (s, 2H); 3.55-3.60 (m, 4H); 3.84 (s, 3H); 3.97 (s, 3H); 4.05-4.10 (m, 2H); 4.20-4.30 (m, 1H); 6.97 (d, J=7.5 Hz, 1H); 7.05-7.18 (m, 5H); 7.42 (dd, J₁=7.5 Hz, J₂=2.5 Hz, 1H); 7.51 (d, J=8 Hz, 2H); 7.72-7.77 (m, 2H);

By treating the product dissolved in dichloromethane with a solution of hydrogen chloride in diethyl ether, a suspension is obtained, which is filtered. The powder obtained is dried at 40° C. under reduced pressure in the presence of phosphorus pentoxide.

### EXAMPLE 5

### 7-Methoxy-4-(4-methoxyphenyl)-N-{1-[4-(2-piperidin-1-ylethoxy)benzyl]piperidin-4-yl}phthalazin-1-amine trihydrochloride (compound 5)

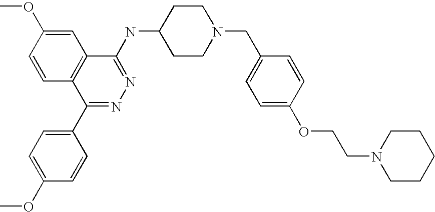

### 5.1. 4-(2-Piperidin-1-ylethoxy)benzaldehyde

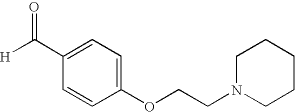

To a solution of 7.54 g (40.9 mmol) of N-(2-chloroethyl)piperidine hydrochloride and 5 g (40.9 mmol) of 4-hydroxybenzaldehyde in 100 mL of DMF are added 11.88 g (86 mmol) of potassium carbonate. The mixture is stirred at 80° C. for 5 hours 30 and, after cooling, is then poured into water and extracted with ethyl acetate. The organic extract is washed with water and then with saturated aqueous sodium chloride, dried over anhydrous sodium sulfate and then evaporated under reduced pressure. 10.5 g of an oily orange-colored product containing 0.3 equivalent of DMF are obtained.

MH⁺=234 (Rt=3.89 minutes, pH 3.1).

¹H NMR (DMSO-d₆, 250 MHz) δ ppm: 1.32-1.45 (m, 2H); 1.45-1.55 (m, 4H); 2.40-2.48 (m, 4H); 2.65-2.72 (m, 2H); 4.17-4.22 (m, 2H); 7.14 (d, J=8 Hz, 2H), 7.86 (d, J=8 Hz, 2H), 9.9 (s, 1H, CHO).

### 5.2. 7-Methoxy-4-(4-methoxyphenyl)-N-{1-[4-(2-piperidin-1-ylethoxy)benzyl]piperidin-4-yl}phthalazin-1-amine trihydrochloride (compound 5)

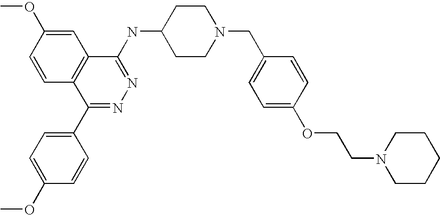

This compound in base form is obtained according to the procedure described in 3.3. by reacting 7-methoxy-4-(4-methoxyphenyl)-N-(piperidin-4-yl)phthalazin-1-amine with 4-(2-piperidin-1-ylethoxy)benzaldehyde.

¹H NMR (DMSO-d₆, 400 MHz) δ ppm: 1.35-1.42 (m, 2H); 1.45-1.55 (m, 4H); 1.60-1.73 (m, 2H); 2.02-2.12 (s, 4H); 2.40-2.48 (m, 2H); 2.60-2.70 (m, 2H); 2.88-2.93 (m, 2H); 3.28-3.33 (m, 2H); 3.44 (s, 2H); 3.84 (s, 3H); 3.97 (s, 3H); 4.03-4.08 (m, 2H); 4.20-4.30 (m, 1H); 6.90 (d, J=8 Hz, 2H); 6.97 (d, J=7.5 Hz, 1H); 7.09 (d, J=8 Hz, 2H); 7.23 (d, J=8 Hz, 2H); 7.41 (dd, J₁=7.5 Hz, J₂=2.5 Hz, 1H), 7.51 (d, J=8 Hz, 2H); 7.72-7.75 (m, 2H);

The trihydrochloride is obtained by means of the treatment described in 4.3.

### EXAMPLE 6

### N-[1-(1,3-Benzodioxol-5-ylmethyl)piperidin-4-yl]-4-ethyl-7-methoxy-N-methylphthalazin-1-amine dihydrochloride (compound 6)

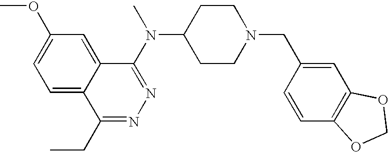

### 6.1. 5-Methoxy-2-propionylbenzoic Acid

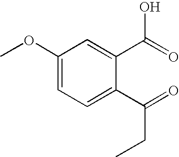

This compound is synthesized according to the method described in 2.4. by reacting 2-bromo-5-methoxybenzoic acid, treated beforehand with n-butyllithium, with N-methoxy-N-methylpropionamide. It is used crude in the following reaction.

### 6.2. 4-Ethyl-7-methoxy-2H-phthalazin-1-one

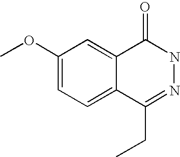

This compound is obtained according to the procedure described in 2.5. by reacting unpurified 5-methoxy-2-propionylbenzoic acid with hydrazine hydrate.

LC/MS: MH⁺=205 (Rt=6.32 minutes pH 3.1)

¹H NMR (DMSO-d₆, 250 MHz) δ ppm: 1.25 (t, J=7.5 Hz, 3H); 2.92 (q, J=7.5 Hz, 2H); 3.94 (s, 3H); 7.50 (dd, J₁=9 Hz, J₂=2.7 Hz, 1H); 7.60 (d, J=2.7 Hz, 1H); 7.94 (d, J=9 Hz, 1H); 12.38 (s, 1H, NH).

### 6.3. 1-Chloro-4-ethyl-7-methoxyphthalazine

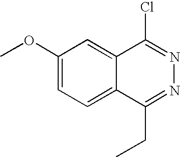

This compound is obtained according to the procedure described in 2.6. by reacting 4-ethyl-7-methoxy-2H-phthalazin-1-one with phosphoryl chloride.

m.p.=191° C. (Mettler FP62)

LC/MS: MH⁺=223 (Rt=6.84 minutes pH 3.1)

¹H NMR (DMSO-d₆, 250 MHz) δ ppm: 1.36 (t, J=7.5 Hz, 3H); 3.30 (q, J=7.5 Hz, 2H); 4.04 (s, 3H); 7.50 (d, J=2.7 Hz, 1H); 7.71 (dd, J₁=9 Hz, J₂=2.7 Hz, 1H); 8.30 (d, J=9 Hz, 1H).

### 6.4 1-(1,3-Benzodioxol-5-ylmethyl)-N-methylpiperidin-4-amine

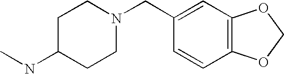

A solution of 1.94 g (5.8 mmol) of tert-butyl [1-(1,3-benzodioxol-5-ylmethyl)piperidin-4-yl]carbamate (synthesis described in 1.1) in 10 mL of THF is added slowly to a suspension of 0.45 g of LiAlH₄ (11.8 mmol) in 10 mL of THF. The mixture is stirred at reflux for 2 hours and, after cooling, 0.45 mL of water, 0.45 mL of 15% NaOH and 1.45 mL of water are then added successively. The suspension obtained is filtered through Celite and the Celite is rinsed with dichloromethane. The filtrate is evaporated under reduced pressure. 1.35 g of an oily product are obtained.

¹H NMR (DMSO-d₆, 400 MHz) δ ppm: 1.10-1.37 (m, 2H); 1.70-1.80 (m, 2H); 1.88-1.98 (m, 2H); 2.18-2.22 (m, 1H); 2.24 (s, 3H); 2.67-2.72 (m, 2H); 3.33 (s, 2H); 5.98 (s, 2H); 6.72 (m, 1H); 6.81-6.85 (m, 2H).

### 6.5. N-[1-(1,3-Benzodioxol-5-ylmethyl)piperidin-4-yl]-4-ethyl-7-methoxy-N-methylphthalazin-1-amine dihydrochloride (compound 6)

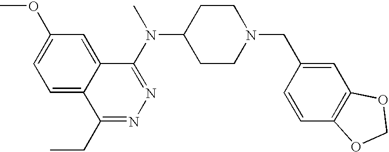

This compound in base form is obtained according to the procedure described in 1.9. by reacting 1-chloro-4-ethyl-7-methoxyphthalazine with 1-(1,3-benzodioxol-5-ylmethyl)-N-methylpiperidin-4-amine.

¹H NMR (DMSO-d₆, 400 MHz) δ ppm: 1.36 (t, J=7.5 Hz, 3H); 1.70-1.80 (m, 2H); 1.88-2.00 (m, 4H); 2.82-2.90 (m, 2H); 2.92 (s, 3H); 3.18 (q, J=7.5 Hz, 2H); 3.35 (s, 2H); 3.48-3.58 (m, 1H); 3.97 (s, 3H); 5.99 (s, 2H); 6.71-6.75 (m, 1H); 6.81-6.86 (m, 2H), 7.27 (d, J=2.5 Hz, 1H); 7.53 (dd, J₁=8.5 Hz, J₂=2.5 Hz, 1H), 8.11 (d, J=8.5 Hz, 1H).

The dihydrochloride is obtained after treating the product dissolved in dichloromethane with a solution of hydrogen chloride in diethyl ether. After concentrating and adding diisopropyl ether, a suspension is obtained, which is filtered. The powder obtained is dried at 40° C. under reduced pressure in the presence of phosphorus pentoxide.

### EXAMPLE 7

### N-[1-(1,3-Benzodioxol-5-ylmethyl)piperidin-4-yl]-7-methoxy-4-(methoxymethyl)-N-methylphthalazin-1-amine dihydrochloride (compound 7)

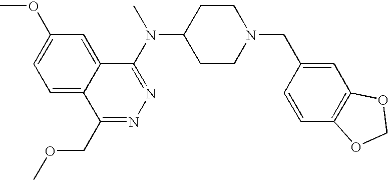

### 7.1 5-Methoxy-2-(methoxyacetyl)benzoic Acid

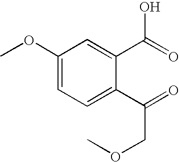

This compound is synthesized according to the method described in 2.4. by reacting 2-bromo-5-methoxybenzoic acid pretreated with n-butyllithium with 2,N-dimethoxy-N-methylacetamide. It is used crude in the following reaction.

### 7.2. 7-Methoxy-4-methoxymethyl-2H-phthalazin-1-one

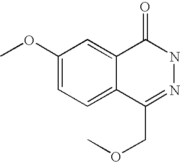

This compound is obtained according to the procedure described in 2.5. by reacting unpurified 5-methoxy-2-(methoxyacetyl)benzoic acid with hydrazine hydrate.

m.p.=184° C. (Mettler FP62)

LC/MS: MH⁺=221 (Rt=5.33 minutes, pH 3.1)

¹H NMR (DMSO-d₆, 250 MHz) δ ppm: 3.33 (s, 3H); 3.96 (s, 3H); 4.62 (s, 2H); 7.53 (dd, J₁=8.8 Hz, J₂=2.7 Hz, 1H); 7.66 (d, J=2.7 Hz, 1H); 8.0 (d, J=8.8 Hz, 1H); 12.61 (s, 1H, NH).

### 7.3. 1-Chloro-7-methoxy-4-methoxymethylphthalazine

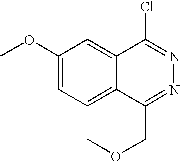

This compound is obtained according to the procedure described in 2.6. by reacting 7-methoxy-4-methoxymethyl-2H-phthalazin-1-one with phosphoryl chloride.

¹H NMR (DMSO-d₆, 250 MHz) δ ppm: 3.33 (s, 3H); 4.06 (s, 3H); 5.02 (s, 2H); 7.56 (d, J=2.7 Hz, 1H); 7.77 (dd, J₁=9.2 Hz, J₂=2.7 Hz, 1H); 8.35 (d, J=9.2 Hz, 1H).

### 7.4. N-[1-(1,3-Benzodioxol-5-ylmethyl)piperidin-4-yl]-7-methoxy-4-(methoxymethyl)-N-methylphthalazin-1-amine dihydrochloride (compound 7)

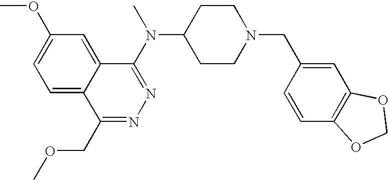

This compound in base form is obtained according to the procedure described in 1.9. by reacting 1-chloro-7-methoxy-4-methoxymethylphthalazine with 1-(1,3-benzodioxol-5-yl-methyl)-4-methylaminopiperidine.

¹H NMR (DMSO-d₆, 400 MHz) δ ppm: 1.72-1.82 (m, 2H); 1.88-2.02 (m, 4H); 2.82-2.90 (m, 2H); 2.96 (s, 3H); 3.32 (s, 3H); 3.35 (s, 2H); 3.60-3.70 (m, 1H); 3.98 (s, 3H); 4.87 (s, 2H); 5.98 (s, 2H); 6.71-6.75 (m, 1H); 6.81-6.86 (m, 2H), 7.27 (d, J=2.5 Hz, 1H); 7.54 (dd, J₁=8.5 Hz, J₂=2.5 Hz, 1H), 8.13 (d, J=8.5 Hz, 1H).

The dihydrochloride is obtained by treating the product dissolved in dichloromethane with a 2 M solution of hydrogen chloride in diethyl ether. After concentrating and adding ethyl ether, a suspension is obtained, which is filtered. The powder obtained is dried at 40° C. under reduced pressure in the presence of phosphorus pentoxide.

### EXAMPLE 8

### N-(1-{4-[3-(Dimethylamino)propoxy]-3-fluorobenzyl}piperidin-4-yl)-7-methoxy-4-(4-methoxyphenyl)phthalazin-1-amine trihydrochloride (compound 8)

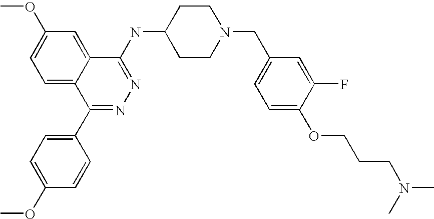

### 8.1. 4-(3-Dimethylaminopropoxy)-3-fluorobenzaldehyde

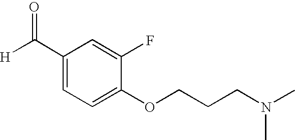

This compound is obtained according to the procedure described in 4.2. by reacting N,N-dimethyl-3-chloropropylamine with 3-fluoro-4-hydroxybenzaldehyde.

¹H NMR (DMSO-d₆, 250 MHz) δ ppm: 1.85-1.98 (m, 2H); 2.15 (s, 6H); 2.34-2.40 (m, 2H); 4.19-4.24 (m, 2H); 7.36-7.43 (m, 1H); 7.70 (d, J=11 Hz, 1H), 7.77 (d, J=7.5 Hz, 1H), 9.87 (s, 1H, CHO).

### 8.2. N-(1-{4-[3-(Dimethylamino)propoxy]-3-fluorobenzyl}piperidin-4-yl)-7-meth-oxy-4-(4-methoxyphenyl)phthalazin-1-amine trihydrochloride (compound 8)

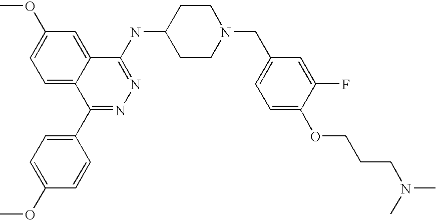

To a solution of 300 mg (0.82 mmol) of 7-methoxy-4-(4-methoxyphenyl)-N-(piperidin-4-yl)phthalazin-1-amine in 8 mL of 1,2-dichloroethane are added 185 mg (0.82 mmol) of 4-(3-dimethylaminopropoxy)-3-fluorobenzaldehyde. The mixture is stirred for one hour under nitrogen and 226 mg (1.07 mmol) of sodium triacetoxyborohydride are then added. The reaction medium is stirred for 20 hours at room temperature, hydrolyzed with water and 1 N sodium hydroxide and then extracted with dichloromethane. The organic extract is washed with saturated aqueous sodium chloride, dried over anhydrous sodium sulfate and then evaporated under reduced pressure. The residue is purified on a column of alumina (eluent: dichloromethane/methanol from 100/0 to 98/2 (v/v)). 278 mg of an oily orange-colored product are obtained.

¹H NMR (DMSO-d₆, 400 MHz) δ ppm: 1.62-1.73 (m, 2H); 1.80-1.88 (m, 2H); 2.04-2.14 (m, 4H); 2.14 (s, 6H), 2.30-2.38 (m, 2H); 2.87-2.90 (m, 2H); 3.46 (s, 2H); 3.84 (s, 3H); 3.97 (s, 3H); 4.02-4.08 (m, 2H); 4.20-4.30 (m, 1H); 6.97 (d, J=7.5 Hz, 1H); 7.05-7.18 (m, 5H); 7.42 (dd, J₁=7.5 Hz, J₂=2.5 Hz, 1H); 7.51 (d, J=8 Hz, 2H); 7.72-7.77 (m, 2H).

The trihydrochloride is obtained by means of the treatment described in 7.4.

### EXAMPLE 9

### 7-Methoxy-4-(4-methoxyphenyl)-N-{1-[4-(2-pyrrolidin-1-ylethoxy)benzyl]piperidin-4-yl}phthalazin-1-amine trihydrochloride (compound 9)

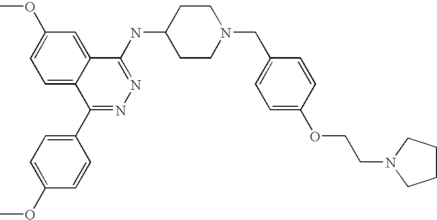

### 9.1 4-(2-Pyrrolidin-1-ylethoxy)benzaldehyde

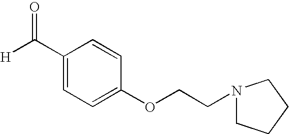

This compound is obtained according to the procedure described in 5.1. By reacting N-2-chloroethylpyrrolidine hydrochloride with 4-hydroxybenzaldehyde.

MH⁺=220 (Rt=2.41 minutes, pH 3.1).

¹H NMR (DMSO-d₆, 400 MHz) δ ppm: 1.65-1.72 (m, 4H); 2.50-2.56 (m, 4H); 2.78-2.82 (m, 2H); 4.15-4.20 (m, 2H); 7.13 (d, J=8 Hz, 2H), 7.85 (d, J=8 Hz, 2H), 9.87 (s, 1H, CHO).

### 9.2. 7-methoxy-4-(4-methoxyphenyl)-N-{1-[4-(2-pyrrolidin-1-ylethoxy)benzyl]-piperidin-4-yl}phthalazin-1-amine trihydrochloride (compound 9)

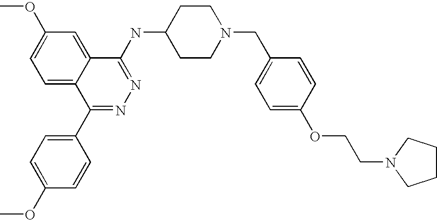

This compound in base form is obtained according to the procedure described in 3.3. by reacting 7-methoxy-4-(4-methoxyphenyl)-N-(piperidin-4-yl)phthalazin-1-amine with 4-(2-pyrrolidin-1-ylethoxy)benzaldehyde.

¹H NMR (DMSO-d₆, 250 MHz) δ ppm: 1.60-1.80 (m, 6H); 2.0-2.20 (m, 4H); 2.45-2.60 (m, 3H); 2.72-2.98 (m, 5H); 3.45 (s, 2H); 3.85 (s, 3H); 3.98 (s, 3H); 4.03-4.10 (m, 2H); 4.20-4.30 (m, 1H); 6.85-7.0 (m, 3H); 7.09 (d, J=8 Hz, 2H); 7.23 (d, J=8 Hz, 2H); 7.41 (dd, J₁=7.5 Hz, J₂=2.5 Hz, 1H), 7.51 (d, J=8 Hz, 2H); 7.73-7.76 (m, 2H);

The trihydrochloride is obtained by means of the treatment described in 7.4.

### EXAMPLE 10

### 7-Methoxy-4-(4-methoxyphenyl)-N-{1-[4-(2-morpholin-4-ylethoxy)benzyl]piperidin-4-yl}phthalazin-1-amine trihydrochloride (compound 10)

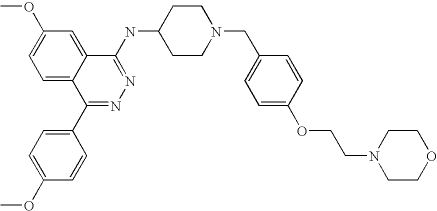

### 10.1. 4-(2-Morpholin-4-ylethoxy)benzaldehyde

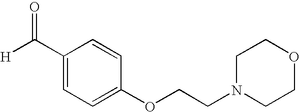

This compound is obtained according to the procedure described in 5.1. by reacting 4-(2-chloroethyl)morpholine hydrochloride with 4-hydroxybenzaldehyde.

MH⁺=236 (Rt=3.30 minutes, pH 3.1).

¹H NMR (DMSO-d₆, 400 MHz) δ ppm: 2.45-2.52 (m, 4H); 2.70-2.75 (m, 2H); 3.55-3.60 (m, 4H); 4.18-4.25 (m, 2H); 7.14 (d, J=8 Hz, 2H), 7.86 (d, J=8 Hz, 2H), 9.87 (s, 1H, CHO).

### 10.2. 7-Methoxy-4-(4-methoxyphenyl)-N-{1-[4-(2-morpholin-4-ylethoxy)benzyl]-piperidin-4-yl}phthalazin-1-amine trihydrochloride (compound 10)

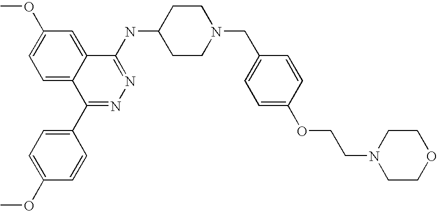

This compound in base form is obtained according to the procedure described in 3.3. by reacting 7-methoxy-4-(4-methoxyphenyl)-N-(piperidin-4-yl)phthalazin-1-amine with 4-(2-morpholin-4-ylethoxy)benzaldehyde.

¹H NMR (DMSO-d₆, 400 MHz) δ ppm: 1.60-1.75 (m, 2H); 2.0-2.15 (m, 4H); 2.45-2.60 (m, 4H); 2.68-2.72 (m, 2H); 2.88-2.98 (m, 2H); 3.45 (s, 2H); 3.58-3.62 (m, 4H); 3.84 (s, 3H); 3.97 (s, 3H); 4.02-4.10 (m, 2H); 4.20-4.30 (m, 1H); 6.88-7.04 (m, 3H); 7.09 (d, J=8.8 Hz, 2H); 7.24 (d, J=8 Hz, 2H); 7.42 (dd, J₁=9 Hz, J₂=2.5 Hz, 1H), 7.51 (d, J=8.8 Hz, 2H); 7.73-7.76 (m, 2H);

The trihydrochloride is obtained by means of the treatment described in 7.4.

### EXAMPLE 11

### 7-Methoxy-4-(4-methoxyphenyl)-N-{1-[4-(3-piperidin-1-ylpropoxy)benzyl]piperidin-4-yl}phthalazin-1-amine trihydrochloride (compound 11)

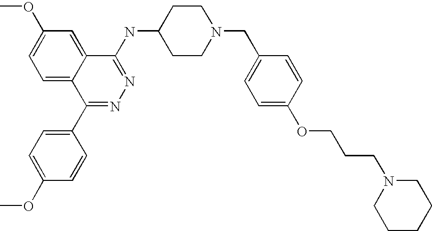

### 11.1 4-(3-Piperidin-1 ylpropoxy)benzaldehyde

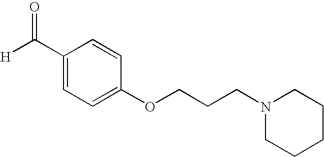

This compound is obtained according to the procedure described in 5.1. by reacting N-(3-chloropropyl)piperidine hydrochloride with 4-hydroxybenzaldehyde.

¹H NMR (DMSO-d₆, 250 MHz) δ ppm: 1.40-1.55 (m, 6H); 1.84-1.87 (m, 2H); 2.30-2.45 (m, 6H); 4.10-4.18 (m, 2H); 7.13 (d, J=8 Hz, 2H), 7.86 (d, J=8 Hz, 2H), 9.87 (s, 1H, CHO).

MH⁺=248 (Rt=4.43 minutes, pH 3.1).

### 11.2. 7-Methoxy-4-(4-methoxyphenyl)-N-{1-[4-(3-piperidin-1-ylpropoxy)benzyl]piperidin-4-yl}phthalazin-1-amine trihydrochloride (compound 11)

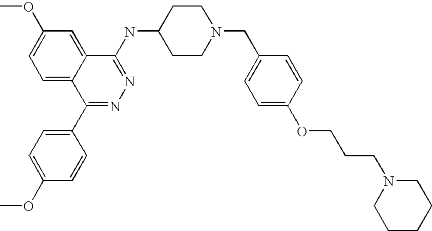

This compound in base form is obtained according to the procedure described in 3.3. by reacting 7-methoxy-4-(4-methoxyphenyl)-N-(piperidin-4-yl)phthalazin-1-amine with 4-(2-piperidin-1-ylpropoxy)benzaldehyde.

¹H NMR (DMSO-d₆, 400 MHz) δ ppm: 1.35-1.42 (m, 2H); 1.48-1.55 (m, 4H); 1.62-1.75 (m, 2H); 1.82-1.92 (m, 2H); 2.03-2.14 (m, 4H); 2.30-2.40 (m, 6H); 2.88-2.95 (m, 2H); 3.44 (s, 2H); 3.84 (s, 3H); 3.96 (s, 3H); 3.96-4.01 (m, 2H); 4.20-4.30 (m, 1H); 6.88 (d, J=8.8 Hz, 2H), 6.96 (d, J=7.6 Hz, 1H, NH); 7.08 (d, J=8.8 Hz, 2H); 7.22 (d, J=8.4 Hz, 2H); 7.41 (dd, J₁=9 Hz, J₂=2.5 Hz, 1H), 7.51 (d, J=8.4 Hz, 2H); 7.72-7.75 (m, 2H);

The trihydrochloride is obtained by means of the treatment described in 7.4.

### EXAMPLE 12

### N-[1-(1,3-Benzodioxol-5-ylmethyl)piperidin-4-yl]-N-ethyl-7-methoxy-4-(4-methoxy-phenyl)phthalazin-1-amine dihydrochloride (compound 12)

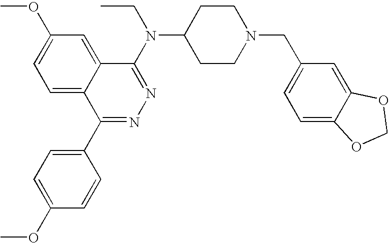

### 12.1 N-[1-(1,3-Benzodioxol-5-ylmethyl)piperidin-4-yl]-7-methoxy-4-(4-methoxy-phenyl)phthalazin-1-amine

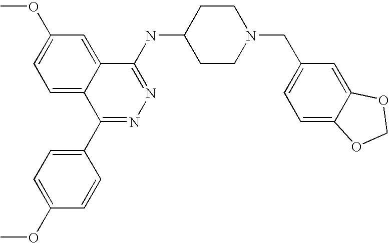

This compound is obtained according to the procedure described in 3.3. by reacting 7-methoxy-4-(4-methoxyphenyl)-N-(piperidin-4-yl)phthalazin-1-amine with piperonal.

MH⁺=499 (Rt=8.32 minutes, pH 7)

¹H NMR (DMSO-d₆, 250 MHz) δ ppm: 1.60-1.80 (m, 2H); 2.0-2.20 (m, 4H); 2.85-2.95 (m, 2H); 3.44 (s, 2H); 3.85 (s, 3H); 3.98 (s, 3H); 4.20-4.30 (m, 1H), 6.01 (s, 2H); 6.78-6.91 (m, 3H); 6.99 (d, J=7.5 Hz, 1H), 7.09 (d, J=8 Hz, 2H); 7.43 (dd, J₁=8.5 Hz, J₂=2.5 Hz, 1H), 7.51 (d, J=8 Hz, 2H); 7.72-7.76 (m, 2H).

### 12.2 N-[1-(1,3-Benzodioxol-5-ylmethyl)piperidin-4-yl]-N-ethyl-7-methoxy-4-(4-methoxyphenyl)phthalazin-1-amine dihydrochloride (compound 12)

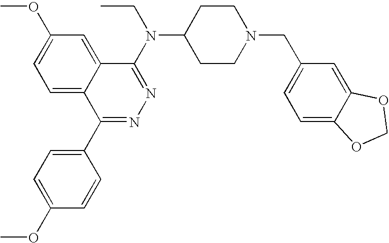

A solution of 200 mg (0.39 mmol) of N-[1-(1,3-benzodioxol-5-ylmethyl)piperidin-4-yl]-7-methoxy-4-(4-methoxyphenyl)phthalazin-1-amine in 15 mL of THF is stirred at 0° C. under nitrogen and 155 mg of a 60% suspension of sodium hydride in oil (0.39 mmol) are added. The mixture is stirred for one hour and 0.062 mL (0.78 mmol) of iodoethane is then added. The mixture is stirred for 24 hours at room temperature and then cooled on an ice bath, water is added and the mixture is extracted with dichloromethane. The organic extract is washed with brine, dried over anhydrous sodium sulfate and then evaporated under reduced pressure. The residue obtained is purified on a column of neutral alumina (eluent: dichloromethane/methanol from 100/0 to 98/2 (v/v)) and then on a column of silica gel (solvent: dichloromethane/methanol from 100/0 to 85/15 (v/v)).

¹H NMR (DMSO-d₆, 250 MHz) δ ppm: 1.02 (t, J=6.7 Hz, 3H), 1.75-2.0 (m, 6H); 2.82-2.90 (m, 2H); 3.34 (s, 2H); 3.42-3.60 (m, 3H), 3.85 (s, 3H); 3.96 (s, 3H); 5.99 (s, 2H); 6.73 (d, J=7.7 Hz, 1H), 6.82-6.86 (m, 2H); 7.13 (d, J=9 Hz, 2H); 7.37 (d, J=2.5 Hz, 1H); 7.52 (dd, J₁=9 Hz, J₂=2.5 Hz, 1H), 7.64 (d, J=9 Hz, 2H), 7.88 (d, J=9 Hz, 1H).

The dihydrochloride is obtained by means of the treatment described in 6.5.

### EXAMPLE 13

### 4-[(4-{[7-Methoxy-4-(4-methoxyphenyl)phthalazin-1-yl]amino}piperidin-1-yl)methyl]-N-(2-piperidin-1-ylethyl)benzamide trihydrochloride (compound 13)

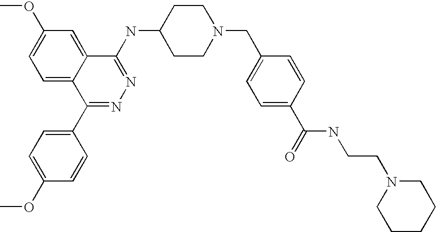

### 13.1 4-Chloromethyl-N-(2-piperidin-1-ylethyl)benzamide

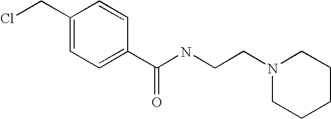

A solution of 5 g (26.4 mmol) of 4-chloromethylbenzoyl chloride in 100 mL of dichloromethane is stirred at 0° C. and a solution of 3.39 g (26.4 mmol) of 1-(2-aminoethyl)piperidine and 5.53 mL (39.7 mmol) of triethylamine in 10 mL of dichloromethane is added thereto. The mixture is stirred for 5 hours at 0° C. and then at room temperature overnight. Water is added and the mixture is then extracted with dichloromethane. The organic extract is washed with brine, dried over anhydrous sodium sulfate and then evaporated under reduced pressure. The residue obtained is purified on a column of silica gel (solvent: dichloromethane/methanol from 100/0 to 85/15 (v/v)). 1.3 g of solid product are obtained

MH⁺=281 (Rt=4.72 minutes, pH 3.1)

¹H NMR (DMSO-d₆, 250 MHz) δ ppm: 1.34-1.44 (m, 2H); 1.44-1.58 (m, 4H); 2.35-2.48 (m, 6H); 3.30-3.42 (m, 2H); 4.83 (s, 2H); 7.53 (d, J=8 Hz, 2H), 7.83 (d, J=8 Hz, 2H), 8.40 (m, 1H, NH).

### 13.2. 4-[(4-{[7-Methoxy-4-(4-methoxyphenyl)phthalazin-1-yl]amino}piperidin-1-yl)methyl]-N-(2-piperidin-1-ylethyl)benzamide trihydrochloride (compound 13)

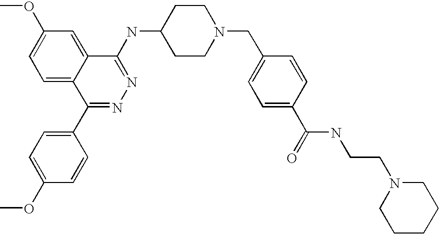

To a solution of 300 mg (0.82 mmol) of 7-methoxy-4-(4-methoxyphenyl)-N-(piperidin-4-yl)phthalazin-1-amine and 311 g (0.82 mmol) of 4-chloromethyl-N-(2-piperidin-1-ylethyl)benzamide in 20 mL of DMF are added 114 mg (0.82 mmol) of potassium carbonate. The mixture is stirred at room temperature for 18 hours, water is then added and the mixture is extracted with ethyl acetate. The organic extract is washed with water and then with saturated aqueous sodium chloride, dried over anhydrous sodium sulfate and then evaporated under reduced pressure. The residue obtained is purified on a column of neutral alumina (eluent: dichloromethane/methanol from 100/0 to 95/5 (v/v)) and then on a column of basic alumina under the same conditions. 130 mg of gummy yellow product are obtained.

¹H NMR (DMSO-d₆, 400 MHz) δ ppm: 1.32-1.42 (m, 2H); 1.45-1.55 (m, 4H); 1.63-1.75 (m, 2H); 2.05-2.20 (m, 4H); 2.32-2.44 (m, 6H); 2.90-2.95 (m, 2H); 3.33-3.40 (m, 2H); 3.57 (s, 2H); 3.84 (s, 3H); 3.97 (s, 3H); 4.20-4.30 (m, 1H); 6.98 (d, J=7.2 Hz, 1H, NH), 7.08 (d, J=8.8 Hz, 2H); 7.40-7.43 (m, 3H); 7.51 (d, J=8.8 Hz, 2H); 7.73 (d, J=8.8 Hz, 2H); 7.80 (d, J=8.8 Hz, 2H); 8.30-8.35 (m, 1H, CONH)

The trihydrochloride is obtained by means of the treatment described in 4.3.

### EXAMPLE 14

### 7-Methoxy-4-(4-methoxyphenyl)-N-(1-{4-[(1-methylazepan-3-yl)oxy]benzyl}piperidin-4-yl)phthalazin-1-amine trihydrochloride (compound 14)

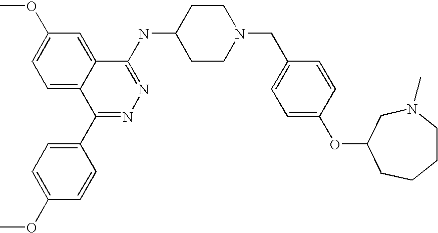

### 14.1. 4-[(1-Methylazepan-3-yl)oxy]benzaldehyde and

### 4-[(1-methylpiperidin-2-yl)methoxy]benzaldehyde

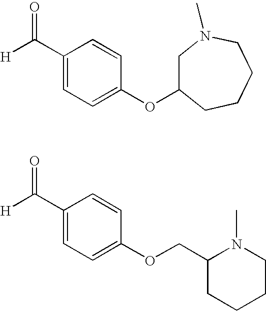

To a solution of 2.59 g (21.2 mmol) 2-chloromethyl-1N-methylpiperidine (obtained by reacting 1N-methylpiperidine-2-ylmethanol with SOCl₂, hydrolysis with N NaOH and then extraction with dichloromethane) and 3.13 g (21.2 mmol) of 4-hydroxybenzaldehyde in 42 mL of DMF are added 2.93 g (21.2 mmol) of potassium carbonate. The mixture is stirred at 80° C. for 4 hours and, after cooling, is then poured into water and extracted with ethyl acetate. The organic extract is washed with water and then with saturated aqueous sodium chloride, dried over anhydrous sodium sulfate and then evaporated under reduced pressure. 900 mg of the residue obtained are purified on a column of silica gel (solvent: dichloromethane/methanol from 100/0 to 95/5 (v/v)). 360 mg of 4-[(1-methylazepan-3-yl)oxy]benzaldehyde and 192 mg of 4-[(1-methylpiperidin-2-yl)methoxy]benzaldehyde are obtained. These products are oily.

4-[(1-Methylazepan-3-yl)oxy]benzaldehyde:

¹H NMR (DMSO-d₆, 400 MHz) δ ppm: 1.48-1.80 (m, 5H); 2.0-2.10 (m, 1H); 2.31 (s, 3H); 2.45-2.55 (m, 1H); 2.55-2.70 (m, 2H); 2.82-2.90 (m, 1H); 4.18-4.23 (m, 1H); 7.14 (d, J=9 Hz, 2H), 7.84 (d, J=9 Hz, 2H), 9.86 (s, 1H, CHO).

4-[(1-Methylpiperidin-2-yl)methoxy]benzaldehyde:

¹H NMR (DMSO-d₆, 400 MHz) δ ppm: 1.20-1.60 (m, 4H); 1.68-1.80 (m, 2H); 2.02-2.10 (m, 1H); 2.20-2.27 (m, 1H); 2.24 (s, 3H); 2.74-2.80 (m, 1H); 4.0-4.05 (m, 1H); 4.18-4.22 (m, 1H); 7.13 (d, J=9 Hz, 2H), 7.85 (d, J=9 Hz, 2H), 9.88 (s, 1H, CHO).

### 14.2. 7-methoxy-4-(4-methoxyphenyl)-N-(1-{4-[(1-methylazepan-3-yl)oxy]benzyl}-piperidin-4-yl)phthalazin-1-amine trihydrochloride (compound 14)

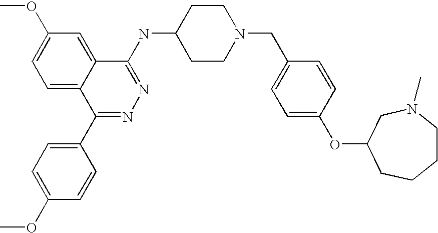

This compound in base form is obtained according to the procedure described in 3.3. by reacting 7-methoxy-4-(4-methoxyphenyl)-N-(piperidin-4-yl)phthalazin-1-amine with 4-[(1-methylazepan-3-yl)oxy]benzaldehyde.

¹H NMR (DMSO-d₆, 400 MHz) δ ppm: 1.50-1.76 (m, 8H); 1.96-2.10 (m, 5H); 2.30 (s, 3H); 2.55-2.65 (m, 2H); 2.80-2.95 (m, 3H); 3.43 (s, 2H); 3.84 (s, 3H); 3.97 (s, 3H); 4.20-4.30 (m, 1H); 4.45-4.55 (m, 1H); 6.85 (d, J=8.4 Hz, 2H), 6.96 (d, J=7.6 Hz, 1H, NH), 7.08 (d, J=8.8 Hz, 2H); 7.21 (d, J=8.4 Hz, 2H); 7.41 (dd, J₁=8.8 Hz, J₂=2.5 Hz, 1H), 7.51 (d, J=8.8 Hz, 2H); 7.70-7.78 (m, 2H)

The trihydrochloride is obtained by means of the treatment described in 7.4.

### EXAMPLE 15

### 7-Methoxy-4-(4-methoxyphenyl)-N-(1-{4-[(1-methylpiperidin-2-yl)methoxy]benzyl}-piperidin-4-yl)phthalazin-1-amine trihydrochloride (compound 15)

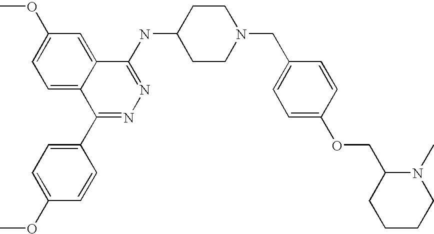

This compound in base form is obtained according to the procedure described in 3.3. by reacting 7-methoxy-4-(4-methoxyphenyl)-N-(piperidin-4-yl)phthalazin-1-amine with 4-[(1-methylpiperidin-2-yl)methoxy]benzaldehyde.

¹H NMR (DMSO-d₆, 400 MHz) δ ppm: 1.20-1.80 (m, 8H); 2.0-2.12 (m, 5H); 2.15-2.23 (m, 1H); 2.23 (s, 3H); 2.72-2.80 (m, 1H); 2.88-2.93 (m, 2H); 3.44 (s, 2H); 3.84 (s, 3H); 3.84-3.88 (m, 1H); 3.97 (s, 3H); 4.02-4.07 (m, 1H); 4.20-4.30 (m, 1H); 6.90 (d, J=8.4 Hz, 2H); 6.96 (d, J=7.6 Hz, 1H, NH), 7.08 (d, J=8.8 Hz, 2H); 7.23 (d, J=8.4 Hz, 2H); 7.41 (dd, J₁=9 Hz, J₂=2.5 Hz, 1H), 7.51 (d, J=8.8 Hz, 2H); 7.70-7.76 (m, 2H)

The trihydrochloride is obtained by means of the treatment described in 7.4.

### EXAMPLE 16

### N-[7-Methoxy-4-(4-methoxyphenyl)phthalazin-1-yl]-N-[1-(2-naphthylmethyl)piperidin-4-yl]acetamide dihydrochloride (compound 16)

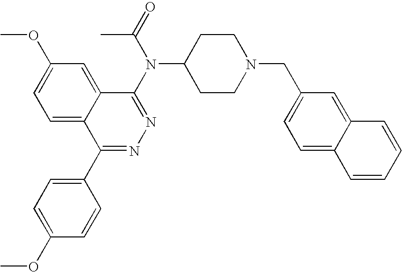

### 16.1. 7-Methoxy-4-(4-methoxyphenyl)-N-[1-(naphthalen-2-ylmethyl)piperidin-4-yl]phthalazin-1-amine

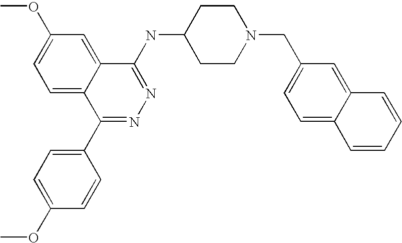

To a solution of 1.46 g (4 mmol) of 7-methoxy-4-(4-methoxyphenyl)-N-(piperidin-4-yl)phthalazin-1-amine (synthesis: see 3.2) in 20 mL of dichloro-1,2-ethane is added 0.63 g (4 mmol) of naphthalene-2-aldehyde. The mixture is stirred for 30 minutes under nitrogen and 1.1 g (5.2 mmol) of sodium triacetoxyborohydride are then added. The reaction medium is stirred for 24 hours at room temperature and then hydrolyzed with 20 mL of 1N and extracted with dichloromethane. The organic extract is washed with saturated aqueous sodium chloride, dried over anhydrous sodium sulfate and then evaporated under reduced pressure. The residue is purified on a column of silica (eluent: dichloromethane/methanol from 100/0 to 90/10 (v/v)). 1.74 g of solid product, recrystallized from isopropyl ether, are obtained

m.p.=195° C. (M)

LC/MS: MH⁺=505 (Rt=5.73 minutes pH 3.1)

¹H NMR (DMSO-d₆, 300 MHz) δ ppm: 1.65-1.80 (m, 2H); 2.03-2.28 (m, 4H); 2.90-3.02 (m, 2H); 3.70 (s, 2H); 3.84 (s, 3H); 3.97 (s, 3H); 4.2-4.3 (m, 1H); 6.99 (d, J=7.2 Hz. 1H, NH); 7.08 (d, J=8.5 Hz, 2H); 7.42 (dd; J₁=9.0 Hz; J₂=2.5 Hz, 1H); 7.48-7.58 (m, 5H); 7.70-7.76 (m, 2H); 7.80-7.92 (m, 4H).

### 16.2. N-[7-Methoxy-4-(4-methoxyphenyl)phthalazin-1-yl]-N-[1-(2-naphthyl-methyl)piperidin-4-yl]acetamide dihydrochloride (compound 16)

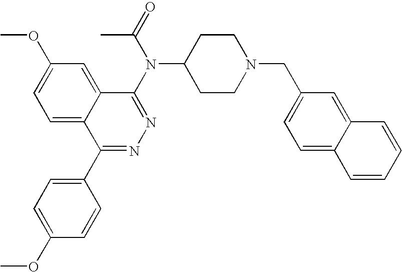

A mixture of 625 mg (1.23 mmol) of 7-methoxy-4-(4-methoxyphenyl)-N-[1-(naphthalen-2-ylmethyl)piperidin-4-yl]phthalazin-1-amine and 5 mL of acetic anhydride is stirred at room temperature for 22 hours and then at 80° C. for 3 hours. The reaction medium is hydrolyzed by addition of water and then basified with 2 N NaOH. The mixture is extracted with dichloromethane and the organic extract is washed with saturated aqueous sodium chloride, dried over anhydrous sodium sulfate and then evaporated under reduced pressure. The residue obtained is purified on a column of silica (eluent: dichloromethane/methanol from 100/0 to 85/15 (v/v)). 395 mg of solid product are obtained.

¹H NMR (DMSO-d₆, 600 MHz) δ ppm: 1.0-1.08 (m, 1H); 1.63 (s, 3H); 1.72-1.98 (m, 2H); 2.0-2.15 (m, 3H); 2.72-2.78 (m, 1H); 2.85-3.92 (m, 1H); 3.52-3.57 (m, 2H); 3.89 (s, 3H); 3.99 (s, 3H); 4.52-4.62 (m, 1H); 7.18 (d, J=9 Hz, 2H); 7.34 (d, J=1.8 Hz. 1H); 7.37 (d, J=8.4 Hz. 1H); 7.43-7.47 (m, 2H); 7.65 (dd, J₁=9 Hz; J₂=1.8 Hz, 1H); 7.69-7.80 (m, 6H); 8.04 (d, J=9 Hz. 1H).

The dihydrochloride is obtained by means of the treatment described in 4.3.

### EXAMPLE 17

### 7-Methoxy-4-(4-methoxyphenyl)-N-(1-{4-[(1-methylpiperidin-4-yl)oxy]benzyl}piperidin-4-yl)phthalazin-1-amine trihydrochloride (compound 17)

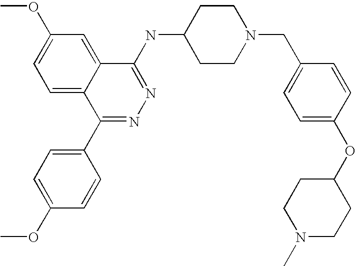

### 17.1. 4-[(1-Methylpiperidin-4-yl)oxy]benzaldehyde

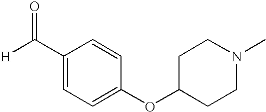

A mixture of 1 g (8.19 mmol) of 4-hydroxybenzaldehyde, 2.79 g (10.65 mmol) of triphenylphosphine and 0.943 g (8.19 mmol) of 4-hydroxy-1-methylpiperidine in 15 mL of THF is stirred at 0° C. a solution of 1.76 mL (9.0 mmol) of diisopropyl azodicarboxylate in 5 mL of THF and is added slowly thereto. The reaction medium is stirred 15 minutes at 0° C. and then overnight at room temperature. After evaporating under reduced pressure, the residue is purified on a column of silica gel (eluent: dichloromethane/methanol from 100/0 to 85/15 (v/v)). 1.4 g of an oily product are obtained.

¹H NMR (DMSO-d₆, 250 MHz) δ ppm: 1.60-1.76 (m, 2H); 1.90-2.05 (m, 2H); 2.21 (s, 3H); 2.20-2.30 (m, 2H); 2.60-2.70 (m, 2H); 4.50-4.60 (m, 1H); 7.14 (d, J=9 Hz, 2H), 7.85 (d, J=9 Hz, 2H), 9.80 (s, 1H, CHO).

### 17.2. 7-Methoxy-4-(4-methoxyphenyl)-N-(1-{4-[(1-methylpiperidin-4-yl)oxy]-benzyl}piperidin-4-yl)phthalazin-1-amine trihydrochloride (compound 17)

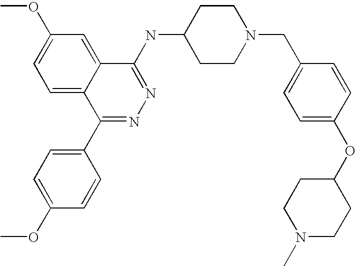

This compound in base form is obtained according to the procedure described in 3.3. by reacting 7-methoxy-4-(4-methoxyphenyl)-N-(piperidin-4-yl)phthalazin-1-amine with 4-[(1-methylpiperidin-4-yl)oxy]benzaldehyde.

The trihydrochloride is obtained by treating the product dissolved in ethanol with a 2 M solution of hydrogen chloride in diethyl ether. After concentrating and adding ethyl ether, a suspension is obtained, which is filtered. The powder obtained is dried at 40° C. under reduced pressure in the presence of phosphorus pentoxide.

### EXAMPLE 18

### N-{1-[4-(1-Azabicyclo[2.2.2]oct-3-yloxy)benzyl]piperidin-4-yl}-7-methoxy-4-(4-meth-oxyphenyl)phthalazin-1-amine trihydrochloride (compound 18)

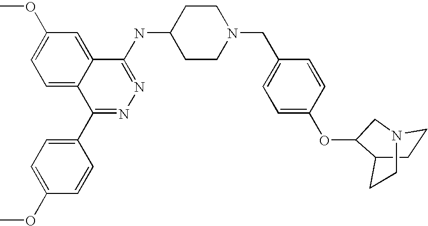

### 18.1. 4-(1-Azabicyclo[2.2.2]oct-3-yloxy)benzaldehyde

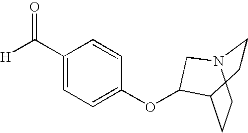

A mixture consisting of 2 g (10.98 mmol) of 3-chloroquinuclidine hydrochloride, 1.34 g (10.98 mmol) of 4-hydroxybenzaldehyde, 3.036 g (22 mmol) of potassium carbonate, 22 mL of NMP and 10 mL of DMF is stirred at 165° C. for 8 hours. Water and ethyl acetate are added to the cooled reaction medium. The organic phase is washed successively with 1N sodium hydroxide, with water and with saturated aqueous sodium chloride. It is dried over anhydrous sodium sulfate and then evaporated under reduced pressure. The oily residue obtained (0.92 g) is heated in a bulb oven at 50° C. under a pressure of 1 mbar to distil off the residual 3-chloroquinuclidine. 0.46 g of gummy aldehyde is obtained.

¹H NMR (DMSO-d₆, 250 MHz) δ ppm: 1.22-1.42 (m, 1H); 1.50-1.88 (m, 3H); 2.03-2.12 (m, 1H); 2.55-2.82 (m, 4H); 3.22-3.30 (m, 2H); 4.57-4.65 (m, 1H); 7.10 (d, J=9 Hz, 2H), 7.85 (d, J=9 Hz, 2H), 9.88 (s, 1H, CHO).

### 18.2. N-{1-[4-(1-Azabicyclo[2.2.2]oct-3-yloxy)benzyl]piperidin-4-yl}-7-methoxy-4-(4-methoxyphenyl)phthalazin-1-amine trihydrochloride (compound 18)

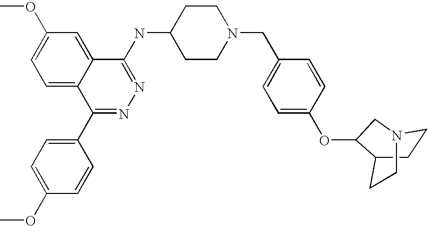

This compound in base form is obtained according to the procedure described in 3.3. by reacting 7-methoxy-4-(4-methoxyphenyl)-N-(piperidin-4-yl)phthalazin-1-amine with 4-(1-azabicyclo[2.2.2]oct-3-yloxy)benzaldehyde.

The trihydrochloride is obtained by means of the treatment described in 7.4.

### EXAMPLE 19

### N-[1-(1,3-Benzodioxol-5-ylmethyl)piperidin-4-yl]-7-methoxy-4-(tetrahydro-2H-pyran-4-yl)phthalazin-1-amine dihydrochloride (compound 19)

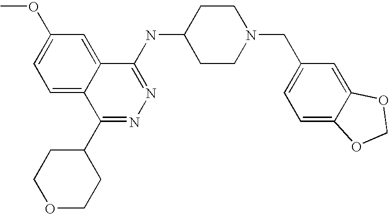

### 19.1. N-Methoxy-N-methyltetrahydro-2H-pyran-4-carboxamide

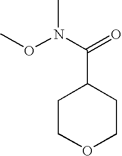

This oily compound is obtained according to the procedure described in 2.3. by reacting tetrahydro-2H-pyran-4-carbonyl chloride with N,O-dimethylhydroxylamine hydrochloride.

¹H NMR (DMSO-d₆, 250 MHz) δ ppm: 1.53-1.62 (m, 4H); 2.85-3.0 (m, 1H); 3.10 (s, 3H); 3.30-3.42 (m, 2H); 3.69 (s, 3H); 3.82-3.91 (m, 2H).

### 19.2. 5-Methoxy-2-(tetrahydro-2H-pyran-4-ylcarbonyl)benzoic Acid

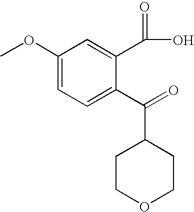

This compound is obtained according to the procedure described in 2.4. by reacting unpurified 2-bromo-5-methoxybenzoic acid pretreated with n-butyllithium with N-methoxy-N-methyltetrahydro-2H-pyran-4-carboxamide. It is used crude in the following reaction.

### 19.3. 7-Methoxy-4-(tetrahydro-2H-pyran-4-yl)phthalazin-I (2H)-one

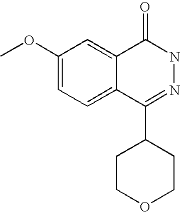

This compound is obtained according to the procedure described in 2.5. by reacting 5-methoxy-2-(tetrahydro-2H-pyran-4-ylcarbonyl)benzoic acid with hydrazine hydrate, followed by purification on a column of silica gel (eluent: dichloromethane/methanol from 100/0 to 90/10 (v/v)).

m.p.=276° C. (M)

LC/MS: MH⁺=261 (Rt=6.23 minutes pH 3.1)

¹H NMR (DMSO-d₆, 250 MHz) δ ppm: 1.72-1.82 (m, 4H); 3.40-3.62 (m, 3H); 3.90-4.0 (m, 3H); 3.97 (s, 3H); 7.49 (dd, J₁=9 Hz, J₂=2.7 Hz, 1H); 7.68 (d, J=2.7 Hz, 1H); 8.08 (d, J=9.0 Hz, 1H).

### 19.4. 4-Chloro-6-methoxy-1-(tetrahydro-2H-pyran-4-yl)phthalazine

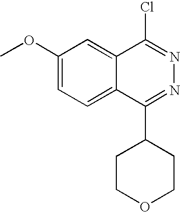

This compound is obtained according to the procedure described in 2.6. by reacting 7-methoxy-4-(tetrahydro-2H-pyran-4-yl)phthalazin-1(2H)-one with phosphoryl chloride.

m.p.=146° C. (M)

LC/MS: MH⁺=279 (Rt=7.08 minutes pH 3.1)

¹H NMR (DMSO-d₆, 250 MHz) δ ppm: 1.78-1.88 (m, 2H); 1.90-2.10 (m, 2H); 3.58-3.68 (m, 2H); 3.82-4.02 (m, 3H); 4.04 (s, 3H); 7.53 (d, J=2.5 Hz, 1H); 7.73 (dd, J₁=9 Hz, J₂=2.5 Hz, 1H); 8.46 (d, J=9.0 Hz, 1H).

### 19.5. N-[1-(1,3-Benzodioxol-5-ylmethyl)piperidin-4-yl]-7-methoxy-4-(tetrahydro-2H-pyran-4-yl)phthalazin-1-amine Dihydrochloride (Compound 19)

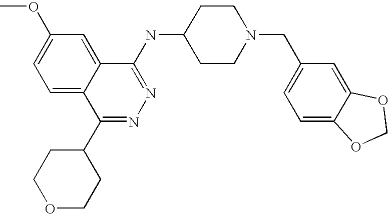

This compound in base form is obtained according to the procedure described in 2.7. by reacting 4-chloro-6-methoxy-1-(tetrahydro-2H-pyran-4-yl)phthalazine with 1-(1,3-benzo-dioxol-5-ylmethyl)piperidin-4-ylamine.

The dihydrochloride is obtained by means of the treatment described in 4.3.

### EXAMPLE 20

### 7-Methoxy-4-(4-methoxyphenyl)-N-{1-[4-(3-methoxypropoxy)benzyl]piperidin-4-yl}-phthalazin-1-amine trihydrochloride (compound 20)

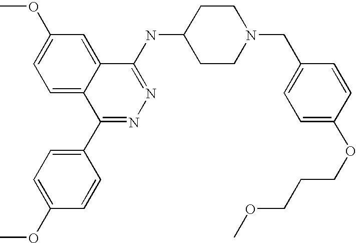

### 20.1. 4-(3-Methoxypropoxy)benzaldehyde

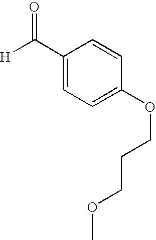

This compound is obtained via the Mitsunobu reaction according to the procedure described in 17.1 by reacting 4-hydroxybenzaldehyde and 3-methoxy-propan-1-ol.

¹H NMR (DMSO-d₆, 250 MHz) δ ppm: 1.92-2.05 (m, 2H); 3.26 (s, 3H); 3.46-3.51 (m, 2H); 4.11-4.18 (m, 2H); 7.13 (d, J=9 Hz, 2H); 7.86 (d, J=9 Hz, 2H); 9.87 (s, 1H, CHO).

### 20.2. 7-Methoxy-4-(4-methoxyphenyl)-N-{1-[4-(3-methoxypropoxy)benzyl]-piperidin-4-yl}phthalazin-1-amine dihydrochloride (compound 20)

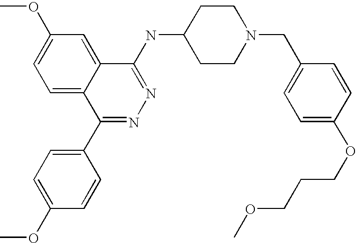

This compound in base form is obtained according to the procedure described in 3.3. by reacting 7-methoxy-4-(4-methoxyphenyl)-N-(piperidin-4-yl)phthalazin-1-amine with 4-(3-methoxypropoxy)benzaldehyde.

The dihydrochloride is obtained by means of the treatment described in 4.3.

### EXAMPLE 21

### N-(1-{4-[3-(Dimethylamino)propoxy]-3-fluorobenzyl}piperidin-4-yl)-4-ethyl-7-meth-oxyphthalazin-1-amine trihydrochloride (compound 21)

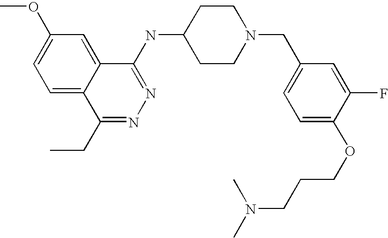

### 21.1. N-(1-Benzylpiperidin-4-yl)-4-ethyl-7-methoxyphthalazin-1-amine

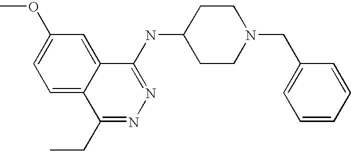

This compound is obtained according to the procedure described in 3.1. by reacting 1-chloro-4-ethyl-7-methoxyphthalazine (synthesis described in 6.3) with 4-amino-1-benzyl-piperidine.

LC/MS: MH⁺=377 (Rt=4.82 minutes pH 3.1)

¹H NMR (DMSO-d₆, 250 MHz) δ ppm: 1.28 (t, J=7.5 Hz, 3H); 1.57-1.75 (m, 2H); 1.98-2.18 (m, 4H); 2.85-2.95 (m, 2H); 3.03 (q, J=7.5 Hz, 2H); 3.52 (s, 2H); 3.99 (s, 3H); 4.10-4.22 (m, 1H); 6.77 (d, J=7.2 Hz, 1H); 7.22-7.37 (m, 5H), 7.44 (dd, J₁=9 Hz, J₂=2.5 Hz, 1H), 7.69 (d, J=2.5 Hz, 1H); 7.96 (d, J=9 Hz, 1H).

### 21.2. 4-Ethyl-7-methoxy-N-(piperidin-4-yl)phthalazin-1-amine

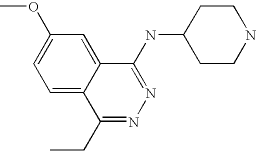

This compound is obtained by debenzylation of N-(1-benzylpiperidin-4-yl)-4-ethyl-7-meth-oxyphthalazin-1-amine according to the procedure described in 3.2.

m.p.=203° C. (B)

LC/MS: MH⁺=287 (Rt=3.84 and 3.96 minutes, pH 3.1)

¹H NMR (DMSO-d₆, 250 MHz) δ ppm: 1.28 (t, J=7.5 Hz, 3H); 1.35-1.57 (m, 2H); 1.92-2.04 (m, 2H); 2.10-2.25 (m, 1H); 2.55-2.65 (m, 2H); 2.96-3.10 (m, 4H); 3.96 (s, 3H); 4.12-4.25 (m, 1H); 6.76 (d, J=7.2 Hz, 1H); 7.44 (dd, J₁=9 Hz, J₂=2.5 Hz, 1H), 7.71 (d, J=2.5 Hz, 1H); 7.96 (d, J=9 Hz, 1H).

### 21.3. N-(1-{4-[3-(Dimethylamino)propoxy]-3-fluorobenzyl}piperidin-4-yl)-4-ethyl-7-methoxyphthalazin-1-amine trihydrochloride (compound 21)

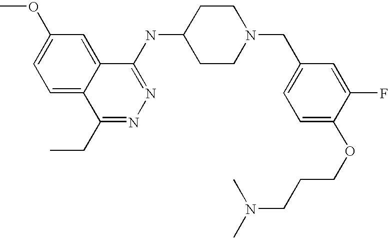

This compound in base form is obtained according to the procedure described in 8.2. by reacting 4-ethyl-7-methoxy-N-(piperidin-4-yl)phthalazin-1-amine with 4-(3-dimethyl-aminopropoxy)-3-fluorobenzaldehyde prepared in 8.1.

The trihydrochloride is obtained by means of the treatment described in 6.5.

### EXAMPLE 22

### N-(1-{4-[3-(Dimethylamino)propoxy]-3-fluorobenzyl}piperidin-4-yl)-7-methoxy-4-(methoxymethyl)phthalazin-1-amine trihydrochloride (compound 22)

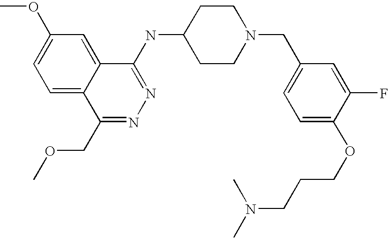

### 22.1. N-(1-Benzylpiperidin-4-yl)-7-methoxy-4-methoxymethylphthalazin-1-amine

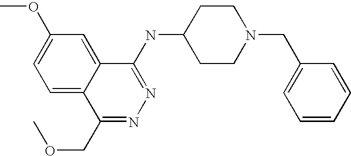

This compound is obtained according to the procedure described in 3.1. by reacting 1-chloro-7-methoxy-4-methoxymethylphthalazine (synthesis described in 7.3) with 4-amino-1-benzylpiperidine.

¹H NMR (DMSO-d₆, 250 MHz) δ ppm: 1.57-1.77 (m, 2H); 1.98-2.20 (m, 4H); 2.85-2.98 (m, 2H); 3.28 (s, 3H); 3.52 (s, 2H); 3.96 (s, 3H); 4.12-4.30 (m, 1H); 4.77 (s, 2H); 6.99 (d, J=7.2 Hz, 1H); 7.22-7.38 (m, 5H), 7.47 (dd, J₁=9 Hz, J₂=2.5 Hz, 1H), 7.71 (d, J=2.5 Hz, 1H); 8.01 (d, J=9 Hz, 1H).

### 22.2. 7-Methoxy-4-methoxymethyl-N-(piperidin-4-yl)phthalazin-1-amine

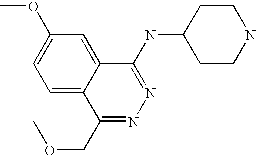

This compound is obtained by debenzylation of N-(1-benzylpiperidin-4-yl)-7-methoxy-4-methoxymethylphthalazin-1-amine according to the procedure described in 3.2.

m.p.=195° C. (B)

LC/MS: MH⁺=303 (Rt=3.82 minutes, pH 3.1)

¹H NMR (DMSO-d₆, 250 MHz) δ ppm: 1.42-1.55 (m, 2H); 1.93-2.02 (m, 2H); 2.05-2.20 (m, 1H); 2.53-2.63 (m, 2H); 2.98-3.08 (m, 2H); 3.28 (s, 3H); 3.96 (s, 3H); 4.20-4.30 (m, 1H); 4.76 (s, 2H); 6.98 (d, J=7.2 Hz, 1H); 7.45 (dd, J₁=9 Hz, J₂=2.5 Hz, 1H), 7.72 (d, J=2.5 Hz, 1H); 8.00 (d, J=9 Hz, 1H).

### 22.3. N-(1-{4-[3-(Dimethylamino)propoxy]-3-fluorobenzyl}piperidin-4-yl)-7-methoxy-4-(methoxymethyl)phthalazin-1-amine trihydrochloride (compound 22)

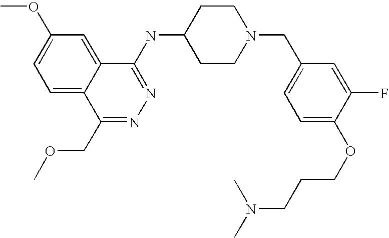

This compound in base form is obtained according to the procedure described in 8.2. by reacting 7-methoxy-4-methoxymethyl-N-(piperidin-4-yl)phthalazin-1-amine with 4-(3-dimethylaminopropoxy)-3-fluorobenzaldehyde prepared in 8.1.

The trihydrochloride is obtained by means of the treatment described in 7.4.

### EXAMPLE 23

### N-{1-[3-Fluoro-4-(3-pyrrolidin-1-ylpropoxy)benzyl]piperidin-4-yl}-7-methoxy-4-(4-methoxyphenyl)phthalazin-1-amine trihydrochloride (compound 23)

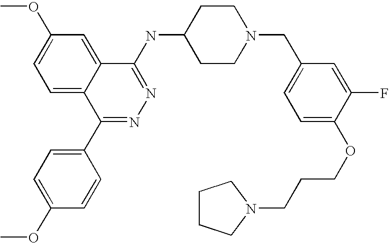

### 23.1 3-Fluoro-4-(3-pyrrolidin-1-ylpropoxy)benzaldehyde

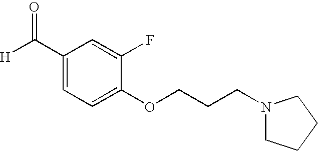

This compound is obtained according to the procedure described in 4.2. by reacting N-(3-chloropropyl)pyrrolidine with 3-fluoro-4-hydroxybenzaldehyde.

¹H NMR (DMSO-d₆, 250 MHz) δ ppm: 1.60-1.80 (m, 4H); 1.86-2.02 (m, 2H); 2.34-2.58 (m, 6H); 4.18-4.28 (m, 2H); 7.36-7.45 (m, 1H); 7.70 (d, J=11 Hz, 1H), 7.77 (d, J=9 Hz, 1H), 9.88 (s, 1H, CHO).

### 23.3. N-{1-[3-Fluoro-4-(3-pyrrolidin-1-ylpropoxy)benzyl]piperidin-4-yl}-7-meth-oxy-4-(4-methoxyphenyl)phthalazin-1-amine trihydrochloride (compound 23)

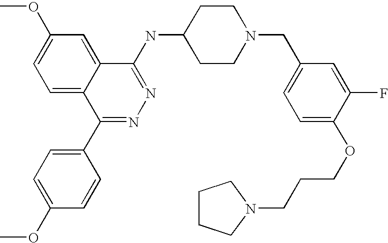

This compound in base form is obtained according to the procedure described in 3.3. by reacting 7-methoxy-4-(4-methoxyphenyl)-N-(piperidin-4-yl)phthalazin-1-amine with 3-fluoro-4-(3-pyrrolidin-1-ylpropoxy)benzaldehyde.

The trihydrochloride is obtained by means of the treatment described in 6.5.

### EXAMPLE 24

### 1-(3-{4-[(4-{[7-Methoxy-4-(4-methoxyphenyl)phthalazin-1-yl]amino}piperidin-1-yl)-methyl]phenoxy}propyl)pyrrolidin-2-one trihydrochloride (compound 24)

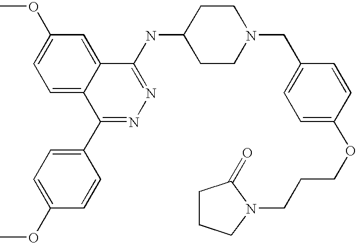

### 24.1. 4-[3-(2-Oxopyrrolidin-1-yl)propoxy]benzaldehyde

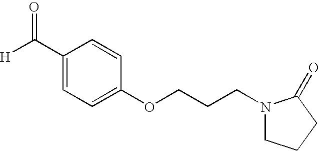

This compound is obtained according to the procedure described in 4.2. by reacting 4-hydroxybenzaldehyde with 1-(3-chloropropyl)pyrrolidin-2-one, which was obtained beforehand by reacting 1-(3-hydroxypropyl)pyrrolidin-2-one with thionyl chloride.

LC/MS: MH⁺=248 (Rt=6.31 minutes, pH 3.1)

### 24.2 1-(3-{4-[(4-{[7-Methoxy-4-(4-methoxyphenyl)phthalazin-1-yl]amino}piperidin-1-yl)methyl]phenoxy}propyl)pyrrolidin-2-one trihydrochloride (compound 24)

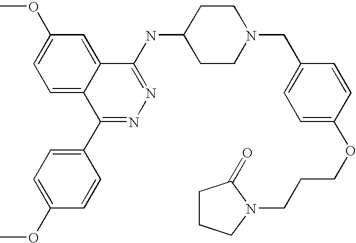

This compound in base form is obtained according to the procedure described in 8.2. by reacting 7-methoxy-4-(4-methoxyphenyl)-N-(piperidin-4-yl)phthalazin-1-amine with 4-[3-(2-oxopyrrolidin-1-yl)propoxy]benzaldehyde.

¹H NMR (DMSO-d₆, 400 MHz) δ ppm: 1.62-1.78 (m, 2H); 1.88-1.98 (m, 4H); 2.0-2.15 (m, 4H); 2.20-2.28 (m, 2H); 2.85-2.95 (m, 2H); 2.30-2.40 (m, 4H); 3.48 (s, 2H); 3.85 (s, 3H); 3.92-4.00 (m, 2H); 4.0 (s, 3H); 4.20-4.30 (m, 1H); 6.89 (d, J=8 Hz, 2H); 6.98 (d, J=7.5 Hz, 1H); 7.08 (d, J=8 Hz, 2H); 7.23 (d, J=8 Hz, 2H); 7.42 (dd, J₁=7.5 Hz, J₂=2.5 Hz, 1H); 7.50 (d, J=8 Hz, 2H); 7.72-7.78 (m, 2H);

The trihydrochloride is obtained by means of the treatment described in 6.5.

### EXAMPLE 25

### N-(1-{4-[2-(Dimethylamino)ethoxy]benzyl}piperidin-4-yl)-7-methoxy-4-(4-methoxy-phenyl)phthalazin-1-amine trihydrochloride (compound 25)

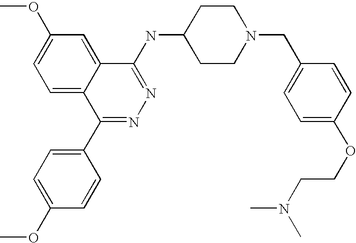

### 25.1. 4-[2-(Dimethylamino)ethoxy]benzaldehyde

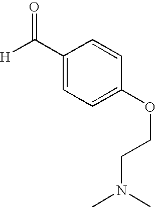

This compound is obtained according to the procedure described in 5.1. by reacting 2-chloro-N,N-dimethylethylamine hydrochloride with 4-hydroxybenzaldehyde.

¹H NMR (DMSO-d₆, 250 MHz) δ ppm: 2.22 (s, 6H); 2.65 (t, J=5.7 Hz, 2H); 4.17 (t, J=5.7 Hz, 2H); 7.14 (d, J=6.8 Hz, 2H), 7.86 (d, J=6.8 Hz, 2H), 9.88 (s, 1H, CHO).

25.2. N-(1-{4-[2-(Dimethylamino)ethoxy]benzyl}piperidin-4-yl)-7-methoxy-4-(4-methoxyphenyl)phthalazin-1-amine trihydrochloride (compound 25)

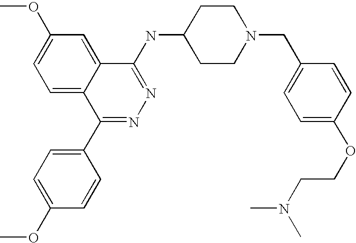

This compound in base form is obtained according to the procedure described in 8.2. by reacting 7-methoxy-4-(4-methoxyphenyl)-N-(piperidin-4-yl)phthalazin-1-amine with 4-[2-(dimethylamino)ethoxy]benzaldehyde.

The trihydrochloride is obtained by means of the treatment described in 17.2.

### EXAMPLE 26

### 7-Methoxy-4-(4-methoxyphenyl)-N-(1-{4-[2-(1-methylpyrrolidin-2-yl)ethoxy]benzyl}-piperidin-4-yl)phthalazin-1-amine trihydrochloride (compound 27)

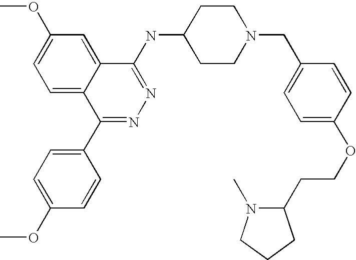

### 26.1 4-[2-(1-Methylpyrrolidin-2-yl)ethoxy]benzaldehyde and 4-[(1-methylazepan-4-yl)oxy]benzaldehyde

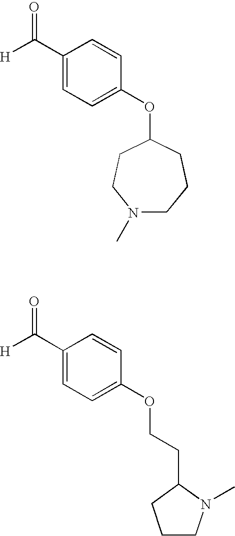

These compounds are obtained according to the procedure described in 14.1. by reacting 2-(2-chloroethyl)-N-methylpyrrolidine hydrochloride with 4-hydroxybenzaldehyde. The two compounds are separated by chromatography on silica gel (eluent: toluene/methanol 50/50 (v/v)).

The first compound eluted is 4-[2-(1-methylpyrrolidin-2-yl)ethoxy]benzaldehyde:

¹H NMR (DMSO-d₆, 250 MHz) δ ppm: 1.15-1.25 (m, 1H); 1.40-1.75 (m, 4H); 1.85-2.20 (m, 4H); 2.23 (s, 3H); 2.92-3.0 (m, 1H); 4.10-4.18 (m, 1H); 7.13 (d, J=9 Hz, 2H), 7.85 (d, J=9 Hz, 2H), 9.88 (s, 1H, CHO).

The second compound eluted is 4-[(1-methylazepan-4-yl)oxy]benzaldehyde:

¹H NMR (DMSO-d₆, 250 MHz) δ ppm: 1.50-1.92 (m, 4H); 2.0-2.18 (m, 2H); 2.22 (s, 3H); 2.45-2.68 (m, 4H); 4.72-4.80 (m, 1H); 7.14 (d, J=9 Hz, 2H), 7.84 (d, J=9 Hz, 2H), 9.86 (s, 1H, CHO).

### 26.2 7-Methoxy-4-(4-methoxyphenyl)-N-(1-{4-[2-(1-methylpyrrolidin-2-yl)eth-oxy]benzyl}piperidin-4-yl)phthalazin-1-amine trihydrochloride (compound 27)

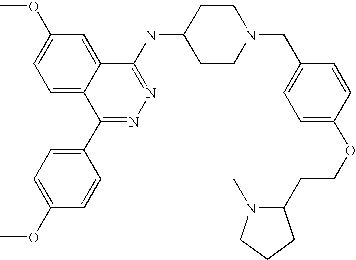

To a solution of 300 mg (0.82 mmol) of 7-methoxy-4-(4-methoxyphenyl)-N-(piperidin-4-yl)phthalazin-1-amine in 10 mL of 1,2-dichloroethane are added 172 mg (0.74 mmol) of 4-[2-(1-methylpyrrolidin-2-yl)ethoxy]benzaldehyde. The mixture is stirred for 1 hour 30 minutes under nitrogen and 226 mg (1.07 mmol) of sodium triacetoxyborohydride are then added. The reaction medium is stirred for 48 hours at room temperature, 20 mL of 1N sodium hydroxide are added, and the mixture is then extracted with dichloromethane. The organic phase is extracted with 40 mL of N hydrochloric acid. The acidic aqueous phase obtained is basified with 2 N sodium hydroxide and then extracted with dichloromethane. The organic extract is washed with saturated aqueous sodium chloride, dried over anhydrous sodium sulfate and then evaporated under reduced pressure. The residue is purified on a column of basic alumina (eluent: dichloromethane/methanol from 100/0 to 98/2 (v/v)). 220 mg of an oily product are obtained.

The trihydrochloride is obtained by means of the treatment described in 4.3.

### EXAMPLE 27

### 7-Methoxy-4-(4-methoxyphenyl)-N-(1-{4-[(1-methylazepan-4-yl)oxy]benzyl}piperidin-4-yl)phthalazin-1-amine trihydrochloride (compound 26)

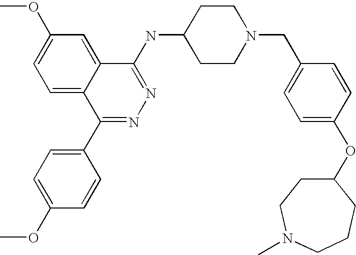

This compound in base form is obtained according to the procedure described in 26.2. by reacting 7-methoxy-4-(4-methoxyphenyl)-N-(piperidin-4-yl)phthalazin-1-amine with 4-[(1-methylazepan-4-yl)oxy]benzaldehyde.

The trihydrochloride is obtained by means of the treatment described in 4.3.

### EXAMPLE 28

### 4-Ethyl-N-[1-(4-ethynylbenzyl)piperidin-4-yl]-7-methoxyphthalazin-1-amine dihydro-chloride (compound 28)

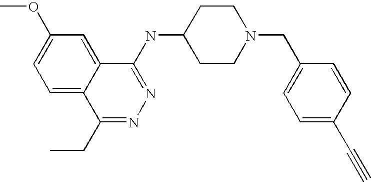

### 28.1. 4-Ethyl-7-methoxy-N-(1-{4-[(trimethylsilyl)ethynyl]benzyl}piperidin-4-yl)phthalazin-1-amine

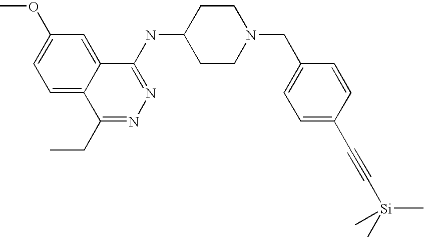

This compound is obtained according to the procedure described in 4.3. by reacting 4-ethyl-7-methoxy-N-(piperidin-4-yl)phthalazin-1-amine with 4-(trimethylsilyl)ethynylbenzaldehyde.

LC/MS: MH⁺=473 (Rt=6.05 minutes pH 3.1)

¹H NMR (DMSO-d₆, 400 MHz) δ ppm: 0.29 (s, 9H); 1.26 (t, J=7.5 Hz, 3H); 1.60-1.70 (m, 2H); 1.98-2.18 (m, 4H); 2.83-2.90 (m, 2H); 3.05 (q, J=7.5 Hz, 2H); 3.53 (s, 2H); 3.95 (s, 3H); 4.10-4.20 (m, 1H); 6.75 (d, J=7.2 Hz, 1H); 7.32-7.36 (m, 2H), 7.40-7.45 (m, 3H), 7.67 (d, J=2.5 Hz, 1H); 7.95 (d, J=9 Hz, 1H).

### 28.2. 4-Ethyl-N-[1-(4-ethynylbenzyl)piperidin-4-yl]-7-methoxyphthalazin-1-amine dihydrochloride (compound 28)

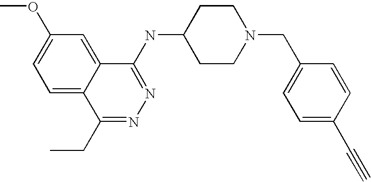

To a solution of 0.85 g (1.8 mmol) of 4-ethyl-7-methoxy-N-(1-{4-[(trimethylsilyl)-ethynyl]benzyl}piperidin-4-yl)phthalazin-1-amine in 90 mL of methanol are added 0.55 g (4 mmol) of potassium carbonate. The mixture is stirred 24 hours at room temperature, followed by addition of water and 1N hydrochloric acid to neutral pH, and the mixture is extracted with dichloromethane. The organic extract is washed with saturated aqueous sodium chloride, dried over anhydrous sodium sulfate and then evaporated under reduced pressure. 0.75 g of gummy product is obtained.

LC/MS: MH⁺=401 (Rt=4.77 minutes pH 3.1)

¹H NMR (DMSO-d₆, 250 MHz) δ ppm: 1.28 (t, J=7.5 Hz, 3H); 1.58-1.72 (m, 2H); 1.98-2.18 (m, 4H); 2.83-2.92 (m, 2H); 3.05 (q, J=7.5 Hz, 2H); 3.53 (s, 2H); 3.96 (s, 3H); 4.14 (s, 1H); 4.10-4.20 (m, 1H); 6.76 (d, J=7.2 Hz, 1H); 7.33-7.38 (m, 2H), 7.42-7.50 (m, 3H), 7.69 (d, J=2.5 Hz, 1H); 7.96 (d, J=9 Hz, 1H).

The dihydrochloride is obtained by means of the treatment described in 6.5.

### EXAMPLE 29

### N-(1-{4-[3-(Dimethylamino)prop-1-yn-1-yl]benzyl}piperidin-4-yl)-4-ethyl-7-methoxy-phthalazin-1-amine trihydrochloride (compound 29)

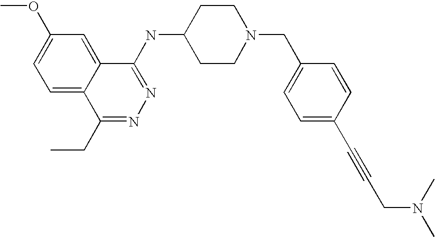

A mixture consisting of 250 mg (0.62 mmol) of 4-ethyl-N-[1-(4-ethynylbenzyl)piperidin-4-yl]-7-methoxyphthalazin-1-amine, 120 mg (1.28 mmol) of N,N-dimethylmethyleneiminium chloride and 6 mg (0.06 mmol) of copper (I) chloride in 6.3 mL of dioxane. The mixture is stirred 12 hours at 70° C. During this heating period, twice 60 mg of N,N-dimethylmethyleneiminium chloride are added to the reaction medium. After cooling, water and 1N sodium hydroxide are added and the mixture is then filtered through Celite. The organic phase is washed with saturated aqueous sodium chloride, dried over anhydrous sodium sulfate and then evaporated under reduced pressure. The residue obtained is purified on a column of silica (eluent: dichloromethane/methanol from 100/0 to 85/15 (v/v)). 220 mg of an oily product are obtained.

¹H NMR (DMSO-d₆, 400 MHz) δ ppm: 1.25 (t, J=7.5 Hz, 3H); 1.60-1.70 (m, 2H); 1.98-2.15 (m, 4H); 2.24 (s, 6H); 2.85-2.90 (m, 2H); 3.05 (q, J=7.5 Hz, 2H); 3.45 (s, 2H); 3.95 (s, 3H); 4.10-4.20 (m, 1H); 6.76 (d, J=7.2 Hz, 1H); 7.33-7.38 (m, 2H), 7.40-7.48 (m, 3H), 7.68 (d, J=2.5 Hz, 1H); 7.96 (d, J=9 Hz, 1H).

The trihydrochloride is obtained by means of the treatment described in 6.5.

### EXAMPLE 30

### 3-{4-[(4-{[7-Methoxy-4-(4-methoxyphenyl)phthalazin-1-yl]amino}piperidin-1-yl)-methyl]phenoxy}propan-1-ol dihydrochloride (compound 30)

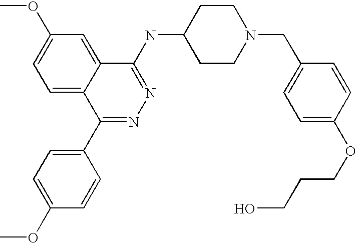

### 30.1 4-[3-(Tetrahydro-2H-pyran-2-yloxy)propoxy]benzaldehyde

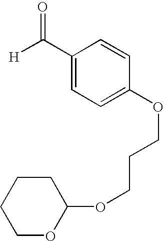

To a solution of 1.31 g (5.9 mmol) of 2-(3-bromopropoxy)tetrahydro-2H-pyran and 0.94 g (7.7 mmol) of 4-hydroxybenzaldehyde in 23 mL of DMF are added 1.63 g (11.8 mmol) of potassium carbonate. The mixture is stirred at room temperature for 20 hours, followed by addition of water, and is extracted with ethyl acetate. The organic extract is washed with water and then with saturated aqueous sodium chloride, dried over anhydrous sodium sulfate and then evaporated under reduced pressure. 1.52 g of an oily product are obtained.

¹H NMR (DMSO-d₆, 250 MHz) δ ppm: 1.45-1.80 (m, 6H); 1.92-2.08 (m, 2H); 3.38-3.58 (m, 2H); 3.70-3.83 (m, 2H); 4.15-4.22 (m, 2H); 4.55-4.60 (m, 1H); 7.13 (d, J=9 Hz, 2H), 7.86 (d, J=9 Hz, 2H), 9.88 (s, 1H, CHO).

### 30.2 3-{4-[(4-{[7-Methoxy-4-(4-methoxyphenyl)phthalazin-1-yl]amino}piperidin-1-yl)methyl]phenoxy}propan-1-ol dihydrochloride (compound 30)

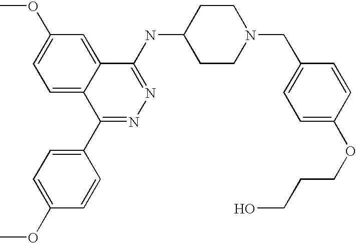

To a solution of 528 mg (2 mmol) of 7-methoxy-4-(4-methoxyphenyl)-N-(piperidin-4-yl)phthalazin-1-amine in 20 mL of 1,2-dichloroethane are added 875 mg (2.4 mmol) of 4-[3-(tetrahydro-2H-pyran-2-yloxy)propoxy]benzaldehyde. The mixture is stirred for 30 minutes under nitrogen and 550 mg (2.6 mmol) of sodium triacetoxyborohydride are then added. The reaction medium is stirred for 24 hours at room temperature, hydrolyzed with water and 1 N sodium hydroxide and then extracted with dichloromethane. The organic extract is washed with saturated aqueous sodium chloride, dried over anhydrous sodium sulfate and then evaporated under reduced pressure. The residue is purified on a column of silica (eluent: dichloromethane/methanol from 100/0 to 90/10 (v/v)). 943 mg of an oily product are obtained. 350 mg (0.57 mmol) of this product are dissolved in 7 mL of methanol 3.5 mL (2.45 mmol) of Dowex 50×2-200 resin prewashed with methanol are added. The mixture is stirred for 24 hours and then filtered. The resin is washed with methanol and is then suspended in 4 mL of methanol and 3.5 mL of 7 N ammoniacal methanol. The mixture is stirred for at least 4 hours and then filtered. The filtrate is evaporated under reduced pressure. The product obtained is sufficiently pure. The dihydrochloride is obtained by means of the treatment described in 6.5.

### EXAMPLE 31

### 4-Ethyl-7-methoxy-N-{1-[4-(4-pyrrolidin-1-ylpiperidin-1-yl)benzyl]piperidin-4-yl}-phthalazin-1-amine trihydrochloride (compound 31)

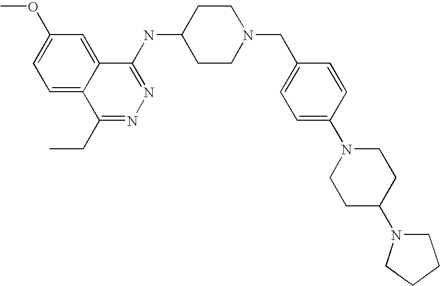

### 31.1 4-(4-Pyrrolidin-1-ylpiperidin-1-yl)benzaldehyde

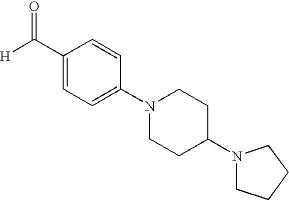

A mixture consisting of 1 g (5.4 mmol) of 4-bromobenzaldehyde and 1 g (6.5 mmol) of 4-(1-pyrrolidinyl)piperidine in 10 mL of anhydrous toluene is stirred at room temperature under an argon atmosphere, and 2.46 g (7.56 mmol) of cesium carbonate, 50 mg of tris(dibenzylideneacetone)dipalladium(0) and 50 mg of 2,2′-bis(diphenylphosphino)-1,1′-binaphthyl (racemic) are added. The mixture is stirred at 80° C. for 40 hours, and, after cooling, is then filtered through Celite. The solid is rinsed with ethyl acetate and the filtrate is evaporated to dryness. The residue obtained is purified on a column of silica (eluent: dichloromethane/methanol from 100/0 to 85/15 (v/v)). 0.94 g of an orange-colored oil is obtained, from which the product crystallizes.

¹H NMR (DMSO-d₆, 250 MHz) δ ppm: 1.35-1.52 (m, 2H); 1.60-1.75 (m, 4H); 1.85-1.95 (m, 2H); 2.20-2.32 (m, 1H); 2.45-2.60 (m, 4H); 2.95-3.05 (m, 2H); 3.85-3.96 (m, 2H); 7.04 (d, J=9 Hz, 2H), 7.70 (d, J=9 Hz, 2H), 9.70 (s, 1H, CHO).

### 31.2 4-Ethyl-7-methoxy-N-{1-[4-(4-pyrrolidin-1-ylpiperidin-1-yl)benzyl]piperidin-4-yl}phthalazin-1-amine trihydrochloride (compound 31)

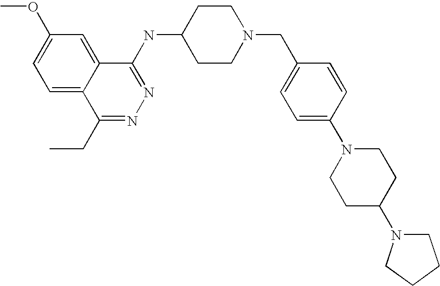

This compound in base form is obtained according to the procedure described in 26.2. by reacting 4-ethyl-7-methoxy-N-(piperidin-4-yl)phthalazin-1-amine with 4-(4-pyrrolidin-1-ylpiperidin-1-yl)benzaldehyde. The trihydrochloride is obtained by treating the product dissolved in ethyl acetate with a 2 N solution of hydrogen chloride in diethyl ether. A suspension forms, which is filtered. The powder obtained is dried at 40° C. under reduced pressure.

### EXAMPLE 32

### N-(1-{4-[3-(Dimethylamino)propoxy]benzyl}piperidin-4-yl)-7-methoxy-4-(methoxy-methyl)phthalazin-1-amine trihydrochloride (compound 32)

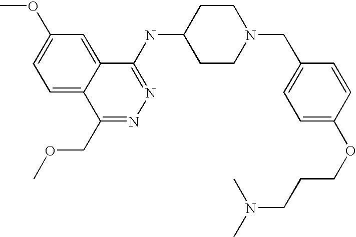

This compound in base form is obtained according to the procedure described in 8.2. by reacting 7-methoxy-4-methoxymethyl-N-(piperidin-4-yl)phthalazin-1-amine (prepared in 22.2) with 4-(3-dimethylaminopropoxy)benzaldehyde.

The trihydrochloride is obtained by means of the treatment described in 7.4.

### EXAMPLE 33

### N-(1-{4-[(3R)-3-(Dimethylamino)pyrrolidin-1-yl]benzyl}piperidin-4-yl)-7-methoxy-4-(methoxymethyl)phthalazin-1-amine trihydrochloride (compound 34)

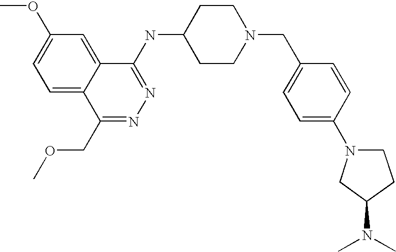

### 33.1 4-[(3R)-3-(Dimethylamino)pyrrolidin-1-yl]benzaldehyde

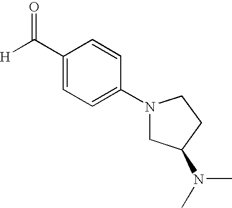

This compound is obtained according to the procedure described in 31.1 by reacting 4-bromobenzaldehyde with (3R)-3-(dimethylamino)pyrrolidine.

[α]_{D}²²=−3.1 (c=0.49, methanol)

¹H NMR (DMSO-d₆, 250 MHz) δ ppm: 1.75-1.95 (m, 1H); 2.10-2.21 (m, 1H); 2.22 (s, 6H); 2.72-2.88 (m, 1H); 3.09-3.18 (m, 1H); 3.29-3.40 (m, 1H); 3.47-3.62 (m, 2H); 6.66 (d, J=9 Hz, 2H), 7.68 (d, J=9 Hz, 2H), 9.66 (s, 1H, CHO).

### 33.2. N-(1-{4-[(3R)-3-(Dimethylamino)pyrrolidin-1-yl]benzyl}piperidin-4-yl)-7-meth-oxy-4-(methoxymethyl)phthalazin-1-amine trihydrochloride (compound 34)

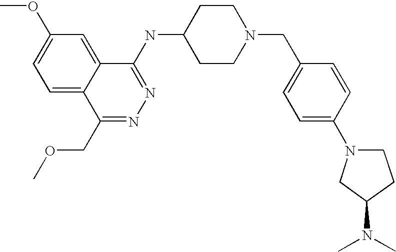

This compound in base form is obtained according to the procedure described in 26.2 by reacting 7-methoxy-4-methoxymethyl-N-(piperidin-4-yl)phthalazin-1-amine (preparation 22.2) with 4-[(3R)-3-(dimethylamino)pyrrolidin-1-yl]benzaldehyde.

The trihydrochloride is obtained by means of the treatment described in 6.5.

### EXAMPLE 34

### N-(1-{4-[(3S)-3-(Dimethylamino)pyrrolidin-1-yl]benzyl}piperidin-4-yl)-7-methoxy-4-(methoxymethyl)phthalazin-1-amine trihydrochloride (compound 33)

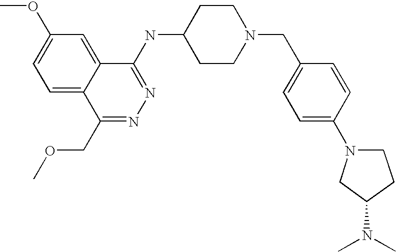

### 34.1 4-[(3S)-3-(Dimethylamino)pyrrolidin-1-yl]benzaldehyde

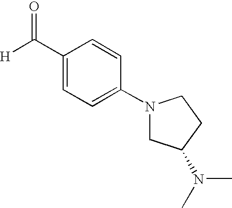

This compound is obtained according to the procedure described in 31.1 by reacting 4-bromobenzaldehyde with (3S)-3-(dimethylamino)pyrrolidine.

[α]_{D}²²=+2.8 (c=0.67, methanol).

### 34.2. N-(1-{4-[(3S)-3-(Dimethylamino)pyrrolidin-1-yl]benzyl}piperidin-4-yl)-7-meth-oxy-4-(methoxymethyl)phthalazin-1-amine trihydrochloride (compound 33)

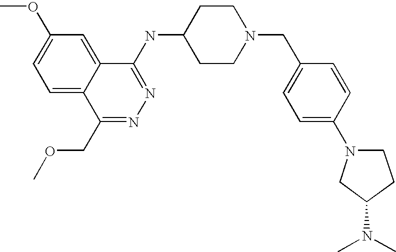

This compound in base form is obtained according to the procedure described in 26.2 by reacting 7-methoxy-4-methoxymethyl-N-(piperidin-4-yl)phthalazin-1-amine (preparation 22.2) with 4-[(3S)-3-(dimethylamino)pyrrolidin-1-yl]benzaldehyde.

The trihydrochloride is obtained by means of the treatment described in 6.5.

### EXAMPLE 35

### N-[1-(1,3-Benzodioxol-5-ylmethyl)piperidin-4-yl]-7-methoxy-4-[4-(2-methyl-2H-tetrazol-5-yl)phenyl]phthalazin-1-amine dihydrochloride (compound 35)

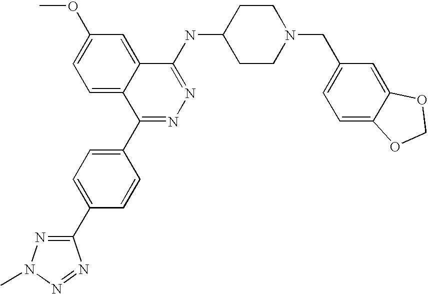

### 35.1. N-Methoxy-N-methyl-4-(2H-tetrazol-5-yl)benzamide

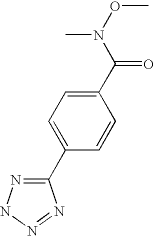

To a solution of 5 g (26.3 mmol) of 4-cyano-N-methoxy-N-methylbenzamide in 100 mL of THF are added 13.96 mL (106 mmol) of trimethylsilylazide and 3.27 g (13.14 mmol) of dibutyltin oxide and the mixture is then stirred for six hours at reflux. A further 10 mL of trimethylsilylazide are added and the mixture is refluxed for a further eight hours. After cooling, the mixture is evaporated under reduced pressure and the residue obtained is purified on a column of silica (eluent: dichloromethane/methanol from 100/0 to 85/15 (v/v)). 5.1 g of a white solid are obtained.

m.p.=173° C. (M)

LC/MS: MH⁺=234 (Rt=5.26 minutes pH 3.1)

¹H NMR (DMSO-d₆, 250 MHz) δ ppm: 3.30 (s, 3H); 3.58 (s, 3H); 7.81 (d, J=8.5 Hz, 2H); 8.11 (d, J=8.5 Hz, 2H).

### 35.2. N-Methoxy-N-methyl-4-(2-methyl-2H-tetrazol-5-yl)benzamide

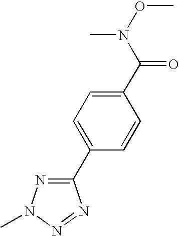

To a solution of 5.1 g (21.8 mmol) of N-Methoxy-N-methyl-4-(2H-tetrazol-5-yl)benzamide in 100 mL of DMF are added 3.32 g (24 mmol) of potassium carbonate, followed by 2.72 mL (44 mmol) of iodomethane. The mixture is stirred at room temperature for 16 hours and water is then added. A precipitate forms, which is filtered off, washed with ethyl acetate and dried in an oven under reduced pressure. 2.7 g of a white powder are obtained.

m.p.=173° C. (K)

¹H NMR (DMSO-d₆, 250 MHz) δ ppm: 3.30 (s, 3H); 3.58 (s, 3H); 4.46 (s, 3H); 7.77 (d, J=8.5 Hz, 2H); 8.12 (d, J=8.5 Hz, 2H).

### 35.3. 5-Methoxy-2-[4-(2-methyl-2H-tetrazol-5-yl)benzoyl]benzoic acid

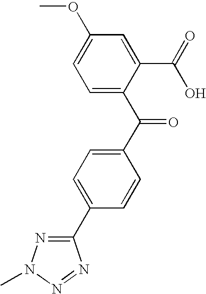

This compound is synthesized according to the method described in 2.4. by reacting 2-bromo-5-methoxybenzoic acid pretreated with n-butyllithium with N-Methoxy-N-methyl-4-(2-methyl-2H-tetrazol-5-yl)benzamide. It is used crude in the following reaction.

### 35.4. 7-Methoxy-4-[4-(2-methyl-2H-tetrazol-5-yl)phenyl]phthalazin-1(2H)-one

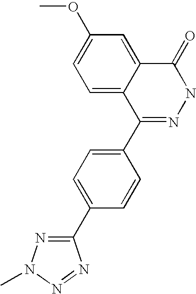

This compound is obtained according to the procedure described in 2.5. by reacting unpurified 5-methoxy-2-[4-(2-methyl-2H-tetrazol-5-yl)benzoyl]benzoic acid with hydrazine hydrate.

m.p.=289° C. (M)

LC/MS: MH⁺=335 (Rt=7.34 minutes pH 3.1)

¹H NMR δ in ppm (DMSO d 6): 3.98 (s, 3H); 4.48 (s, 3H); 7.50 (dd, J₁=9 Hz, J₂=2.7 Hz, 1H); 7.68-7.81 (m, 4H); 8.23 (d, J=8.5 Hz, 2H); 12.8 (s, 1H, NH).

### 35.5. 4-Chloro-6-methoxy-1-[4-(2-methyl-2H-tetrazol-5-yl)phenyl]phthalazine

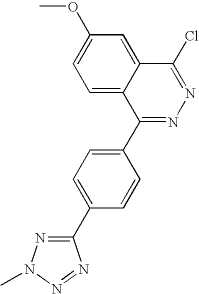

This compound is obtained according to the procedure described in 2.6. by reacting 7-methoxy-4-[4-(2-methyl-2H-tetrazol-5-yl)phenyl]phthalazin-1(2H)-one with phosphoryl chloride.

m.p.=235° C. (M)

LC/MS: MH⁺=353 (Rt=8.29 minutes pH 3.1)

¹H NMR 6 in ppm (DMSO d 6): 4.08 (s, 3H); 4.49 (s, 3H); 7.64 (d, J=2.5 Hz, 1H); 7.73 (dd, J₁=9 Hz, J₂=2.7 Hz, 1H); 7.92 (d, J=8.5 Hz, 2H); 8.03 (d, J=9 Hz, 1H); 8.30 (d, J=8.5 Hz, 2H).

### 35.6 N-[1-(1,3-Benzodioxol-5-ylmethyl)piperidin-4-yl]-7-methoxy-4-[4-(2-methyl-2H-tetrazol-5-yl)phenyl]phthalazin-1-amine dihydrochloride (compound 35)

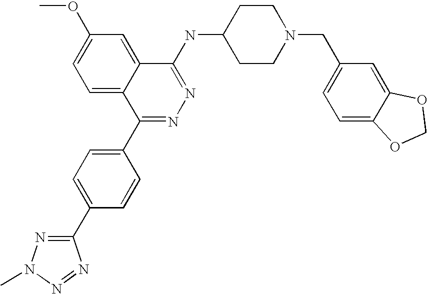

This compound in base form is obtained according to the procedure described in 1.9. by reacting 4-chloro-6-methoxy-1-[4-(2-methyl-2H-tetrazol-5-yl)phenyl]phthalazine with 1-(1,3-benzodioxol-5-ylmethyl)piperidin-4-ylamine. The dihydrochloride is obtained by means of the treatment described in 4.3.

### EXAMPLE 36

### 7-Methoxy-4-(methoxymethyl)-N-(1-{4-[(1-methylpyrrolidin-3-yl)methoxy]benzyl}-piperidin-4-yl)phthalazin-1-amine trihydrochloride (compound 36)

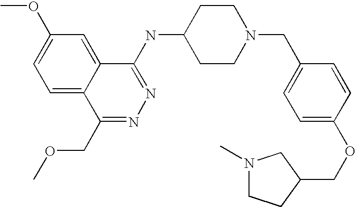

### 36.1. 4-[(1-Methylpyrrolidin-3-yl)methoxy]benzaldehyde

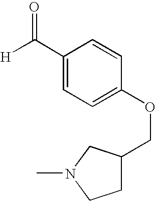

This compound is obtained via the Mitsunobu reaction according to the procedure described in 17.1. by reacting 4-hydroxybenzaldehyde with (1-methylpyrrolidin-3-yl)methanol synthesized via the reaction described in J. Org. Chem. 1961 pp. 1519-1524. LC/MS: MH⁺=220 (Rt=4.00 minutes pH 3.1)

### 36.2 7-Methoxy-4-(methoxymethyl)-N-(1-{4-[(1-methylpyrrolidin-3-yl)methoxy]-benzyl}piperidin-4-yl)phthalazin-1-amine trihydrochloride (compound 36)

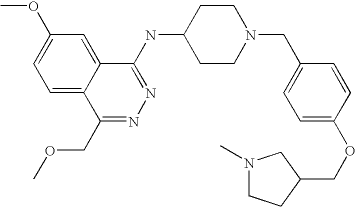

This compound in base form is obtained according to the procedure described in 26.2 by reacting 7-methoxy-4-methoxymethyl-N-(piperidin-4-yl)phthalazin-1-amine (preparation 22.2) with 4-[(1-methylpyrrolidin-3-yl)methoxy]benzaldehyde.

¹H NMR (DMSO-d₆, 250 MHz) δ ppm: 1.43-1.55 (m, 1H); 1.58-1.70 (m, 2H); 1.90-2.10 (m, 5H); 2.23 (s, 3H); 2.32-2.42 (m, 2H); 2.45-2.60 (m, 3H); 2.85-2.91 (m, 2H); 3.28 (s, 3H); 3.43 (s, 3H); 3.80-3.85 (m, 2H); 3.95 (s, 3H); 4.15-4.25 (m, 1H); 4.76 (s, 2H); 6.88 (d, J=8.8 Hz, 2H); 6.99 (d, J=7.6 Hz, 1H); 7.22 (d, J=8.8 Hz, 2H); 7.46 (dd, J₁=8.8 Hz, J₂=2 Hz, 1H); 7.70 (d, J=2 Hz, 1H); 8.0 (d, J=8.8 Hz, 1H).

The trihydrochloride is obtained by means of the treatment described in 4.3.

### EXAMPLE 37

### Trihydrochloride of the beta anomer of 7-methoxy-4-(methoxymethyl)-N-(1-{4-[(8-methyl-8-azabicyclo[3.2.1]oct-3-yl)oxy]benzyl}piperidin-4-yl)phthalazin-1-amine (compound 37)

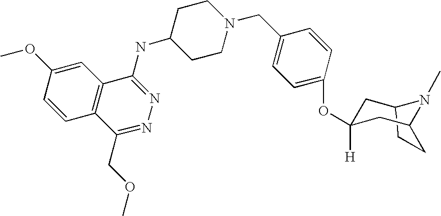

### 37.1. 4-[(8-Methyl-8-azabicyclo[3.2.1]oct-3-yl)oxy]benzaldehyde (beta anomer)

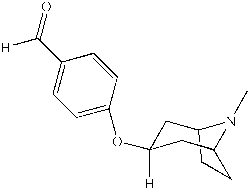

This compound is obtained according to the procedure described in 17.1. by reacting 4-hydroxybenzaldehyde with 8-methyl-8-azabicyclo[3.2.1]octan-3-ol (alpha anomer)

¹H NMR (DMSO-d₆, 250 MHz) δ ppm: 1.10-1.25 (m, 2H); 1.60-1.78 (m, 3H); 1.90-2.02 (m, 3H); 2.23 (s, 3H); 3.10-3.20 (m, 2H); 4.66-4.82 (m, 1H, CH—O axial); 7.14 (d, J=9 Hz, 2H), 8.83 (d, J=9 Hz, 2H), 9.86 (s, 1H, CHO).

### 37.2 Trihydrochloride of the beta anomer of 7-methoxy-4-(methoxymethyl)-N-(1-{4-[(8-methyl-8-azabicyclo[3.2.1]oct-3-yl)oxy]benzyl}piperidin-4-yl)phthalazin-1-amine (compound 37)

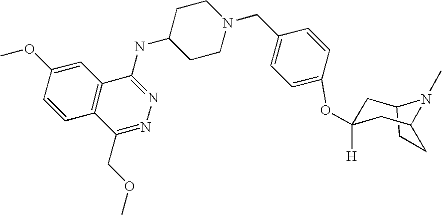

This compound in base form is obtained according to the procedure described in 26.2 by reacting 7-methoxy-4-methoxymethyl-N-(piperidin-4-yl)phthalazin-1-amine (preparation 22.2) with 4-[(8-methyl-8-azabicyclo[3.2.1]oct-3-yl)oxy]benzaldehyde (beta anomer). The trihydrochloride is obtained by means of the treatment described in 4.3.

### EXAMPLE 38

### 2-[{4-[(4-{[7-Methoxy-4-(methoxymethyl)phthalazin-1-yl]amino}piperidin-1-yl)-methyl]phenyl}(methyl)amino]ethanol trihydrochloride (compound 38)

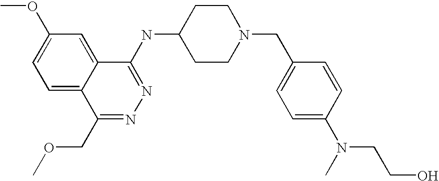

This compound in base form is obtained according to the procedure described in 26.2 by reacting 7-methoxy-4-methoxymethyl-N-(piperidin-4-yl)phthalazin-1-amine (preparation 22.2) with N-methyl-N-(2-hydroxyethyl)-4-aminobenzaldehyde.

The trihydrochloride is obtained by means of the treatment described in 4.3.

### EXAMPLE 39

### 7-Methoxy-4-(methoxymethyl)-N-{1-[4-(2-oxo-2-pyrrolidin-1-ylethoxy)benzyl]piperidin-4-yl}phthalazin-1-amine dihydrochloride (compound 39)

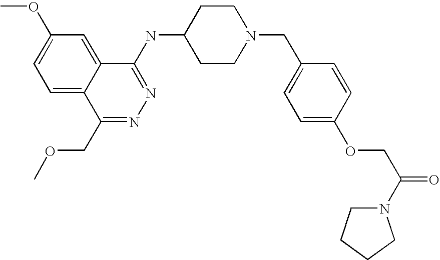

### 39.1. 4-(2-Oxo-2-pyrrolidin-1-ylethoxy)benzaldehyde

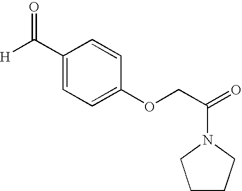

To a mixture consisting of 1 g (5.55 mmol) of 4-formylphenoxyacetic acid and 0.435 g (6.11 mmol) of pyrrolidine in 50 mL of DMF are added 1.117 g (5.83 mmol) of N-(3-dimethylaminopropy)-N′-ethylcarbodiimide hydrochloride (EDCI) and 0.71 g (5.83 mmol) of dimethylaminopyridine (DMAP). The mixture is stirred at room temperature for 20 hours followed by addition of water, and the mixture is extracted with ethyl acetate. The organic phase is washed with 1 N hydrochloric acid, with 1 N sodium hydroxide, with water and then with saturated aqueous sodium chloride. It is dried over anhydrous sodium sulfate and then evaporated under reduced pressure. 0.33 g of a beige-colored solid is obtained.

¹H NMR (DMSO-d₆, 250 MHz) δ ppm: 1.72-1.98 (m, 4H); 3.30-3.39 (m, 2H); 3.45-3.52 (m, 2H); 4.89 (s.2H); 7.10 (d, J=9 Hz, 2H), 8.86 (d, J=9 Hz, 2H), 9.88 (s, 1H, CHO).

### 39.2. 7-Methoxy-4-(methoxymethyl)-N-{1-[4-(2-oxo-2-pyrrolidin-1-ylethoxy)-benzyl]piperidin-4-yl}phthalazin-1-amine dihydrochloride (compound 39)

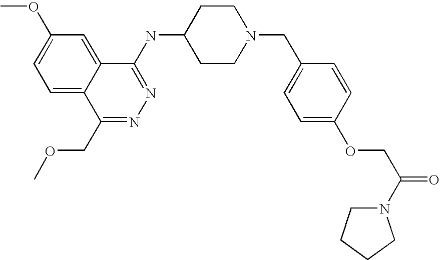

This compound in base form is obtained according to the procedure described in 26.2 by reacting 7-methoxy-4-methoxymethyl-N-(piperidin-4-yl)phthalazin-1-amine (preparation 22.2) with 4-(2-oxo-2-pyrrolidin-1-ylethoxy)benzaldehyde. The dihydrochloride is obtained by means of the treatment described in 4.3.

### EXAMPLE 40

### 3-{4-[(4-{[7-Methoxy-4-(methoxymethyl)phthalazin-1-yl]amino}piperidin-1-yl)methyl]phenoxy}propan-1-ol dihydrochloride (compound 40)

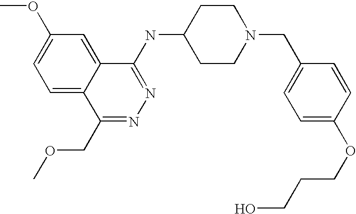

This compound in base form is obtained according to the procedure described in 30.2 by reacting 7-methoxy-4-methoxymethyl-N-(piperidin-4-yl)phthalazin-1-amine (preparation 22.2) with 4-[3-(tetrahydro-2H-pyran-2-yloxy)propoxy]benzaldehyde (preparation 30.1)

The dihydrochloride is obtained by means of the treatment described in 4.3.

### EXAMPLE 41

### 7-Methoxy-4-(methoxymethyl)-N-(1-{4-[(1-methylazepan-4-yl)oxy]benzyl}piperidin-4-yl)phthalazin-1-amine trihydrochloride (compound 41)

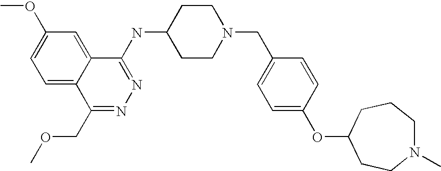

This compound in base form is obtained according to the procedure described in 26.2 by reacting 7-methoxy-4-methoxymethyl-N-(piperidin-4-yl)phthalazin-1-amine (preparation 22.2) with 4-[(1-methylazepan-4-yl)oxy]benzaldehyde (preparation: see 26.1). The trihydrochloride is obtained by means of the treatment described in 4.3.

### EXAMPLE 42

### 7-Methoxy-4-(methoxymethyl)-N-(1-{4-[2-(1-methylpiperidin-2-yl)ethoxy]benzyl}-piperidin-4-yl)phthalazin-1-amine trihydrochloride (compound 42)

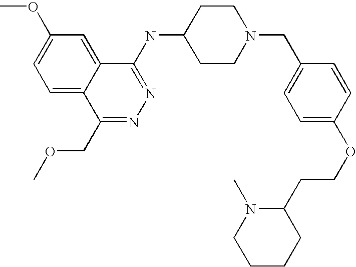

### 42.1. 4-[2-(1-Methylpiperidin-2-yl)ethoxy]benzaldehyde

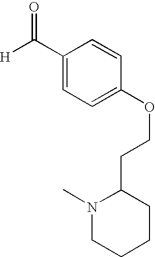

This compound is obtained according to the procedure described in 5.1. by reacting 2-(2-chloroethyl)-1-methylpiperidine hydrochloride with 4-hydroxybenzaldehyde.

LC/MS: MH⁺=248 (Rt=4.70 minutes pH 3.1)

¹H NMR (DMSO-d₆, 250 MHz) δ ppm: 1.15-1.58 (m, 4H); 1.58-1.72 (m, 2H); 1.72-1.92 (m, 1H); 1.92-2.11 (m, 3H); 2.19 (s, 3H); 2.70-2.80 (m, 1H); 4.10-4.18 (m, 2H); 7.13 (d, J=9 Hz, 2H), 8.86 (d, J=9 Hz, 2H), 9.86 (s, 1H, CHO).

### 42.2. 7-Methoxy-4-(methoxymethyl)-N-(1-{4-[2-(1-methylpiperidin-2-yl)ethoxy]-benzyl}piperidin-4-yl)phthalazin-1-amine trihydrochloride (compound 42)

This compound in base form is obtained according to the procedure described in 26.2 by reacting 7-methoxy-4-methoxymethyl-N-(piperidin-4-yl)phthalazin-1-amine (preparation 22.2) with 4-[2-(1-methylpiperidin-2-yl)ethoxy]benzaldehyde.

The trihydrochloride is obtained by means of the treatment described in 4.3.

### EXAMPLE 43

### 2-{4-[(4-{[7-Methoxy-4-(methoxymethyl)phthalazin-1-yl]amino}piperidin-1-yl)-methyl]phenoxy}ethanol dihydrochloride (compound 43)

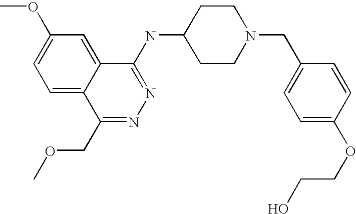

### 43.1. 4-[2-(Tetrahydro-2H-pyran-2-yloxy)ethoxy]benzaldehyde

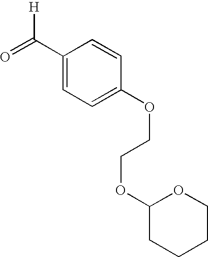

This compound is obtained according to the procedure described in 30.1. by reacting 4-hydroxybenzaldehyde with 2-(2-bromoethoxy)tetrahydro-2H-pyran.

¹H NMR (DMSO-d₆, 250 MHz) ppm: 1.35-1.80 (m, 6H); 3.40-3.50 (m, 1H); 3.65-3.84 (m, 2H); 3.85-4.0 (m, 1H); 4.24-4.30 (m, 2H); 4.65-4.70 (m, 1H); 7.16 (d, J=9 Hz, 2H), 7.86 (d, J=9 Hz, 2H), 9.90 (s, 1H, CHO).

### 43.2 2-{4-[(4-{[7-Methoxy-4-(methoxymethyl)phthalazin-1-yl]amino}piperidin-1-yl)methyl]phenoxy}ethanol dihydrochloride (compound 43)

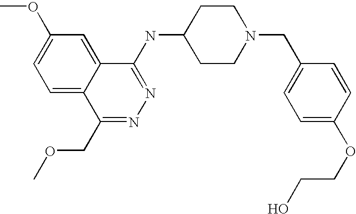

This compound in base form is obtained according to the procedure described in 30.2 by reacting 7-methoxy-4-methoxymethyl-N-(piperidin-4-yl)phthalazin-1-amine (preparation 22.2) with 4-[2-(tetrahydro-2H-pyran-2-yloxy)ethoxy]benzaldehyde.

The dihydrochloride is obtained by means of the treatment described in 4.3.

### EXAMPLE 44

### 7-Methoxy-4-methyl-N-(1-{4-[(1-methylazepan-4-yl)oxy]benzyl}piperidin-4-yl)phthalazin-1-amine trihydrochloride (compound 44)

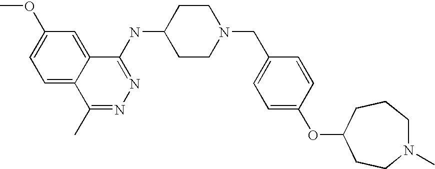

### 44.1 2-Acetyl-5-methoxybenzoic acid

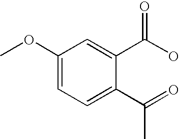

This compound is synthesized according to the method described in 2.4. by reacting 2-bromo-5-methoxybenzoic acid pretreated with n-butyllithium with N-methoxy-N-methylacetamide. It is used crude in the following reaction.

### 44.2. 7-Methoxy-4-methyl-2H-phthalazin-1-one

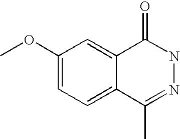

This compound is obtained according to the procedure described in 2.5. by reacting unpurified 2-acetyl-5-methoxybenzoic acid with hydrazine hydrate.

m.p.=202° C. (Mettler FP62)

LC/MS: MH⁺=191 (Rt=5.73 minutes)

¹H NMR (DMSO-d₆, 250 MHz) δ ppm: 2.50 (s, 3H); 3.96 (s, 3H); 7.52 (dd, J₁=8.8 Hz, J₂=2.7 Hz, 1H); 7.66 (d, J=2.7 Hz, 1H); 7.90 (d, J=8.8 Hz, 1H); 12.35 (s, 1H, NH).

### 44.3. 1-Chloro-7-methoxy-4-methylphthalazine

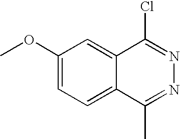

This compound is obtained according to the procedure described in 2.6. by reacting 7-methoxy-4-methyl-2H-phthalazin-1-one with phosphoryl chloride.

LC/MS: MH⁺=209 (Rt=6.26 minutes)

¹H NMR (DMSO-d₆, 250 MHz) δ ppm: 2.92 (s, 3H); 4.06 (s, 3H); 7.54 (d, J=2.5 Hz, 1H); 7.77 (dd, J₁=9.2 Hz, J₂=2.5 Hz, 1H); 8.31 (d, J=9.2 Hz, 1H).

### 44.4. N-(1-Benzylpiperidin-4-yl)-7-methoxy-4-methylphthalazin-1-amine

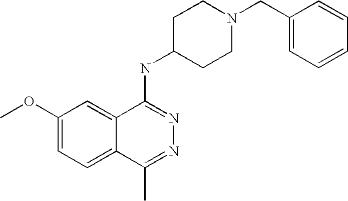

This compound is obtained according to the procedure described in 3.1. by reacting 1-chloro-7-methoxy-4-methylphthalazine with 4-amino-1-benzylpiperidine.

LC/MS: MH⁺=363 (Rt=3.84 minutes pH 3.1)

¹H NMR (DMSO-d₆, 400 MHz) δ ppm: 1.57-1.70 (m, 2H); 1.98-2.12 (m, 4H); 2.62 (s, 3H); 2.85-2.92 (m, 2H); 3.51 (s, 2H); 3.95 (s, 3H); 4.10-4.22 (m, 1H); 6.77 (d, J=7.2 Hz, 1H); 7.22-7.30 (m, 1H); 7.30-7.38 (m, 4H); 7.44 (dd, J₁=9 Hz, J₂=2.5 Hz, 1H); 7.68 (d, J=2.5 Hz, 1H); 7.99 (d, J=9 Hz, 1H).

### 44.5. 7-Methoxy-4-methyl-N-(piperidin-4-yl)phthalazin-1-amine

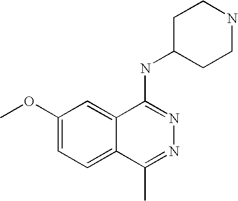

This compound is obtained by debenzylation of N-(1-benzylpiperidin-4-yl)-7-methoxy-4-methylphthalazin-1-amine according to the procedure described in 3.2.

m.p.=221° C. (M)

LC/MS: MH⁺=273 (Rt=5.03 minutes, pH 7)

¹H NMR (DMSO-d₆, 250 MHz) δ ppm: 1.38-1.57 (m, 2H); 1.92-2.04 (m, 3H); 2.55-2.62 (m, 2H); 2.63 (s, 3H); 2.98-3.08 (m, 2H); 3.96 (s, 3H); 4.12-4.28 (m, 1H); 6.74 (d, J=7.2 Hz, 1H); 7.44 (dd, J₁=9 Hz, J₂=2.5 Hz, 1H), 7.71 (d, J=2.5 Hz, 1H); 7.91 (d, J=9 Hz, 1H).

### 44.6. 7-Methoxy-4-methyl-N-(1-{4-[(1-methylazepan-4-yl)oxy]benzyl}piperidin-4-yl)-phthalazin-1-amine trihydrochloride (compound 44)

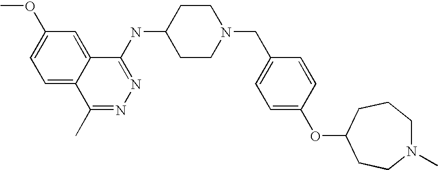

This compound in base form is obtained according to the procedure described in 26.2 by reacting 7-methoxy-4-methyl-N-(piperidin-4-yl)phthalazin-1-amine with 4-[(1-methylazepan-4-yl)oxy]benzaldehyde (synthesis: see 26.1).

The trihydrochloride is obtained by means of the treatment described in 4.3.

### EXAMPLE 45

### 3-{3-[(4-{[7-Methoxy-4-(methoxymethyl)phthalazin-1-yl]amino}piperidin-1-yl)methyl]phenoxy}propan-1-ol dihydrochloride (compound 45)

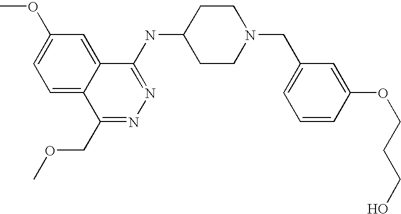

### 45.1 3-[3-(Tetrahydro-2H-pyran-2-yloxy)propoxy]benzaldehyde

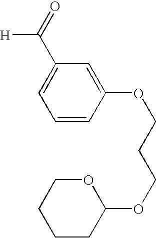

This compound is obtained according to the procedure described in 30.1. by reacting 3-hydroxybenzaldehyde with 2-(3-bromopropoxy)tetrahydro-2H-pyran.

LC/MS: MNa⁺=287 (Rt=8.98 minutes, pH 3.1)

¹H NMR (DMSO-d₆, 250 MHz) δ ppm: 1.40-1.80 (m, 6H); 1.97-2.10 (m, 2H); 3.38-3.58 (m, 2H); 3.70-3.85 (m, 2H); 4.10-4.20 (m, 2H); 4.55-4.60 (m, 1H); 7.26-7.32 (m, 1H), 7.45-7.48 (m, 1H), 7.50-7.55 (m, 2H); 9.90 (s, 1H, CHO).

### 45.2. 3-{3-[(4-{[7-Methoxy-4-(methoxymethyl)phthalazin-1-yl]amino}piperidin-1-yl)-methyl]phenoxy}propan-1-ol dihydrochloride (compound 45)

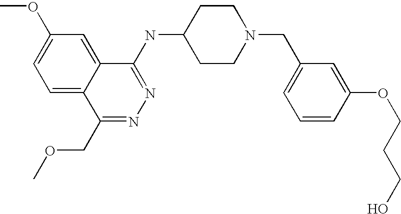

This compound in base form is obtained according to the procedure described in 30.2 by reacting 7-methoxy-4-methoxymethyl-N-(piperidin-4-yl)phthalazin-1-amine (preparation 22.2) with 3-[3-(tetrahydro-2H-pyran-2-yloxy)propoxy]benzaldehyde.

The dihydrochloride is obtained by means of the treatment described in 4.3.

### EXAMPLE 46

### 3-[4-({4-[(7-Methoxy-4-methylphthalazin-1-yl)amino]piperidin-1-yl}methyl)phenoxy]propan-1-ol dihydrochloride (compound 46)

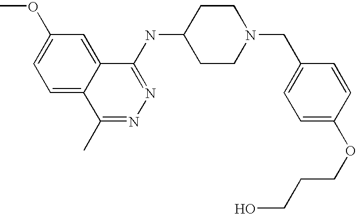

This compound in base form is obtained according to the procedure described in 30.2 by reacting 7-methoxy-4-methyl-N-(piperidin-4-yl)phthalazin-1-amine (preparation 44.5) with 4-[3-(tetrahydro-2H-pyran-2-yloxy)propoxy]benzaldehyde (preparation 30.1)

The dihydrochloride is obtained by means of the treatment described in 4.3.

### EXAMPLE 47

### 7-Chloro-4-methyl-N-(1-{4-[(1-methylazepan-4-yl)oxy]benzyl}piperidin-4-yl)phthalazin-1-amine trihydrochloride (compound 47)

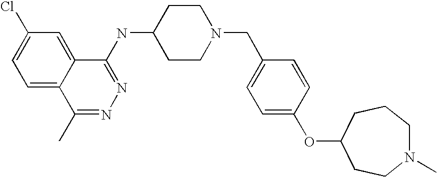

### 47.1. 2-Acetyl-5-chlorobenzoic acid

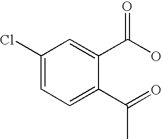

This compound is synthesized according to the method described in 2.4. by reacting 2-bromo-5-chlorobenzoic acid pretreated with n-butyllithium with N-methoxy-N-methylacetamide. It is used crude in the following reaction.

### 47.2. 7-Chloro-1-hydroxy-4-methylphthalazine

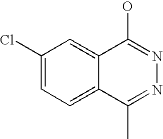

This compound is obtained according to the procedure described in 2.5. by reacting unpurified 2-acetyl-5-chlorobenzoic acid with hydrazine hydrate.

m.p.=195° C. (M)

LC/MS: MH⁺=195 (Rt=6.14 minutes pH 3.1)

¹H NMR δ in ppm (DMSO-d₆, 500 MHz): 2.53 (s, 3H); 7.97-8.01 (m, 2H); 8.15 (s, 1H); 12.55 (s, 1H, OH).

### 47.3. 1,7-Dichloro-4-methylphthalazine

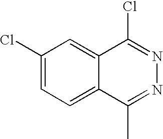

This compound is obtained according to the procedure described in 2.6. by reacting 7-chloro-1-hydroxy-4-methylphthalazine with phosphoryl chloride.

LC/MS: MH⁺=213 (Rt=7.18 minutes pH 3.1)

¹H NMR δ in ppm (DMSO-d₆, 250 MHz): 2.93 (s, 3H); 8.20 (dd, J₁=9 Hz, J₂=2 Hz, 1H); 8.30 (d, J=2 Hz, 1H); 8.37 (d, J=9 Hz, 1H).

### 47.4. 7-Chloro-4-methyl-N-(piperidin-4-yl)phthalazin-1-amine

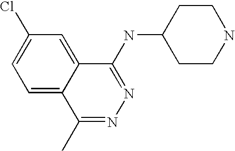

To a suspension of 2.7 g (12.6 mmol) of 1,7-dichloro-4-methylphthalazine in 18 mL of n-butanol are added 2.62 g (15.2 mmol) of ethyl 4-aminopiperidin-1-ylcarboxylate and 0.68 g (12.7 mmol) of ammonium chloride. The mixture is heated at 140° C. for 8 hours. The reaction medium is cooled to room temperature, saturated aqueous sodium hydrogen carbonate solution is added and the mixture is extracted with ethyl acetate. The organic phase is washed with brine, dried over anhydrous sodium sulfate and then evaporated under reduced pressure. The residue obtained is purified by chromatography on a column of silica gel (solvent: dichloromethane/methanol from 100/0 to 95/5 (v/v)). 3.2 g of gummy product are obtained in the form of the ethyl carbamate.

48 mL of a 33% solution of hydrogen bromide in acetic acid are added and the mixture is stirred at 115° C. for 3 hours. After cooling, the precipitate is taken up in isopropanol and the suspension obtained is filtered. The partially dried precipitate is dissolved in 120 mL of water and the solution obtained is basified with 60 mL of 1N sodium hydroxide. A suspension forms, which is extracted with dichloromethane. The organic phase is washed with saturated aqueous sodium chloride, dried over anhydrous sodium sulfate and then evaporated under reduced pressure. 2.29 g of beige-colored product are obtained.

m.p.=214° C. (M)

¹H NMR (DMSO-d₆, 250 MHz) δ ppm: 1.38-1.55 (m, 2H); 1.92-2.02 (m, 2H); 2.55-2.62 (m, 2H); 2.67 (s, 3H); 2.98-3.08 (m, 2H); 3.20-3.35 (m, 1H); 4.10-4.28 (m, 1H); 6.99 (d, J=7.2 Hz, 1H); 7.89 (dd, J₁=9 Hz, J₂=2 Hz, 1H), 8.01 (d, J=9 Hz, 1H); 8.55 (d, J=2 Hz, 1H).

### 47.5. 7-Chloro-4-methyl-N-(1-{4-[(1-methylazepan-4-yl)oxy]benzyl}piperidin-4-yl)phthalazin-1-amine trihydrochloride (compound 47)

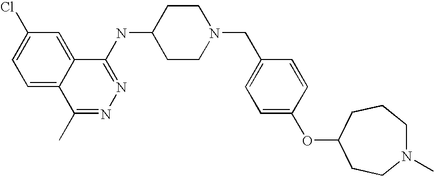

This compound in base form is obtained according to the procedure described in 26.2 by reacting 7-chloro-4-methyl-N-(piperidin-4-yl)phthalazin-1-amine with 4-[(1-methylazepan-4-yl)oxy]benzaldehyde (preparation 26.1)

The trihydrochloride is obtained by means of the treatment described in 4.3.

### EXAMPLE 48

### 3-[4-({4-[(7-Chloro-4-methylphthalazin-1-yl)amino]piperidin-1-yl}methyl)phenoxy]-propan-1-ol dihydrochloride (compound 48)

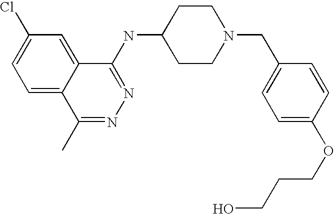

This compound in base form is obtained according to the procedure described in 30.2 by reacting 7-chloro-4-methyl-N-(piperidin-4-yl)phthalazin-1-amine (preparation 47.4) with 4-[3-(tetrahydro-2H-pyran-2-yloxy)propoxy]benzaldehyde (synthesis: see 30.1) followed by purification on a column of silica (eluent: dichloromethane/methanol from 100/0 to 85/15 (v/v)).

The dihydrochloride is obtained by means of the treatment described in 4.3.

### EXAMPLE 49

### (3S)-1-{4-[(4-{[7-Methoxy-4-(methoxymethyl)phthalazin-1-yl]amino}piperidin-1-yl)-methyl]phenyl}pyrrolidin-3-ol trihydrochloride (compound 49)

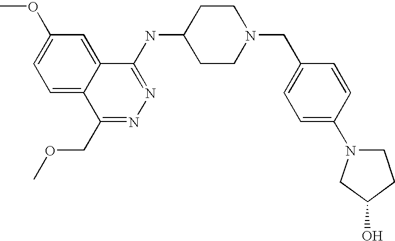

### 49.1 4-[(3S)-3-Hydroxypyrrolidin-1-yl]benzaldehyde

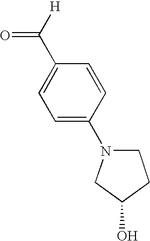

This compound is obtained according to the procedure described in 31.1 by reacting 4-bromobenzaldehyde with (S)-3-hydroxypyrrolidine.

This compound is too colored to be able to measure its optical rotation.

LC/MS: MH⁺=192 (Rt=5.21 minutes pH 3.1)

¹H NMR (DMSO-d₆, 250 MHz) δ ppm: 1.88-2.14 (m, 2H); 3.18-3.25 (m, 1H); 3.40-3.55 (m, 3H); 4.40-4.50 (m, 1H); 5.04 (d, J=3.7 Hz, 1H), 6.64 (d, J=9 Hz, 2H), 7.68 (d, J=9 Hz, 2H), 9.66 (s, 1H, CHO).

### 49.2. (3S)-1-{4-[(4-{[7-Methoxy-4-(methoxymethyl)phthalazin-1-yl]amino}piperidin-1-yl)methyl]phenyl}pyrrolidin-3-ol trihydrochloride (compound 49)

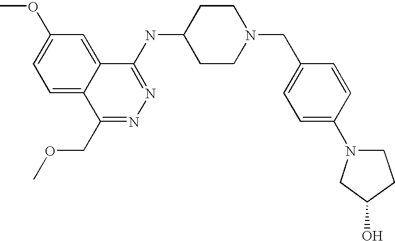

This compound in base form is obtained according to the procedure described in 26.2 by reacting 7-methoxy-4-methoxymethyl-N-(piperidin-4-yl)phthalazin-1-amine (preparation 22.2) with 4-[(3S)-3-hydroxypyrrolidin-1-yl]benzaldehyde.

The dihydrochloride is obtained by means of the treatment described in 4.3.

### EXAMPLE 50

### (3R)-1-{4-[(4-{[7-Methoxy-4-(methoxymethyl)phthalazin-1-yl]amino}piperidin-1-yl)-methyl]phenyl}pyrrolidin-3-ol trihydrochloride (compound 50)

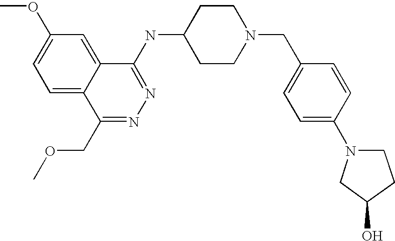

### 50.1 4-[(3R)-3-Hydroxypyrrolidin-1-yl]benzaldehyde

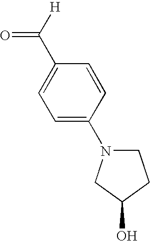

This compound is obtained according to the procedure described in 31.1 by reacting 4-bromobenzaldehyde with (R)-3-hydroxypyrrolidine.

This compound is too colored to be able to measure its optical rotation.

LC/MS: MH⁺=192 (Rt=5.20 minutes pH 3.1)

¹H NMR (DMSO-d₆, 250 MHz) δ ppm: 1.88-2.15 (m, 2H); 3.18-3.26 (m, 1H); 3.40-3.55 (m, 3H); 4.40-4.50 (m, 1H); 5.04 (d, J=3.7 Hz, 1H), 6.64 (d, J=9 Hz, 2H), 7.68 (d, J=9 Hz, 2H), 9.66 (s, 1H, CHO).

### 50.1 (3R)-1-{4-[(4-{[7-Methoxy-4-(methoxymethyl)phthalazin-1-yl]amino}piperidin-1-yl)methyl]phenyl}pyrrolidin-3-ol trihydrochloride (compound 50)

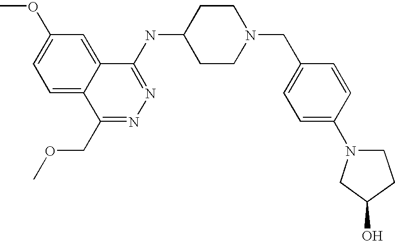

This compound in base form is obtained according to the procedure described in 26.2 by reacting 7-methoxy-4-methoxymethyl-N-(piperidin-4-yl)phthalazin-1-amine (preparation 22.2) with 4-[(3R)-3-hydroxypyrrolidin-1-yl]benzaldehyde.

The dihydrochloride is obtained by means of the treatment described in 4.3.

### EXAMPLE 51

### 3-{4-[(4-{[7-Methoxy-4-(methoxymethyl)phthalazin-1-yl]amino}piperidin-1-yl)methyl]phenyl}prop-2-yn-1-ol (compound 51)

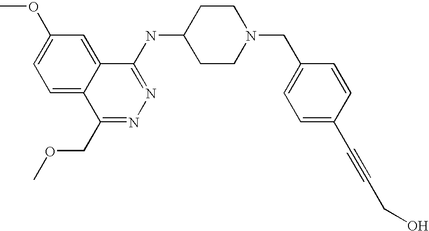

This compound is obtained according to the procedure described in 8.2. by reacting 7-methoxy-4-methoxymethyl-N-(piperidin-4-yl)phthalazin-1-amine (preparation 22.2) with 4-(3-hydroxypropy-1-yn-1-yl)benzaldehyde. It was purified on a column of silica (eluent: dichloromethane/methanol from 100/0 to 90/10 (v/v)) and solidified in isopropyl ether.

¹H NMR (DMSO-d₆, 400 MHz) δ ppm: 1.60-1.72 (m, 2H); 2.0-2.17 (m, 4H); 2.85-2.90 (m, 2H); 3.28 (s, 3H); 3.53 (s, 2H); 3.95 (s, 3H); 4.15-4.25 (m, 1H); 4.30 (d, J=6 Hz, 2H); 4.76 (s, 2H); 5.30 (t, J=6 Hz, 1H, OH); 6.98 (d, J=7.2 Hz. 1H, NH); 7.31-7.41 (m, 4H); 7.46 (dd, J₁=8.8 Hz; J₂=2 Hz, 1H); 7.71 (d, J=2 Hz, 1H); 8.0 (d, J=8.8 Hz, 1H).

### EXAMPLE 52

### 4-{4-[(4-{[7-Methoxy-4-(methoxymethyl)phthalazin-1-yl]amino}piperidin-1-yl)methyl]phenyl}-2-methylbut-3-yn-2-ol (compound 52)

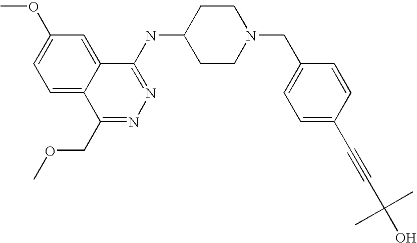

### 52.1. 4-(3-Hydroxy-3-methylbut-1-yn-1-yl)benzaldehyde

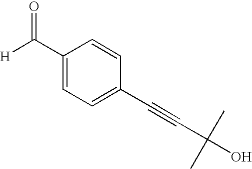

A mixture consisting of 3 g (16.2 mmol) of 4-bromobenzaldehyde, 0.15 g of copper (I) iodide, 0.66 g of palladium-on-charcoal, 0.84 g of triphenylphosphine and 3 mL (49 mmol) of ethanolamine in 120 mL of water is stirred under nitrogen at room temperature for 30 minutes, followed by dropwise addition of 2.4 mL (24.3 mmol) of 2-methyl-3-butyne-2-ol. The reaction mixture is stirred at 70° C. for 7 hours and then overnight at room temperature. Ethyl acetate is added to the mixture is then filtered through Celite. The organic phase is washed with saturated aqueous sodium chloride, dried over anhydrous sodium sulfate and then evaporated under reduced pressure. 4 g of an oily product are obtained, which product is subsequently used without purification.

LC/MS: MH⁺=279 (Rt=7.03 minutes pH 3.1)

¹H NMR (DMSO-d₆, 400 MHz) δ ppm: 1.48 (s, 6H); 5.51 (s, 1H, OH); 7.47 (d, J=8.4 Hz, 2H), 7.71 (d, J=8.4 Hz, 2H), 8.31 (s, 1H, CHO).

### 52.2. 4-{4-[(4-{[7-Methoxy-4-(methoxymethyl)phthalazin-1-yl]amino}piperidin-1-yl)methyl]phenyl}-2-methylbut-3-yn-2-ol (compound 52)

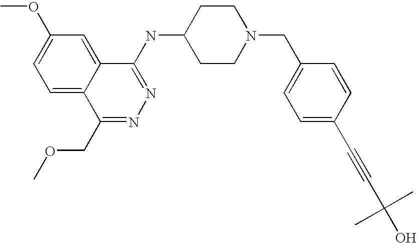

This compound is obtained according to the procedure described in 8.2. by reacting 7-methoxy-4-methoxymethyl-N-(piperidin-4-yl)phthalazin-1-amine (preparation 22.2) with 4-(3-hydroxy-3-methylbut-1-yn-1-yl)benzaldehyde. It was purified on a column of silica (eluent: dichloromethane/methanol from 100/0 to 90/10(v/v)) and solidified in isopropyl ether.

¹H NMR (DMSO-d₆, 400 MHz) δ ppm: 1.46 (s, 6H); 1.60-1.72 (m, 2H); 2.0-2.20 (m, 4H); 2.85-2.92 (m, 2H); 3.28 (s, 3H); 3.53 (s, 2H); 3.95 (s, 3H); 4.18-4.28 (m, 1H); 4.76 (s, 2H); 5.43 (s, 1H, OH); 6.98 (d, J=7.2 Hz. 1H, NH); 7.30-7.38 (m, 4H); 7.46 (dd, J₁=8.8 Hz; J₂=2 Hz, 1H); 7.71 (d, J=2 Hz, 1H); 8.0 (d, J=8.8 Hz, 1H).

### EXAMPLE 53

### 7-Methoxy-4-(methoxymethyl)-N-{1-[4-(4-pyrrolidin-1-ylbutyl)benzyl]piperidin-4-yl}-phthalazin-1-amine trioxalate (compound 53)

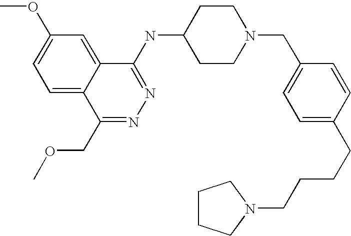

### 53.1 Ethyl 4-{4-[(methoxysulfonyl)oxy]butyl}benzoate

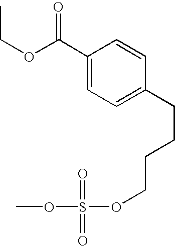

A solution of 0.79 g (3.6 mmol) of ethyl 4-(4-hydroxybutyl)benzoate (synthetic method described in J. Med. Chem., 1983 26 (3) pp. 335-341) and 0.5 mL (3.6 mmol) of triethylamine in 15 mL of dichloromethane is stirred at 0° C. and 0.33 mL (4.32 mmol) of methyl chloride is added. The mixture is stirred at room temperature for 2 hours and is then hydrolyzed and extracted with dichloromethane. The organic phase is washed with saturated aqueous sodium chloride, dried over anhydrous sodium sulfate and then evaporated under reduced pressure. 1.073 g of an oily product are obtained.

¹H NMR (DMSO-d₆, 250 MHz) δ ppm: 1.32 (t, J=7 Hz, 3H); 1.64-1.74 (m, 4H); 2.65-2.75 (m, 2H); 3.16 (s, 3H); 4.18-4.28 (m, 2H); 4.31 (q, J=7 Hz, 2H); 7.37 (d, J=8 Hz, 2H), 7.90 (d, J=8 Hz, 2H).

### 53.2 Ethyl 4-(4-pyrrolidin-1-ylbutyl)benzoate

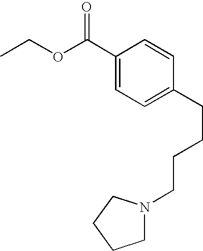

To a solution of 0.96 g (3.2 mmol) of ethyl 4-{4-[(methoxysulfonyl)oxy]butyl}benzoate in 100 mL of isopropanol are added 8 mL (130 mmol) of pyrrolidine. The mixture is refluxed for 23 hours. After cooling, the isopropanol is evaporated off under reduced pressure, water is added to the evaporation residue and the mixture is extracted with ethyl acetate. The organic phase is washed with saturated aqueous sodium chloride, dried over anhydrous sodium sulfate and then evaporated under reduced pressure. The residue obtained is purified on a column of silica (eluent: dichloromethane/methanol from 100/0 to 90/10 (v/v)). The product is obtained quantitatively in oily form.

¹H NMR (DMSO-d₆, 250 MHz) δ ppm: 1.32 (t, J=7.2 Hz, 3H); 1.60-1.70 (m, 4H); 1.88-1.98 (m, 4H); 2.68-2.75 (m, 2H); 3.03-3.30 (m, 6H); 4.31 (q, J=7.2 Hz, 2H); 7.38 (d, J=8.2 Hz, 2H); 7.90 (d, J=8.2 Hz, 2H).

### 53.3 [4-(4-Pyrrolidin-1-ylbutyl)phenyl]methanol

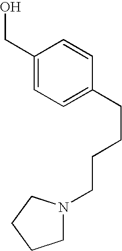

To a suspension of 154 mg (4 mmol) of lithium aluminum hydride in 20 mL of THF is added a solution of 746 mg (2.7 mmol) of ethyl 4-(4-pyrrolidin-1-ylbutyl)benzoate in 10 mL of THF. The mixture is stirred at room temperature for three days, and 0.2 mL of water, 0.2 mL of 6N sodium hydroxide and 0.6 mL of water are then added slowly and successively. A precipitate forms, which is filtered off through Celite and rinsed with ethyl acetate. The filtrate is evaporated under reduced pressure. 597 mg of an oily product are obtained.

¹H NMR (DMSO-d₆, 250 MHz) δ ppm: 1.35-1.70 (m, 8H); 2.30-2.40 (m, 6H); 2.52-2.60 (m, 2H); 4.45 (d, J=5.5 Hz, 2H); 5.09 (t, J=5.5 Hz, 1H, OH); 7.13 (d, J=8 Hz, 2H); 7.22 (d, J=8 Hz, 2H).

### 53.4 4-(4-Pyrrolidin-1-ylbutyl)benzaldehyde

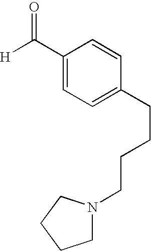

To a solution of 590 mg (2.5 mmol) of [4-(4-pyrrolidin-1-ylbutyl)phenyl]methanol in 15 mL of dioxane are added 1.1 g (12.6 mmol) of manganese dioxide. The mixture is stirred at 80° C. for 4 hours. After cooling, it is filtered through Celite, rinsing with dichloromethane. The filtrate is evaporated under reduced pressure. 545 mg of product are obtained.

¹H NMR (DMSO-d₆, 250 MHz) δ ppm: 1.38-1.52 (m, 2H); 1.52-1.70 (m, 6H); 2.30-2.40 (m, 6H); 2.67-2.72 (m, 2H); 7.44 (d, J=8 Hz, 2H); 7.83 (d, J=8 Hz, 2H); 9.97 (s, 1H, CHO).

### 53.5 7-Methoxy-4-(methoxymethyl)-N-{1-[4-(4-pyrrolidin-1-ylbutyl)benzyl]piperidin-4-yl}phthalazin-1-amine trioxalate (compound 53)

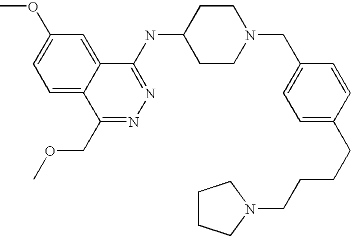

This compound is obtained according to the procedure described in 26.2. by reacting 7-methoxy-4-methoxymethyl-N-(piperidin-4-yl)phthalazin-1-amine (preparation 22.2) with 4-(4-pyrrolidin-1-ylbutyl)benzaldehyde.

¹H NMR (DMSO-d₆, 400 MHz) δ ppm: 1.40-1.48 (m, 2H); 1.55-1.70 (m, 8H); 2.0-2.12 (m, 4H); 2.32-2.40 (m, 4H); 2.52-2.60 (m, 2H); 2.85-2.92 (m, 2H); 3.28 (s, 3H); 3.47 (s, 3H); 3.96 (s, 3H); 4.15-4.27 (m, 1H); 4.76 (s, 2H); 6.98 (d, J=7.2 Hz, 1H); 7.13 (d, J=8 Hz, 2H); 7.22 (d, J=8 Hz, 2H); 7.46 (dd, J₁=8.8 Hz, J₂=2.4 Hz, 1H); 7.70 (d, J=2.4 Hz, 1H); 8.0 (d, J=8.8 Hz, 1H).

The trioxalate is obtained by treating the product is dissolved in acetone with 3 equivalents of oxalic acid. A precipitate forms, which is filtered off. The powder obtained is dried at 40° C. under reduced pressure in the presence of phosphorus pentoxide.

### EXAMPLE 54

### 2-{3-[(4-{[7-Methoxy-4-(4-methoxyphenyl)phthalazin-1-yl]amino}piperidin-1-yl)-methyl]phenoxy}ethanol dihydrochloride (compound 54)

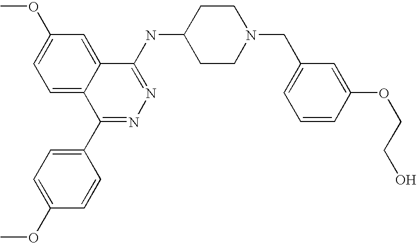

### 54.1 3-[2-(Tetrahydro-2H-pyran-2-yloxy)ethoxy]benzaldehyde

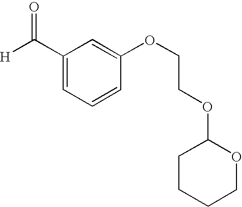

This compound is obtained according to the procedure described in 30.1. by reacting 3-hydroxybenzaldehyde with 2-(2-bromoethoxy)tetrahydro-2H-pyran.

¹H NMR (DMSO-d₆, 250 MHz) δ ppm: 1.40-1.80 (m, 6H); 3.40-3.50 (m, 1H); 3.68-3.40 (m, 3H); 4.20-4.25 (m, 2H); 4.65-4.70 (m, 1H); 7.30-7.35 (m, 1H), 7.45-7.48 (m, 1H), 7.50-7.55 (m, 2H); 9.99 (s, 1H, CHO).

### 54.2 2-{3-[(4-{[7-Methoxy-4-(4-methoxyphenyl)phthalazin-1-yl]amino}piperidin-1-yl)methyl]phenoxy}ethanol dihydrochloride (compound 54)

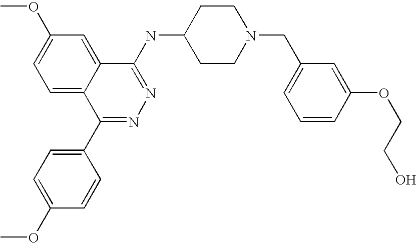

This compound in base form is obtained according to the procedure described in 30.2 by reacting 7-methoxy-4-(4-methoxyphenyl)-N-(piperidin-4-yl)phthalazin-1-amine (preparation 3.2) with 3-[2-(tetrahydro-2H-pyran-2-yloxy)ethoxy]benzaldehyde. The dihydrochloride is obtained by means of the treatment described in 4.3.

### EXAMPLE 55

### 2-{3-[(4-{[7-Methoxy-4-(methoxymethyl)phthalazin-1-yl]amino}piperidin-1-yl)methyl]phenoxy}ethanol dihydrochloride (compound 55)

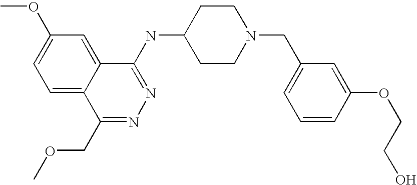

This compound in base form is obtained according to the procedure described in 30.2 by reacting 7-methoxy-4-methoxymethyl-N-(piperidin-4-yl)phthalazin-1-amine (preparation 22.2) with 3-[2-(tetrahydro-2H-pyran-2-yloxy)ethoxy]benzaldehyde (preparation 54.1).

The dihydrochloride is obtained by means of the treatment described in 4.3.

### EXAMPLE 56

### 3-{3-[(4-{[7-Methoxy-4-(4-methoxyphenyl)phthalazin-1-yl]amino}piperidin-1-yl)methyl]-phenoxy}propan-1-ol dihydrochloride (compound 56)

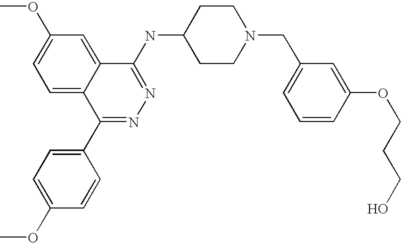

This compound in base form is obtained according to the procedure described in 30.2 by reacting 7-methoxy-4-(4-methoxyphenyl)-N-(piperidin-4-yl)phthalazin-1-amine (preparation 3.2) with 3-[3-(tetrahydro-2H-pyran-2-yloxy)propoxy]benzaldehyde (preparation 45.1).

The dihydrochloride is obtained by means of the treatment described in 6.5.

### EXAMPLE 57

### 3-{4-[(4-{[7-Methoxy-4-(trifluoromethyl)phthalazin-1-yl]amino}piperidin-1-yl)methyl]-phenoxy}propan-1-ol dihydrochloride (compound 57)

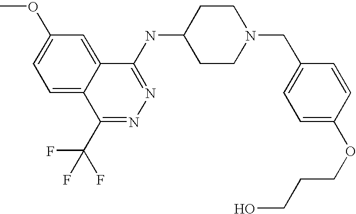

### 57.1. 5-Methoxy-2-trifluoroacetylbenzoic acid

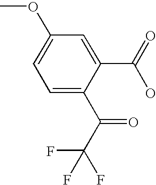

A solution of 5 g (21.64 mmol) of 2-bromo-5-methoxybenzoic acid in 100 mL of THF is stirred under nitrogen at −78° C. followed by dropwise addition of 27 mL (43.28 mmol) of a 1.6 m solution of n-butyllithium in hexane. The mixture is stirred at −78° C. for one hour, followed by dropwise addition of 2.58 mL (21.64 mmol) of ethyl trifluoroacetate. The reaction medium is stirred at −78° C. for one hour and then at room temperature for 18 hours. Water is added and the mixture is extracted with tert-butyl methyl ether. The aqueous phase is acidified with 2N hydrochloric acid solution and extracted with dichloromethane. The dichloromethane phase is washed with brine, dried over anhydrous sodium sulfate, filtered and evaporated. The crude product in the form of a yellow oil is used in the following reaction without purification.

### 57.2. 7-Methoxy-4-trifluoromethyl-2H-phthalazin-1-one

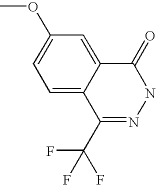

This compound is obtained according to the procedure described in 2.5. by reacting unpurified 5-methoxy-2-trifluoroacetylbenzoic acid with hydrazine hydrate. The product is purified on a column of silica (eluent: dichloromethane/methanol from 100/0 to 95/5 (v/v)).

m.p.=205° C. (M)

LC/MS: MH⁺=245 (Rt=7.57 minutes pH 3.1)

¹H NMR (DMSO-d₆, 300 MHz) δ ppm: 3.98 (s, 3H); 7.62 (dd, J₁=9 Hz, J₂=2.7 Hz, 1H); 7.74 (d, J=2.7 Hz, 1H); 7.90 (d, J=9 Hz, 1H); 13.25 (s, 1H, NH)

### 57.3. 1-Chloro-7-methoxy-4-trifluoromethylphthalazine

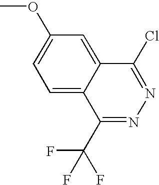

This compound is obtained according to the procedure described in 2.6. by reacting 7-methoxy-4-trifluoromethyl-2H-phthalazin-1-one with phosphoryl chloride. The product is purified on a column of silica (eluent: dichloromethane/methanol from 100/0 to 95/5 (v/v)).

LC/MS: MH⁺=263 (Rt=8.53 minutes)

¹H NMR (DMSO-d₆, 250 MHz) δ ppm: 4.10 (s, 3H); 7.72 (d, J=2.5 Hz, 1H); 7.92 (dd, J₁=9.2 Hz, J₂=2.5 Hz, 1H); 8.27 (d, J=9.2 Hz, 1H).

### 57.4. N-(1-Benzylpiperidin-4-yl)-7-methoxy-4-trifluoromethylphthalazin-1-amine

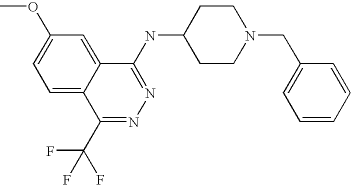

This compound is obtained according to the procedure described in 3.1. by reacting 1-chloro-7-methoxy-4-trifluoromethylphthalazine with 4-amino-1-benzylpiperidine.

LC/MS: MH⁺=417 (Rt=6.13 minutes pH 3.1)

¹H NMR (DMSO-d₆, 250 MHz) δ ppm: 1.62-1.80 (m, 2H); 1.98-2.18 (m, 4H); 2.87-2.98 (m, 2H); 3.53 (s, 2H); 3.99 (s, 3H); 4.27-4.40 (m, 1H); 7.24-7.36 (m, 5H); 7.58-7.67 (m, 2H); 7.86-7.95 (m, 2H).

### 57.5. 7-Methoxy-N-(piperidin-4-yl)-4-trifluoromethylphthalazin-1-amine

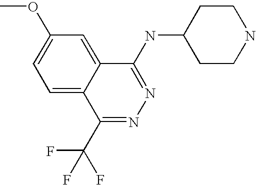

This compound is obtained by debenzylation of N-(1-benzylpiperidin-4-yl)-7-methoxy-4-trifluoromethylphthalazin-1-amine according to the procedure described in 3.2.

m.p.=203° C. (M)

LC/MS: MH⁺=327 (Rt=4.43 minutes, pH 3.1)

¹H NMR (DMSO-d₆, 250 MHz) δ ppm: 1.48-1.63 (m, 2H); 1.93-2.06 (m, 3H); 2.56-2.68 (m, 2H); 3.02-3.10 (m, 2H); 4.0 (s, 3H); 4.30-4.45 (m, 1H); 7.58-7.70 (m, 2H); 7.88-7.94 (m, 2H).

### 57.6. 3-{4-[(4-{[7-Methoxy-4-(trifluoromethyl)phthalazin-1-yl]amino}piperidin-1-yl)methyl]phenoxy}propan-1-ol dihydrochloride (compound 57)

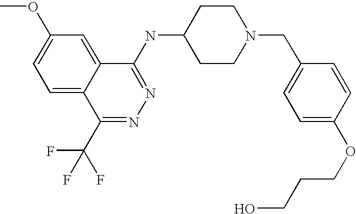

This compound in base form is obtained according to the procedure described in 30.2 by reacting 7-methoxy-N-(piperidin-4-yl)-4-trifluoromethylphthalazin-1-amine with 4-[3-(tetra-hydro-2H-pyran-2-yloxy)propoxy]benzaldehyde (synthesis: see 30.1).

The dihydrochloride is obtained by means of the treatment described in 4.3.

### EXAMPLE 58

### 7-Methoxy-4-(methoxymethyl)-N-{1-[4-(octahydro-2H-pyrido[1,2-a]pyrazin-2-yl)-benzyl]piperidin-4-yl}phthalazin-1-amine trihydrochloride (compound 58)

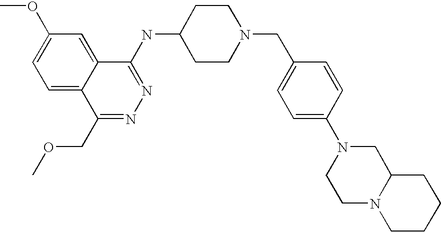

### 58.1 4-(Octahydro-2H-pyrido[1,2-a]pyrazin-2-yl)benzaldehyde

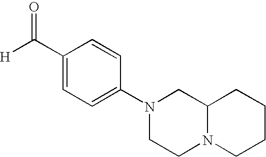

This compound is obtained according to the procedure described in 31.1. by reacting 4-bromobenzaldehyde with commercial (±)-1-4-diazabicyclo[4.4.0]decane. This aldehyde, which is difficult to purify, is used in impure form in the following reaction.

### 58.2. 7-Methoxy-4-(methoxymethyl)-N-{1-[4-(octahydro-2H-pyrido[1,2-a]pyrazin-2-yl)benzyl]piperidin-4-yl}phthalazin-1-amine trihydrochloride (compound 58)

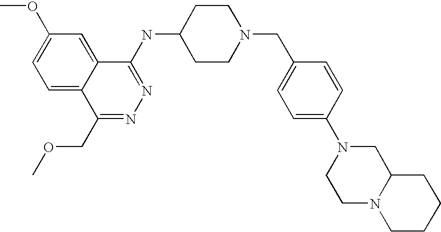

This compound in base form is obtained according to the procedure described in 26.2 by reacting 7-methoxy-4-methoxymethyl-N-(piperidin-4-yl)phthalazin-1-amine (preparation 22.2) with 4-(octahydro-2H-pyrido[1,2-a]pyrazin-2-yl)benzaldehyde.

¹H NMR (DMSO-d₆, 400 MHz) δ ppm: 1.13-1.32 (m, 2H); 1.42-1.75 (m, 6H); 1.90-2.09 (m, 6H); 2.18-2.24 (m, 1H); 2.29-2.35 (m, 1H); 2.66-2.82 (m, 3H); 2.85-2.90 (m, 2H); 3.28 (s, 3H); 3.39 (s, 2H); 3.47-3.59 (m, 2H); 3.95 (s, 3H); 4.13-4.23 (m, 1H); 4.76 (s, 2H); 6.89 (d, J=8.8 Hz, 2H); 6.98 (d, J=7.6 Hz. 1H, NH); 7.15 (d, J=8.8 Hz, 2H); 7.46 (dd, J₁=8.8 Hz; J₂=2.4 Hz, 1H); 7.70 (d, J=2.4 Hz, 1H); 8.0 (d, J=8.8 Hz, 1H).

The trihydrochloride is obtained by means of the treatment described in 6.5.

### EXAMPLE 59

### 4-Ethyl-N-[1-(4-ethynylbenzyl)piperidin-4-yl]-5,7-dimethoxyphthalazin-1-amine dihydrochloride (compound 59)

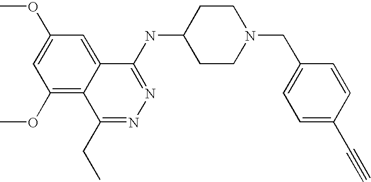

### 59.1. N-(1-Benzylpiperidin-4-yl)-5,7-dimethoxy-4-ethylphthalazin-1-amine

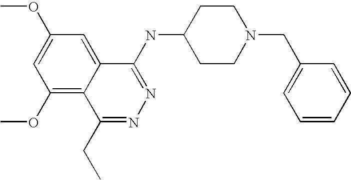

This compound is obtained according to the procedure described in 3.1. by reacting 1-chloro-5,7-dimethoxy-4-ethylphthalazine (described in patent application WO05/103033) with 4-amino-1-benzylpiperidine.

LC/MS: MH⁺=407 (Rt=4.34 minutes pH 3.1)

### 59.2. 5,7-Dimethoxy-4-ethyl-N-(piperidin-4-yl)phthalazin-1-amine

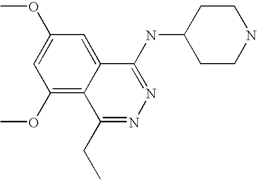

This compound is obtained by debenzylation of N-(1-Benzylpiperidin-4-yl)-5,7-dimethoxy-4-ethylphthalazin-1-amine according to the procedure described in 3.2.

m.p.=211° C. (M)

LC/MS: MH⁺=317 (Rt=4.30 minutes, pH 3.1)

¹H NMR (DMSO-d₆, 250 MHz) δ ppm: 1.19 (t, J=7.5 Hz, 3H); 1.40-1.58 (m, 2H); 1.92-2.04 (m, 2H); 2.53-2.68 (m, 2H); 2.98-3.10 (m, 2H); 3.14 (q, J=7.5 Hz, 2H); 3.3 (m, 1H, NH); 3.95 (s, 6H); 4.12-4.30 (m, 1H); 6.61 (d, J=7.5 Hz, 1H, NH); 6.92 (s, 1H); 7.22 (s, 1H).

### 59.3. 4-Ethyl-N-[1-(4-ethynylbenzyl)piperidin-4-yl]-5,7-dimethoxyphthalazin-1-amine dihydrochloride (compound 59)

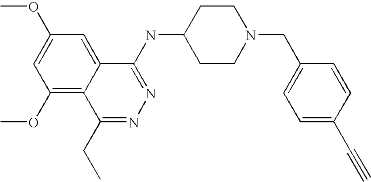

This compound in base form is obtained according to the procedure described in 4.3. by reacting 5,7-dimethoxy-4-ethyl-N-(piperidin-4-yl)phthalazin-1-amine with 4-(trimethylsilyl)-ethynylbenzaldehyde followed by deprotection of the trimethylsilyl group via the method described in 28.2.

¹H NMR (DMSO-d₆, 400 MHz) δ ppm: 1.18 (t, J=7.6 Hz, 3H); 1.58-1.70 (m, 2H); 1.98-2.08 (m, 2H); 2.08-2.25 (m, 2H); 2.83-2.90 (m, 2H); 3.15 (q, J=7.6 Hz, 2H); 3.52 (s, 2H); 3.97 (s, 6H); 4.14 (s, 1H); 4.10-4.20 (m, 1H); 6.59 (d, J=7.2 Hz, 1H); 6.91 (d, J=1.8 Hz, 1H); 7.19 (d, J=1.8 Hz, 1H); 7.35 (d, J=8 Hz, 2H); 7.45 (d, J=8 Hz, 2H).

The dihydrochloride is obtained by means of the treatment described in 6.5.

### EXAMPLE 60

### 3-{2-Methoxy-4-[(4-{[7-methoxy-4-(4-methoxyphenyl)phthalazin-1-yl]amino}piperidin-1-yl)methyl]phenoxy}propan-1-ol dihydrochloride (compound 60)

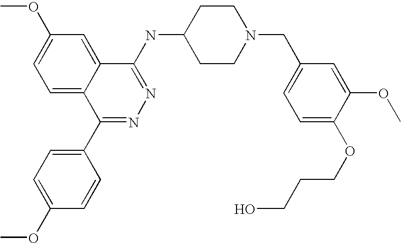

### 60.1. 4-[3-(tetrahydro-2H-pyran-2-yloxy)propoxy]-3-methoxybenzaldehyde

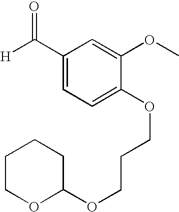

This compound is obtained according to the procedure described in 30.1. by reacting 4-hydroxy-3-methoxybenzaldehyde with 2-(3-bromopropoxy)tetrahydro-2H-pyran.

LC/MS: MNa⁺=317 (Rt=8.20 minutes, pH 3.1)

¹H NMR (DMSO-d₆, 250 MHz) δ ppm: 1.35-1.80 (m, 6H); 1.95-2.08 (m, 2H); 3.37-3.58 (m, 2H); 3.68-3.83 (m, 2H); 3.84 (s, 3H); 4.12-4.20 (m, 2H); 4.55-4.60 (m, 1H); 7.20 (d, J=8.2 Hz, 1H); 7.41 (d, J=1.8 Hz, 1H); 7.55 (dd, J₁=8.2 Hz; J₂=1.8 Hz, 1H); 9.88 (s, 1H, CHO).

### 60.2. 3-{2-Methoxy-4-[(4-{[7-methoxy-4-(4-methoxyphenyl)phthalazin-1-yl]amino}piperidin-1-yl)methyl]phenoxy}propan-1-ol dihydrochloride (compound 60)

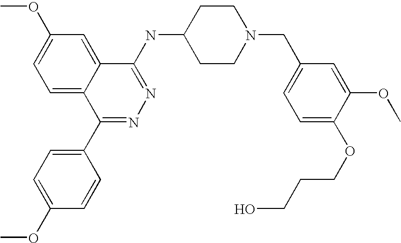

This compound in base form is obtained according to the procedure described in 30.2 by reacting 7-methoxy-4-(4-methoxyphenyl)-N-(piperidin-4-yl)phthalazin-1-amine (preparation 3.2) with 4-[3-(tetrahydro-2H-pyran-2-yloxy)propoxy]-3-methoxybenzaldehyde.

The dihydrochloride is obtained by means of the treatment described in 6.5.

### EXAMPLE 61

### 3-{2-Methoxy-4-[(4-{[7-methoxy-4-(methoxymethyl)phthalazin-1-yl]amino}piperidin-1-yl)methyl]phenoxy}propan-1-ol dihydrochloride (compound 61)

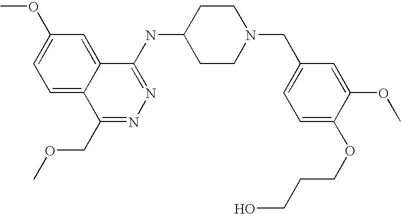

This compound in base form is obtained according to the procedure described in 30.2 by reacting 7-methoxy-4-methoxymethyl-N-(piperidin-4-yl)phthalazin-1-amine (preparation 22.2) with 4-[3-(tetrahydro-2H-pyranyloxy)propoxy]-3-methoxybenzaldehyde (preparation 60.1).

The dihydrochloride is obtained by means of the treatment described in 7.4.

### EXAMPLE 62

### 1-{4-[(4-{[7-Methoxy-4-(methoxymethyl)phthalazin-1-yl]amino}piperidin-1-yl)methyl]phenyl}piperidin-4-ol trihydrochloride (compound 62)

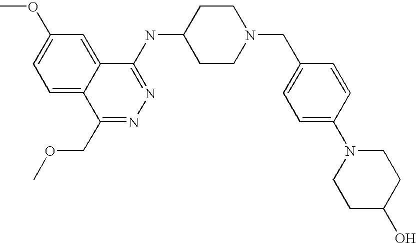

### 62.1 4-(4-Hydroxypiperidin-1-yl)benzaldehyde

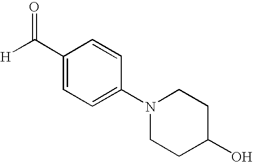

This compound is obtained according to the procedure described in 31.1. by reacting 4-bromobenzaldehyde with 4-hydroxypiperidine.

¹H NMR (DMSO-d₆, 250 MHz) δ ppm: 1.32-1.50 (m, 2H); 1.75-1.88 (m, 2H); 3.08-3.20 (m, 2H); 3.69-3.84 (m, 3H); 4.75 (d, J=4.2 Hz, 1H); 7.04 (d, J=9 Hz, 2H); 7.69 (d, J=9 Hz, 2H); 9.69 (s, 1H, CHO).

### 62.2 1-{4-[(4-{[7-Methoxy-4-(methoxymethyl)phthalazin-1-yl]amino}piperidin-1-yl)methyl]phenyl}piperidin-4-ol trihydrochloride (compound 62)

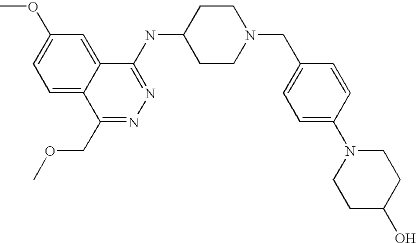

This compound in base form is obtained according to the procedure described in 26.2. by reacting 7-methoxy-4-methoxymethyl-N-(piperidin-4-yl)phthalazin-1-amine (preparation 22.2) with 4-(4-hydroxypiperidin-1-yl)benzaldehyde.

The trihydrochloride is obtained by means of the treatment described in 4.3.

### EXAMPLE 63

### 3-[4-({4-[(7-Methoxyphthalazin-1-yl)amino]piperidin-1-yl}methyl)phenoxy]propan-1-ol dihydrochloride (compound 63)

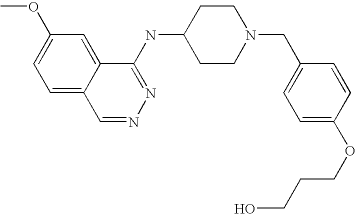

### 63.1. N-(1-Benzylpiperidin-4-yl)-7-methoxyphthalazin-1-amine

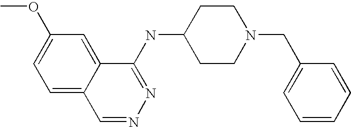

A mixture consisting of 500 mg (2.6 mmol) of 1-chloro-7-methoxyphthalazine (synthesis described in patent application WO05/103033) and 0.63 mL (3.1 mmol) of 4-amino-1-benzylpiperidine in 2.5 mL of dimethoxyethane is stirred at room temperature under an argon atmosphere, and 346 mg (3.6 mmol) of sodium tert-butoxide, 23 mg of tris(dibenzylideneacetone)dipalladium(0), 16 mg of 2,2′-bis(diphenylphosphino)-1,1′-binaphthyl (racemic) and 67 mg of lithium bromide are added. The mixture is stirred at 100° C. for 18 hours, and, after cooling 10 mL of 1N sodium hydroxide are added. The mixture is extracted with ethyl acetate. The organic phase is washed with saturated aqueous sodium chloride, dried over anhydrous sodium sulfate and then evaporated under reduced pressure. The residue obtained is purified on a column of silica (eluent: dichloromethane/methanol from 100/0 to 90/10 (v/v)). 620 mg an orange-colored solid are obtained.

¹H NMR (DMSO-d₆, 400 MHz) δ ppm: 1.60-1.72 (m, 2H); 2.0-2.16 (m, 4H); 2.87-2.92 (m, 2H); 3.51 (s, 2H); 3.95 (s, 3H); 4.15-4.25 (m, 1H); 6.91 (d, J=7.6 Hz, 1H, NH); 7.23-7.30 (m, 1H); 7.30-7.35 (m, 4H); 7.44 (dd, J=8.8 Hz; J₂=2.4 Hz, 1H); 7.70 (d, J=2.4 Hz, 1H); 7.83 (d, J=8.8 Hz, 1H); 8.28 (s, 1H).

### 63.2. 7-Methoxy-N-(piperidin-4-yl)-4-trifluoromethylphthalazin-1-amine

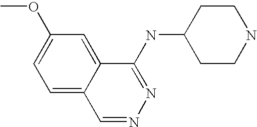

This compound is obtained by debenzylation of N-(1-benzylpiperidin-4-yl)-7-methoxy-phthalazin-1-amine according to the procedure described in 3.2.

m.p.=252° C. (M)

LC/MS: MH⁺=327 (Rt=4.43 minutes, pH 3.1)

¹H NMR (DMSO-d₆, 250 MHz) δ ppm: 1.20-1.30 (m, 1H); 1.40-1.60 (m, 2H); 1.95-2.10 (m, 2H); 2.55-2.68 (m, 2H); 2.98-3.10 (m, 2H); 3.96 (s, 3H); 4.20-4.35 (m, 1H); 6.92 (d, J=7.5 Hz, 1H, NH); 7.44 (dd, J₁=8.8 Hz; J₂=2.2 Hz, 1H); 7.73 (d, J=2.2 Hz, 1H); 7.84 (d, J=8.8 Hz, 1H); 8.76 (s, 1H).

### 63.3. 3-[4-({4-[(7-Methoxyphthalazin-1-yl)amino]piperidin-1-yl}methyl)phenoxy]-propan-1-ol dihydrochloride (compound 63)

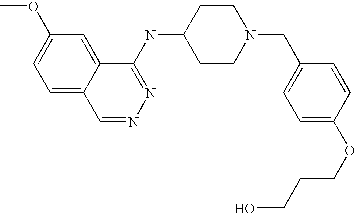

This compound in base form is obtained according to the procedure described in 30.2 by reacting 7-methoxy-N-(piperidin-4-yl)phthalazin-1-amine with 4-[3-(tetrahydro-2H-pyran-2-yloxy)propoxy]benzaldehyde (synthesis: see 30.1). The dihydrochloride is obtained by means of the treatment described in 4.3.

### EXAMPLE 64

### 7-Methoxy-4-(methoxymethyl)-N-{1-[4-(6-methyloctahydro-1H-pyrrolo[3,4-B]pyridin-1-yl)benzyl]piperidin-4-yl}phthalazin-1-amine trihydrochloride (compound 64)

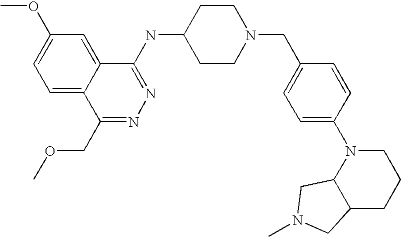

### 64.1. 4-(6-Methyloctahydro-1H-pyrrolo[3,4-B]pyridin-1-yl)benzaldehyde

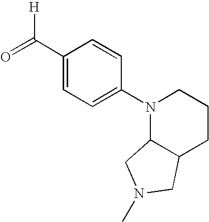

This compound is obtained according to the procedure described in 31.1. by reacting 4-bromobenzaldehyde with commercial 6-methyloctahydropyrrolo[3,4-b]pyridine.

LC/MS: MH⁺=245 (Rt=4.43 minutes, pH 3.1)

¹H NMR (DMSO-d₆, 250 MHz) δ ppm: 1.50-1.92 (m, 4H); 2.29 (s, 3H); 2.30-2.55 (m, 3H); 2.70-2.85 (m, 2H); 2.90-3.02 (m, 1H); 3.60-3.70 (m, 1H); 4.45-4.57 (m, 1H); 6.98 (d, J=8.8 Hz, 2H); 7.69 (d, J=8.8 Hz, 2H); 9.69 (s, 1H, CHO).

### 64.2. 7-Methoxy-4-(methoxymethyl)-N-{1-[4-(6-methyloctahydro-1H-pyrrolo[3,4-b]-pyridin-1-yl)benzyl]piperidin-4-yl}phthalazin-1-amine trihydrochloride (compound 64)

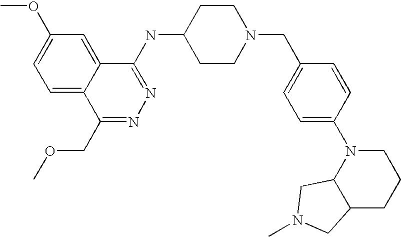

This compound is obtained according to the procedure described in 26.2. by reacting 7-methoxy-4-methoxymethyl-N-(piperidin-4-yl)phthalazin-1-amine (preparation 22.2) with 4-(6-methyloctahydro-1H-pyrrolo[3,4-B]pyridin-1-yl)benzaldehyde.

LC/MS: MH⁺=531 (Rt=4.60 minutes, pH 3.1)

RMN ¹H (DMSO-d₆, 400 MHz) δ ppm: 1.48-1.85 (m, 6H); 1.98-2.10 (m, 2H); 2.24 (s, 3H); 2.22-2.30 (m, 1H); 2.41-2.48 (m, 2H); 2.60-2.68 (m, 2H); 2.80-2.92 (m, 3H); 3.28 (s, 3H); 3.38 (s, 2H); 3.84 (s, 3H); 3.96 (s, 3H); 4.13-4.26 (m, 1H); 4.28-4.36 (m, 1H); 4.33 (s, 2H); 6.83 (d, J=8.8 Hz, 2H); 6.98 (d, J=7.2 Hz, 1H, NH); 7.12 (d, J=8.8 Hz, 2H); 7.46 (dd, J₁=9.2 Hz; J₂=2.4 Hz, 1H); 7.70 (d, J=2.4 Hz, 1H); 8.0 (d, J=9.2 Hz, 1H).

The trihydrochloride is obtained according to the procedure described in 4.3.

### EXAMPLE 65

### 7-Methoxy-4-(4-methoxyphenyl)-N-{1-[4-(7-methyl-2,7-diazaspiro[4.4]non-2-yl)-benzyl]piperidin-4-yl}phthalazin-1-amine trihydrochloride (compound 65)

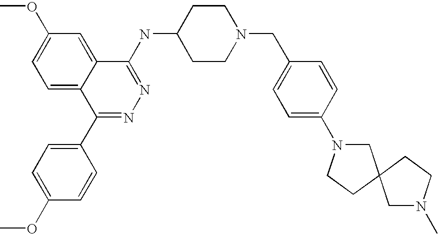

### 65.1. tert-Butyl 7-(4-formylphenyl)-2,7-diazaspiro[4.4]nonane-2-carboxylate

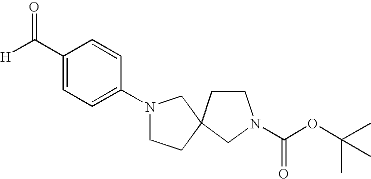

This compound is obtained according to the procedure described in 31.1. by reacting 4-bromobenzaldehyde with commercial tert-butyl 2,7-diazaspiro[4.4]nonane-2-carboxylate.

LC/MS: MH⁺=331 (Rt=9.18 minutes, pH 3.1)

### 65.2. [4-(7-Methyl-2,7-diazaspiro[4.4]non-2-yl)phenyl]methanol

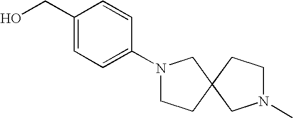

To a suspension of 512 mg (13.5 mmol) of lithium aluminum hydride in 5 mL of THF is added a solution of 445 mg (1.35 mmol) of tert-butyl 7-(4-formylphenyl)-2,7-diazaspiro[4.4]nonane-2-carboxylate in 5 mL of THF. The mixture is stirred at 60° C. for 4 hours and overnight at room temperature, followed by slow and successive addition of 0.5 mL of water, 0.5 mL of 6 N sodium hydroxide and 1.5 mL of water. A precipitate forms, which is filtered off through Celite and rinsed with ethyl acetate. The filtrate is evaporated under reduced pressure. 347 mg of an oily product are obtained.

LC/MS: MH⁺=247 (Rt=4.08 minutes, pH 3.1)

### 65.3. 4-(7-Methyl-2,7-diazaspiro[4.4]non-2-yl)benzaldehyde

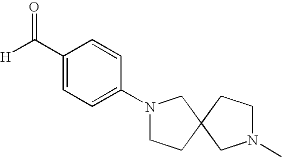

This compound is obtained by oxidation with manganese dioxide of [4-(7-methyl-2,7-diazaspiro[4.4]non-2-yl)phenyl]methanol according to the procedure described in 53.4.

LC/MS: MH⁺=245 (Rt=4.38 minutes, pH 3.1)

### 65.4. 7-Methoxy-4-(4-methoxyphenyl)-N-{1-[4-(7-methyl-2,7-diazaspiro[4.4]non-2-yl)benzyl]piperidin-4-yl}phthalazin-1-amine trihydrochloride (compound 65)

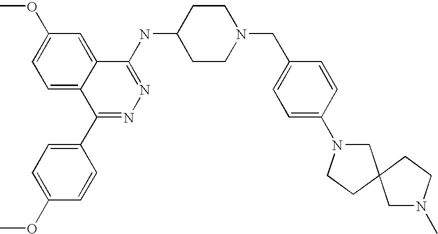

This compound is obtained according to the procedure described in 26.2. by reacting 7-methoxy-4-(4-methoxyphenyl)-N-(piperidin-4-yl)phthalazin-1-amine (preparation 3.2) with 4-(7-methyl-2,7-diazaspiro[4.4]non-2-yl)benzaldehyde.

LC/MS: MH⁺=593 (Rt=4.79 minutes, pH 3.1)

¹H NMR (DMSO-d₆, 400 MHz) δ ppm: 1.58-1.70 (m, 2H); 1.72-1.80 (m, 2H); 1.85-2.10 (m, 6H); 2.25 (s, 3H); 2.38-2.42 (m, 1H); 2.45-2.60 (m, 3H); 2.85-2.92 (m, 2H); 3.10-3.13 (m, 1H); 3.20-3.38 (m, 3H); 3.40 (s, 2H); 3.84 (s, 3H); 3.96 (s, 3H); 4.20-4.30 (m, 1H); 6.46 (d, J=8.8 Hz, 2H); 6.98 (d, J=7.6 Hz. 1H, NH); 7.05-7.13 (m, 4H); 7.42 (dd, J₁=8.8 Hz; J₂=2.4 Hz, 1H); 7.51 (d, J=8.8 Hz, 2H); 7.70-7.77 (m, 2H).

### EXAMPLE 66

### 4-{4-[(4-{[7-Methoxy-4-(methoxymethyl)phthalazin-1-yl]amino}piperidin-1-yl)-methyl]phenyl}butan-1-ol dihydrochloride (compound 66)

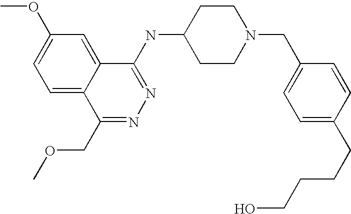

### 66.1. Ethyl 4-[4-(tetrahydro-2H-pyran-2-yloxy)butyl]benzoate

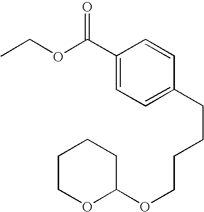

A solution of 1.15 g (56 mmol) of ethyl 4-(4-hydroxybutyl)benzoate (synthesized according to the method described in J. Med. Chem., 1983 26 (3) pp. 335-341) and 2.36 mL (25 mmol) of 3-4-dihydropyran in 20 mL of dichloromethane is stirred at 0° C. and 10 mg of p-toluenesulfonic acid monohydrate are added. The mixture is stirred at room temperature for 4 hours, followed by addition or dichloromethane and saturated aqueous sodium hydrogen carbonate solution. The organic phase is washed with saturated aqueous sodium chloride, dried over anhydrous sodium sulfate and then evaporated under reduced pressure. The residue obtained is purified on a column of silica (eluent: heptane/dichloromethane from 100/0 to 0/100 (v/v)). 1.5 g of oily yellow product are obtained.

¹H NMR (DMSO-d₆, 250 MHz) δ ppm: 1.32 (t, J=7 Hz, 3H); 1.40-1.85 (m, 10H); 2.63-2.73 (m, 2H); 3.30-3.48 (m, 2H); 3.60-3.78 (m, 2H); 4.30 (q, J=7 Hz, 2H); 4.50-4.55 (m, 1H); 7.36 (d, J=8 Hz, 2H); 7.89 (d, J=8 Hz, 2H).

### 66.2. {4-[4-(Tetrahydro-2H-pyran-2-yloxy)butyl]phenyl}methanol

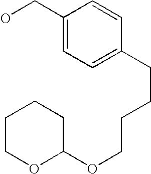

This compound is obtained according to the procedure described in 53.3. By reduction of ethyl 4-[4-(tetrahydro-2H-pyran-2-yloxy)butyl]benzoate.

¹H NMR (DMSO-d₆, 250 MHz) δ ppm: 1.40-1.80 (m, 10H); 2.55-2.62 (m, 2H); 3.30-3.48 (m, 2H); 3.58-3.78 (m, 2H); 4.45 (d, J=5.5 Hz, 2H); 4.50-4.55 (m, 1H); 5.10 (t, J=5.5 Hz, 1H, OH); 7.14 (d, J=8 Hz, 2H); 7.22 (d, J=8 Hz, 2H).

### 66.3. 4-[4-(Tetrahydro-2H-pyran-2-yloxy)butyl]benzaldehyde

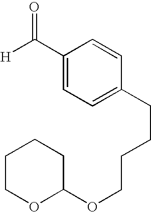

This compound is obtained according to the procedure described in 53.4. by oxidation of {4-[4-(tetrahydro-2H-pyran-2-yloxy)butyl]phenyl}methanol.

¹H NMR (DMSO-d₆, 250 MHz) δ ppm: 1.40-1.80 (m, 10H); 2.65-2.78 (m, 2H); 3.35-3.48 (m, 2H); 3.60-3.78 (m, 2H); 4.51-4.56 (m, 1H); 7.45 (d, J=8 Hz, 2H); 7.85 (d, J=8 Hz, 2H); 10.0 (s, 1H, CHO).

### 66.4. 4-{4-[(4-{[7-Methoxy-4-(methoxymethyl)phthalazin-1-yl]amino}piperidin-1-yl)methyl]phenyl}butan-1-ol dihydrochloride (compound 66)

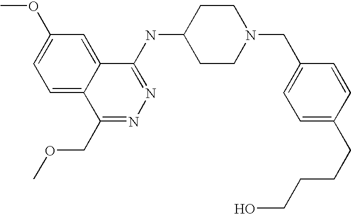

This compound in base form is obtained according to the procedure described in 30.2. by reacting 7-methoxy-4-methoxymethyl-N-(piperidin-4-yl)phthalazin-1-amine (preparation 22.2) with 4-[4-(tetrahydro-2H-pyran-2-yloxy)butyl]benzaldehyde.

The dihydrochloride is obtained by means of the treatment described in 4.3.

### EXAMPLE 67

### 7-Methoxy-4-(methoxymethyl)-N-[1-({1-[4-(trifluoromethyl)phenyl]-1H-pyrrol-3-yl}methyl)piperidin-4-yl]phthalazin-1-amine dihydrochloride (compound 67)

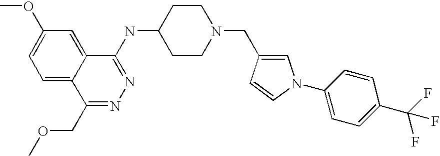

This compound in base form is obtained according to the procedure described in 26.2. by reacting 7-methoxy-4-methoxymethyl-N-(piperidin-4-yl)phthalazin-1-amine (preparation 22.2) with 1-[4-(trifluoromethyl)phenyl]-1H-pyrrole-3-carbaldehyde synthesized according to the method described in Synthetic Communications 1994, 24(13) 1855-1857.

The dihydrochloride is obtained by means of the treatment described in 4.3.

### EXAMPLE 68

### N-(1-{4-[2-(1H-Imidazol-1-yl)ethoxy]benzyl}piperidin-4-yl)-7-methoxy-4-(4-methoxyphenyl)phthalazin-1-amine trihydrochloride (compound 68)

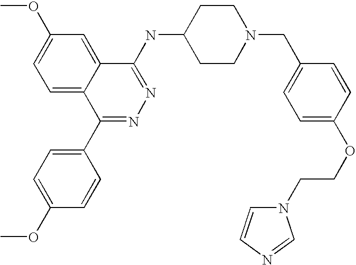

### 68.1. 4-(2-Chloroethoxy)benzaldehyde

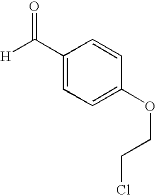

A solution of 2.5 g (20.5 mmol) of 4-hydroxybenzaldehyde in 200 mL of butan-2-one is stirred at room temperature under nitrogen and 7.33 g (22.5 mmol) of cesium carbonate are added. The mixture is stirred for one hour at room temperature, followed by addition of 11.7 g (61.4 mmol) of 2-chloro-1-iodoethane and 2.5 mg of potassium iodide. The mixture is stirred at 80° C. for 8 hours and then at room temperature overnight. Water and ethyl acetate are added to the reaction medium. The organic phase is washed with saturated aqueous sodium chloride, dried over anhydrous sodium sulfate and then evaporated under reduced pressure. The residue obtained is purified on a column of silica (eluent: dichloromethane/methanol from 100/0 to 80/20 (v/v)). 1.3 g of gummy product are obtained.

LC/MS: MH⁺=185 (Rt=7.78 minutes, pH 3.1)

¹H NMR (DMSO-d₆, 250 MHz) δ ppm: 3.98-4.03 (m, 2H); 4.38-4.43 (m, 2H); 7.18 (d, J=8 Hz, 2H); 7.89 (d, J=8 Hz, 2H); 9.90 (s, 1H, CHO).

### 68.2. 4-[2-(1H-Imidazol-1-yl)ethoxy]benzaldehyde

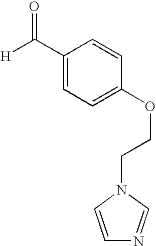

A solution of 1.2 g (6.5 mmol) of 4-(2-chloroethoxy)benzaldehyde in 10 mL of DMF is stirred at 0° C. under nitrogen and 374 mg of a 60% suspension of sodium hydride in oil (9.35 mmol) are added. The mixture is stirred at 0° C. for one hour, followed by addition of a solution of 0.487 g (7.15 mmol) of imidazole in 10 mL of DMF. The mixture is stirred for 18 hours at room temperature and is then heated at 60° C. for 4 hours 30 minutes. After cooling to room temperature, and addition of 50 mL of water, the mixture is extracted with ethyl acetate. The organic phase is washed with water and then with saturated aqueous sodium chloride, dried over anhydrous sodium sulfate and then evaporated under reduced pressure. The residue obtained is purified on a column of silica (eluent: dichloromethane/methanol from 100/0 to 95/5 (v/v)).

LC/MS: MH⁺=217 (Rt=3.48 minutes, pH 3.1)

¹H NMR (DMSO-d₆, 250 MHz) δ ppm: 4.33-4.43 (m, 4H); 6.90 (d, J=1 Hz, 1H); 7.14 (d, J=8.8 Hz, 2H); 7.27 (dd, J₁=J₂=1 Hz, 1H); 7.70 (d, J=1 Hz, 1H); 7.88 (d, J=8.8 Hz, 2H); 9.88 (s, 1H, CHO).

### 68.3. N-(1-{4-[2-(1H-Imidazol-1-yl)ethoxy]benzyl}piperidin-4-yl)-7-methoxy-4-(4-methoxyphenyl)phthalazin-1-amine trihydrochloride (compound 68)

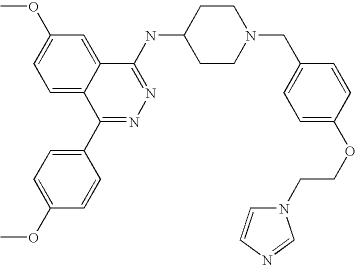

This compound in base form is obtained according to the procedure described in 8.2. by reacting 7-methoxy-4-(4-methoxyphenyl)-N-(piperidin-4-yl)phthalazin-1-amine (preparation 3.2) with 4-[2-(1H-imidazol-1-yl)ethoxy]benzaldehyde.

The trihydrochloride is obtained by means of the treatment described in 4.3.

### EXAMPLE 69

### 7-Methoxy-4-(4-methoxyphenyl)-N-{1-[4-(pyridin-4-ylmethoxy)benzyl]piperidin-4-yl}phthalazin-1-amine trihydrochloride (compound 69)

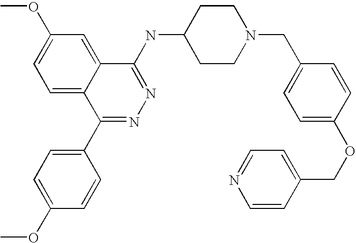

### 69.1. 4-(Pyridin-4-ylmethoxy)benzaldehyde

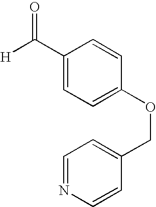

A mixture of 5 g (40.9 mmol) of 4-hydroxybenzaldehyde, 16.1 g (61.4 mmol) of triphenylphosphine and 0.943 g (8.19 mmol) of pyridin-4-ylmethanol in 170 mL of THF is stirred at 0° C. and 12.09 mL (61.4 mmol) of diisopropyl azodicarboxylate are added slowly. The reaction medium is stirred 15 minutes at 0° C. and then overnight at room temperature. 2N sodium hydroxide is added and the mixture is extracted with tert-butyl methyl ether. The organic phase is extracted with 1 N HCl solution. The acidic aqueous phase obtained is basified with 6 N sodium hydroxide and then extracted with tert-butyl methyl ether. The organic phase is washed with saturated aqueous sodium chloride, dried over anhydrous sodium sulfate and then evaporated under reduced pressure. The residue obtained is chromatographed on a column of silica (eluent: dichloro-methane/methanol from 100/0 to 85/15 (v/v)). The oily brown product, which is still impure, is used as obtained.

LC/MS: MH⁺=214 (Rt=4.36 minutes, pH 3.1)

### 69.2. 7-Methoxy-4-(4-methoxyphenyl)-N-{1-[4-(pyridin-4-ylmethoxy)-benzyl]piperidin-4-yl}phthalazin-1-amine trihydrochloride (compound 69)

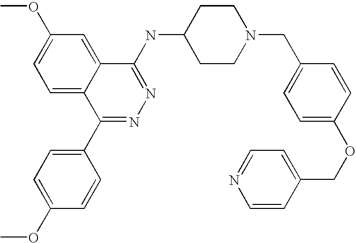

This compound in base form is obtained according to the procedure described in 26.2. by reacting 7-methoxy-4-(4-methoxyphenyl)-N-(piperidin-4-yl)phthalazin-1-amine (preparation 3.2) with 4-(pyridin-4-ylmethoxy)benzaldehyde.

¹H NMR (DMSO-d₆, 250 MHz) δ ppm: 1.60-1.78 (m, 2H); 2.0-2.18 (m, 4H); 2.88-2.96 (m, 2H); 3.46 (s, 2H); 3.85 (s, 3H); 3.97 (s, 3H); 4.20-4.30 (m, 1H); 5.18 (s, 2H); 6.95-7.04 (m, 3H); 7.09 (d, J=8.8 Hz, 2H); 7.26 (d, J=8.8 Hz, 2H); 7.40-7.50 (m, 3H); 7.52 (d, J=8.8 Hz, 2H); 7.72-7.79 (m, 2H); 8.59 (d, J=6 Hz, 2H).

The trihydrochloride is obtained by means of the treatment described in 4.3.

### EXAMPLE 70

### 7-Methoxy-4-(4-methoxyphenyl)-N-{1-[4-(tetrahydrofuran-2-ylmethoxy)benzyl]-piperidin-4-yl}phthalazin-1-amine dihydrochloride (compound 70)

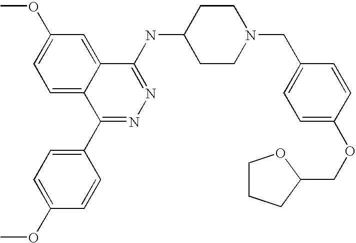

### 70.1. 4-(Tetrahydrofuran-2-ylmethoxy)benzaldehyde

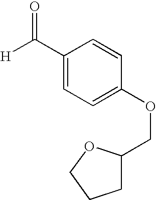

This compound is obtained via the Mitsunobu reaction according to the procedure described in 17.1. by reacting 4-hydroxybenzaldehyde with (R,S)-(tetrahydro-furan-2-yl)methanol.

LC/MS: MH⁺=207 (Rt=6.97 minutes, pH 3.1)

¹H NMR (DMSO-d₆, 250 MHz) δ ppm: 1.64-1.72 (m, 1H); 1.80-1.93 (m, 2H); 1.98-2.08 (m, 1H); 3.17-3.22 (m, 1H); 3.25-3.30 (m, 1H); 4.01-4.12 (m, 2H); 4.15-4.20 (m, 1H); 7.13 (d, J=8.8 Hz, 2H); 7.85 (d, J=8.8 Hz, 2H); 9.87 (s, 1H, CHO).

### 70.2. 7-Methoxy-4-(4-methoxyphenyl)-N-{1-[4-(tetrahydrofuran-2-yl-methoxy)benzyl]piperidin-4-yl}phthalazin-1-amine dihydrochloride (compound 70)

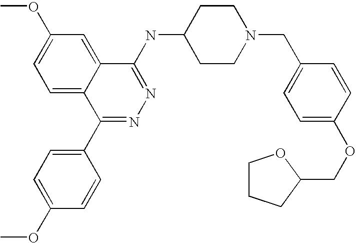

This compound in base form is obtained according to the procedure described in 26.2. by reacting 7-methoxy-4-(4-methoxyphenyl)-N-(piperidin-4-yl)phthalazin-1-amine (preparation 3.2) with 4-(tetrahydrofuran-2-ylmethoxy)benzaldehyde.

The dihydrochloride is obtained by means of the treatment described in 4.3.

TABLE 1

| | |
|---|---|
| | (I) |
| 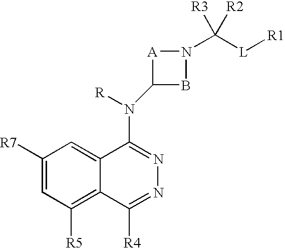 | |

| No. | 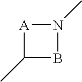 | 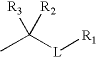 | R | R₄ | R₅ | R₇ | Salt and/or Solvates | m.p. (°C.) [α]²⁰_{D} | LC/MS (M + H)⁺; Rt (minutes); pH |
|---|---|---|---|---|---|---|---|---|---|
| 1 | 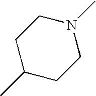 | 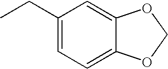 | H | 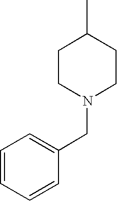 | H | OMe | HCl (3/1) H₂O (2.4/1) | 290 (M) | 566; Rt = 4.47; pH 3.1 |
| 2 | 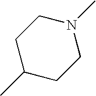 | 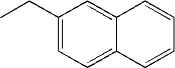 | Me | 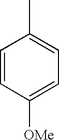 | H | OMe | HCl (2/1) H₂O (2/1) | 235 (M) | 519; Rt = 6.07; pH 3.1 |
| 3 | 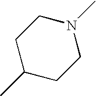 | 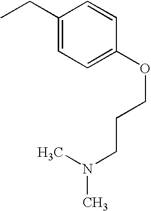 | H | 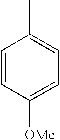 | H | OMe | HCl (3/1) H₂O (4.2/1) | 190 (K) | 556; Rt = 4.52; pH 3.1 |
| 4 | 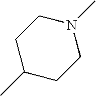 | 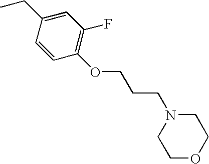 | H | 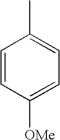 | H | OMe | HCl (3/1) H₂O (5.3/1) | 276 (M) | 616; Rt = 4.57; pH 3.1 |
| 5 | 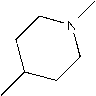 | 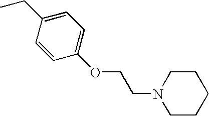 | H | 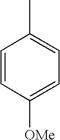 | H | OMe | HCl (3/1) H₂O (2.4/1) | 274 (M) | 582; Rt = 4.67; pH 3.1 |
| 6 | 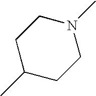 | 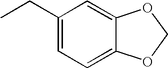 | Me | Et | H | OMe | HCl (2/1) H₂O (2/1) | 222 (M) | 435; Rt = 4.65; pH 3.1 |
| 7 | 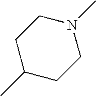 | 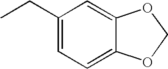 | Me | 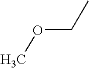 | H | OMe | HCl (2/1) H₂O (1.15/1) | 170 (K) | 451; Rt = 4.87; pH 3.1 |
| 8 | 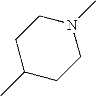 | 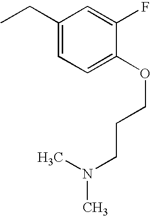 | H | 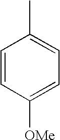 | H | OMe | HCl (3/1) H₂O (2.3/1) | 157 (M) | 574; Rt = 4.62; pH 3.1 |
| 9 | 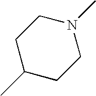 | 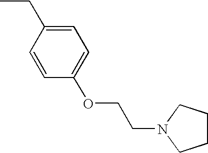 | H | 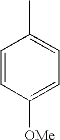 | H | OMe | HCl (3/1) H₂O (1.9/1) | 224 (K) | 568; Rt = 4.58; pH 3.1 |
| 10 | 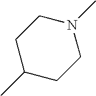 | 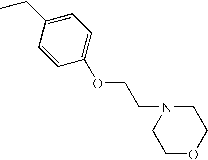 | H | 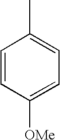 | H | OMe | HCl (3/1) H₂O (2.5/1) | 289 (M) | 584; Rt = 4.55; pH 3.1 |
| 11 | 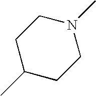 | 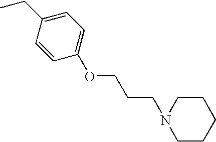 | H | 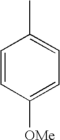 | H | OMe | HCl (3/1) H₂O (3.9/1) | 140 (M) | 596; Rt = 4.73; pH 3.1 |
| 12 | 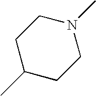 | 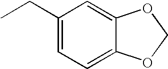 | Et | 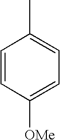 | H | OMe | HCl (2/1) H₂O (2.15/1) | 162 (M) | 527; Rt = 5.86; pH 3.1 |
| 13 | 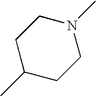 | 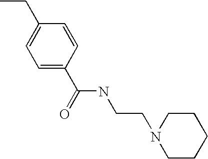 | H | 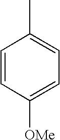 | H | OMe | HCl (3/1) H₂O (1.8/1) | 289 (M) | 609 Rt = 4.58; pH 3.1 |
| 14 | 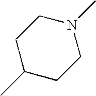 | 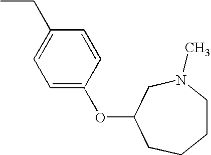 | H | 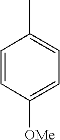 | H | OMe | HCl (3/1) H₂O (3/1) Et₂O (0.12/1) | 260 (B) | 582; Rt = 4.7; pH 3.1 |
| 15 | 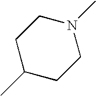 | 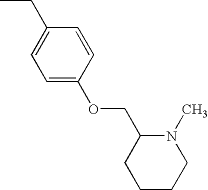 | H | 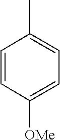 | H | OMe | HCl (3/1) H₂O (3/1) Et₂O (0.2/1) | 260 (B) | 582; Rt = 4.69; pH 3.1 |
| 16 | 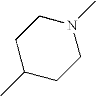 | 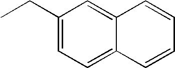 | 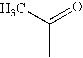 | 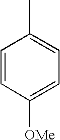 | H | OMe | HCl (2/1) H₂O (2.6/1) | 251 (M) | 547; Rt = 6.56; pH 3.1 |
| 17 | 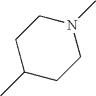 | 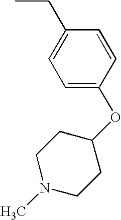 | H | 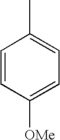 | H | OMe | HCl (3/1) H₂O (3/1) | 232 (M) | 568; Rt = 4.56; pH 3.1 |
| 18 | 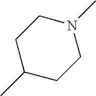 | 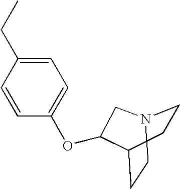 | H | 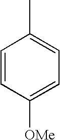 | H | OMe | HCl (3/1) H₂O (2.5/1) | 265 (B) | 580; Rt = 4.77; pH 3.1 |
| 19 | 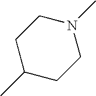 | 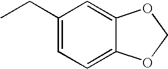 | H | 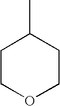 | H | OMe | HCl (2/1) H₂O (2/1) Et₂O (0.33/1) | 172 (K) | 447; Rt = 4.67; pH 3.1 |
| 20 | 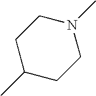 | 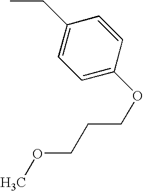 | H | 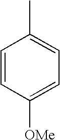 | H | OMe | HCl (2/1) H₂O (1/1) | 219 (M) | 543; Rt = 5.48; pH 3.1 |
| 21 | 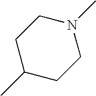 | 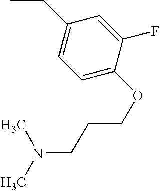 | H | Et | H | OMe | HCl (3/1) H₂O (1.9/1) Et₂O (0.12/1) | 200 (B) | 496; Rt = 7.24; pH 7 |
| 22 | 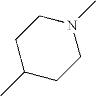 | 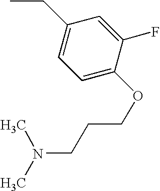 | H | 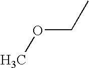 | H | OMe | HCl (3/1) H₂O (2.2/1) | 195 (B) | 512; Rt = 8.77; pH 2.2 |
| 23 | 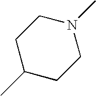 | 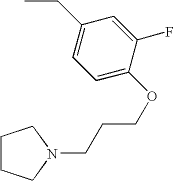 | H | 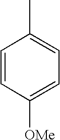 | H | OMe | HCl (3/1) H₂O (2.4/1) | 190 (K) | 600; Rt = 4.72; pH 3.1 |
| 24 | 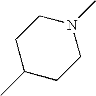 | 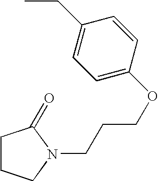 | H | 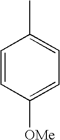 | H | OMe | HCl (3/1) H₂O (2/1) Et₂O (0.23/1) | 143 (M) | 596; Rt = 5.31; pH 3.1 |
| 25 | 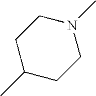 | 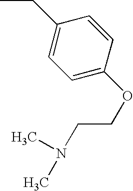 | H | 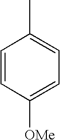 | H | OMe | HCl (3/1) H₂O (3.8/1) iPr₂O (0.21/1) | 206 (K) | 542; Rt = 4.44; pH 3.1 |
| 26 | 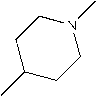 | 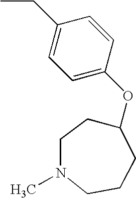 | H | 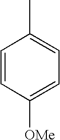 | H | OMe | HCl (3/1) H₂O (4.3/1) Et₂O (0.22/1) | 220 (K) | 582; Rt = 4.71; pH 3.1 |
| 27 | 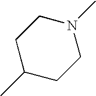 | 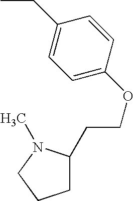 | H | 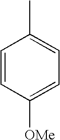 | H | OMe | HCl (3/1) H₂O (4.9/1) Et₂O (0.36/1) | 249 (M) | 582; Rt = 4.71; pH 3.1 |
| 28 | 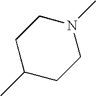 | 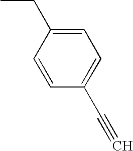 | H | Et | H | OMe | HCl (2/1) H₂O (2/1) | 214 (K) | 401; Rt = 4.76; pH 3.1 |
| 29 | 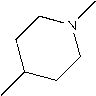 | 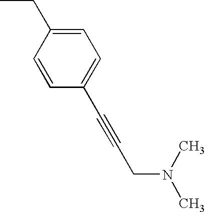 | H | Et | H | OMe | HCl (3/1) H₂O (3.5/1) | 198 (K) | 458; Rt = 3.92 and 3.99; pH 3.1 |
| 30 | 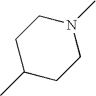 | 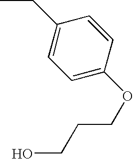 | H | 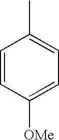 | H | OMe | HCl (2/1) H₂O (1.5/1) | 222 (K) | 529; Rt = 5.20; pH 3.1 |
| 31 | 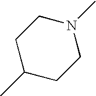 | 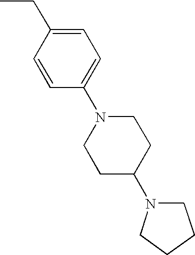 | H | Et | H | OMe | HCl (3/1) H₂O (4.1/1) | 282 (M) | 529; Rt = 4.14; pH 3.1 |
| 32 | 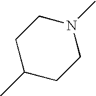 | 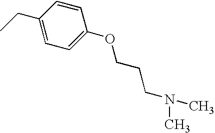 | H | 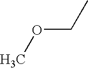 | H | OMe | HCl (3/1) H₂O (2.3/1) Et₂O (0.1/1) | 195 (K) | 494; Rt = 3.87 and 3.95; pH 3.1 |
| 33 (S) | 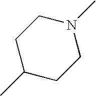 | 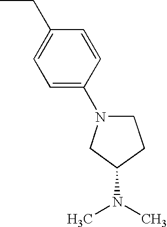 | H | 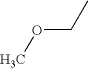 | H | OMe | HCl (3/1) H₂O (4.5/1) | 283 (M) | 505; Rt = 3.88 and 3.96; pH 3.1 |
| 34 (R) | 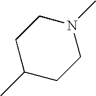 | 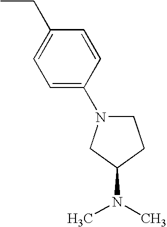 | H | 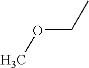 | H | OMe | HCl (3/1) H₂O (4/1) | 250 (K) [α]²⁰_{D} = −3° (c = 0.53 MeOH) | 505; Rt = 3.79 and 3.90; pH 3.1 |
| 35 | 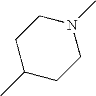 | 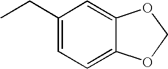 | H | 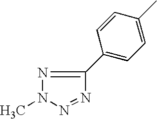 | H | OMe | HCl (2/1) H₂O (3/1) | 232 (K) | 551; Rt = 5.4; pH 3.1 |
| 36 | 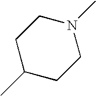 | 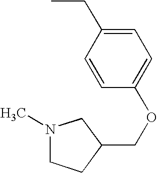 | H | 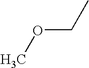 | H | OMe | HCl (3/1) H₂O (2/1) | 266 (M) | 506; Rt = 10.53; pH 7 elution over 30 minutes |
| 37 | 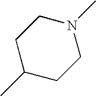 | 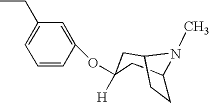 | H | 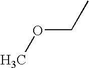 | H | OMe | HCl (3/1) H₂O (1.3/1) | 230 (B) | 532; Rt = 8.64; pH 2.2 |
| 38 | 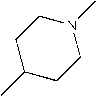 | 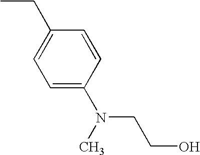 | H | 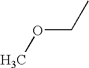 | H | OMe | HCl (3/1) H₂O (3/1) Et₂O (0.27/1) | 108 (B) | 466; Rt = 4.45; pH 3.1 |
| 39 | 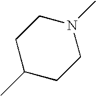 | 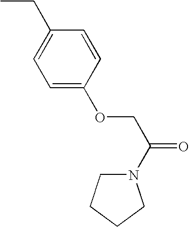 | H | 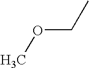 | H | OMe | HCl (2/1) H₂O (2.2/1) | 216 (M) | 520; Rt = 4.6; pH 3.1 |
| 40 | 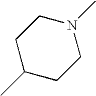 | 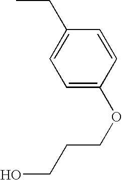 | H | 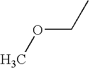 | H | OMe | HCl (2/1) H₂O (3.1/1) | 205 (M) | 467; Rt = 4.51; pH 3.1 |
| 41 | 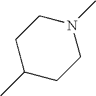 | 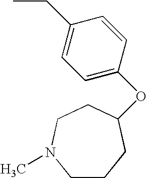 | H | 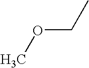 | H | OMe | HCl (3/1) H₂O (2.8/1) Et₂O (0.18/1) | 206 (K) | 520; Rt = 3.63; pH 3.1 |
| 42 | 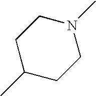 | 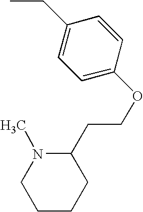 | H | 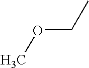 | H | OMe | HCl (3/1) H₂O (4.8/1) | 186 (K) | 534; Rt = 4.3; pH 3.1 |
| 43 | 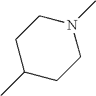 | 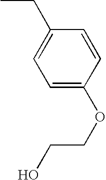 | H | 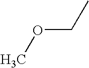 | H | OMe | HCl (2/1) H₂O (1.2/1) | 231 (M) | 453; Rt = 4.33; pH 3.1 |
| 44 | 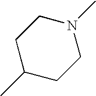 | 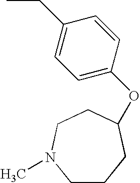 | H | Me | H | OMe | HCl (3/1) H₂O (2.5/1) Et₂O (0.3/1) | 287 (M) | 490; Rt = 3.58; pH 3.1 |
| 45 | 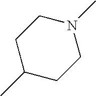 | 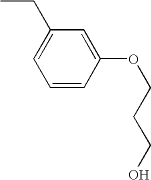 | H | 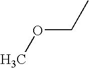 | H | OMe | HCl (2/1) H₂O (2/1) Et₂O (0.3/1) | 228 (K) | 467; Rt = 4.17; pH 3.1 |
| 46 | 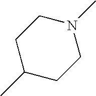 | 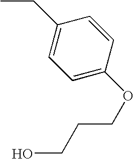 | H | Me | H | OMe | HCl (2/1) H₂O (2/1) Et₂O (0.25/1) | 186 (K) | 437; Rt = 3.99; pH 3.1 |
| 47 | 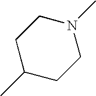 | 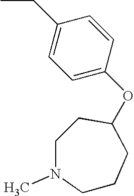 | H | Me | H | Cl | HCl (3/1) H₂O (2.9/1) | 218 (K) | 494; Rt = 3.59; pH 3.1 |
| 48 | 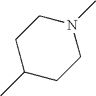 | 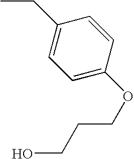 | H | Me | H | Cl | HCl (2/1) H₂O (1/1) Et₂O (0.35/1) | 185 (K) | 441; Rt = 4.05; pH 3.1 |
| 49 (S) | 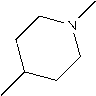 | 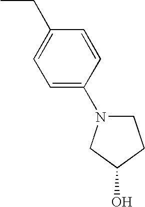 | H | 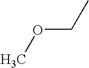 | H | OMe | HCl (3/1) H₂O (0.5/1) Et₂O (0.45/1) | 214 (K) [α]²⁰_{D} = +0.8° (c) = 0.5 MeOH) | 478; Rt = 4.17; pH 3.1 |
| 50 (R) | 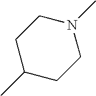 | 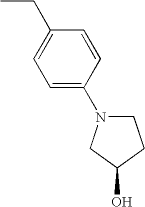 | H | 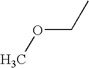 | H | OMe | HCl (3/1) H₂O (1/1) Et₂O (0.4/1) | 206 (K) [α]²⁰_{D} = −0.8° (c = 0.5 MeOH) | 478; Rt = 4.16; pH 3.1 |
| 51 | 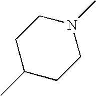 | 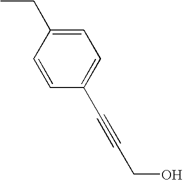 | H | 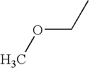 | H | OMe | H₂O (2.4/1) | 222 (M) | 447; Rt = 4.58; pH 3.1 |
| 52 | 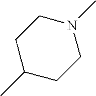 | 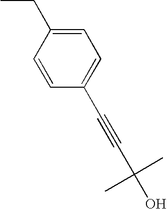 | H | 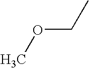 | H | OMe | H₂O (0.75/1) | 165 (M) | 475; Rt = 4.83; pH 3.1 |
| 53 | 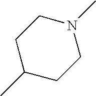 | 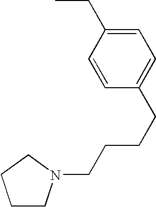 | H | 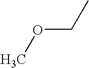 | H | OMe | (COOH)_{2 (3/1) H}₂O (0.8/1) | 204 (M) | 518; Rt = 4.23; pH 3.1 |
| 54 | 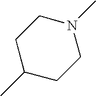 | 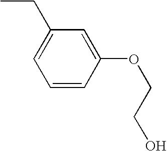 | H | 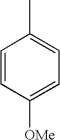 | H | OMe | HCl (2/1) H₂O (1.7/1) | 276 (M) | 515; Rt = 5.13; pH 3.1 |
| 55 | 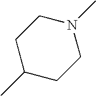 | 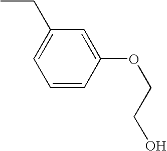 | H | 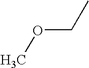 | H | OMe | HCl (2/1) H₂O (2.25/1) | 247 (M) | 453; Rt = 4.4; pH 3.1 |
| 56 | 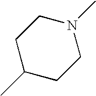 | 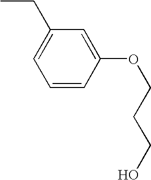 | H | 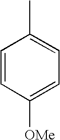 | H | OMe | HCl (2/1) H₂O (3/1) Ipr₂O (0.15/1) | 205 (M) | 529; Rt = 5.26; pH 3.1 |
| 57 | 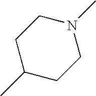 | 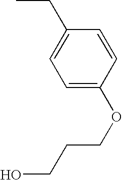 | H | 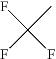 | H | OMe | HCl (2/1) H₂O (2.5/1) | 227 (M) | 491; Rt = 6.05; pH 3.1 |
| 58 | 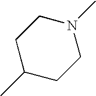 | 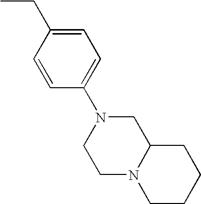 | H | 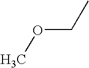 | H | OMe | HCl (3/1) H₂O (6/1) | 240 (K) | 531; Rt = 3.94 and 4.04; pH 3.1 |
| 59 | 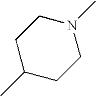 | 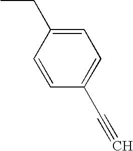 | H | Et | OMe | OMe | HCl (2/1) H₂O (3.2/1) | 210 (B) | 431; Rt = 6.01; pH 3.1 |
| 60 | 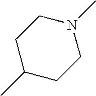 | 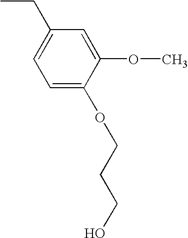 | H | 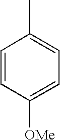 | H | OMe | HCl (2/1) H₂O (2/1) | 203 (B) | 559; Rt = 6.13; pH 3.1 |
| 61 | 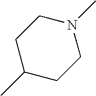 | 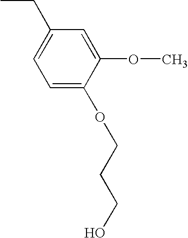 | H | 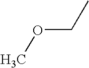 | H | OMe | HCl (2/1) H₂O (1.5/1) | 194 (B) | 497; Rt = 5.18; pH 3.1 |
| 62 | 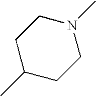 | 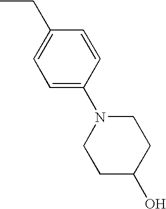 | H | 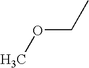 | H | OMe | HCl (3/1) H₂O (3.8/1) | 279 (M) | 492; Rt = 4.33; pH 3.1 |
| 63 | 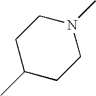 | 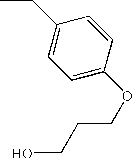 | H | H | H | OMe | HCl (2/1) H₂O (2.2/1) | 251 (M) | 423; Rt = 4.31; pH 3.1 |
| 64 | 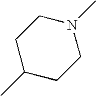 | 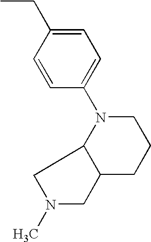 | H | 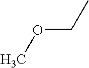 | H | OMe | HCl (3/1) H₂O (5.5/1) | 227 (M) | 531; Rt = 4.24; pH 3.1 |
| 65 | 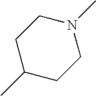 | 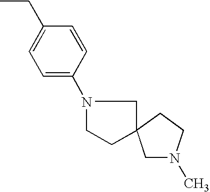 | H | 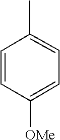 | H | OMe | HCl (3/1) H₂O (5.5/1) CH₂Cl₂ (0.35/1) | 294 (M) | 593 Rt = 4.79 pH 3.1 |
| 66 | 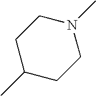 | 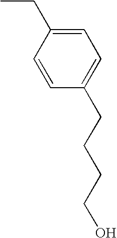 | H | 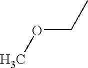 | H | OMe | HCl (2/1) H₂O (2/1) | 230 (M) | 465 Rt = 4.47 pH = 3.1 |
| 67 | 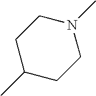 | 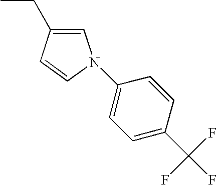 | H | 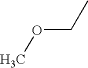 | H | OMe | HCl (2/1) H₂O (2/1) | 196 (B) | 526; Rt = 5.73; pH = 3.1 |
| 68 | 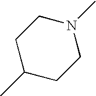 | 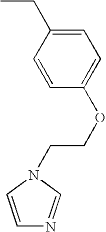 | H | 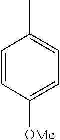 | H | OMe | HCl (3/1) H₂O (1.5/1) | 219 (M) | 565; Rt = 7.62 pH = 7 |
| 69 | 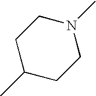 | 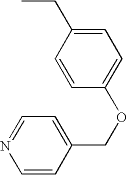 | H | 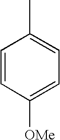 | H | OMe | HCl (3/1) H₂O (3.1/1) Et₂O (0.3/1) | 281 (M) | 562; Rt = 4.93 pH = 3.1 |
| 70 | 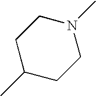 | 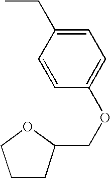 | H | 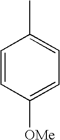 | H | OMe | HCl (2/1) H₂O (1.6/1) | 292 (M) | 555; Rt = 5.54; pH = 3.1 |

The compounds according to the invention underwent pharmacological tests. Their affinity towards the receptor 1 of Melanin-Concentrating Hormone (MCH), MCH₁, was thus determined.

The tests consisted in measuring the in vitro activity of the compounds of the invention on the MCH₁ receptors of MCH.

### Binding Studies

The measurement of the affinity of the compounds of the invention for the MCH receptors was performed by studying the displacement of the binding of a radiolabelled derivative of MCH to the MCH₁ receptors. This study was performed on rat and/or mouse brain membrane preparations according to the protocol described below.

In anticipation of the binding studies, the brains are diluted in HEPES buffer 25 mM (pH: 7.4) containing MgCl₂ 5 mM, CaCl₂ 1 mM, homogenized using a Polytron blender for 3 times 20 seconds (speed 25), and are then ultracentrifuged at 22000 rpm and at +4° C. for 30 minutes. The centrifugation pellet is taken up in the same buffer and the membranes are divided into aliquots and stored frozen at −80° C. until the time of use. The membranes are warmed to room temperature and then incubated in the presence of the test compounds, and of 50 μM of an MCH-based radiolabelled molecule, [¹²⁵I]-Tyr-S36057 (8-amino-3,6-dioxyoctanoyl MCH 6-17 sold by Perkin-Elmer), in HEPES buffer 25 mM (pH: 7.4) containing MgCl₂ 5 mM, CaCl₂ 1 mM, bacitracin 140 mg/L, phenanthroline 1 mM and 0.2% bovine serum albumin. The incubation is performed at room temperature for 30 minutes, and then stopped by rapid addition of ice-cold HEPES buffer 25 mM (pH: 7.4) supplemented with 0.2% bovine serum albumin, and by filtration through GF/B glass fiber filters preincubated for 2 hours in an aqueous 0.1% polyethyleneimine solution. The radioactivity retained on the filters is measured using a Gamma scintillation counter. The non-specific binding is determined in the presence of 1 μM of non-radiolabelled S36057. The specific binding is obtained by difference between the total binding and the non-specific binding. The inhibitory activity of the compounds of the invention is expressed by means of the concentration that inhibits 50% of the specific binding (IC₅₀).

In the context of the invention, the IC₅₀ values of the compounds are generally less than 10 μM.

The compounds of formula (I) advantageously have IC₅₀ values of less than 1 μM, more advantageously less than or equal to 100 nM and even more advantageously less than or equal to 10 nM.

By way of example:
the compound according to Example 1 has an IC₅₀ of 0.5 nM in rats and of 0.2 nM in mice;
the compound according to Example 3 has an IC₅₀ of 52 nM in rats and of 38 nM in mice.

### Pharmacological Feeding Model:

The activity of an MCH receptor 1 antagonist may be controlled pharmacologically by means of a test performed on rats (young rats weighing 80 to 150 g).

This test consists in inducing feeding behavior via an i.c.v. (intracerebroventricular) injection of MCH performed manually. The i.c.v. Injection of peptide dissolution buffer (without MCH) to a first control group makes it possible to quantify the size of the effect due to MCH.

A compound according to the invention is administered orally 1 or 2 hours before the i.c.v. treatment.

The effect of a compound according to the invention is measured by means of the reduction it may cause in the taking of food, induced beforehand by the i.c.v. injection of MCH.

The specificity of action of the product may be evaluated by using another orexigenic peptide, for instance NPY, also injected via the i.c.v. route. Thus, a product that is specific for the MCH₁ receptor will have no effect on the taking of food induced by another orexigenic peptide, for instance NPY.

The compounds according to the invention may be used for the preparation of medicaments, in particular medicaments that are antagonists of the MCH₁ receptor of MCH.

Although the invention has been illustrated by certain of the preceding examples, it is not to be construed as being limited thereby; but rather, the invention encompasses the generic area as hereinbefore disclosed. Various modifications and embodiments can be made without departing from the spirit and scope thereof.

What is claimed is:

1. A compound of the formula (I): 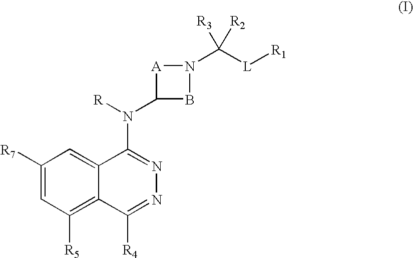
in which:
A represents a C₁₋₄-alkylene group optionally substituted with one or more groups R₉, which may be identical or different;
B represents a C₁₋₄-alkylene group optionally substituted with one or more groups R₁₀, which may be identical or different;
R₉ and R₁₀ represent, independently of each other, a hydrogen atom or a C₁₋₅-alkyl group,
or alternatively R₉ and R₁₀ together form a single bond or a C₁₋₄-alkylene group;
L represents a single bond or a C₁₋₂-alkylene, —CH═CH— or —C≡C— group; the C₁₋₂-alkylene and —CH═CH— groups being optionally substituted with one or more C₁₋₂-alkyl substituents,
or alternatively L represents a cycloprop-1,2-diyl group;
R represents a hydrogen atom or a C₁₋₅-alkyl, C₁₋₃-fluoroalkyl, C₃₋₆-cycloalkyl, —C(O)C₁₋₃-alkyl, C₁₋₃-alkylene-C₃₋₆-cycloalkyl, —CH₂—C≡CH, C₂₋₃-alkylene-NRₐR_{b} or C₁₋₃-alkylene-X—C₁₋₃-alkyl group in which X represents O or SO₂;
R₁ represents an aryl or a heteroaryl group; the aryl and heteroaryl groups being optionally substituted with one or more radicals Z, which may be identical or different;
R₂ and R₃ represent, independently of each other, a hydrogen atom or a C₁₋₃-alkyl or C₁₋₃-fluoroalkyl group,
or alternatively R₂ and R₃ form, together with the carbon atom that bears them, a cycloprop-1,1-diyl group;
R₄ represents:
a hydrogen atom or a C₁₋₅-alkyl, C₁₋₃-fluoroalkyl, C₃₋₄-cycloalkyl, C₁₋₃-alkylene-C₃₋₆-cycloalkyl, C₁₋₃-alkylene-O—C₁₋₃-alkyl or C₁₋₃-alkylene-O—C₄₋₆-cycloalkyl group,
a C₁₋₃-alkylene-(OH) or C₁₋₃-alkylene-X—C₁₋₃-alkyl group in which X represents S, SO or SO₂,
a heterocyclic group; the said heterocyclic group being optionally substituted with a C₁₋₃-alkyl, —C(O)C₁₋₃-alkyl, —C(O)C₁₋₃-fluoroalkyl, C₁₋₃-alkylene-C₃₋₆-cycloalkyl, C₁₋₃-alkylene-aryl or C₁₋₃-alkylene-heteroaryl group; the C₁₋₃-alkylene-aryl and C₁₋₃-alkylene-heteroaryl groups being optionally substituted with one or more radicals Z, which may be identical or different,
or alternatively R₄ represents a C₁₋₃-alkylene-NRₐR_{b}, aryl, C₁₋₃-alkylene-aryl, —O-aryl, C₁₋₃-alkylene-O-aryl, C₁₋₃-alkylene-O—C₁₋₃-alkylene-aryl, heteroaryl or C₁₋₃-alkylene-heteroaryl group; the aryl, C₁₋₃-alkylene-aryl, —O-aryl, C₁₋₃-alkylene-O-aryl, C₁₋₃-alkylene-O—C₁₋₃-alkylene-aryl, heteroaryl and C₁₋₃-alkylene-heteroaryl groups being optionally substituted with one or more radicals Z, which may be identical or different;
R₅ represents a hydrogen or halogen atom or a C₁₋₅-alkyl, C₁₋₃-fluoroalkyl, C₁₋₅-alkoxy, C₁₋₃-fluoroalkoxy, C₁₋₃-alkylene-(OH), —CN or —X—C₁₋₃-alkyl group in which X represents S, SO or SO₂,
or alternatively R₅ represents an —NRₐR_{b}, C₁₋₃-alkylene-NRₐR_{b}, aryl, C₁₋₃-alkylene-aryl, —O-aryl or heteroaryl group; the aryl, C₁₋₃-alkylene-aryl, —O-aryl and heteroaryl groups being optionally substituted with one or more radicals Z, which may be identical or different;
R₇ represents a hydrogen or halogen atom or a C₁₋₅-alkyl, C₁₋₃-fluoroalkyl, C₁₋₅-alkoxy, —COOH, —C(O)OC₁₋₃-alkyl, C₁₋₃-fluoroalkoxy, C₁₋₃-alkylene-(OH), —CN or —X—C₁₋₃-alkyl group in which X represents S, SO or SO₂,
or alternatively R₇ represents an —NRₐR_{b}, C₁₋₃-alkylene-NRₐR_{b} or —C(O)—NRₐR_{b}, —C(O)—C₁₋₃-alkyl, aryl, —O-aryl or heteroaryl group; the aryl, —O-aryl and heteroaryl groups being optionally substituted with one or more radicals Z, which may be identical or different;
Z represents a halogen atom or a C₁₋₅-alkyl, C₁₋₃-fluoroalkyl, C₃₋₆-cycloalkyl, C₁₋₃-alkylene-C₃₋₄-cycloalkyl, C₁₋₅-alkoxy, C₁₋₃-fluoroalkoxy, C₁₋₃-alkylene-O—C₁₋₃-alkyl, C₁₋₃-alkylene-(OH), NO₂, —CN, —SO₂NRₐR_{b}, —X—C₁₋₃-alkyl or C₁₋₃-alkylene-X—C₁₋₃-alkyl group in which X represents S, SO or SO₂,
or alternatively Z represents a phenyl optionally substituted with a halogen atom or a C₁₋₅-alkyl, C₁₋₅-alkoxy or C₁₋₃-fluoroalkyl group,
or alternatively Z represents an alkynyl radical of the type —CE≡C—Rₑ, in which R_{c} represents a hydrogen atom or a C₁₋₃-alkyl, C₁₋₆-alkylene-O—R_{d} or C₁₋₆-alkylene-NRₐR_{b} group,
or alternatively Z represents a group —C₄₋₆-alkylene-OR_{d},
or alternatively Z represents a tetrazole group substituted with a C₁₋₃-alkyl group,
or alternatively Z represents an —NRₐR_{b}, C₁₋₃-alkylene-NRₐR_{b}, —C(O)—NRₐR_{b}, —C(O)—C₁₋₃-alkyl, —CO₂—C₁₋₄-alkyl or —C(O)—C₃₋₆-cycloalkyl group,
or alternatively Z represents a group —C₄₋₆-alkylene-NRₐR_{b},
or alternatively Z represents an oxo radical,
or alternatively Z represents an —O—C₁₋₅-alkylene-NRₐR_{b} group,
or alternatively Z represents an —O—C₀₋₃-alkylene-heterocycle group, optionally substituted with one or more C₁₋₃-alkyl, oxo or —C(O)—C₁₋₃-alkyl groups,
or alternatively Z represents a group —O—C₁₋₅-alkylene-O—R_{d},
or alternatively Z represents an —O—C₀₋₃-alkylene-C₅₋₇-cycloalkyl group optionally substituted with a group —O—R_{d},
or alternatively Z represents an —NRₑ—C₀₋₃-alkylene-heterocycle group optionally substituted with one or more C₁₋₃-alkyl, oxo or —C(O)—C₁₋₃-alkyl groups,
or alternatively Z represents a group —NRₑ—C₂₋₅-alkylene-O—R_{d},
or alternatively Z represents an —O—C₁₋₃-alkylene-heteroaryl group optionally substituted with one or more C₁₋₃-alkyl groups,
or alternatively Z represents a group —CONH—C₁₋₅-alkylene-NRₐR_{b},
or alternatively Z represents a group —CONH—C₁₋₅-alkylene-O—R_{d},
or alternatively Z represents a group —O—C₁₋₃-alkylene-C(O)—NRₐR_{b},
or alternatively Z represents a fused or spirane bicyclic diamino heterocycle,
or alternatively two adjacent radicals Z together form a C₁₋₃-alkylenedioxy group;
Rₐ and R_{b} represent, independently of each other, a hydrogen atom or a C₁₋₃-alkyl or —C(O)—C₁₋₃-alkyl group,
or alternatively Rₐ and R_{b} form, together with the nitrogen atom to which they are attached, a heterocycle optionally substituted with one or more C₁₋₃-alkyl, oxo, —NRₐR_{b}, hydroxyl, C₁₋₃-alkoxy, C₁₋₃-alkylene-(OH) or —C(O)—C₁₋₃-alkyl groups;
R_{d} represents a hydrogen atom or a C₁₋₃-alkyl group;
Rₑ represents a hydrogen atom or a C₁₋₃-alkyl group;
with the proviso that when R represents a hydrogen atom, then at least one of the conditions defined below is satisfied:
R₄ represents a heterocyclic group, the said heterocycle being optionally substituted with a C₁₋₃-alkyl, —C(O)C₁₋₃-alkyl, —C(O)C₁₋₃-fluoroalkyl, C₁₋₃-alkylene-C₃₋₆-cycloalkyl, C₁₋₃-alkylene-aryl or C₁₋₃-alkylene-heteroaryl group; the C₁₋₃-alkylene-aryl and C₁₋₃-alkylene-heteroaryl groups being optionally substituted with one or more radicals Z, which may be identical or different, as defined above; or alternatively
Z represents a phenyl substituted with a halogen atom or a C₁₋₅-alkyl, C₁₋₅-alkoxy or C₁₋₃-fluoroalkyl group, or alternatively
Z represents an alkynyl radical of the type —C≡C—R_{c} in which R_{c} represents a hydrogen atom or a C₁₋₃-alkyl, C₁₋₆-alkylene-O—R_{d} or C₁₋₆-alkylene-NRₐR_{b} group, or alternatively
Z represents a group —C₄₋₆-alkylene-OR_{d}, or alternatively
Z represents a tetrazole group substituted with a C₁₋₃-alkyl group, or alternatively
Z represents a group —C₄₋₆-alkylene-NRₐR_{b}, or alternatively
Z represents a group —SO₂—NRₐR_{b1} or alternatively
Z represents a group —O—C₁₋₅-alkylene-NRₐR_{b}, or alternatively
Z represents an —O—C₀₋₃-alkylene-heterocycle group optionally substituted with one or more C₁₋₃-alkyl, oxo or —C(O)—C₁₋₃-alkyl groups, or alternatively
Z represents a group —O—C₁₋₅-alkylene-O—R_{d}, or alternatively
Z represents an —O—C₀₋₃-alkylene-C₅₋₇-cycloalkyl group optionally substituted with an —O—R_{d} group, or alternatively
Z represents an —NRₑ—C₀₋₃-alkylene-heterocycle group optionally substituted with one or more C₁₋₃-alkyl, oxo or —C(O)—C₁₋₃-alkyl groups, or alternatively
Z represents a group —NRₑ—C₂₋₅-alkylene-O—R_{d}, or alternatively
Z represents an —O—C₁₋₃-alkylene-heteroaryl group optionally substituted with one or more C₁₋₃-alkyl groups, or alternatively
Z represents a group —CONH—C₁₋₅-alkylene-NRₐR_{b}, or alternatively
Z represents a group —CONH—C₁₋₅-alkylene-O—R_{d}, or alternatively
Z represents a group —O—C₁₋₃-alkylene-C(O)—NRₐR_{b}, and wherein
Rₐ, and R_{b} represent, independently of each other, a hydrogen atom or a C₁₋₃-alkyl or —C(O)—C₁₋₃-alkyl,
or alternatively Rₐ and R_{b} form, together with the nitrogen atom to which they are attached, a heterocycle optionally substituted with one or more C₁₋₃-alkyl, oxo, —NRₐR_{b}, hydroxyl, C₁₋₃-alkoxy, C₁₋₃-alkylene-(OH) or —C(O)—C₁₋₃-alkyl groups;
R_{d} represents a hydrogen atom or a C₁₋₃-alkyl group;
Rₑ represents a hydrogen atom or a C₁₋₃-alkyl group;
or alternatively Z represents a fused or spirane bicyclic diamino heterocycle,
or alternatively Z represents a group —NRₐR_{b} in which Rₐ and R_{b} form, together with the nitrogen atom to which they are attached, a heterocycle substituted with one or more —NRₐR_{b}, hydroxyl, C₁₋₃-alkoxy, C₁₋₃-alkylene-(OH) or —C(O)—C₁₋₃-alkyl groups; or
an acid-addition salt, a hydrate or a solvate thereof, or an enantiomer, or a diastereoisomer or a mixture thereof.

2. The compound of formula (I) according to claim 1, wherein:
A represents a C₁₋₄-alkylene group, optionally substituted with one or more groups R₉, which may be identical or different;
B represents a C₁₋₄-alkylene group, optionally substituted with one or more groups R₁₀, which may be identical or different;
R₉ and R₁₀ represent, independently of each other, a hydrogen atom;
L represents a single bond;
R represents a hydrogen atom or a C₁₋₅-alkyl or a group —C(O)C₁₋₃-alkyl group;
R₁ represents an aryl or R₁ represents a heteroaryl group; the aryl and heteroaryl groups being optionally substituted with one or more radicals Z, which may be identical or different;
R₂ and R₃ represent, independently of each other, a hydrogen atom;
R₄ represents:
a hydrogen atom or a C₁₋₅-alkyl, C₁₋₃-fluoroalkyl or C₁₋₃-alkylene-O—C₁₋₃-alkyl group,
a heterocyclic group; the said heterocyclic group being optionally substituted with a C₁₋₃-alkylene-aryl group,
or alternatively R₄ represents an aryl group; the said aryl group being optionally substituted with one or more radicals Z, which may be identical or different;
R₅ represents a hydrogen atom or a C₁₋₅-alkoxy group;
R₇ represents a halogen atom or a C₁₋₅-alkoxy group;
Z represents a halogen atom or a C₁₋₅-alkoxy or phenyl group optionally substituted with a C₁₋₃-fluoroalkyl group,
or alternatively Z represents an alkynyl radical of the type —C≡C—R_{c}, in which R_{c} represents a hydrogen atom, C₁₋₆-alkylene-O—R_{d} or C₁₋₆-alkylene-NRₐR_{b},
or alternatively Z represents a group —C₄₋₆-alkylene-OR_{d},
or alternatively Z represents a tetrazole group substituted with a C₁₋₃-alkyl group,
or alternatively Z represents a group —NRₐR_{b},
or alternatively Z represents a group —C₄₋₆-alkylene-NRₐR_{b},
or alternatively Z represents a group —O—C₁₋₅-alkylene-NRₐR_{b},
or alternatively Z represents an —O—C₀₋₃-alkylene-heterocycle group, the said —O—C₀₋₃-alkylene-heterocycle group being optionally substituted with one or more C₁₋₃-alkyl groups or an oxo group,
or alternatively Z represents a group —O—C₁₋₅-alkylene-O—R_{d},
or alternatively Z represents a group —NRₑ—C₂₋₅-alkylene-O—R_{d},
or alternatively Z represents a —O—C₁₋₃-alkylene-heteroaryl group,
or alternatively Z represents a group —CONH—C₁₋₅-alkylene-NRₐR_{b},
or alternatively Z represents a group —O—C₁₋₃-alkylene-C(O)—NRₐR_{b},
or alternatively Z represents a fused or spirane bicyclic diamino heterocycle chosen from: 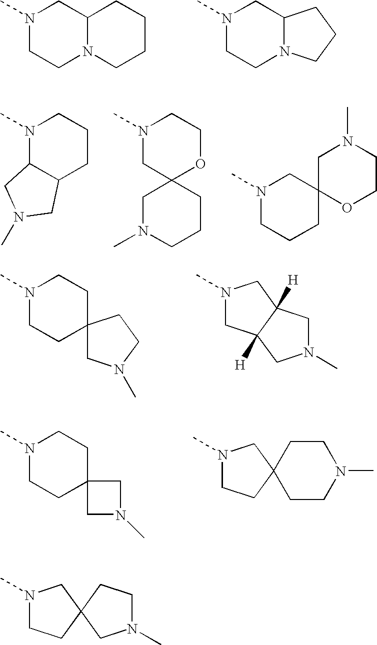
in which the dashed lines represent the point of attachment to the rest of the molecule of formula (I) and the solid lines represent a methyl substituent;
Rₐ and R_{b} represent, independently of each other a C₁₋₃-alkyl group,
or alternatively Rₐ and R_{b} form, together with the nitrogen atom to which they are attached, a heterocycle, optionally substituted with one or more oxo, —NRₐR_{b} or hydroxyl groups;
R_{d} represents a hydrogen atom or a C₁₋₃-alkyl group;
Rₑ represents a C₁₋₃-alkyl group;
with the proviso that when R represents a hydrogen atom, then at least one of the conditions defined below is satisfied:
R₄ represents a heterocyclic group, the said heterocycle being optionally substituted with a C₁₋₃-alkylene-aryl group, or alternatively
Z represents a phenyl group substituted with a C₁₋₃-fluoroalkyl group, or alternatively
Z represents an alkynyl radical of the type —C≡C—R_{c}, in which R_{c}, represents a hydrogen atom or a C₁₋₆-alkylene-O—R_{d} or C₁₋₆-alkylene-NRₐR_{b} group, or alternatively
Z represents a group —C₄₋₆-alkylene-OR_{d}, or alternatively
Z represents a tetrazole group substituted with a C₁₋₃-alkyl group, or alternatively
Z represents a group —C₄₋₆-alkylene-NRₐR_{b}, or alternatively
Z represents a group —O—C₁₋₅-alkylene-NRₐR_{b}, or alternatively
Z represents an —O—C₀₋₃-alkylene-heterocycle group, the said —O—C₀₋₃-alkylene-heterocycle group being optionally substituted with one or more C₁₋₃-alkyl groups or an oxo group, or alternatively
Z represents a group —O—C₁₋₅-alkylene-O—R_{d}, or alternatively
Z represents a group —NRₑ—C₂₋₅-alkylene-O—R_{d}, or alternatively
Z represents an —O—C₁₋₃-alkylene-heteroaryl group, or alternatively
Z represents a group —CONH—C₁₋₅-alkylene-NRₐR_{b}, or alternatively
Z represents a group —O—C₁₋₃-alkylene-C(O)—NRₐR_{b}, and wherein
Rₐ and R_{b} represent, independently of each other a C₁₋₃-alkyl group, or alternatively Rₐ and R_{b} form, together with the nitrogen atom to which they are attached, a heterocycle, the said heterocyclic group being optionally substituted with one or more oxo, —NRₐR_{b} or hydroxyl groups;
R_{d} represents a hydrogen atom or a C₁₋₃-alkyl group;
Rₑ represents a C₁₋₃-alkyl group;
or alternatively Z represents a fused or spirane bicyclic diamino heterocycle chosen from: 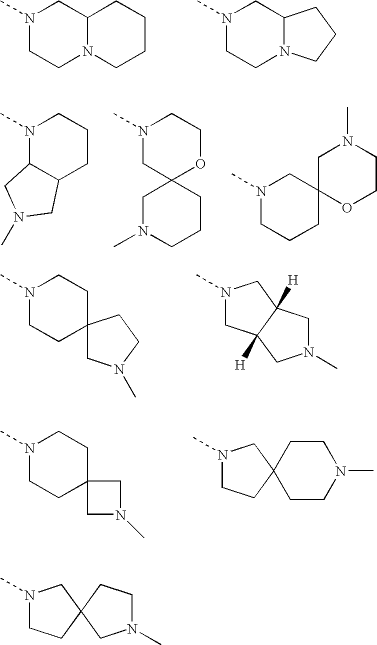
in which the dashed lines represent the point of attachment to the rest of the molecule of formula (I) and the solid lines represent a methyl substituent;
or alternatively Z represents a group —NRₐR_{b} in which Rₐ and R_{b} form, together with the nitrogen atom to which they are attached, a heterocycle substituted with one or more —NRₐR_{b} or hydroxyl groups; or
an acid-addition salt, a hydrate or a solvate thereof, an enantiomer, a diastereoisomer or a mixture thereof.

3. The compound of formula (I) according to claim 1, wherein:
A represents an ethylene group;
B represents an ethylene group;
L represents a single bond;
R represents a hydrogen atom, a methyl or ethyl group or a —C(O)-methyl group;
R₁ represents a phenyl, naphthyl, benzo-1,3-dioxolyl or pyrrolyl group; the said phenyl, naphthyl, benzo-1,3-dioxolyl and pyrrolyl groups being optionally substituted with one or more radicals Z, which may be identical or different;
R₂ and R₃ represent, independently of each other, a hydrogen atom;
R₄ represents:
a hydrogen atom or a methyl, ethyl, trifluoromethyl or methylene-O-methyl group,
a piperidyl or tetrahydropyranyl group; the said piperidyl or tetrahydropyranyl groups being optionally substituted with a methylene-phenyl group,
or alternatively R₄ represents a phenyl group; the said phenyl group being optionally substituted with one or more radicals Z, which may be identical or different;
R₅ represents a hydrogen atom or a methoxy group;
R₇ represents a chlorine atom or a methoxy group;
Z represents a hydrogen atom or a fluorine atom, a methoxy group or a phenyl group optionally substituted with a trifluoromethyl,
or alternatively Z represents a radical —C≡C—R_{c}, in which R_{c} represents a hydrogen atom, C₁₋₆-alkylene-O—R_{d} or C₁₋₆-alkylene-NRₐR_{b},
or alternatively Z represents a group butylene-OR_{d},
or alternatively Z represents a tetrazole group substituted with a methyl group
or alternatively Z represents a group —NRₐR_{b},
or alternatively Z represents a group butylene-NRₐR_{b},
or alternatively Z represents an —O—C₁₋₅-propylene-NRₐR_{b} or —O—C₁₋₅-ethylene-NRₐR_{b} group,
or alternatively Z represents an —O-heterocycle, —O-methylene-heterocycle,
—O-ethylene-heterocycle group, the said —O-heterocycle, —O-methylene-heterocycle, —O— ethylene-heterocycle groups being optionally substituted with one or more methyl groups or an oxo group,
or alternatively Z represents a group —O-ethylene-OR_{d} or —O-propylene-OR_{d},
or alternatively Z represents a group —NRₑ-ethylene-OR_{d},
or alternatively Z represents an —O-methylene-heteroaryl or —O-ethylene-heteroaryl group,
or alternatively Z represents a group —CONH-ethylene-NRₐR_{b},
or alternatively Z represents a group —O-methylene-C(O)—NRₐR_{b},
or alternatively Z represents a fused or spirane bicyclic diamino heterocycle chosen from: 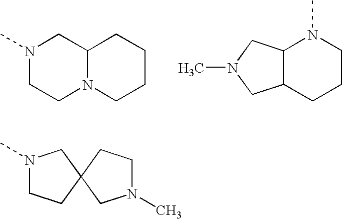
in which the dashed lines represent the point of attachment to the rest of the molecule of formula (I) and the solid lines represent a methyl substituent;
Rₐ and R_{b} represent, independently of each other, a methyl group,
or alternatively Rₐ and R_{b} form, together with the nitrogen atom to which they are attached, a morpholinyl, piperidyl or pyrrolidinyl group, optionally substituted with one or more oxo, —NRₐR_{b} or hydroxyl groups;
R_{d} represents a hydrogen atom or a methyl group;
Rₑ represents a methyl group;
with the proviso that when R represents a hydrogen atom, then at least one of the conditions defined below is satisfied:
R₄ represents a piperidyl or tetrahydropyranyl group, the said piperidyl or tetrahydropyranyl groups being optionally substituted with a methylene-phenyl group, or alternatively
Z represents a phenyl group substituted with a trifluoromethyl group, or alternatively
Z represents an alkynyl radical of the type —C≡C—R_{c}, in which R_{c}, represents a hydrogen atom, isopropylene-O—R_{d} or methylene-NRₐR_{b}, or alternatively
Z represents a butylene-OR_{d} group, or alternatively
Z represents a tetrazole group substituted with a methyl group, or alternatively
Z represents a butylene-NRₐR_{b} group, or alternatively
Z represents a group —O-propylene-NRₐR_{b}, or alternatively
Z represents an —O-heterocycle, —O-methylene-heterocycle, —O-ethylene-heterocycle group, the said groups being optionally substituted with one or more methyl groups or an oxo group, or alternatively
Z represents a group —O-ethylene-O—R_{d} or —O-propylene-O—R_{d}, or alternatively
Z represents a group —NRₑ-ethylene-O—R_{d}, or alternatively
Z represents an —O-methylene-heteroaryl or —O-ethylene-heteroaryl group, or alternatively
Z represents a group —CONH-ethylene-NRₐR_{b}, or alternatively
Z represents a group —O-methylene-C(O)—NRₐR_{b}, and wherein
Rₐ and R_{b} represent, independently of each other, a methyl group, or alternatively Rₐ and R_{b} form, together with the nitrogen atom to which they are attached, a piperidyl, morpholinyl or pyrrolidinyl group, the said piperidyl, morpholinyl and pyrrolidinyl groups being optionally substituted with one or more oxo, —NRₐR_{b} or hydroxyl groups;
R_{d} represents a hydrogen atom or a methyl group;
Rₑ represents a methyl;
or alternatively Z represents a fused or spirane bicyclic diamino heterocycle chosen from: 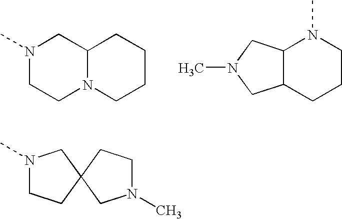
in which the dashed lines represent the point of attachment to the rest of the molecule of formula (I) and the solid lines represent a methyl substituent;
or alternatively Z represents a group —NRₐR_{b} in which Rₐ and R_{b} form, together with the nitrogen atom to which they are attached, a piperidyl or pyrrolidinyl group optionally substituted with one or more —NRₐR_{b} or hydroxyl groups; or
an acid-addition salt, a hydrate or a solvate thereof, or an enantiomer, diastereoisomer or a mixture thereof.

4. The compound of formula (I) according to claim 1, wherein:
A represents a C₁₋₄-alkylene group optionally substituted with one or more groups R₉, which may be identical or different;
B represents a C₁₋₄-alkylene group optionally substituted with one or more groups R₁₀, which may be identical or different;
R₉ and R₁₀ represent, independently of each other, a hydrogen atom or a C₁₋₅-alkyl group,
or alternatively R₉ and R₁₀ together form a single bond or a C₁₋₄-alkylene group;
L represents a single bond or a C₁₋₂-alkylene, —CH═CH— or —C≡C— group; the C₁₋₂-alkylene and —CH═CH— groups being optionally substituted with one or more C₁₋₂-alkyl substituents,
or alternatively L represents a cycloprop-1,2-diyl group;
R represents a hydrogen atom or a C₁₋₅-alkyl, C₁₋₃-fluoroalkyl, C₃₋₆-cycloalkyl, —C(O)C₁₋₃-alkyl, C₁₋₃-alkylene-C₃₋₆-cycloalkyl, —CH₂—C≡CH, C₂₋₄-alkylene-NRₐR_{b} or C₁₋₃-alkylene-X—C₁₋₃-alkyl group in which X represents O or SO₂;
R₁ represents an aryl or a heteroaryl group; the aryl and heteroaryl groups being optionally substituted with one or more radicals Z, which may be identical or different;
R₂ and R₃ represent, independently of each other, a hydrogen atom or a C₁₋₃-alkyl or C₁₋₃-fluoroalkyl group,
or alternatively R₂ and R₃ form, together with the carbon atom that bears them, a cycloprop-1,1-diyl group;
R₄ represents:
a hydrogen atom or a C₁₋₅-alkyl, C₁₋₃-fluoroalkyl, C₃₋₆-cycloalkyl, C₁₋₃-alkylene-C₃₋₄-cycloalkyl or C₁₋₃-alkylene-O—C₁₋₃-alkyl group,
a C₁₋₃-alkylene-(OH) or C₁₋₃-alkylene-X—C₁₋₃-alkyl group in which X represents S, SO or SO₂,
a heterocyclic group; the said heterocyclic group being optionally substituted with a C₁₋₃-alkyl, —C(O)C₁₋₃-alkyl, —C(O)C₁₋₃-fluoroalkyl, C₁₋₃-alkylene-C₃₋₄-cycloalkyl, C₁₋₃-alkylene-aryl or C₁₋₃-alkylene-heteroaryl group; the C₁₋₃-alkylene-aryl and C₁₋₃-alkylene-heteroaryl groups being optionally substituted with one or more radicals Z, which may be identical or different,
or alternatively R₄ represents a C₁₋₃-alkylene-NRₐR_{b}, aryl, C₁₋₃-alkylene-aryl, —O-aryl, C₁₋₃-alkylene-O-aryl, C₁₋₃-alkylene-O—C₁₋₃-alkylene-aryl, heteroaryl or C₁₋₃-alkylene-heteroaryl group; the aryl, C₁₋₃-alkylene-aryl, —O-aryl, C₁₋₃-alkylene-O-aryl, C₁₋₃-alkylene-O—C₁₋₃-alkylene-aryl, heteroaryl and C₁₋₃-alkylene-heteroaryl groups being optionally substituted with one or more radicals Z, which may be identical or different;
R₅ represents a hydrogen or halogen atom or a C₁₋₅-alkyl, C₁₋₃-fluoroalkyl, C₁₋₅-alkoxy, C₁₋₃-fluoroalkoxy, C₁₋₃-alkylene-(OH), —CN or —X—C₁₋₃-alkyl group in which X represents S, SO or SO₂,
or alternatively R₅ represents an —NRₐR_{b}, C₁₋₃-alkylene-NRₐR_{b}, aryl, C₁₋₃-alkylene-aryl, —O-aryl or heteroaryl group; the aryl, C₁₋₃-alkylene-aryl, —O-aryl and heteroaryl groups being optionally substituted with one or more radicals Z, which may be identical or different;
R₇ represents a hydrogen or halogen atom or a C₁₋₅-alkyl, C₁₋₃-fluoroalkyl, C₁₋₅-alkoxy, —COOH, —C(O)OC₁₋₃-alkyl, C₁₋₃-fluoroalkoxy, C₁₋₃-alkylene-(OH), —CN or —X—C₁₋₃-alkyl group in which X represents S, SO or SO₂,
or alternatively R₇ represents an —NRₐR_{b}, C₁₋₃-alkylene-NRₐR_{b}, C(O)—NRₐR_{b}, —C(O)—C₁₋₃-alkyl, aryl, —O-aryl or heteroaryl group; the aryl, —O-aryl and heteroaryl groups being optionally substituted with one or more radicals Z, which may be identical or different;
Z represents a halogen atom or a C₁₋₅-alkyl, C₁₋₃-fluoroalkyl, C₃₋₄-cycloalkyl, C₁₋₃-alkylene-C₃₋₆-cycloalkyl, phenyl, C₁₋₅-alkoxy, C₁₋₃-fluoroalkoxy, C₁₋₃-alkylene-O—C₁₋₃-alkyl, C₁₋₃-alkylene-(OH), NO₂, —CN, —SO₂NRₐR_{b}, —X—C₁₋₃-alkyl or C₁₋₃-alkylene-X—C₁₋₃-alkyl group in which X represents S, SO or SO₂,
or alternatively Z represents an —NRₐR_{b}, C₁₋₃-alkylene-NRₐR_{b}, C(O)—NRₐR_{b}, —C(O)—C₁₋₃-alkyl, —C(O)O—C₁₋₄-alkyl or —C(O)—C₃₋₆-cycloalkyl group,
or alternatively Z represents an oxo radical,
or alternatively Z represents a group —O—C₁₋₅-alkylene-NRₐR_{b},
or alternatively two adjacent radicals Z together form a C₁₋₃-alkylenedioxy group;
Rₐ and R_{b} represent, independently of each other, a hydrogen atom or a C₁₋₃-alkyl or —C(O)—C₁₋₃-alkyl group,
or alternatively Rₐ and R_{b} form, together with the nitrogen atom that bears them, a heterocycle optionally substituted with one or more C₁₋₃-alkyl or oxo groups;
with the proviso that when R represents a hydrogen atom, at least one of the following two conditions is satisfied:
R₄ represents a heterocyclic group; the said heterocycle being optionally substituted, and
Z represents a group —SO₂—NRₐR_{b} or a group —O—C₁₋₅-alkylene-NRₐR_{b}; or an acid-addition salt, a hydrate or a solvate thereof, an enantiomer, a diastereoisomer or a mixture thereof.

5. The compound of formula (I) according to claim 4, wherein:
A represents a C₁₋₄-alkylene group optionally substituted with one or more groups R₉, which may be identical or different;
B represents a C₁₋₄-alkylene group optionally substituted with one or more groups R₁₀, which may be identical or different;
R₉ and R₁₀ represent, independently of each other, a hydrogen atom or a C₁₋₅-alkyl group and in particular a methyl,
or alternatively R₉ and R₁₀ together form a C₁₋₄-alkylene group;
L represents a single bond or —CH═CH—;
R represents a hydrogen atom or a C₁₋₅-alkyl, C₁₋₃-fluoroalkyl, C₃₋₆-cycloalkyl or C₁₋₃-alkylene-C₃₋₆-cycloalkyl group;
R₁ represents an aryl or a heteroaryl group; the aryl and heteroaryl groups being optionally substituted with one or more radicals Z, which may be identical or different;
R₂ and R₃ represent, independently of each other, a hydrogen atom or a C₁₋₃-alkyl group;
R₄ represents:
a hydrogen atom or a C₁₋₅-alkyl, C₃₋₄-cycloalkyl, C₁₋₃-alkylene-C₃₋₆-cycloalkyl or C₁₋₃-alkylene-O—C₁₋₃-alkyl group,
a C₁₋₃-alkylene-(OH) group,
a heterocyclic group; the said heterocyclic group being optionally substituted with a C₁₋₃-alkyl, —C(O)C₁₋₃-alkyl, —C(O)C₁₋₃-fluoroalkyl, C₁₋₃-alkylene-C₃₋₆-cycloalkyl or C₁₋₃-alkylene-aryl group; the C₁₋₃-alkylene-aryl group being optionally substituted with one or more radicals Z, which may be identical or different,
or alternatively R₄ represents a C₁₋₃-alkylene-NRₐR_{b}, aryl, C₁₋₃-alkylene-aryl, C₁₋₃-alkylene-O-aryl, C₁₋₃-alkylene-O—C₁₋₃-alkylene-aryl, heteroaryl or C₁₋₃-alkylene-heteroaryl group; the aryl, C₁₋₃-alkylene-aryl, C₁₋₃-alkylene-O-aryl, C₁₋₃-alkylene-O—C₁₋₃-alkylene-aryl, heteroaryl and C₁₋₃-alkylene-heteroaryl groups being optionally substituted with one or more radicals Z, which may be identical or different;
R₅ represents a hydrogen atom or a C₁₋₅-alkyl, C₁₋₅-alkoxy or aryl group; the aryl group being optionally substituted with one or more radicals Z, which may be identical or different;
R₇ represents a halogen atom or a C₁₋₅-alkyl group and in particular a methyl, C₁₋₅-alkoxy and in particular a methoxy, CN, COOH or —C(O)OC₁₋₃-alkyl group;
Z represents a halogen atom or a C₁₋₅-alkyl, C₁₋₃-fluoroalkyl, C₃₋₆-cycloalkyl, C₁₋₃-alkylene-C₃₋₆-cycloalkyl, phenyl, C₁₋₅-alkoxy, C₁₋₃-fluoroalkoxy, C₁₋₃-alkylene-O—C₁₋₃-alkyl, C₁₋₃-alkylene-(OH), NO₂, —CN, —SO₂NRₐR_{b}, —X—C₁₋₃-alkyl or C₁₋₃-alkylene-X—C₁₋₃-alkyl group in which X represents S, SO or SO₂,
or alternatively Z represents a group —NRₐR_{b}, C₁₋₃-alkylene-NRₐR_{b}, —C(O)—NRₐR_{b}, —C(O)—C₁₋₃-alkyl, —C(O)O—C₁₋₄-alkyl or —C(O)—C₃₋₆-cycloalkyl,
or alternatively Z represents an oxo radical,
or alternatively Z represents a group —O—C₁₋₅-alkylene-NRₐR_{b},
or alternatively two adjacent radicals Z together form a C₁₋₃-alkylenedioxy group;
Rₐ and R_{b} represent, independently of each other, a hydrogen atom or a C₁₋₃-alkyl or —C(O)—C₁₋₃-alkyl group,
or alternatively Rₐ and R_{b} form, together with the nitrogen atom that bears them, a heterocycle optionally substituted with one or more C₁₋₃-alkyl or oxo groups;
with the proviso that when R represents a hydrogen atom, at least one of the following two conditions is satisfied:
R₄ represents a heterocyclic group; the said heterocycle being optionally substituted, and
Z represents a group —SO₂—NRₐR_{b} or a group —O—C₁₋₅-alkylene-NRₐR_{b}; or an acid-addition salt, a hydrate or a solvate thereof, an enantiomer, a diastereoisomer or a mixture thereof.

6. The Compound of formula (I) according to claim 1, wherein:
A represents a C₁₋₄-alkylene group and in particular an ethylene, optionally substituted with one or more groups R₉;
B represents a C₁₋₄-alkylene group and in particular an ethylene, optionally substituted with one or more groups R₁₀;
R₉ and R₁₀ represent, independently of each other, a hydrogen atom or a methyl, or alternatively R₉ and R₁₀ together form a C₁₋₄-alkylene group;
L represents a single bond or —CH═CH—;
R represents a hydrogen atom or a C₁₋₅-alkyl group and in particular an ethyl, or C₁₋₃-alkylene-C₃₋₄-cycloalkyl group;
R₁ represents an aryl or a heteroaryl group; the aryl and heteroaryl groups being optionally substituted with one or more radicals Z, which may be identical or different;
R₂ and R₃ represent, independently of each other, a hydrogen atom or a C₁₋₃-alkyl group and in particular a methyl;
R₄ represents:
a hydrogen atom or a C₁₋₅-alkyl, C₃₋₆-cycloalkyl, C₁₋₃-alkylene-C₃₋₆-cycloalkyl or
C₁₋₃-alkylene-O—C₁₋₃-alkyl group and in particular a methylene-O-methyl group,
a C₁₋₃-alkylene-(OH),
a heterocyclic group optionally substituted with a C₁₋₃-alkyl, —C(O)C₁₋₃-alkyl, —C(O)C₁₋₃-fluoroalkyl, C₁₋₃-alkylene-C₃₋₄-cycloalkyl or C₁₋₃-alkylene-aryl group and in particular a methylene-phenyl; the C₁₋₃-alkylene-aryl group being optionally substituted with one or more radicals Z, which may be identical or different,
or alternatively R₄ represents a C₁₋₃-alkylene-NRₐR_{b}, aryl and in particular a phenyl, C₁₋₃-alkylene-aryl and in particular a C₁₋₃-alkylene-phenyl, C₁₋₃-alkylene-O-aryl and in particular a C₁₋₃-alkylene-O-phenyl, C₁₋₃-alkylene-O—C₁₋₃-alkylene-aryl and in particular a C₁₋₃-alkylene-O—C₁₋₃-alkylene-phenyl, heteroaryl and in particular a thienyl or a pyridyl; the aryl, C₁₋₃-alkylene-aryl, C₁₋₃-alkylene-O-aryl, C₁₋₃-alkylene-O—C₁₋₃-alkylene-aryl and heteroaryl groups being optionally substituted with one or more radicals Z, which may be identical or different;
R₅ represents a hydrogen atom;
R₇ represents a halogen atom, a methyl, a methoxy, CN, COOH or —C(O)O—C₁₋₃-alkyl;
Z represents a halogen atom or a C₁₋₅-alkyl, phenyl, C₁₋₅-alkoxy and in particular methoxy, C₁₋₃-alkylene-O—C₁₋₃-alkyl, C₁₋₃-alkylene-(OH), —CN, —SO₂NRₐR_{b}, —X—C₁₋₃-alkyl or C₁₋₃-alkylene-X—C₁₋₃-alkyl group in which X represents S, SO or SO₂;
or alternatively Z represents a group —NRₐR_{b}, C₁₋₃-alkylene-NRₐR_{b}, —C(O)—NRₐR_{b} or —C(O)—C₁₋₃-alkyl,
or alternatively Z represents an oxo radical,
or alternatively Z represents a group —O—C₁₋₅-alkylene-NRₐR_{b},
or alternatively two adjacent radicals Z together form a C₁₋₃-alkylenedioxy group, and in particular a methylenedioxy;
Rₐ, and R_{b} represent, independently of each other, a hydrogen atom or a C₁₋₃-alkyl or —C(O)—C₁₋₃-alkyl group,
or alternatively Rₐ, and R_{b} form, together with the nitrogen atom that bears them, a heterocycle optionally substituted with one or more C₁₋₃-alkyl or oxo groups;
with the proviso that when R represents a hydrogen atom, at least one of the following two conditions is satisfied:
R₄ represents a heterocyclic group; the said heterocycle being optionally substituted, and
Z represents a group —SO₂—NRₐR_{b} or a group —O—C₁₋₅-alkylene-NRₐR_{b}; or an acid-addition salt, a hydrate or a solvate thereof, an enantiomer, a diastereoisomer or a mixture thereof.

7. The compound of formula (I) according to claim 1, wherein:
A represents an ethylene, optionally substituted with one or more groups R₉;
B represents an ethylene, optionally substituted with one or more groups R₁₀;
R₉ and R₁₀ represent, independently of each other, a hydrogen atom or a methyl, L represents a single bond;
R represents a hydrogen atom or an ethyl or C₁₋₃-alkylene-C₃₋₆-cycloalkyl group;
R₁ represents an aryl or a heteroaryl group; the aryl and heteroaryl groups being optionally substituted with one or more radicals Z, which may be identical or different;
R₂ and R₃ represent, independently of each other, a hydrogen atom or a methyl group;
R₄ represents:
a hydrogen atom or a C₁₋₅-alkyl, C₃₋₆-cycloalkyl, C₁₋₃-alkylene-C₃₋₆-cycloalkyl or methylene-O-methyl group,
a C₁₋₃-alkylene-(OH) group,
a heterocyclic group optionally substituted with a C₁₋₃-alkyl, —C(O)C₁₋₃-alkyl, —C(O)C₁₋₃-fluoroalkyl, C₁₋₃-alkylene-C₃₋₄-cycloalkyl or methylene-phenyl group; the methylene-phenyl group being optionally substituted with one or more radicals Z, which may be identical or different,
or alternatively R₄ represents a C₁₋₃-alkylene-NRₐR_{b}, a phenyl, a C₁₋₃-alkylene-phenyl, a C₁₋₃-alkylene-O-phenyl, a C₁₋₃-alkylene-O—C₁₋₃-alkylene-phenyl, a thienyl or a pyridyl group; the phenyl, C₁₋₃-alkylene-phenyl, C₁₋₃-alkylene-O-phenyl, C₁₋₃-alkylene-O—C₁₋₃-alkylene-phenyl, thienyl and pyridyl groups being optionally substituted with one or more radicals Z, which may be identical or different;
R₅ represents a hydrogen atom;
R₇ represents a halogen atom, a methyl, a methoxy or CN;
Z represents a halogen atom or a C₁₋₅-alkyl, a phenyl, a methoxy, C₁₋₃-alkylene-O—C₁₋₃-alkyl, C₁₋₃-alkylene-(OH), —CN, —SO₂NRₐR_{b}, —X—C₁₋₃-alkyl or C₁₋₃-alkylene-X—C₁₋₃-alkyl group in which X represents S, SO or SO₂,
or alternatively Z represents a group —NRₐR_{b}, C₁₋₃-alkylene-NRₐR_{b}, —C(O)—NRₐR_{b} or —C(O)—C₁₋₃-alkyl,
or alternatively Z represents an oxo radical,
or alternatively Z represents a group —O—C₁₋₅-alkylene-NRₐR_{b},
or alternatively two adjacent radicals Z together form a methylenedioxy group;
Rₐ and R_{b} represent, independently of each other, a hydrogen atom or a C₁₋₃-alkyl or —C(O)—C₁₋₃-alkyl group,
or alternatively Rₐ and R_{b} form, together with the nitrogen atom that bears them, a heterocycle optionally substituted with one or more C₁₋₃-alkyl or oxo groups;
with the proviso that when R represents a hydrogen atom, at least one of the following two conditions is satisfied:
R₄ represents a heterocyclic group; the said heterocycle being optionally substituted,
Z represents a group —SO₂—NRₐR_{b} or a group —O—C₁₋₅-alkylene-NRₐR_{b}; or an acid-addition salt, a hydrate or a solvate thereof, an enantiomer, a diastereoisomer or a mixture thereof.

8. The compound of formula (I) according to claim 1, which is chosen from:
N-[1-(1,3-benzodioxol-5-ylmethyl)piperidin-4-yl]-4-(1-benzylpiperidin-4-yl)-7-meth-oxyphthalazin-1-amine trihydrochloride;
N-(1-{4-[3-(dimethylamino)propoxy]benzyl}piperidin-4-yl)-7-methoxy-4-(4-meth-oxyphenyl)phthalazin-1-amine trihydrochloride;
N-{1-[3-fluoro-4-(3-morpholin-4-ylpropoxy)benzyl]piperidin-4-yl}-7-methoxy-4-(4-methoxyphenyl)phthalazin-1-amine trihydrochloride;
7-methoxy-4-(4-methoxyphenyl)-N-{1-[4-(2-piperidin-1-ylethoxy)benzyl]piperidin-4-yl}phthalazin-1-amine trihydrochloride;
N-(1-{4-[3-(dimethylamino)propoxy]-3-fluorobenzyl}piperidin-4-yl)-7-methoxy-4-(4-methoxyphenyl)phthalazin-1-amine trihydrochloride;
7-methoxy-4-(4-methoxyphenyl)-N-{1-[4-(2-pyrrolidin-1-ylethoxy)benzyl]piperidin-4-yl}phthalazin-1-amine trihydrochloride;
7-methoxy-4-(4-methoxyphenyl)-N-{1-[4-(2-morpholin-4-ylethoxy)benzyl]piperidin-4-yl}phthalazin-1-amine trihydrochloride;
7-methoxy-4-(4-methoxyphenyl)-N-{1-[4-(3-piperidin-1-ylpropoxy)benzyl]piperidin-4-yl}phthalazin-1-amine trihydrochloride;
N-[1-(1,3-benzodioxol-5-ylmethyl)piperidin-4-yl]-N-ethyl-7-methoxy-4-(4-methoxy-phenyl)phthalazin-1-amine dihydrochloride;
4-[(4-{[7-methoxy-4-(4-methoxyphenyl)phthalazin-1-yl]amino}piperidin-1-yl)-methyl]-N-(2-piperidin-1-ylethyl)benzamide trihydrochloride;
N-[1-(1,3-benzodioxol-5-ylmethyl)piperidin-4-yl]-7-methoxy-4-(tetrahydro-2H-pyran-4-yl)phthalazin-1-amine dihydrochloride;
7-methoxy-4-(4-methoxyphenyl)-N-{1-[4-(3-methoxypropoxy)benzyl]piperidin-4-yl}phthalazin-1-amine dihydrochloride;
N-(1-{4-[3-(dimethylamino)propoxy]-3-fluorobenzyl}piperidin-4-yl)-4-ethyl-7-methoxyphthalazin-1-amine trihydrochloride;
N-(1-{4-[3-(dimethylamino)propoxy]-3-fluorobenzyl}piperidin-4-yl)-7-methoxy-4-(methoxymethyl)phthalazin-1-amine trihydrochloride;
N-{1-[3-fluoro-4-(3-pyrrolidin-1-ylpropoxy)benzyl]piperidin-4-yl}-7-methoxy-4-(4-methoxyphenyl)phthalazin-1-amine trihydrochloride;
1-(3-{4-[(4-{[7-methoxy-4-(4-methoxyphenyl)phthalazin-1-yl]amino}piperidin-1-yl)methyl]phenoxy}propyl)pyrrolidin-2-one trihydrochloride;
N-(1-{4-[2-(dimethylamino)ethoxy]benzyl}piperidin-4-yl)-7-methoxy-4-(4-methoxy-phenyl)phthalazin-1-amine trihydrochloride;
7-methoxy-4-(4-methoxyphenyl)-N-(1-{4-[(1-methylazepan-4-yl)oxy]benzyl}-piperidin-4-yl)phthalazin-1-amine trihydrochloride;
7-methoxy-4-(4-methoxyphenyl)-N-(1-{4-[2-(1-methylpyrrolidin-2-yl)ethoxy]-benzyl}piperidin-4-yl)phthalazin-1-amine trihydrochloride;
4-ethyl-N-[1-(4-ethynylbenzyl)piperidin-4-yl]-7-methoxyphthalazin-1-amine dihydrochloride;
N-(1-{4-[3-(dimethylamino)prop-1-yn-1-yl]benzyl}piperidin-4-yl)-4-ethyl-7-meth-oxyphthalazin-1-amine trihydrochloride;
3-{4-[(4-{[7-methoxy-4-(4-methoxyphenyl)phthalazin-1-yl]amino}piperidin-1-yl)-methyl]phenoxy}propan-1-ol dihydrochloride;
4-ethyl-7-methoxy-N-{1-[4-(4-pyrrolidin-1-ylpiperidin-1-yl)benzyl]piperidin-4-yl}-phthalazin-1-amine trihydrochloride;
N-(1-{4-[3-(dimethylamino)propoxy]benzyl}piperidin-4-yl)-7-methoxy-4-(methoxy-methyl)phthalazin-1-amine trihydrochloride;
N-(1-{4-[(3S)-3-(dimethylamino)pyrrolidin-1-yl]benzyl}piperidin-4-yl)-7-methoxy-4-(methoxymethyl)phthalazin-1-amine trihydrochloride;
N-(1-{4-[(3R)-3-(dimethylamino)pyrrolidin-1-yl]benzyl}piperidin-4-yl)-7-methoxy-4-(methoxymethyl)phthalazin-1-amine trihydrochloride;
N-[1-(1,3-benzodioxol-5-ylmethyl)piperidin-4-yl]-7-methoxy-4-[4-(2-methyl-2H-tetrazol-5-yl)phenyl]phthalazin-1-amine dihydrochloride;
7-methoxy-4-(methoxymethyl)-N-(1-{4-[(1-methylpyrrolidin-3-yl)methoxy]benzyl}-piperidin-4-yl)phthalazin-1-amine trihydrochloride;
7-methoxy-4-(methoxymethyl)-N-(1-{4-[(8-methyl-8-azabicyclo[3.2.1]oct-3-yl)oxy]-benzyl}piperidin-4-yl)phthalazin-1-amine trihydrochloride;
2-[{4-[(4-{[7-methoxy-4-(methoxymethyl)phthalazin-1-yl]amino}piperidin-1-yl)-methyl]phenyl}(methyl)amino]ethanol trihydrochloride;
7-methoxy-4-(methoxymethyl)-N-{1-[4-(2-oxo-2-pyrrolidin-1-ylethoxy)benzyl]-piperidin-4-yl}phthalazin-1-amine dihydrochloride;
3-{4-[(4-{[7-methoxy-4-(methoxymethyl)phthalazin-1-yl]amino}piperidin-1-yl)-methyl]phenoxy}propan-1-ol dihydrochloride;
7-methoxy-4-(methoxymethyl)-N-(1-{4-[(1-methylazepan-4-yl)oxy]benzyl}piperidin-4-yl)phthalazin-1-amine trihydrochloride;
7-methoxy-4-(methoxymethyl)-N-(1-{4-[2-(1-methylpiperidin-2-yl)ethoxy]benzyl}-piperidin-4-yl)phthalazin-1-amine trihydrochloride;
2-{4-[(4-{[7-methoxy-4-(methoxymethyl)phthalazin-1-yl]amino}piperidin-1-yl)-methyl]phenoxy}ethanol dihydrochloride;
7-methoxy-4-methyl-N-(1-{4-[(1-methylazepan-4-yl)oxy]benzyl}piperidin-4-yl)-phthalazin-1-amine trihydrochloride;
3-{3-[(4-{[7-methoxy-4-(methoxymethyl)phthalazin-1-yl]amino}piperidin-1-yl)-methyl]phenoxy}propan-1-ol dihydrochloride;
3-[4-({4-[(7-methoxy-4-methylphthalazin-1-yl)amino]piperidin-1-yl}methyl)phenoxy]propan-1-ol dihydrochloride;
7-chloro-4-methyl-N-(1-{4-[(1-methylazepan-4-yl)oxy]benzyl}piperidin-4-yl)-phthalazin-1-amine trihydrochloride;
3-[4-({4-[(7-chloro-4-methylphthalazin-1-yl)amino]piperidin-1-yl}methyl)phenoxy]-propan-1-ol dihydrochloride;
(3S)-1-{4-[(4-{[7-methoxy-4-(methoxymethyl)phthalazin-1-yl]amino}piperidin-1-yl)methyl]phenyl}pyrrolidin-3-ol trihydrochloride;
(3R)-1-{4-[(4-{[7-methoxy-4-(methoxymethyl)phthalazin-1-yl]amino}piperidin-1-yl)methyl]phenyl}pyrrolidin-3-ol trihydrochloride;
7-methoxy-4-(methoxymethyl)-N-{1-[4-(4-pyrrolidin-1-ylbutyl)benzyl]piperidin-4-yl}phthalazin-1-amine trioxalate;
2-{3-[(4-{[7-methoxy-4-(4-methoxyphenyl)phthalazin-1-yl]amino}piperidin-1-yl)-methyl]phenoxy}ethanol dihydrochloride;
2-{3-[(4-{[7-methoxy-4-(methoxymethyl)phthalazin-1-yl]amino}piperidin-1-yl)-methyl]phenoxy}ethanol dihydrochloride;
3-{3-[(4-{[7-methoxy-4-(4-methoxyphenyl)phthalazin-1-yl]amino}piperidin-1-yl)-methyl]phenoxy}propan-1-ol dihydrochloride;
3-{4-[(4-{[7-methoxy-4-(trifluoromethyl)phthalazin-1-yl]amino}piperidin-1-yl)-methyl]phenoxy}propan-1-ol dihydrochloride;
7-methoxy-4-(methoxymethyl)-N-{1-[4-(octahydro-2H-pyrido[1,2-a]pyrazin-2-yl)-benzyl]piperidin-4-yl}phthalazin-1-amine trihydrochloride;
3-[4-({4-[(7-methoxyphthalazin-1-yl)amino]piperidin-1-yl}methyl)phenoxy]propan-1-ol dihydrochloride;
7-methoxy-4-(methoxymethyl)-N-{1-[4-(6-methyloctahydro-1H-pyrrolo[3,4-b]-pyridin-1-yl)benzyl]piperidin-4-yl}phthalazin-1-amine trihydrochloride;
7-methoxy-4-(4-methoxyphenyl)-N-{1-[4-(7-methyl-2,7-diazaspiro[4.4]non-2-yl)-benzyl]piperidin-4-yl}phthalazin-1-amine trihydrochloride;
4-{4-[(4-{[7-methoxy-4-(methoxymethyl)phthalazin-1-yl]amino}piperidin-1-yl)-methyl]phenyl}butan-1-ol dihydrochloride;
7-methoxy-4-(methoxymethyl)-N-[1-({1-[4-(trifluoromethyl)phenyl]-1H-pyrrol-3-yl}-methyl)piperidin-4-yl]phthalazin-1-amine dihydrochloride;
N-(1-{4-[2-(1H-imidazol-1-yl)ethoxy]benzyl}piperidin-4-yl)-7-methoxy-4-(4-meth-oxyphenyl)phthalazin-1-amine trihydrochloride;
7-methoxy-4-(4-methoxyphenyl)-N-{1-[4-(pyridin-4-ylmethoxy)benzyl]piperidin-4-yl}phthalazin-1-amine trihydrochloride;
7-methoxy-4-(4-methoxyphenyl)-N-{1-[4-(tetrahydrofuran-2-ylmethoxy)benzyl]-piperidin-4-yl}phthalazin-1-amine dihydrochloride.

9. A process for preparing a compound of formula (I) according to claim 1, comprising:
reacting a compound of formula (III): 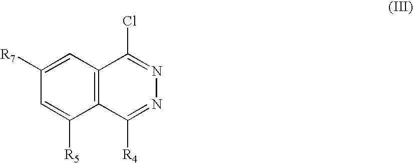
in which R₄, R₅ and R₇ are as defined in claim 1, with a compound of formula (II): 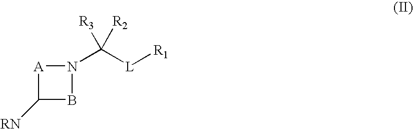
in which R, R₁, R₂, R₃, L, A and B are as defined in claim 1.

10. A pharmaceutical composition comprising at least one compound of formula (I) according to claim 1 or a pharmaceutically acceptable salt, a hydrate or a solvate thereof, in combination with at least one pharmaceutically acceptable excipient.

11. A pharmaceutical composition comprising at least one compound of formula (I) according to claim 2 or a pharmaceutically acceptable salt, a hydrate or a solvate thereof, in combination with at least one pharmaceutically acceptable excipient.

12. A pharmaceutical composition comprising at least one compound of formula (I) according to claim 3 or a pharmaceutically acceptable salt, a hydrate or a solvate thereof, in combination with at least one pharmaceutically acceptable excipient.

13. A pharmaceutical composition comprising at least one compound of formula (I) according to claim 4 or a pharmaceutically acceptable salt, a hydrate or a solvate thereof, in combination with at least one pharmaceutically acceptable excipient.

14. A pharmaceutical composition comprising at least one compound of formula (I) according to claim 5 or a pharmaceutically acceptable salt, a hydrate or a solvate thereof, in combination with at least one pharmaceutically acceptable excipient.

15. A pharmaceutical composition comprising at least one compound of formula (I) according to claim 6 or a pharmaceutically acceptable salt, a hydrate or a solvate thereof, in combination with at least one pharmaceutically acceptable excipient.

16. A pharmaceutical composition comprising at least one compound of formula (I) according to claim 7 or a pharmaceutically acceptable salt, a hydrate or a solvate thereof, in combination with at least one pharmaceutically acceptable excipient.

17. A pharmaceutical composition comprising at least one compound of formula (I) according to claim 8 or a pharmaceutically acceptable salt, a hydrate or a solvate thereof, in combination with at least one pharmaceutically acceptable excipient.

18. A method of treating obesity, cellulite, urinary incontinence, diabetes, cardiovascular disorders, syndrome X, anxiety and depression comprising administering to a patient in need of said treatment a therapeutically effective amount of a compound of formula (I) according to claim 1 or a pharmaceutically acceptable salt, a hydrate or a solvate thereof.

19. A method of treating obesity, cellulite, urinary incontinence, diabetes, cardiovascular disorders, syndrome X, anxiety and depression comprising administering to a patient in need of said treatment a therapeutically effective amount of a compound of formula (I) according to claim 2 or a pharmaceutically acceptable salt, a hydrate or a solvate thereof.

20. A method of treating obesity, cellulite, urinary incontinence, diabetes, cardiovascular disorders, syndrome X, anxiety and depression comprising administering to a patient in need of said treatment a therapeutically effective amount of a compound of formula (I) according to claim 3 or a pharmaceutically acceptable salt, a hydrate or a solvate thereof.

21. A method of treating obesity, cellulite, urinary incontinence, diabetes, cardiovascular disorders, syndrome X, anxiety and depression comprising administering to a patient in need of said treatment a therapeutically effective amount of a compound of formula (I) according to claim 4 or a pharmaceutically acceptable salt, a hydrate or a solvate thereof.

22. A method of treating obesity, cellulite, urinary incontinence, diabetes, cardiovascular disorders, syndrome X, anxiety and depression comprising administering to a patient in need of said treatment a therapeutically effective amount of a compound of formula (I) according to claim 5 or a pharmaceutically acceptable salt, a hydrate or a solvate thereof.

23. A method of treating obesity, cellulite, urinary incontinence, diabetes, cardiovascular disorders, syndrome X, anxiety and depression comprising administering to a patient in need of said treatment a therapeutically effective amount of a compound of formula (I) according to claim 6 or a pharmaceutically acceptable salt, a hydrate or a solvate thereof.

24. A method of treating obesity, cellulite, urinary incontinence, diabetes, cardiovascular disorders, syndrome X, anxiety and depression comprising administering to a patient in need of said treatment a therapeutically effective amount of a compound of formula (I) according to claim 7 or a pharmaceutically acceptable salt, a hydrate or a solvate thereof.

25. A method of treating obesity, cellulite, urinary incontinence, diabetes, cardiovascular disorders, syndrome X, anxiety and depression comprising administering to a patient in need of said treatment a therapeutically effective amount of a compound of formula (I) according to claim 8 or a pharmaceutically acceptable salt, a hydrate or a solvate thereof.
